(19) United States
(12) Patent Application Publication
(10) Pub. No.: US 2026/0242800 A1
(43) Pub. Date: Aug. 20, 2026

(54) USE OF PRIME EDITING IN CORRECTING MUTATIONS IN CDKL5

(71) Applicants: The Broad Institute, Inc., Cambridge, MA (US); President and Fellows of Harvard College, Cambridge, MA (US)

(72) Inventors: David R. Liu, Cambridge, MA (US); KeIcee Everette, Cambridge, MA (US)

(73) Assignees: The Broad Institute, Inc., Cambridge, MA (US); President and Fellows of Harvard College, Cambridge, MA (US)

(21) Appl. No.: 19/665,160

(22) Filed: May 1, 2026

Related U.S. Application Data
(63) Continuation of application No. PCT/US2024/054111, filed on Nov. 1, 2024.
(60) Provisional application No. 63/596,037, filed on Nov. 3, 2023.

(51) Int. Cl. C12N 15/113; A61K 31/7105; A61K 48/00; A61P 25/00; C12N 7/04; C12N 9/12; C12N 9/22; C12N 15/10; C12N 15/864

(52) U.S. Cl. CPC C12N 15/1137; A61K 31/7105; A61K 48/005; A61P 25/00; C12N 7/045; C12N 9/1276; C12N 9/226; C12N 15/102; C12N 15/8645; C12Y 207/07049; C12N 2310/20; C12N 2750/14123; C12N 2750/14143

(57) ABSTRACT

The present disclosure provides methods of using a prime editor in combination with prime editing guide RNAs (pegRNAs) and, optionally, nicking gRNAs to carry out prime editing to directly correct mutations (i.e., c.1412delA) in the CDKL5 gene that cause CDKL5 deficiency disorder. The present disclosure also provides compositions, complexes, and systems comprising the pegRNAs, nicking gRNAs, and/or prime editors disclosed herein, as well as polynucleotides and vectors encoding the same, and cells, kits, and pharmaceutical compositions comprising the same.

### RELATED APPLICATIONS

This application claims priority under 35 U.S.C. §§ 120 and 365 (c) to International PCT Application, PCT/US2024/054111, filed on Nov. 1, 2024, which claims priority under 35 U.S.C. § 119 (e) to U.S. Provisional Application, U.S. Ser. No. 63/596,037, filed Nov. 3, 2023, the contents of each of which are incorporated herein by reference.

### GOVERNMENT SUPPORT

This invention was made with government support under grant numbers U01 AI142756, RM1 HG009490, R01 EB022376, and R35 GM118062, awarded by the National Institutes of Health. The government has certain rights in the invention.

### REFERENCE TO AN ELECTRONIC SEQUENCE LISTING

The contents of the electronic sequence listing (B119570194US01-SEQ-GJM.xml; Size: 604,815 bytes; and Date of Creation: Apr. 24, 2026) are herein incorporated by reference in their entirety.

### BACKGROUND OF THE INVENTION

CDKL5 Deficiency Disorder (CDD) is a developmental encephalopathy that is caused by dominant negative mutations in the cyclin-dependent kinase-like 5 (CDKL5) gene, such as the single “A” nucleotide deletion in Asp471 (c.1412delA), as well as other deletions, truncations, splice variants, and missense mutations, which result in pathogenic variants of the CDKL5 protein. See Olson et al., “Cyclin-dependent kinase-like 5 (CDKL5) deficiency disorder: clinical review,” Pediatr Neurol, 2019, 97:18-25. The CDKL5 protein is widely expressed in the brain, predominantly in neurons, with roles in cell proliferation, neuronal migration, axonal outgrowth, dendritic morphogenesis, and synapse development. CDD is a rare disease which occurs in about 1 in 42,000 live births. Symptoms include seizures, intellectual disability, developmental delay, feeding difficulties, sleeping difficulties, and low muscle tone. Typically, symptoms begin within a few months after birth. Treatment for CDD is limited to symptom management. Symptom management options include broad-range anti-seizure medications (ASMs) such as levetiracetam, the only FDA-approved ASM specifically for CDD called ganaxolone, ketogenic diet, and non-FDA approved cannabidiol derivatives^{198,199}.

As a precise and versatile genome editing technology, prime editing (PE) enables all base-to-base changes, insertions, deletions, and any combination thereof in living cells. As such, PE has the potential to correct the majority of disease-causing mutations, including CDD-causing pathogenic mutations, such as the c.1412delA mutation. PE requires a prime editing guide RNA (pegRNA) and a prime editor protein, which comprises a programmable nickase and a reverse transcriptase (RT). In contrast to nucleases, PEs do not require the formation of a double stranded break (DSB) intermediates and thus offer a higher ratio of the desired to undesired editing products (i.e., the intended edit versus unwanted insertions and deletions or indels). However, standard prime editors (e.g., PEmax) are large and, as a result, often require a triple-AAV delivery strategy for in vivo studies. Recently evolved and engineered PE6a, PE6b, PE6c, PE6d prime editors (as reported in Doman et al., “Phage-assisted evolution and protein engineering yield compact, efficient prime editors,” Cell, 2023 Aug. 31; 186 (18): 3983-4002, and International PCT Application No. PCT/US2024/030786, filed May 23, 2024, each of which is incorporated herein by reference), a series of prime editors that are sufficiently compact for dual-AAV delivery, address this issue while retaining or exceeding the high editing efficiency afforded by the previous state-of-the-art prime editor, PEmax. Since CDD is caused by dominant negative mutations in the CDKL5 gene (i.e., mutations which result in a pathogenic variant of CDKL5 protein that interferes with the normal function of wild-type CDKL5), prime editing strategies that can specifically target and correct pathogenic mutations are needed.

### SUMMARY OF THE INVENTION

The present disclosure describes prime editing compositions, systems, and components thereof, constructs encoding the prime editing systems and/or components thereof, delivery vehicles (e.g., AAV delivery vectors or LNP delivery systems) encoding the prime editing systems and/or components thereof, amino acid and nucleotide sequences of the prime editing systems and/or components thereof, cells expressing or containing the prime editing systems and/or components thereof, and methods of gene editing using the prime editing systems and/or components, including ex vivo and in vivo methods of editing. In certain embodiments, the prime editing systems are capable of editing a cyclin-dependent kinase-like 5 (CDKL5) gene in order to correct and/or repair a CDD-associated pathogenic mutation, including, but not limited to, the single “A” nucleotide deletion in Asp471 (i.e., the c.1412delA mutation) known to be causative of CDD, as well as the c.826-1 G-to-A point mutation in CDKL5 and clusters of mutations in exon 8 of CDKL5.

In embodiments, the prime editing systems described herein comprise prime editing guide RNAs (pegRNAs) and prime editors, including pegRNAs and modified prime editors based on PE6, herein referred to as “PE6b-NRCH,” “PE6c-NRCH,” and “PE6d-NRCH”, which are capable of correcting pathogenic mutations in the CDKL5 gene, including, but not limited to, the c.1412delA mutation.

In certain embodiments, the prime editing systems described herein have been shown to achieve over 40% correction of the c.1412delA mutation in a heterozygous mouse N2a cell line with less than 2% indel byproducts.

In one aspect, the present disclosure provides pegRNAs for correcting a pathogenic mutation in the CDKL5 gene, including, but not limited to, the c.1412delA mutation, and therefore treating CDD. In some embodiments, the provided pegRNAs comprise reverse transcription templates (RTTs) that further introduce one or more silent edits into the CDKL5 gene to increase the efficiency at which the mutation is corrected (e.g., by impairing a cell's DNA mismatch repair mechanisms).

In some embodiments, the present disclosure provides pegRNAs for targeting the c.1412delA mutation comprising a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5).

In some embodiments, the pegRNA comprises the sequence of any of the pegRNAs provided in Tables 1, 2, and 4 below, or a sequence at least 80%, at least 85%, at least 90%, at least 95%, at least 96%, at least 97%, at least 98%, or at least 99% identical to any of the pegRNAs provided in Tables 1 and 2 below.

In another aspect, the present disclosure provides compositions comprising any of the pegRNAs provided herein, including pegRNAs comprising the sequence of any of the pegRNAs provided in Tables 1, 2, and 4 below, or a sequence at least 80%, at least 85%, at least 90%, at least 95%, at least 96%, at least 97%, at least 98%, or at least 99% identical to any of the pegRNAs provided in Tables 1, 2, and 4 below.

In some embodiments, the composition further comprises a nicking guide RNA (ngRNA). In some embodiments, the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13).

In certain embodiments, the ngRNA comprises the sequence of any one of the ngRNAs provided in Table 3 below, or a sequence at least 80%, at least 85%, at least 90%, at least 95%, at least 96%, at least 97%, at least 98%, or at least 99% identical to any one of the ngRNAs provided in Table 3 below.

In another embodiment, the present disclosure provides compositions comprising any of the nicking guides (ngRNA) provided in Table 3 or a sequence at least 80%, at least 85%, at least 90%, at least 95%, at least 96%, at least 97%, at least 98%, or at least 99% identical to any of the ngRNAs provided in Table 3 below.

In some embodiments, the composition further comprises a prime editor. In certain embodiments, the prime editor is PE4max-NRCH, or a variant thereof. In some embodiments, the prime editor is a PE6 prime editor, or a variant thereof. In certain embodiments, the prime editor is PE6b-NRCH, PE6c-NRCH, PE6d-NRCH, or a variant thereof.

In another aspect, the present disclosure provides systems comprising a pegRNA, prime editor, and ngRNA described herein.

In another aspect, the present disclosure provides one or more polynucleotides encoding any of the pegRNAs and/or ngRNAs provided herein. In some embodiments, the one or more polynucleotides encode any of the prime editors provided herein.

In another aspect, the present disclosure provides one or more vectors comprising the one or more polynucleotides provided herein. In some embodiments, the vectors are AAV vectors.

In another aspect, the present disclosure provides virus-like particles (VLPs) or engineered virus-like particles (eVLPs) comprising any of the pegRNAs, ngRNAs, and/or prime editors disclosed herein.

In another aspect, the present disclosure provides compositions comprising any of the polynucleotides and/or vectors provided herein.

In another aspect, the present disclosure provides one or more AAV particles comprising any of the pegRNAs, compositions, polynucleotides, and/or vectors provided herein.

In another aspect, the present disclosure provides virus-like particles (VLPs) comprising any of the pegRNAs, compositions, polynucleotides and/or vectors provided herein.

In another aspect, the present disclosure provides pharmaceutical compositions comprising any of the pegRNAs, compositions, polynucleotides, vectors, AAV particles, or VLPs provided herein, and a pharmaceutically acceptable excipient.

In another aspect, the present disclosure provides cells comprising any of the pegRNAs, compositions, polynucleotides, vectors, AAV particles, or VLPs provided herein.

In another aspect, the present disclosure provides kits comprising any of the pegRNAs, compositions, polynucleotides, vectors, AAV particles, or VLPs provided herein.

In another aspect, the present disclosure provides methods of using a prime editor in combination with a pegRNA (and optionally an ngRNA) to carry out prime editing to directly correct mutations in the CDKL5 gene that cause CDKL5 deficiency disorder. In one aspect, the present disclosure provides methods of correcting a 1412delA mutation in a CDKL5 gene by prime editing comprising contacting a target DNA sequence comprising the CDKL5 gene with a prime editor and a prime editing guide RNA (pegRNA), wherein the pegRNA comprises a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), or GCAGAGTCGGCATAGCTATAT (SEQ ID NO: 5).

In some embodiments, the contacting further comprises using a nicking guide RNA (ngRNA). In some embodiments, the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13).

In certain embodiments, the prime editor is PE4max-NRCH, or a variant thereof. In some embodiments, the prime editor is a PE6 prime editor, or a variant thereof. In certain embodiments, the prime editor is PE6b-NRCH, PE6c-NRCH, PE6d-NRCH, or a variant thereof.

In some embodiments, the method is performed in a subject. In some embodiments, the method is a method of treating CDKL5 deficiency disorder in the subject.

In another aspect, the present disclosure provides for the use of any of the pegRNAs, compositions, polynucleotides, vectors, AAV particles, VLPs, or pharmaceutical compositions provided herein in the treatment of CDKL5 deficiency disorder.

In another aspect, the present disclosure provides for the use of any of the PE editing systems, prime editors, pegRNAs, ngRNAs, compositions, expression constructs, polynucleotides, vectors, AAV particles, VLPs, or pharmaceutical compositions provided herein in the manufacture of a medicament for the treatment of CDKL5 deficiency disorder.

The foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show further developments of an improved prime editor-NRCH strategy to specifically target the c.1412delA variant allele in CDKL5 and limit potential indel formation in the wildtype allele. A PE4max-NRCH strategy that specifically targets the pathogenic allele was developed. The NRCH PAM is disrupted when the pathogenic deletion is reverted. The strategy also disrupts the protospacer with a seed edit. Additional strategies incorporating PE6 prime editors and variants, nicking guide RNA (ngRNAs), use of a flip+extension (F+E) scaffold, and a shortened PBS were also tested.

FIG. 2 shows screening for the best epegRNA. PE6b-NRCH and PE6c-NRCH perform as well as PE4-max NRCH with nicking guide 1 (NG1) as provided in the first row of Table 3 below.

FIG. 3 shows that the F+E scaffold improved editing with epegRNA primer binding site (PBS) 11 and reverse transcriptase template (RTT) 15 as shown in Tables 1 and 2 below. Shortening the PBS to 9 or 10 nucleotides improved editing even further. PBS10 and RTT15 were used in subsequent screens in N2a cells.

FIG. 4 shows that screening PBS10 and RTT15 in a similar manner as the previous best epegRNA again revealed that PE6b-NRCH and PE6c-NRCH are nearly as active as PE5max-NRCH with NG1.

FIG. 5 shows that the desired edit-to-indel ratio with PE6b-NRCH is better than with PE6c-NRCH.

FIG. 6 shows establishment of homozygous and 2/3 heterozygous HEK293T cell lines used for epegRNA PE-NRCH screening.

FIG. 7 shows screening of epegRNAs with PE4max-NRCH in HEK293T cells.

FIG. 8 shows screening of PE4max-SpRY (recognizing an NRN PAM). Highest editing efficiencies of 2.4% of all alleles and 4.8% of available alleles was observed.

FIG. 9 shows screening of epegRNAs with a PE6 prime editor comprising an evolved Cas9 variant. Highest editing efficiencies of 6.6% of all alleles and 13.2% of available alleles was observed.

FIG. 10 shows screening of epegRNAs with a PE6 prime editor comprising an evolved reverse transcriptase variant. Highest editing efficiencies of 6.2% of all alleles and 12.4% of available alleles was observed.

FIGS. 11A-11D show prime editing optimization for the correction of c.1412delA in murine Cdkl5. Bars reflect mean±s.d. of n=2-4 independent biological replicates from different thaws of a monoclonal heterozygous N2a cell line harboring the Cdkl5 equivalent of c.1412delA in 50% of alleles, with replicate values shown as individual dots. Editing percentages represent desired prime editing outcomes with no indels in the 50% of targetable alleles with the pathogenic mutation. Indel frequencies reflect indel formation across all sequencing reads. FIG. 11A shows that PE-NRCH can revert the c.1412delA mutation (underlined) back to the wildtype allele. The pathogenic allele makes an NRCH PAM which allows for allele-specific targeting in addition to eliminating potential re-targeting of the corrected allele as the PAM is disrupted when the allele is corrected. The addition of a silent seed edit (+2 G>T, double underline) may also help the prime editing intermediate evade MMR86. FIG. 11B shows editing efficiencies for corrective epegRNAs of various PBS and RTT lengths in a PE4max-NRCH screen. FIG. 11C shows editing efficiencies of epegRNAs with PBS lengths of 9-11 nt and an RTT length of 15 nt with either the standard sgRNA scaffold or the “flip and extension” (F+E) scaffold203. FIG. 11D shows editing efficiencies for various PE systems when using an epegRNA with a standard scaffold, a PBS of 10 nt, and an RTT of 15 nt, and with the optional inclusion of a nicking sgRNA that nicks at 87 nt (NG1), 63 nt (NG2), or 47 nt (NG3) 3′ of the epegRNA nick site.

FIGS. 12A-12E show optimizing prime editing outcomes for c.1412delA in human CDKL5. Bars reflect mean±s.d. of n=2-3 independent biological replicates from monoclonal homozygous HEK293T cell lines harboring the c.1412delA in CDKL5, with replicate values shown as individual dots. FIG. 12A shows that initial efforts to optimize an SpCas9-NRCH-based prime editing strategy for the correction of the pathogenic c.1412delA mutation (underline) required the inclusion of a non-silent seed edit. A corrective prime editing strategy was previously described with PEmax and a pegRNA which includes a silent seed edit (double underline) 86. FIG. 12B shows editing efficiencies for an epegRNA screen for the PE-NRCH strategy. All epegRNAs encoded a non-silent I470L mutation. FIG. 12C shows editing efficiencies from a PE-NRCH variant screen utilizing the most efficient epegRNA from FIG. 12B. Each prime editor variant was screened with either no nicking sgRNA, a nicking sgRNA designed not to nick until after the desired edit has already been made in attempt to minimize indels3 (NG4), or a nick-to-nick distance of 22 nt (NG5) or 76 nt (NG6). FIG. 12D shows editing outcomes after screening PE variants with the epegRNA version of the corrective pegRNA86 either with or without an 8 nt linker designed to minimize interactions between the structured 3′ motif of the epegRNA and the RTT89. All conditions utilized the nicking sgRNA86.

FIG. 13 shows prime editing of patient primary fibroblasts. Quantification of editing frequencies from PE3max, PE6c, and untreated patient primary fibroblasts heterozygous for c.1412delA. Bars represent average desired editing frequency of the 50% of editable alleles with the c.1412delA mutation. Dots represent n=2 independent replicates and error bars represent±s.d. Indel frequencies represent indels observed from all reads. 2.5×10⁵ fibroblasts isolated from a patient heterozygous for the c.1412delA mutation were electroporated and cultured for 6 days. At day 3, the cells underwent media exchange, and genomic DNA was extracted at day 6.

FIGS. 14A-14B show off-target editing in prime-edited primary patient fibroblasts. Bars represent average±s.d. of n=2 independent biological replicates. FIG. 14A shows HTS quantification of the first epegRNA-encoded mismatch at Cas-OFFinder-nominated off-target loci in patient primary fibroblasts heterozygous for the c.1412delA mutation. FIG. 14B shows HTS quantification of indels at epegRNA- and nicking sgRNA-dependent off-target loci nominated by Cas-OFFinder in patient primary fibroblasts heterozygous for the c.1412delA mutation. Nick OT3 was not analyzed due to its location on the Y chromosome and the fibroblasts being isolated from a female patient.

FIGS. 15A-15C shows screening of prime editing strategies for the correction of c.826-1 G>A. Development of a corrective prime editing strategy for c.826-1 G>A. For bar graphs, bars reflect mean±s.d. of n=2 independent biological replicates from heterozygous HEK293T cell lines harboring the c.826-1G>A mutation in approximately 30% of CDKL5 alleles, with replicate values shown as individual dots. FIG. 15A shows design of the corrective prime editing strategy for c.826-1 G>A (underline). The epegRNA (protospacer in pink, PAM in light blue) was designed to also encode silent edits (double underline) which may help the prime editing intermediate evade MMR86. FIG. 15B shows editing efficiencies from an epegRNA screen to determine the epegRNAs resulting in the highest percentage of desired c.826-1 G>A correction. FIG. 15C shows a screen for various prime editors and for multiple nicking sgRNAs, including an edit-specific nicking sgRNA (NG3) as this leads to fewer indels, using the epegRNA with a PBS length of 11 nt and an RTT length of 33 nt from FIG. 15B.

FIGS. 16A-16B shows installation of pathogenic CDD mutations in exon 8 of the CDKL5 catalytic domain. FIG. 16A shows a location of various pathogenic CDKL5 mutations in exon 8 and related seed edits that could be introduced with a single epegRNA. FIG. 16B shows editing efficiencies of the installation of pathogenic CDKL5 mutations or related seed edits in exon 8 from two epegRNAs that have not been completely optimized. Bars reflect mean±s.d. of n=3 independent biological replicates from wildtype HEK293T cell lines, with replicate values shown as individual dots.

FIG. 17 shows CDKL5 function is important for brain function and development.

FIG. 18 shows that pathogenic mutations causing CDKL5 deficiency disorder (CDD) are typically spontaneous and result in loss of CDKL5 function.

FIGS. 19A-19B show that pathogenic mutations causing CDKL5 deficiency disorder (CDD) are typically spontaneous and result in loss of CDKL5 function.

FIGS. 20A-20C show that CDD is clinically distinct from other neurological disorders like Rett syndrome, West syndrome, and X-linked dominant infantile spasm syndrome. Clinical features highly suggestive of CDD include: Seizures within the first year of life (90% by 3 months); Global developmental delay; Severely impaired gross motor function; Sleep disturbances; Abnormal muscle tone; Bruxism (grinding or gnashing of teeth); and Gastrointestinal issues.

FIG. 21 shows that treatments for CDD are limited to symptom management. None of the therapies shown are disease-modifying, nor do they address the root genetic cause of CDD.

FIGS. 22A-22B show that treatments for CDD are limited to symptom management. With gene therapy, endogenous transcriptional control is lost, consequences of overexpression of CDKL5 are unknown, patients with dominant negative mutations would be ineligible, and the genetic cause of CDD would still not be addressed.

FIGS. 23A-23C show that genome editing could enable disease-modifying therapeutic development for CDD by directly correcting pathogenic mutations. Given their versatility, prime editors are uniquely suited to address pathogenic CDKL5 mutations.

FIGS. 24A-24B show efficiency of PE3 strategies for installation of c.1412delA in mCDKL5.

FIGS. 25A-25B show that use of a PBS+RTT length combination that showed initial success in hCDKL5 did not translate to efficient editing in mCDKL5.

FIGS. 26A-26B show that development of epegRNAs and MLH1dn benefited c.1412delA installation in mCDKL5.

FIG. 27 shows that PE3max and epegRNAs with a silent seed edit can enable c.1412delA installation at mCDKL5 in a knock-in HEK293T cell line.

FIG. 28 shows that allele-specific targeting with an SpCas9-NRCH DNA targeting domain may reduce indels.

FIG. 29 shows that PE-NRCH can target the c.1412delA allele specifically, reducing indel formation in a knock-in HEK293T cell line.

FIGS. 30A-30B show that female MEFs harboring c.1412delA could not be efficiently electroporated with either prime editing strategy.

FIG. 31 shows PE3max-NRCH editing in c.1412delA mice.

FIG. 32 shows a protocol for PE3max-NRCH editing in c.1412delA mice.

FIGS. 33A-33D show expression of full-length CDKL5 in male mice 4 weeks post-injection. Western blot analysis of forebrain CDKL5 expression in male Cdk15-FS and WT littermate mice, 4 weeks post-administration of a single dose. Mice were injected at PO days with either AAV9-epegRNA+nicking guide (GFP), AAV9-PE3max-NRCH (TX), or saline (SAL).

FIG. 34 shows behavioral responses of treated and untreated female mice 8 weeks post-injection. FWT, female WT; FFS, female FS.

FIG. 35 shows allele-specific PE3max-NRCH editing in a triple AAV system.

FIG. 36 shows that efficient prime editing for the generation of an N2a cell line with c.1412delA in 50% of alleles enabled extensive screening.

FIGS. 37A-37D show screening of epegRNAs with the indicated prime editor.

FIG. 38 shows that PE3max and PE6c enable 25-27% c.1412delA correction in primary patient fibroblasts.

FIGS. 39A-39B show that off-target editing is minimal in prime-edited primary patient fibroblasts. FIG. 39A shows Cas-OFFinder pegRNA-nominated off-targets. FIG. 39B shows Cas-OFFinder nicking sgRNA-nominated off-targets. Both preliminary “off-targets” are in intragenic regions.

FIGS. 40A-40C show that precise editing is required for c.826-1 G>A correction to avoid changes to bystander adenines.

FIG. 41 shows that missense pathogenic variants cluster in the catalytic domain.

FIG. 42 shows screening of optiprime epegRNAs with PEmax-NRCH.

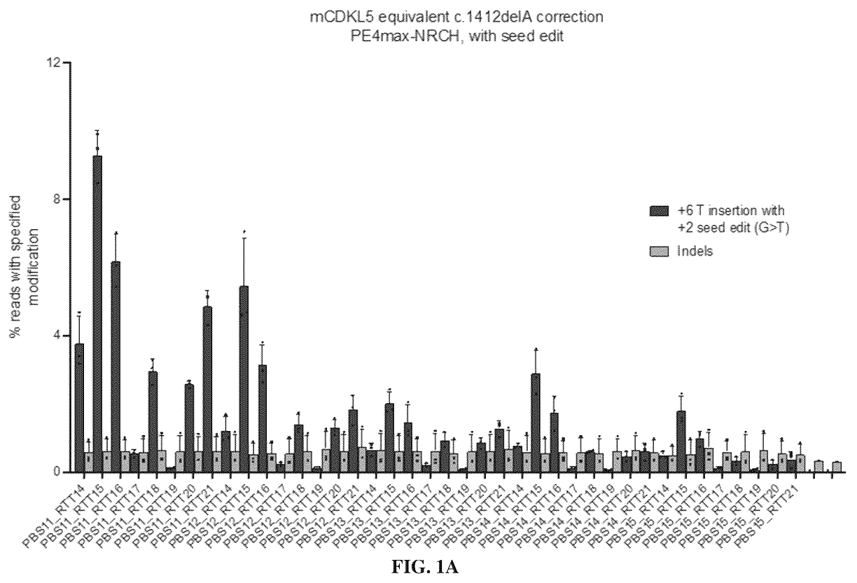

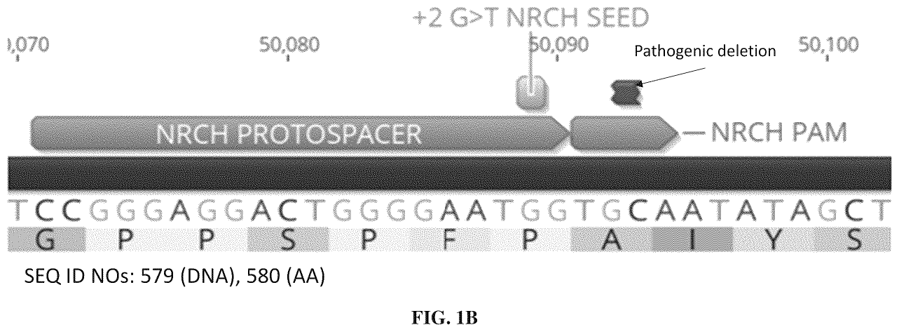

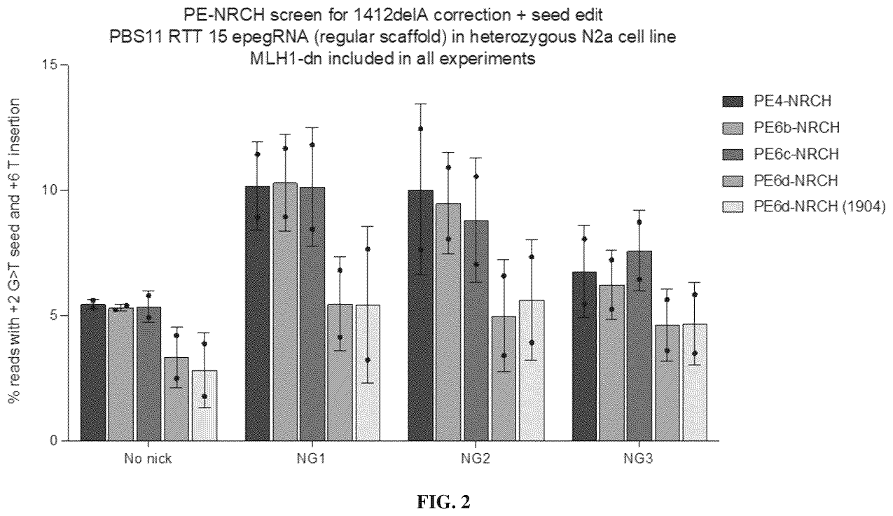

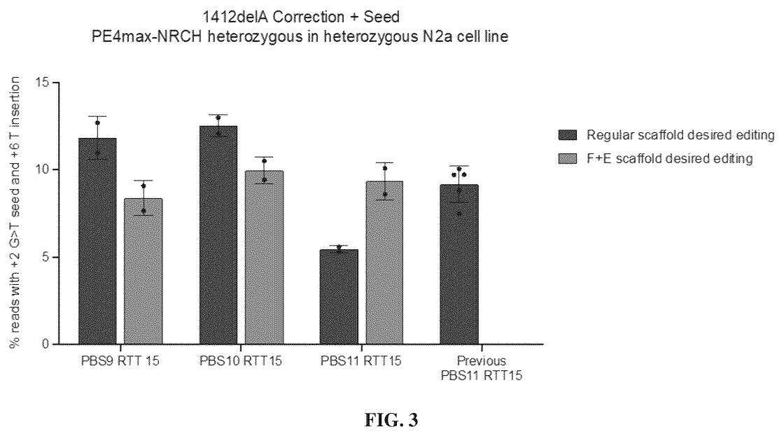

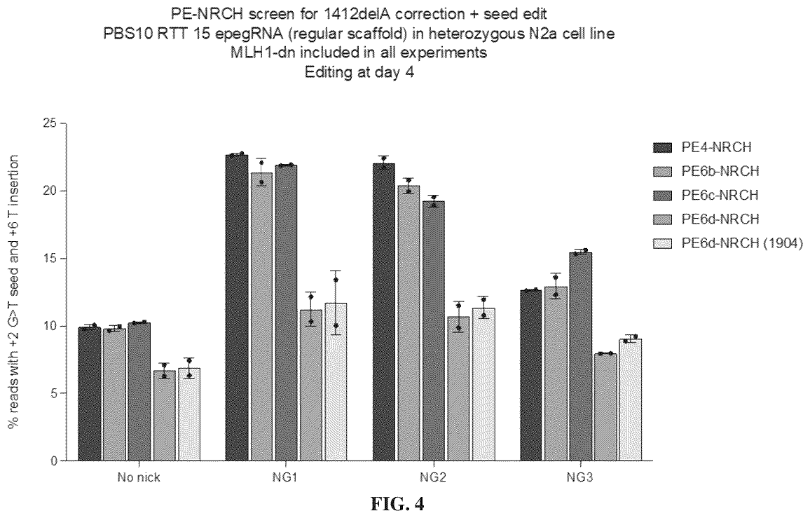

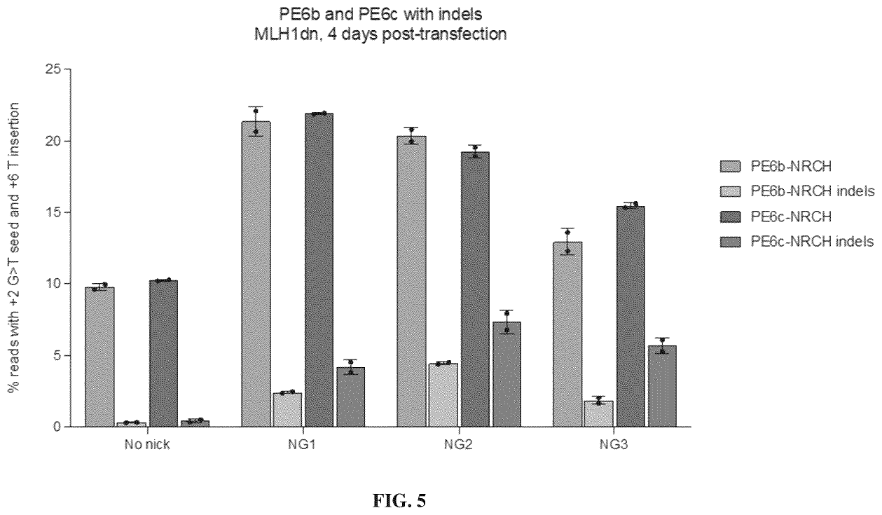

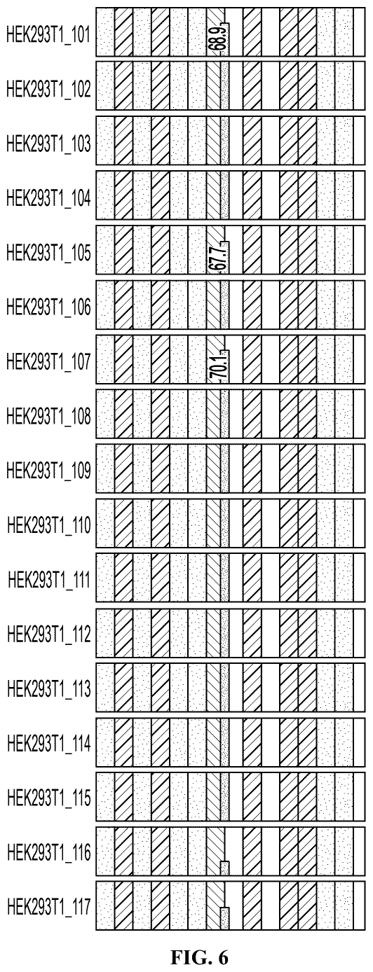

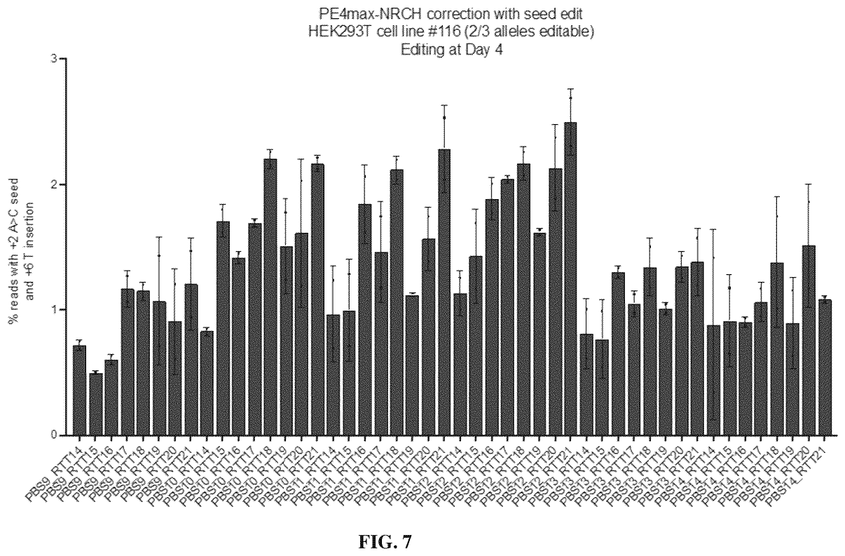

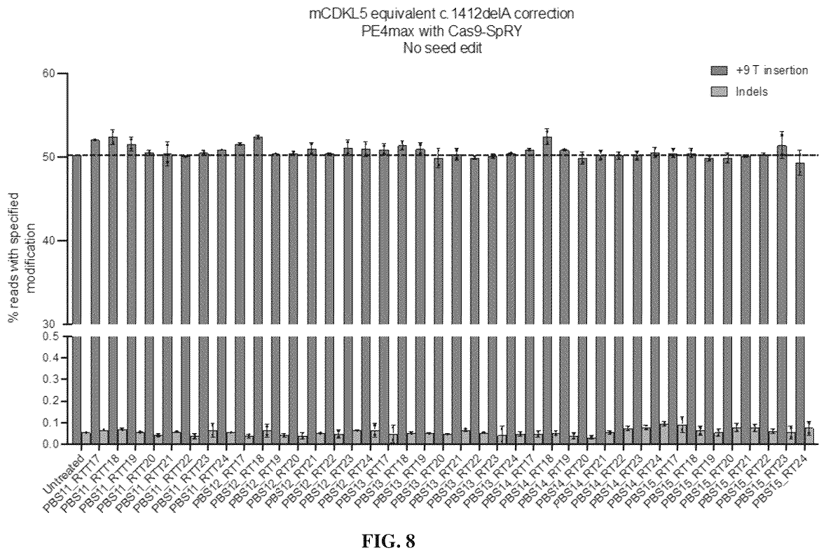

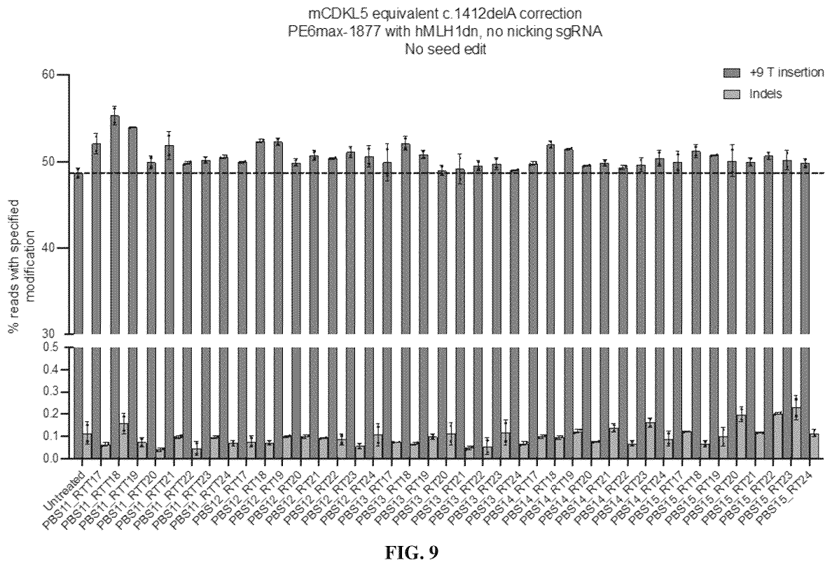

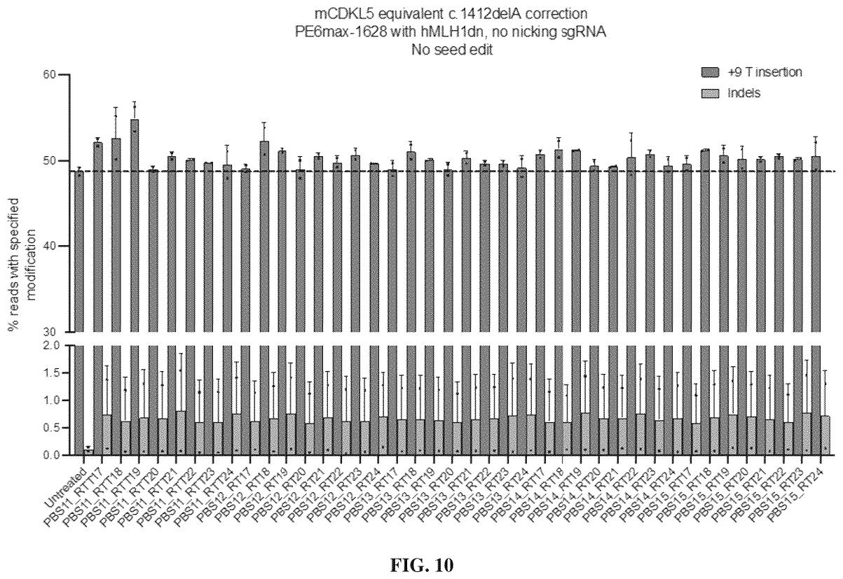

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or fully or partly inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The strand in the target DNA not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J.A., Charpentier E. Science 337:816-821 (2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J.A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28; 152 (5): 1173-83 (2013)). In some embodiments, proteins comprising fragments of a Cas9 protein are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9.

In some embodiments, a Cas9 protein used in a prime editor is Cas9-NRCH.

### CDKL5 Deficiency Disorder (CDD)

“CDKL5 deficiency disorder (CDD)” is a rare genetic disorder caused by pathogenic variants in the CDKL5 gene (e.g., c.1412delA as described herein), which encodes for cyclin-dependent kinase-like 5. Symptoms of CDD include epilepsy, hypotonia, developmental, intellectual, and motor disabilities, and cortical visual impairment. CDD occurs in approximately 1 in 42,000 live births, and each case of CDD is caused by a variety of mutations in the CDKL5 gene.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. Exemplary sequences are and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “pegRNAs”) which have been invented for the prime editing methods and composition disclosed herein.

Guide RNAs or pegRNAs may comprise various structural elements that include, but are not limited to:

Spacer sequence—the sequence in the guide RNA or pegRNA (having about 20 nts in length) which binds to the protospacer in the target DNA.

gRNA core (or gRNA scaffold or backbone sequence)—refers to the sequence within the gRNA that is responsible for Cas9 binding, it does not include the 20 bp spacer/targeting sequence that is used to guide Cas9 to target DNA.

Extension arm—a single strand extension at the 3′ end or the 5′ end of the pegRNA which comprises a primer binding site and a DNA synthesis template sequence that encodes via a polymerase (e.g., a reverse transcriptase) a single stranded DNA flap containing the genetic change of interest, which then integrates into the endogenous DNA by replacing the corresponding endogenous strand, thereby installing the desired genetic change.

Transcription terminator—the guide RNA or pegRNA may comprise a transcriptional termination sequence at the 3′ of the molecule.

napDNAbp

As used herein, the term “nucleic acid programmable DNA binding protein” or “napDNAbp,” of which Cas9 is an example, refers to a protein that uses RNA: DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein. In some embodiments, a napDNAbp has nickase activity in a RuvC domain and/or an HNH domain. In some embodiments, a napDNAbp has nickase activity in a RuvC domain or an HNH domain.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical SpCas9 sequence or to an equivalent amino acid position in other Cas9 variants or Cas9 equivalents. In some embodiments, the nickase is a Cas9 that comprises an H840A, N854A, and/or N863A mutation relative to a canonical SpCas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CaΦ), and Argonaute and optionally has a nickase activity such that one DNA strand is cut preferentially to the other DNA strand.

Nicking Guide RNA (ngRNA)

In some embodiments, a guide RNA is a “nicking guide RNA.” Nicking guide RNAs may be used to nick the non-edited strand of a target nucleic acid molecule, which may facilitate incorporation of the edit by cellular DNA repair mechanisms.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO 2001/038547 on May 31, 2001, the contents of which are incorporated herein by reference for its disclosure of exemplary nuclear localization sequences. In some embodiments, an NLS is included in a fusion protein (e.g., in a prime editor as described herein). In certain embodiments, an NLS comprises the amino acid sequence PKKKRKV (SEQ ID NO: 21), MDSLLMNRRKFLYQFKNVRWAKGRRETYLC (SEQ ID NO: 22), KRTADGSEFESPKKKRKV (SEQ ID NO: 23), KRTADGSEFEPKKKRKV (SEQ ID NO: 24), NLSKRPAAIKKAGQAKKKK (SEQ ID NO: 25), PAAKRVKLD (SEQ ID NO: 26), RQRRNELKRSF (SEQ ID NO: 27), or NQSSNFGPMKGGNFGGRSSGPYGGGGQYFAKPRNQGGY (SEQ ID NO: 28).

pegRNA

As used herein, the terms “prime editing guide RNA,” “PEgRNA,” “pegRNA,” or “extended guide RNA” refer to a specialized form of a guide RNA that has been modified to include one or more additional sequences for implementing the prime editing as described herein. As described herein, the prime editing guide RNAs comprise one or more “extended regions,” also referred to herein as “extension arms,” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” or “reverse transcriptase template” that encodes (by the polymerase/reverse transcriptase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “linker” sequence, or other structural elements, such as, but not limited to, aptamers, stem loops, hairpins, toe-loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the pegRNAs have a 3′ extension arm, a spacer, and a gRNA core. The 3′ extension arm further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In certain other embodiments, the pegRNAs have a 5′ extension arm, a spacer, and a gRNA core. The 5′ extension further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In still other embodiments, the pegRNAs have in the 5′ to 3′ direction a spacer, a gRNA core, and an extension arm. The extension arm is at the 3′ end of the pegRNA. The extension arm further comprises in the 5′ to 3′ direction a homology arm, an edit template, and a primer binding site. The extension arm may also comprise an optional modifier region at the 3′ and 5′ ends, which may be the same sequences or different sequences. In addition, the 3′ end of the pegRNA may comprise a transcriptional terminator sequence. These sequence elements of the pegRNAs are further described and defined herein.

In still other embodiments, the pegRNAs have in the 5′ to 3′ direction an extension arm, a spacer, and a gRNA core. The extension arm is at the 5′ end of the pegRNA. The extension arm further comprises in the 3′ to 5′ direction a primer binding site, an edit template, and a homology arm. The extension arm may also comprise an optional modifier region at the 3′ and 5′ ends, which may be the same sequences or different sequences. The pegRNAs may also comprise a transcriptional terminator sequence at the 3′ end. These sequence elements of the pegRNAs are further described and defined herein.

In some embodiments, the spacer sequence of the pegRNA is about 10, about 11, about 12, about 13, about 14, about 15, about 16, about 17, about 18, about 19, about 20, about 21, about 22, about 23, about 24, or about 25 nucleotides in length. In certain embodiments, the spacer sequence of the pegRNA is about 20 nucleotides in length. In some embodiments, the primer binding site is about 4, about 5, about 6, about 7, about 8, about 9, about 10, about 11, about 12, about 13, about 14, about 15, about 16, or about 17 nucleotides in length. In certain embodiments, the primer binding site is about 9, about 10, about 11, about 12, about 13, about 14, or about 15 nucleotides in length. In some embodiments, the homology arm of the pegRNA is about 5, about 6, about 7, about 8, about 9, about 10, about 11, about 12, about 13, about 14, about 15, about 16, about 17, about 18, about 19, or about 20 nucleotides in length. In some embodiments, the DNA synthesis template is from about 14 to about 21 nucleotides in length.

In some embodiments, a pegRNA is an “engineered pegRNA” (“epegRNA”). Relative to a pegRNA, an epegRNA comprises an additional structured motif, for example, attached to its 3′ end. Such additional structured motifs may stabilize the pegRNA or otherwise prevent it from being degraded. Suitable structured motifs include, but are not limited to, toe-loops, hairpins, stem-loops, pseudoknots, aptamers, G-quadruplexes, tRNAs, riboswitches, and ribozymes. In some embodiments, a 3′ structured motif comprises evopreq1. In some embodiments, the 3′ structured motif in the pegRNAs disclosed herein comprises the sequence CGCGGTTCTATCTAGTTACGCGTTAAACCAACTAGAA (SEQ ID NO: 14), or a sequence at least 80%, at least 85%, at least 90%, at least 95%, at least 96%, at least 97%, at least 98%, or at least 99% identical to the sequence CGCGGTTCTATCTAGTTACGCGTTAAACCAACTAGAA (SEQ ID NO: 14).

pegRNAs are further described, e.g., in International Patent Application No. PCT/US2020/023721, filed Mar. 19, 2020, which published as WO 2020/191239; International Patent Application No. PCT/US2021/031439, filed May 7, 2021, which published as WO 2021/226558; International Patent Application No. PCT/2021/052097, filed Sep. 24, 2021, which published as WO 2022/067130; International Patent Application No. PCT/US2022/012054, filed Jan. 11, 2022, which published as WO 2022/150790; International Patent Application No. PCT/US2022/078655, filed Oct. 25, 2022, which published as WO 2023/076898; and International Patent Application No. PCT/US2022/074628, filed Aug. 5, 2022, which published as WO 2023/015309; the contents of each of which is incorporated by reference herein.

### Prime Editing

As used herein, the term “prime editing” refers to an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference. See also International PCT Application, PCT/US2020/023721, filed Mar. 19, 2020, and published as WO 2020/191239, which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered DNA synthesis template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., pegRNA) containing a spacer sequence that anneals to a complementary sequence (the complementary sequence to an endogenous protospacer sequence) in the target DNA. The pegRNA also contains new genetic information in the form of an extension that encodes a replacement strand of DNA containing a desired nucleotide change which is used to replace a corresponding endogenous DNA strand at the target site. To transfer information from the pegRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on pegRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (pegRNA). In various embodiments, the prime editing guide RNA (pegRNA) comprises an extension at the 3′ or 5′ end of the guide RNA, or at an intramolecular location in the guide RNA, and encodes the desired nucleotide change (e.g., single nucleotide substitution, insertion, or deletion). First, the napDNAbp/extended gRNA complex contacts the DNA molecule, and the extended gRNA guides the napDNAbp to bind to a target locus. Next, a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In the next step, the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the pegRNA. In the next step, a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In the next step, the napDNAbp and guide RNA are released. The final two steps relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

### Prime Editor

As used herein, the term “prime editor” in some embodiments refers to the polypeptide or polypeptide components (e.g., a nucleic acid programmable DNA binding protein (“napDNAbp”) working in association with a polymerase, such as a reverse transcriptase, which are in the form of a fusion protein or otherwise provided in trans) involved in prime editing as described herein, including PE1, PE2, PE3 (including PE3b), PE4, PE5 (including PE5b), PE6 (including PE6a, PE6b, PE6c, PE6d, PE6e, PE6f, PE6a-e, PE6a-f, PE6a-g, PE6b-e, PE6b-f, PE6b-g, PE6c-e, PE6c-f, PE6c-g, PE6d-e, PE6d-f, PE6d-g, PE6b-NRCH, PE6c-NRCH, and PE6d-NRCH), PE7, and PEmax, or to one or more nucleotide sequence encoding any of PE1, PE2, PE3 (including PE3b), PE4, PE5 (including PE5b), PE6 (including PE6a, PE6b, PE6c, PE6d, PE6e, PE6f, PE6a-e, PE6a-f, PE6a-g, PE6b-e, PE6b-f, PE6b-g, PE6c-e, PE6c-f, PE6c-g, PE6d-e, PE6d-f, PE6d-g, PE6b-NRCH, PE6c-NRCH, and PE6d-NRCH), PE7, and PEmax). The prime editor is programmed with a prime editing (PE) guide RNA (“pegRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA).

In some embodiments, a prime editor comprises a napDNAbp (e.g., Cas9 nickase, and/or any of the Cas9 variants provided herein) and a reverse transcriptase (e.g., any of the reverse transcriptase variants provided herein). These components may be provided as a fusion protein (or fusion construct) in some embodiments. In other embodiments, these components may be provided in trans.

In some embodiments, a prime editor is capable of carrying out prime editing on a target nucleotide sequence in the presence of a pegRNA (or “extended guide RNA”). In some embodiments, a prime editor comprises a napDNAbp (e.g., Cas9 nickase) and a reverse transcriptase provided in trans, i.e., the napDNAbp and the reverse transcriptase are not fused. The in trans napDNAbp and the reverse transcriptase may be tethered via a non-peptide linkage, e.g., an MS2 RNA-protein binding RNA sequence and a MS2 coat protein fused to either the napDNAbp or the reverse transcriptase, or may be unlinked to each other and simply recruited by the pegRNA.

In some embodiments, a prime editor composition, system, or complex provided herein comprises a fusion protein or a fusion protein complexed with a pegRNA, and/or further complexed with a second-strand nicking sgRNA. In some embodiments, the prime editor system may also refer to the complex comprising a fusion protein (reverse transcriptase fused to a napDNAbp), a pegRNA, and a regular guide RNA capable of directing the second-site nicking step of the non-edited strand as described herein.

### Prime Editor/Editing System

As used here, the expression “prime editor system” (or equivalent term “prime editing system”) refers to a composition or otherwise a set of components needed to conduct prime editing (e.g., in a cell). In embodiments, a prime editing system minimally includes a prime editor (or a nucleotide sequence encoding a prime editor) and a pegRNA (or a nucleotide sequence encoding the pegRNA). In embodiments, the prime editor and the pegRNA form a complex which is capable of binding to a target DNA site that has complementarity to the pegRNA (i.e., the spacer of the pegRNA). In other embodiments, the prime editing system can include a second-strand nicking guide RNA (ngRNA). In still other embodiments, the prime editing system can include a delivery vector (e.g., a lipid nanoparticle (LNP), virus-like particle (VLP) or engineered virus-like particle (eVLP), adeno-associated virus (AAV) vector, or a lentivirus vector (LV). In still other embodiments, the prime editing system can be formulated as a pharmaceutical composition which may include one or more delivery vectors comprising the prime editing system (e.g., protein components, pegRNAs, or nucleotide sequence encoding same, or a mixture of said components) and one or more pharmaceutical excipients. In further embodiments, the prime editing system may also include a device or applicator for administering same.

### Primer Binding Site

The term “primer binding site” or “the PBS” refers to the nucleotide sequence located on a pegRNA as component of the extension arm (typically at the 3′ end of the extension arm) and serves to bind to the primer sequence that is formed after Cas9 nicking of the target sequence by the prime editor. As detailed elsewhere, when the Cas9 nickase component of a prime editor nicks one strand of the target DNA sequence, a 3′-ended ssDNA flap is formed, which serves a primer sequence that anneals to the primer binding site on the pegRNA to prime reverse transcription.

### Protospacer

As used herein, the term “protospacer” refers to the sequence (e.g., of ~20 bp) in DNA adjacent to the PAM (protospacer adjacent motif) sequence. The protospacer shares the same sequence as the spacer sequence of the guide RNA (except that a protospacer contains Thymine and the spacer sequence contains Uracil). The guide RNA anneals to the complement of the protospacer sequence on the target DNA (specifically, one strand thereof, i.e., the “target strand” versus the “non-target strand” of the target DNA sequence). In some embodiments, in order for a Cas nickase component of a prime editor to function, it also requires a specific protospacer adjacent motif (PAM) that varies depending on the Cas protein component itself, e.g., the type of Cas protein and the bacterial species from which it is derived. The most commonly used Cas9 nuclease, derived from S. pyogenes, recognizes a PAM sequence of NGG that is directly downstream of the protospacer sequence in the genomic DNA, on the non-target strand.

### Protospacer Adjacent Motif (PAM)

As used herein, the term “protospacer adjacent motif” or “PAM” refers to a DNA sequence (e.g., an approximately 2-6 nucleotide sequence) that is an important targeting component of a Cas nuclease, e.g., a Cas9. For example, in some embodiments for a Cas9 nuclease, the PAM sequence is on either strand and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′, wherein “N” is any nucleobase followed by two guanine (“G”) nucleobases. In some embodiments, SpCas9 can also recognize additional non-canonical PAMs (e.g., NAG and NGA).

Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes an alternative PAM sequence.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

### Spacer Sequence

As used herein, the term “spacer sequence” in connection with a guide RNA or a pegRNA refers to the portion of the guide RNA or pegRNA of about 20 nucleotides which contains a nucleotide sequence that corresponds to the protospacer sequence in the target DNA sequence. The spacer sequence anneals to the target strand (which is complementary to the protospacer strand) to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### DETAILED DESCRIPTION OF CERTAIN EMBODIMENTS

This disclosure provides prime editing systems, prime editors, pegRNAs, ngRNAs, pharmaceutical compositions, and methods for correcting one or more pathogenic mutations (e.g., c.1412delA, the c.826-1 G-to-A point mutation in CDKL5, and clusters of mutations in exon 8 of CDKL5) in a cyclin-dependent kinase-like 5 (CDKL5) gene for treating CDD (CDKL5 deficiency disorder).

In a first aspect, the disclosure provides prime editing systems and components thereof. In a second aspect, the disclosure provides constructs encoding the prime editing systems and/or components thereof. In a third aspect, the disclosure provides delivery vehicles (e.g., AAV delivery vectors or LNP delivery systems) comprising and/or encoding the prime editing systems and/or components thereof. In a fourth aspect, the disclosure provides amino acid and nucleotide sequences of the prime editing systems and/or components thereof. In a fifth aspect, the disclosure provides cells expressing or containing the prime editing systems and/or components thereof. In a sixth aspect, the disclosure provides methods of gene editing using the prime editing systems and/or components, including ex vivo and in vivo methods of editing. In some embodiments, the prime editing systems are capable of gene editing a cyclin-dependent kinase-like 5 (CDKL5) gene in order to correct and/or repair a CDD-associated pathogenic mutation, including, but not limited to, the single “A” nucleotide deletion in Asp471 (i.e., the c.1412delA mutation) known to be causative of CDD.

In certain embodiments, the prime editing systems described herein comprise prime editing guide RNAs (pegRNAs) and prime editors, including pegRNAs and modified prime editors based on PE6, herein referred to as “PE6b-NRCH,” “PE6c-NRCH,” and “PE6d-NRCH”, which are capable of correcting pathogenic mutations in the CDKL5 gene, including, but not limited to the c.1412delA mutation.

In some embodiments, the prime editing systems described herein have been demonstrated to achieve over 40% correction of the c.1412delA mutation in a heterozygous mouse N2a cell line with less than 2% indel byproducts.

In certain embodiments, the present disclosure provides methods of using a prime editor in combination with prime editing guide RNAs (pegRNAs and, optionally, nicking gRNAs) to carry out prime editing to directly correct mutations (e.g., c.1412delA) in the CDKL5 gene that cause CDKL5 deficiency disorder.

The present disclosure also provides compositions, systems, and complexes comprising pegRNAs, nicking gRNAs, and/or prime editors provided herein, as well as polynucleotides and vectors encoding the same, and cells, kits, and pharmaceutical compositions comprising the same.

pegRNAs and Compositions Thereof

In one aspect, the present disclosure provides pegRNAs for correcting a pathogenic mutation in the CDKL5 gene, including, but not limited to, the c.1412delA mutation, and therefore treating CDKL5 deficiency disorder.

In one embodiment, the present disclosure provides pegRNAs for correcting a c.1412delA mutation in CDKL5 and treating CDKL5 deficiency disorder.

In one aspect, the present disclosure provides pegRNAs for correcting a c.1412delA mutation in CDKL5 gene comprising a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6). In some embodiments, the pegRNA comprises the sequence of any of the pegRNAs provided in Tables 1, 2, and 4 below, or a sequence at least 80%, at least 85%, at least 90%, at least 95%, at least 96%, at least 97%, at least 98%, or at least 99% identical to any of the pegRNAs provided in Tables 1, 2, and 4 below.

In some embodiments, the pegRNAs of the present disclosure comprise a gRNA scaffold sequence of GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence at least 80%, at least 85%, at least 90%, at least 95%, at least 96%, at least 97%, at least 98%, or at least 99% identical to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence comprising one, two, three, four, or five mutations, insertions, or deletions relative to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577).

In some embodiments, the pegRNAs of the present disclosure comprise a reverse transcription sequence of CTATATTGACACAA (SEQ ID NO: 404), GCTATATTGACACAA (SEQ ID NO: 405), AGCTATATTGACACAA (SEQ ID NO: 406), CAGCTATATTGACACAA (SEQ ID NO: 407), ACAGCTATATTGACACAA (SEQ ID NO: 408), CACAGCTATATTGACACAA (SEQ ID NO: 409), GCACAGCTATATTGACACAA (SEQ ID NO: 410), GGCACAGCTATATTGACACAA (SEQ ID NO: 411), GAATTGTGTCAAGA (SEQ ID NO: 412), GGAATTGTGTCAAGA (SEQ ID NO: 413), GGGAATTGTGTCAAGA (SEQ ID NO: 414), GGGGAATTGTGTCAAGA (SEQ ID NO: 415), TGGGGAATTGTGTCAAGA (SEQ ID NO: 416), CTGGGGAATTGTGTCAAGA (SEQ ID NO: 417), ACTGGGGAATTGTGTCAAGA (SEQ ID NO: 418), GACTGGGGAATTGTGTCAAGA (SEQ ID NO: 419), GACTGGGGAATTGTGTCGATG (SEQ ID NO: 420), GACTGGGGAATTGTGTCAATG (SEQ ID NO: 421), GACTGGGGAATTGTGTCGATA (SEQ ID NO: 422), GACTGGGGAATTGTGTCTATG (SEQ ID NO: 423), GACTGGGGAATTGTGTCAATA (SEQ ID NO: 424), GACTGGGGAATTGTGTCTATA (SEQ ID NO: 425), GGACTGGGGAATTGTATCGATG (SEQ ID NO: 426), GGACTGGGGAATTGTATCAATG (SEQ ID NO: 427), GGACTGGGGAATTGTATCTATG (SEQ ID NO: 428), TAGAGGACTGGGGAATCGTGTCAATA (SEQ ID NO: 429), GGACTGGGGAATTGTATCGATA (SEQ ID NO: 430), TAGAGGACTGGGGAATCGTGTCGATA (SEQ ID NO: 431), GGACTGGGGAATTGTATCTATA (SEQ ID NO: 432), GACTCCTAGAGGACTGGGGGATCGTGTCAATA (SEQ ID NO: 433), TCCTAGAGGACTGGGGAATCGTATCAATA (SEQ ID NO: 434), GGACTGGGGAATTGTATCAATA (SEQ ID NO: 435), TAGAGGACTGGGGAATAGTGTCAATA (SEQ ID NO: 436), TAGAGGACTGGGGAATCGTGTCGATG (SEQ ID NO: 437), TAGAGGACTGGGGAATCGTGTCAATG (SEQ ID NO: 438), TAGAGGACTGGGGAATCGTGTCTATA (SEQ ID NO: 439), TAGAGGACTGGGGAATAGTATCAATA (SEQ ID NO: 440), TAGAGGACTGGGGAATGGTGTCAATA (SEQ ID NO: 441), CTCCTAGAGGACTGGGGGATCGTATCAATA (SEQ ID NO: 442), CTCCTAGAGGACTGGGGGATGGTATCAATA (SEQ ID NO: 443), CTCCTAGAGGACTGGGGGATGGTATCAATG (SEQ ID NO: 444), GACTCCTAGAGGACTGGGGGATAGTGTCAATG (SEQ ID NO: 445), CTCCTAGAGGACTGGGGGATCGTATCGATA (SEQ ID NO: 446), TCCTAGAGGACTGGGGAATCGTATCGATA (SEQ ID NO: 447), GACTCCTAGAGGACTGGGGGATGGTGTCAATG (SEQ ID NO: 448), TCCTAGAGGACTGGGGAATCGTATCAATG (SEQ ID NO: 449), TAGAGGACTGGGGAATGGTATCAATA (SEQ ID NO: 450), TAGAGGACTGGGGAATGGTGTCGATA (SEQ ID NO: 451), TAGAGGACTGGGGAATAGTGTCGATG (SEQ ID NO: 452), TAGAGGACTGGGGAATGGTGTCAATG (SEQ ID NO: 453), TAGAGGACTGGGGAATAGTATCGATA (SEQ ID NO: 454), TAGAGGACTGGGGAATAGTGTCAATG (SEQ ID NO: 455), TAGAGGACTGGGGAATAGTATCAATG (SEQ ID NO: 456), CCTAGAGGACTGGGGGATCGTGTCGATA (SEQ ID NO: 457), TAGAGGACTGGGGAATGGTGTCGATG (SEQ ID NO: 458), TCCTAGAGGACTGGGGTATCGTGTCAATA (SEQ ID NO: 459), TAGAGGACTGGGGAATAGTATCGATG (SEQ ID NO: 460), TAGAGGACTGGGGAATAGTGTCGATA (SEQ ID NO: 461), CTCCTAGAGGACTGGGGGATAGTATCAATG (SEQ ID NO: 462), TAGAGGACTGGGGAATGGTATCGATA (SEQ ID NO: 463), CCTAGAGGACTGGGGGATCGTGTCGATG (SEQ ID NO: 464), GACTCCTAGAGGACTGGGGGATGGTGTCAATA (SEQ ID NO: 465), CTCCTAGAGGACTGGGGGATAGTATCAATA (SEQ ID NO: 466), TAGAGGACTGGGGAATGGTATCAATG (SEQ ID NO: 467), TCCTAGAGGACTGGGGAATCGTATCGATG (SEQ ID NO: 468), TAGAGGACTGGGGAATAGTATCTATA (SEQ ID NO: 469), GACTCCTAGAGGACTGGGGGATCGTGTCAATG (SEQ ID NO: 470), TAGAGGACTGGGGAATCGTGTCTATG (SEQ ID NO: 471), TAGAGGACTGGGGAATGGTATCGATG (SEQ ID NO: 472), GACTCCTAGAGGACTGGGGGATAGTGTCAATA (SEQ ID NO: 473), TAGAGGACTGGGGAATGGTGTCTATA (SEQ ID NO: 474), TAGAGGACTGGGGAATGGTATCTATA (SEQ ID NO: 475), TAGAGGACTGGGGAATAGTGTCTATG (SEQ ID NO: 476), CCTAGAGGACTGGGGTATCGTATCAATA (SEQ ID NO: 477), TAGAGGACTGGGGAATAGTGTCTATA (SEQ ID NO: 478), TCCTAGAGGACTGGGGGATGGTGTCGATA (SEQ ID NO: 479), TAGAGGACTGGGGAATCGTATCTATA (SEQ ID NO: 480), CTCCTAGAGGACTGGGGGATCGTATCGATG (SEQ ID NO: 481), TCCTAGAGGACTGGGGGATGGTGTCGATG (SEQ ID NO: 482), TAGAGGACTGGGGAATAGTATCTATG (SEQ ID NO: 483), TAGAGGACTGGGGAATGGTGTCTATG (SEQ ID NO: 484), CTCCTAGAGGACTGGGGGATTGTATCAATA (SEQ ID NO: 485), GACTCCTAGAGGACTGGGGGATAGTGTCGATG (SEQ ID NO: 486), TCCTAGAGGACTGGGGTATCGTATCGATA (SEQ ID NO: 487), CTCCTAGAGGACTGGGGGATAGTATCGATA (SEQ ID NO: 488), CTCCTAGAGGACTGGGGGATCGTATCAATG (SEQ ID NO: 489), CTCCTAGAGGACTGGGGGATTGTATCAATG (SEQ ID NO: 490), CTCCTAGAGGACTGGGGGATGGTATCGATA (SEQ ID NO: 491), GACTCCTAGAGGACTGGGGGATAGTGTCGATA (SEQ ID NO: 492), CTCCTAGAGGACTGGGGGATGGTATCGATG (SEQ ID NO: 493), CTCCTAGAGGACTGGGGGATAGTATCGATG (SEQ ID NO: 494), TAGAGGACTGGGGAATGGTATCTATG (SEQ ID NO: 495), TCCTAGAGGACTGGGGTATCGTGTCGATA (SEQ ID NO: 496), CTCCTAGAGGACTGGGGGATTGTATCGATA (SEQ ID NO: 497), CTCCTAGAGGACTGGGGGATCGTATCTATA (SEQ ID NO: 498), CTCCTAGAGGACTGGGGGATTGTATCGATG (SEQ ID NO: 499), TAGAGGACTGGGGAATCGTATCTATG (SEQ ID NO: 500), CCTAGAGGACTGGGGGATTGTGTCAATG (SEQ ID NO: 501), CCTAGAGGACTGGGGTATGGTATCGATA (SEQ ID NO: 502), CCTAGAGGACTGGGGTATTGTGTCAATA (SEQ ID NO: 503), CTCCTAGAGGACTGGGGGATGGTATCTATA (SEQ ID NO: 504), TCCTAGAGGACTGGGGTATCGTGTCGATG (SEQ ID NO: 505), CTCCTAGAGGACTGGGGTATGGTATCAATA (SEQ ID NO: 506), GACTCCTAGAGGACTGGGGGATCGTGTCTATA (SEQ ID NO: 507), CCTAGAGGACTGGGGGATTGTGTCAATA (SEQ ID NO: 508), CCTAGAGGACTGGGGGATTGTGTCGATG (SEQ ID NO: 509), GACTCCTAGAGGACTGGGGGATCGTGTCTATG (SEQ ID NO: 510), CTCCTAGAGGACTGGGGGATGGTATCTATG (SEQ ID NO: 511), CTCCTAGAGGACTGGGGGATAGTATCTATA (SEQ ID NO: 512), CCTAGAGGACTGGGGTATTGTGTCAATG (SEQ ID NO: 513), TCCTAGAGGACTGGGGTATAGTGTCAATA (SEQ ID NO: 514), CCTAGAGGACTGGGGGATTGTGTCGATA (SEQ ID NO: 515), GACTCCTAGAGGACTGGGGTATAGTATCAATA (SEQ ID NO: 516), TCCTAGAGGACTGGGGTATCGTATCGATG (SEQ ID NO: 517), CCTAGAGGACTGGGGTATGGTATCGATG (SEQ ID NO: 518), GACTCCTAGAGGACTGGGGGATGGTGTCTATG (SEQ ID NO: 519), CTCCTAGAGGACTGGGGGATCGTATCTATG (SEQ ID NO: 520), GACTCCTAGAGGACTGGGGGATGGTGTCTATA (SEQ ID NO: 521), CTCCTAGAGGACTGGGGGATAGTATCTATG (SEQ ID NO: 522), TCCTAGAGGACTGGGGTATAGTGTCAATG (SEQ ID NO: 523), GACTCCTAGAGGACTGGGGGATAGTGTCTATG (SEQ ID NO: 524), GACTCCTAGAGGACTGGGGTATAGTATCAATG (SEQ ID NO: 525), TCCTAGAGGACTGGGGTATCGTGTCAATG (SEQ ID NO: 526), GACTCCTAGAGGACTGGGGGATAGTGTCTATA (SEQ ID NO: 527), CCTAGAGGACTGGGGTATCGTATCAATG (SEQ ID NO: 528), CTCCTAGAGGACTGGGGTATGGTATCAATG (SEQ ID NO: 529), CTCCTAGAGGACTGGGGGATTGTATCTATA (SEQ ID NO: 530), GACTCCTAGAGGACTGGGGTATAGTATCGATA (SEQ ID NO: 531), TCCTAGAGGACTGGGGTATTGTGTCGATG (SEQ ID NO: 532), CCTAGAGGACTGGGGTATTGTATCAATA (SEQ ID NO: 533), TCCTAGAGGACTGGGGTATTGTGTCGATA (SEQ ID NO: 534), CTCCTAGAGGACTGGGGGATTGTATCTATG (SEQ ID NO: 535), GACTCCTAGAGGACTGGGGTATGGTGTCAATA (SEQ ID NO: 536), TCCTAGAGGACTGGGGTATAGTGTCGATA (SEQ ID NO: 537), CCTAGAGGACTGGGGGATTGTGTCTATG (SEQ ID NO: 538), GACTCCTAGAGGACTGGGGTATAGTATCGATG (SEQ ID NO: 539), CCTAGAGGACTGGGGTATTGTATCGATA (SEQ ID NO: 540), CCTAGAGGACTGGGGGATTGTGTCTATA (SEQ ID NO: 541), CCTAGAGGACTGGGGTATTGTATCAATG (SEQ ID NO: 542), TCCTAGAGGACTGGGGTATAGTGTCGATG (SEQ ID NO: 543), GACTCCTAGAGGACTGGGGTATGGTGTCAATG (SEQ ID NO: 544), CTCCTAGAGGACTGGGGTATGGTGTCGATA (SEQ ID NO: 545), CCTAGAGGACTGGGGTATTGTATCGATG (SEQ ID NO: 546), TCCTAGAGGACTGGGGTATCGTGTCTATA (SEQ ID NO: 547), TCCTAGAGGACTGGGGTATCGTATCTATA (SEQ ID NO: 548), CTCCTAGAGGACTGGGGTATGGTGTCGATG (SEQ ID NO: 549), TCCTAGAGGACTGGGGTATAGTGTCTATA (SEQ ID NO: 550), CCTAGAGGACTGGGGTATGGTATCTATA (SEQ ID NO: 551), TCCTAGAGGACTGGGGTATAGTGTCTATG (SEQ ID NO: 552), TCCTAGAGGACTGGGGTATCGTGTCTATG (SEQ ID NO: 553), CCTAGAGGACTGGGGTATTGTGTCTATG (SEQ ID NO: 554), CCTAGAGGACTGGGGTATTGTATCTATA (SEQ ID NO: 555), CCTAGAGGACTGGGGTATTGTGTCTATA (SEQ ID NO: 556), CTCCTAGAGGACTGGGGTATAGTATCTATA (SEQ ID NO: 557), CCTAGAGGACTGGGGTATGGTGTCTATA (SEQ ID NO: 558), TCCTAGAGGACTGGGGTATGGTATCTATG (SEQ ID NO: 559), CCTAGAGGACTGGGGTATTGTATCTATG (SEQ ID NO: 560), CCTAGAGGACTGGGGTATGGTGTCTATG (SEQ ID NO: 561), CTCCTAGAGGACTGGGGTATAGTATCTATG (SEQ ID NO: 562), or TCCTAGAGGACTGGGGTATCGTATCTATG (SEQ ID NO: 563), or a sequence at least 80%, at least 85%, at least 90%, at least 95%, at least 96%, at least 97%, at least 98%, or at least 99% identical to any one of SEQ ID NOs: 404-563, or a sequence comprising one, two, three, four, or five mutations, insertions, or deletions relative to any one of SEQ ID NOs: 404-563.

In some embodiments, the pegRNAs of the present disclosure comprise a primer binding site (PBS) of the sequence TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35), TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence at least 80%, at least 85%, at least 90%, at least 95%, at least 96%, at least 97%, at least 98%, or at least 99% identical to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35), TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence comprising one, two, three, four, or five mutations, insertions, or deletions relative to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35), TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6).

In some embodiments, the pegRNAs of the present disclosure comprise a 3′ structured motif of the sequence CGCGGTTCTATCTAGTTACGCGTTAAACCAACTAGAA (SEQ ID NO: 14), or a sequence at least 80%, at least 85%, at least 90%, at least 95%, at least 96%, at least 97%, at least 98%, or at least 99% identical to CGCGGTTCTATCTAGTTACGCGTTAAACCAACTAGAA (SEQ ID NO: 14), or a sequence comprising one, two, three, four, or five mutations, insertions, or deletions relative to CGCGGTTCTATCTAGTTACGCGTTAAACCAACTAGAA (SEQ ID NO: 14).

In some embodiments, the pegRNAs of the present disclosure comprise a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT. In some embodiments, the pegRNAs comprise 0, 1, 2, 3, 4, 5, or 6 T's of the terminator sequence.

In some embodiments, the pegRNAs of the present disclosure comprise the structure 5′-[spacer]-[gRNA scaffold]-[RTT]-[PBS]-[3′ motif]-[terminator]-3′. In some embodiments, the pegRNAs of the present disclosure comprise the structure 5′-[spacer]-[gRNA scaffold]-[RTT]-[PBS]-[3′ motif]-3′.

In another aspect, the present disclosure provides compositions comprising any of the pegRNAs provided herein. In some embodiments, the composition further comprises a nicking guide RNA (ngRNA). In some embodiments, the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13). In certain embodiments, the ngRNA comprises the sequence of any one of the ngRNAs provided in Table 3 below, or a sequence at least 80%, at least 85%, at least 90%, at least 95%, at least 96%, at least 97%, at least 98%, or at least 99% identical to any one of the ngRNAs provided in Table 3 below.

In some embodiments, the ngRNA comprises a gRNA scaffold sequence of GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence at least 80%, at least 85%, at least 90%, at least 95%, at least 96%, at least 97%, at least 98%, or at least 99% identical to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence comprising one, two, three, four, or five mutations, insertions, or deletions relative to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578).

In some embodiments, the ngRNA comprises a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT.

In some embodiments, the ngRNA comprises the structure 5′-[spacer]-[gRNA scaffold]-[terminator]-3′.

In another aspect, the present disclosure provides pegRNAs for correcting a c.826-1 G-to-A point mutation in a CDKL5 gene by prime editing. In some embodiments, the pegRNA comprises a spacer sequence of GCTGTCAAGTATCTGTCAGCT (SEQ ID NO: 7), or a spacer sequence at least 80%, at least 85%, at least 90%, at least 95%, at least 96%, at least 97%, at least 98%, or at least 99% identical to GCTGTCAAGTATCTGTCAGCT (SEQ ID NO: 7), or a spacer sequence comprising one, two, three, four, or five mutations, insertions, or deletions relative to GCTGTCAAGTATCTGTCAGCT (SEQ ID NO: 7). In certain embodiments, the pegRNA comprises the sequence GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence at least 80%, at least 85%, at least 90%, at least 95%, at least 96%, at least 97%, at least 98%, or at least 99% identical to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence comprising one, two, three, four, or five mutations, insertions, or deletions relative to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39).

In another aspect, the present disclosure provides nicking guide RNAs for use in correcting a c.826-1 G-to-A point mutation in a CDKL5 gene by prime editing. In some embodiments, the nicking guide RNA comprises the sequence GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence at least 80%, at least 85%, at least 90%, at least 95%, at least 96%, at least 97%, at least 98%, or at least 99% identical to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence comprising one, two, three, four, or five mutations, insertions, or deletions relative to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17).

In another aspect, the present disclosure provides pegRNAs for correcting a cluster of mutations in exon 8 of the CDKL5 gene by prime editing. In some embodiments, the pegRNA comprises a spacer sequence of CAGAGTACGTTGCCACCAGA (SEQ ID NO: 8), or a spacer sequence at least 80%, at least 85%, at least 90%, at least 95%, at least 96%, at least 97%, at least 98%, or at least 99% identical to CAGAGTACGTTGCCACCAGA (SEQ ID NO: 8), or a spacer sequence comprising one, two, three, four, or five mutations, insertions, or deletions relative to CAGAGTACGTTGCCACCAGA (SEQ ID NO: 8). In certain embodiments, the pegRNA comprises the sequence CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence at least 80%, at least 85%, at least 90%, at least 95%, at least 96%, at least 97%, at least 98%, or at least 99% identical to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence comprising one, two, three, four, or five mutations, insertions, or deletions relative to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41).

In another aspect, the present disclosure provides nicking guide RNAs for correcting a cluster of mutations in exon 8 of the CDKL5 gene by prime editing. In some embodiments, the nicking guide RNA comprises the sequence GTGGAAATGTCATTCTATTTT (SEQ ID NO: 18), or a sequence at least 80%, at least 85%, at least 90%, at least 95%, at least 96%, at least 97%, at least 98%, or at least 99% identical to GTGGAAATGTCATTCTATTTT (SEQ ID NO: 18), or a sequence comprising one, two, three, four, or five mutations, insertions, or deletions relative to GTGGAAATGTCATTCTATTTT (SEQ ID NO: 18).

In another aspect, the present disclosure provides systems comprising a pegRNA, prime editor, and ngRNA described herein.

In another aspect, the present disclosure provides one or more polynucleotides encoding any of the pegRNAs and/or ngRNAs provided herein. In some embodiments, the one or more polynucleotides encode any of the prime editors provided herein.

In another aspect, the present disclosure provides one or more vectors comprising the one or more polynucleotides provided herein. In some embodiments, the vectors are AAV vectors. In some embodiments, the AAV is AAV1, AAV2, AAV3, AAV4, AAV5, AAV6, AAV7, AAV8, or AAV9. In certain embodiments, the AAV is AAV9.

In another aspect, the present disclosure provides compositions comprising any of the polynucleotides and/or vectors provided herein.

In another aspect, the present disclosure provides one or more AAV particles comprising any of the pegRNAs, compositions, polynucleotides, and/or vectors provided herein. The use of AAVs for delivering gene editing agents, including prime editors and pegRNAs, is described, for example, in Davis et al., Efficient Prime Editing in Mouse Brain, Liver and Heart with Dual AAVs. Biotechnol. 42, 253-264 (2024), which is incorporated herein by reference.

In another aspect, the present disclosure provides virus-like particles (VLPs) comprising any of the pegRNAs, compositions, polynucleotides and/or vectors provided herein. The use of VLPs for the delivery of gene editing agents, including prime editors and pegRNAs, is described, for example, in International Patent Application Publication No. WO 2023/102537, published Jun. 8, 2023; International Patent Application Publication No. WO 2023/102538, published Jun. 8, 2023; International Patent Application Publication No. WO 2023/102550, published Jun. 8, 2023; International Patent Application Publication No. 2024/215652, published Oct. 17, 2024; International Patent Application No. PCT/US2024/032867, filed Jun. 6, 2024; Banskota et al., Engineered Virus-Like Particles for Efficient In Vivo Delivery of Therapeutic Proteins. Cell 185, 250-265, (2022); and An et al., Engineered Virus-Like Particles for Transient Delivery of Prime Editor Ribonucleoprotein Complexes In Vivo. Nat. Biotechnol. 42 (10): 1526-1537 (2024); the contents of which are incorporated by reference herein.

In another aspect, the present disclosure provides pharmaceutical compositions comprising any of the pegRNAs, compositions, polynucleotides, vectors, AAV particles, or VLPs provided herein, and a pharmaceutically acceptable excipient.

In another aspect, the present disclosure provides cells comprising any of the pegRNAs, compositions, polynucleotides, vectors, AAV particles, or VLPs provided herein. In another aspect, the present disclosure provides kits comprising any of the pegRNAs, compositions, polynucleotides, vectors, AAV particles, or VLPs provided herein.

### Prime Editors

Any of the compositions, systems, or prime editing complexes provided herein may comprise a prime editor. In some embodiments, a composition comprises a prime editor. In some embodiments, a system comprises a prime editor. In some embodiments, the prime editor comprises (i) a napDNAbp domain, and (ii) a polymerase domain. In certain embodiments, the napDNAbp domain has a nickase activity. In some embodiments, the napDNAbp domain is a Cas9 protein or a variant thereof (e.g., Cas9-NRCH, or a variant thereof). In certain embodiments, the napDNAbp domain is a Cas9 nickase (nCas9). In certain embodiments, the napDNAbp domain comprises a sequence of any one of the napDNAbps provided herein, or an amino acid sequence at least 80%, at least 85%, at least 90%, at least 95%, at least 96%, at least 97%, at least 98%, or at least 99% identical to any one of the napDNAbps provided herein. In some embodiments, the polymerase domain is a reverse transcriptase domain. In certain embodiments, the polymerase domain comprises a sequence of any one of the polymerases provided herein, or an amino acid sequence at least 80%, at least 85%, at least 90%, at least 95%, at least 96%, at least 97%, at least 98%, or at least 99% identical to any one of the polymerases provided herein. In some embodiments, the Cas9 domain (e.g. a Cas9 nickase) and the polymerase domain (e.g., a reverse transcriptase) are a translationally fused, i.e., form a fusion protein. In other embodiments, the Cas9 domain (e.g., a Cas9 nickase) and the polymerase domain (e.g., a reverse transcriptase) are not translationally fused, i.e., are provided in trans. In such embodiments, the different components may be provided in trans by introduction of separate expression constructs whereby each component is independently expressed. In some embodiments, the independently expressed components each may further comprise a localization or assembly domain so that the components as assembled to form a functional complex through the coupling of the localization or assembly domains.

In some embodiments, any of the following prime editor may be used in the present disclosure:

### PE1

As used herein, “PE1” refers to a PE complex comprising a fusion protein comprising Cas9 (H840A) nickase and a wild type MMLV RT having the following structure: [NLS]-[Cas9 (H840A)]-[linker]-[MMLV_RT(wt)]+a desired pegRNA, wherein the PE fusion has the following amino acid sequence:

(SEQ ID NO: 347)
MKRTADGSEFESPKKKRKVDKKYSIGLDIGTNSVGWAVITDEYKVPSKKFKVLGN
TDRHSIKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDSIDNK
VLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDAT
LIHQSITGLYETRIDLSQLGGDSGGSSGGSSGSETPGTSESATPESSGGSSGGSSTL
NIEDEYRLHETSKEPDVSLGSTWLSDFPQAWAETGGMGLAVRQAPLIIPLKATSTPVSIKQY
PMSQEARLGIKPHIQRLLDQGILVPCQSPWNTPLLPVKKPGTNDYRPVQDLREVNKRVED
IHPTVPNPYNLLSGLPPSHQWYTVLDLKDAFFCLRLHPTSQPLFAFEWRDPEMGISGQLT
WTRLPQGFKNSPTLFDEALHRDLADFRIQHPDLILLQYVDDLLLAATSELDCQQGTRALL
QTLGNLGYRASAKKAQICQKQVKYLGYLLKEGQRWLTEARKETVMGQPTPKTPRQLREF
LGTAGFCRLWIPGFAEMAAPLYPLTKTGTLFNWGPDQQKAYQEIKQALLTAPALGLPDLTK
PFELFVDEKQGYAKGVLTQKLGPWRRPVAYLSKKLDPVAAGWPPCLRMVAAIAVLTKDAG
KLTMGQPLVILAPHAVEALVKQPPDRWLSNARMTHYQALLLDTDRVQFGPVVALNPATLL
PLPEEGLOHNCLDILAEAHGTRPDLTDQPLPDADHTWYTDGSSLLQEGORKAGAAVTTET
EVIWAKALPAGTSAQRAELIALTQALKMAEGKKLNVYTDSRYAFATAHIHGEIYRRRGLLTSE
GKEIKNKDEILALLKALFLPKRLSIIHCPGHQKGHSAEARGNRMADQAARKAAITETPDTS
TLLIENSSPSGGSKRTADGSEFEPKKKRKV

KEY:
NUCLEAR LOCALIZATION SEQUENCE (NLS)
CAS9(H840A)
33-AMINO ACID LINKER
M-MLV reverse transcriptase

### PE2

As used herein, “PE2” refers to a PE complex comprising a fusion protein comprising Cas9 (H840A) nickase and a variant MMLV RT having the following structure: [NLS]-[Cas9 (H840A)]-[linker]-[MMLV_RT (D200N) (T330P) (L603W) (T306K) (W313F)]+a desired pegRNA, wherein the PE fusion has the amino acid sequence of:

(SEQ ID NO: 348)
MKRTADGSEFESPKKKRKVDKKYSIGLDIGTNSVGWAVITDEYKVPSKKFKVLGN
TDRHSIKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDSIDNK
VLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLV
SDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDAT
LIHQSITGLYETRIDLSQLGGDSGGSSGGSSGSETPGTSESATPESSGGSSGGSSTL
NIEDEYRLHETSKEPDVSLGSTWLSDFPQAWAETGGMGLAVRQAPLIIPLKATSTPVSIKQY
PMSQEARLGIKPHIQRLLDQGILVPCQSPWNTPLLPVKKPGTNDYRPVQDLREVNKRVED
IHPTVPNPYNLLSGLPPSHQWYTVLDLKDAFFCLRLHPTSQPLFAFEWRDPEMGISGQLT
WTRLPQGFKNSPTLFNEALHRDLADFRIQHPDLILLQYVDDLLLAATSELDCQQGTRALL
QTLGNLGYRASAKKAQICQKQVKYLGYLLKEGQRWLTEARKETVMGQPTPKTPRQLREF
LGKAGFCRLFIPGFAEMAAPLYPLTKPGTLFNWGPDQQKAYQEIKQALLTAPALGLPDLTK
PFELFVDEKQGYAKGVLTQKLGPWRRPVAYLSKKLDPVAAGWPPCLRMVAAIAVLTKDAG
KLTMGQPLVILAPHAVEALVKQPPDRWLSNARMTHYQALLLDTDRVQFGPVVALNPATLL
PLPEEGLQHNCLDILAEAHGTRPDLTDQPLPDADHTWYTDGSSLLQEGQRKAGAAVTTET
EVIWAKALPAGTSAQRAELIALTQALKMAEGKKLNVYTDSRYAFATAHIHGEIYRRRGWLTS
EGKEIKNKDEILALLKALFLPKRLSIIHCPGHQKGHSAEARGNRMADQAARKAAITETPDT
STLLIENSSPSGGSKRTADGSEFEPKKKRKV

KEY:
NUCLEAR LOCALIZATION SEQUENCE (NLS)
CAS9(H840A)
33-AMINO ACID LINKER
M-MLV reverse transcriptase

### PE3

As used herein, “PE3” refers to PE2 plus a second-strand nicking guide RNA (ngRNA) that complexes with the PE2 and introduces a nick in the non-edited DNA strand in order to induce preferential replacement of the edited strand.

### PE3b

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, referred to hereafter as PE3b, mismatches between the protospacer and the unedited allele should disfavor nicking by the sgRNA until after the editing event on the PAM strand takes place.

### PE4

As used herein, “PE4” refers to a prime editing composition comprising a PE2 and further comprising an MLH1 dominant negative protein variant (i.e., wild-type MLH1 with amino acids 754-756 truncated, which may be referred to herein as “MLH1 Δ754-756” or “MLH1dn”). The MLH1 dominant negative protein variant may be expressed in trans in some embodiments. In some embodiments, a PE4 system comprises a fusion protein comprising a PE2 protein and an MLH1 dominant negative protein joined via an optional linker.

The amino acid sequence of MLH1dn is:

(SEQ ID NO: 351)
MSFVAGVIRRLDETVVNRIAAGEVIQRPANAIKEMIENCLDAKSTSIQVIVKEGGLK
LIQIQDNGTGIRKEDLDIVCERFTTSKLQSFEDLASISTYGFRGEALASISHVAHVTITT
KTADGKCAYRASYSDGKLKAPPKPCAGNQGTQITVEDLFYNIATRRKALKNPSEEY
GKILEVVGRYSVHNAGISFSVKKQGETVADVRTLPNASTVDNIRSIFGNAVSRELIEIG
CEDKTLAFKMNGYISNANYSVKKCIFLLFINHRLVESTSLRKAIETVYAAYLPKNTHP
FLYLSLEISPQNVDVNVHPTKHEVHFLHEESILERVQQHIESKLLGSNSSRMYFTQTLL
PGLAGPSGEMVKSTTSLTSSSTSGSSDKVYAHQMVRTDSREQKLDAFLQPLSKPLSS
QPQAIVTEDKTDISSGRARQQDEEMLELPAPAEVAAKNQSLEGDTTKGTSEMSEKRG
PTSSNPRKRHREDSDVEMVEDDSRKEMTAACTPRRRIINLTSVLSLQEEINEQGHEVL
REMLHNHSFVGCVNPQWALAQHQTKLYLLNTTKLSEELFYQILIYDFANFGVLRLSE
PAPLFDLAMLALDSPESGWTEEDGPKEGLAEYIVEFLKKKAEMLADYFSLEIDEEGN
LIGLPLLIDNYVPPLEGLPIFILRLATEVNWDEEKECFESLSKECAMFYSIRKQYISEES
TLSGQQSEVPGSIPNSWKWTVEHIVYKALRSHILPPKHFTEDGNILQLANLPDLYKVF

### PE5 and PE5b

As used herein, “PE5” refers to a prime editing composition comprising a PE3 prime editor and further comprising an MLH1 dominant negative protein variant (i.e., wild-type MLH1 with amino acids 754-756 truncated, which may be referred to as “MLH1 Δ754-756” or “MLH1dn”). The MLH1 dominant negative variant may be expressed in trans in some embodiments. In some embodiments, a PE5 system comprises a fusion protein comprising a PE2 protein and an MLH1 dominant negative protein joined via an optional linker. “PE5b” refers to a prime editing composition comprising a PE3 and an MLH1 dominant negative protein, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridize with, only the edited strand after installation of the desired nucleotide edit(s), but not the endogenous target DNA sequence.

### PE6

The term “PE6” refers to a suite of next-generation prime editors described herein (PE6a, PE6b, PE6c, PE6d, PE6e, PE6f, and PE6g) comprising improved reverse transcriptase and/or Cas9 variants. PE6a, PE6b, PE6c, PE6d, PE6e, PE6f, and PE6g were described in Doman et al., “Phage-assisted evolution and protein engineering yield compact, efficient prime editors,” Cell, 2023 Aug. 31; 186 (18): 3983-4002, and International PCT Application PCT/US2024/030786, each of which is incorporated herein by reference. The improved reverse transcriptase and Cas9 domains of the PE6 variants can also be combined with each other to offer cumulative benefits. For example, a PE6 prime editor comprising an improved reverse transcriptase variant of PE6a and an improved Cas9 variant of PE6e is referred to herein as the prime editor “PE6a-e” (or “PE6e-a”). Any possible combination of PE6 prime editors is contemplated by the present disclosure including, for example, PE6a-e, PE6a-f, PE6a-g, PE6b-e, PE6b-f, PE6b-g, PE6c-e, PE6c-f, PE6c-g, PE6d-e, PE6d-f, and PE6d-g.

Any of the PE6 prime editors provided herein may also comprise the architecture of the PEmax protein as provided below. In some embodiments, any of the PE6 prime editors provided herein may further comprise additional amino acid mutations, e.g., any of those included in PEmax as provided below.

In some embodiments, a PE6 protein comprises a reverse transcriptase of the following amino acid sequence (the RT domain of “PE6a”), or an amino acid sequence at least 80%, at least 85%, at least 90%, at least 95%, at least 96%, at least 97%, at least 98%, or at least 99% identical to the following amino acid sequence:

(SEQ ID NO: 572)
GRPYVTLNLNGMFMDKFKPYSKSNAPITTLEKLSKALSISVEELKAIAELSLDEKYTL
KKIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALDYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSNRDVA
VIDAAIKSLELSYSKGNQNKHWYKRKYDLTRYKMIILTRSESFKEKLECFKSRLASLK
PL.

In some embodiments, a PE6 protein comprises a reverse transcriptase of the following amino acid sequence (the RT domain of “PE6b”), or an amino acid sequence at least 80%, at least 85%, at least 90%, at least 95%, at least 96%, at least 97%, at least 98%, or at least 99% identical to the following amino acid sequence:

(SEQ ID NO: 573)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPVL
RHFDFSKKILLETDVSDVAVGAVLSQKHDDDKYYPVGYYSAKMSKAQLNYSVSDK
EMLAIIKSLEHWRHYLESTIEPFKILTDHRNLIGRITNESEPENKRLARWOLFLQDENF
EINYRPGSANHIADALSRIVDETEPIPKDNEDNSINFVNQISI.

In some embodiments, a PE6 protein comprises a reverse transcriptase of the following amino acid sequence (the RT domain of “PE6c”), or an amino acid sequence at least 80%, at least 85%, at least 90%, at least 95%, at least 96%, at least 97%, at least 98%, or at least 99% identical to the following amino acid sequence:

(SEQ ID NO: 574)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGIKTAPAHFQYFINTILGEAKESHVVCYMDDILIHSKSESEHVKHVKD
VLQKLKNANLIINQAKCEFHQSQVKFLGYHISEKGLTPCQENIDKVLQWKQPKNQKE
LRQFLGQVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLEHWRHYLESTIEPFKILTDHRNLIGRITNESEPENKRLARWOLFLQDEN
FEINYRPGSANHIADALSRIVDETEPIPKDNEDNSINFVNQISI.

In some embodiments, a PE6 protein comprises a reverse transcriptase comprising the following amino acid sequence (the RT domain of “PE6d”), or an amino acid sequence at least 80%, at least 85%, at least 90%, at least 95%, at least 96%, at least 97%, at least 98%, or at least 99% identical to the following amino acid sequence:

(SEQ ID NO: 575)
TLNIEDEYRLHETSKEPDVSLGSTWLSDFPQAWAETGGMGLAVRQAPLIIPLKATSTP
VSIKQYPMSQEARLGIKPHIQRLLDQGILVPCQSPWNTPLLPVKKPGTNDYRPVQDLR
EVNKRVEDIHPNVPNPYNLLSGLPPSHQWYTVLDLKDAFFCLRLHPTSQPLFAFEWR
DPEMGISGQLTWTRLPQGFKNSPTLFCEALHRDLADFRIQHPDLILLQYYDDLLLAAT
SELDCQQGTRALLQTLGNLGYRASAKKAQICQKQVKYLGYLLKEGQRWLTEARKE
TVMGQPTPKTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPVAYLSKKL
DPVAAGWPPCLRMVAAIAVLTKDAGKLTMGQPLVILAPHAVEALVKQPPDRWLSN
ARMTHYQALLLDTDRVQFGPVVALNPATLLPLPEEGLQHNCLD.

In some embodiments, a PE6 protein comprises a Cas9 protein of the following amino acid sequence (the Cas9 domain of “PE6e”), or an amino acid sequence at least 80%, at least 85%, at least 90%, at least 95%, at least 96%, at least 97%, at least 98%, or at least 99% identical to the following amino acid sequence:

(SEQ ID NO: 352)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQRNSRERMKRIEEGIKELGSQILKEHPVENTQL
QNEKLYLYYLQNGRDMYVDQELDINRLSDYDVDAIVPQSFLKDDSIDNKVLTRSDK
NRGKSDNVPSEEVVKKMKNYWRQLLNAKLITQRKFDNLTKAERGGLSELDKAGFIA
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD.

In some embodiments, a PE6 protein comprises a Cas9 protein of the following amino acid sequence (the Cas9 domain of “PE6f”), or an amino acid sequence at least 80%, at least 85%, at least 90%, at least 95%, at least 96%, at least 97%, at least 98%, or at least 99% identical to the following amino acid sequence:

(SEQ ID NO: 353)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFRRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEKTITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMVEER
LKTYAHLFDNKVMKQLKRRRYTGWGRLSRKLINGIRDKQSGKTILDFLKSDGFANR
NFMQLIHDDSLTFKEDIQKAQVSGQGDSLYEHIANLAGSPAIKKGILQTVKVVDELV
KVMGRHKPENIVIEMARENQTTQKGQKNSRERMKRIEEGIKELGSQILKEHPVENTQ
LQNEKLYLYYLQNGRDMYVDQELDINRLSDYDVDAIVPQSFLKDDSIDNKVLTRSD
KNRGKSDNVPSEEVVKKMKNYWRQLLNAKLITQRKFDNLTKAERGGLSELDKAGFI
ARQLVETRQITKHVAQILDSRMNTKYDENDKLIREVKVITLKSKLVSDFRKDFQFYK
VREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGD.

In some embodiments, a PE6 protein comprises a Cas9 protein of the following amino acid sequence (the Cas9 domain of “PE6g”), or an amino acid sequence at least 80%, at least 85%, at least 90%, at least 95%, at least 96%, at least 97%, at least 98%, or at least 99% identical to the following amino acid sequence:

(SEQ ID NO: 354)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFRRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEKTITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMVEER
LKTYAHLFDNKVMKQLKRCRYTGWGRLSRKLINGIRDKQSGKTILDFLKSDGFANR
NFMQLIHDDSLTFKEDIQKAQVSGQGDSLYEHIANLAGSPAIKKGILQTVKVVDELV
KVMGRHKPENIVIEMARENQTTQKGQKNSRERMKRIEEGIKELGSQILKEHPVENTQ
LQNEKLYLYYLQNGRDMYVDQELDINRLSDYDVDAIVPQSFLKDDSIDNKVLTRSD
KNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYK
VREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGD.

In some embodiments, a PE6 protein comprises a reverse transcriptase variant disclosed herein and a Cas9 protein that recognizes a non-canonical PAM sequence, e.g., Cas9-NRCH of the following amino acid sequence, or at least 80%, at least 85%, at least 90%, at least 95%, at least 96%, at least 97%, at least 98%, or at least 99% identical to the following amino acid sequence:

(SEQ ID NO: 349)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRKLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMVKRYDEHHQDLTLLKALVRQQLPE
KYKEIFFDQSKNGYAGYIDGGASQEEFYKFIKPILEKMDGTEELLVKLKREDLLRKQR
TFDNGIIPHQIHLGELHAILRRQGDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRLRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGGHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKGNSDKLIARKKDWDPKKYGGFNSPTVAYSVLVV
AKVEKGKSKKLKSVKELLGITIMERSSFEKNPIDFLEAKGYKEVKKDLIIKLPKYSLFE
LENGRKRMLASAGVLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTINRKQYNTTKEVLDATLIRQSITGLYETRIDLSQLGGD.

For example, the prime editor “PE6b-NRCH” comprises the reverse transcriptase of PE6b and the NRCH-Cas9 protein. PE6b-NRCH comprises the amino acid sequence:

(SEQ ID NO: 355)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRDK
PIREQAENIIHLFTLTNLGAPAAFKYFDTTINRKQYNTTKEVLDATLIRQSITGLYETRI
DLSQLGGDSGGSSGGSKRTADGSEFESPKKKRKVSGGSSGGSISSSKHTLSQMNKVS
NIVKEPELPDIYKEFKDITADTNTEKLPKPIKGLEFEVELTQENYRLPIRNYPLTPVKM
QAMNDEINQGLKGGIIRESKAINACPVIFVPRKEGTLRMVVDYRPLNKYVKPNVYPL
PLIEQLLAKIQGSTIFTKLDLKSAYHQIRVRKGDEHKLAFRCPRGVFEYLVMPYGISTA
PAHFQYFINTILGEAKESHVVCYMDDILIHSKSESEHVKHVKDVLQKLKNANLIINQA
KCEFHQSQVKFIGYHISEKGLTPCQENIDKVLQWKQPKNRKELRQFLGSVNYLRKFIP
KTSQLTHPLNKLLKKDVRWKWTPTQTQAIENIKQCLVSPPVLRHFDFSKKILLETDVS
DVAVGAVLSQKHDDDKYYPVGYYSAKMSKAQLNYSVSDKEMLAIIKSLEHWRHYL
ESTIEPFKILTDHRNLIGRITNESEPENKRLARWQLFLQDFNFEINYRPGSANHIADALS
RIVDETEPIPKDNEDNSINFVNQISIKRTADGSEFESPKKKRKVPAAKRVKLD.

The prime editor “PE6c-NRCH” comprises the reverse transcriptase of PE6c and the NRCH-Cas9 protein.

The NRCH-Cas9 protein comprises the amino acid sequence:

(SEQ ID NO: 349)
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRKLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMVKRYDEHHQDLTLLKALVRQQLPE
KYKEIFFDQSKNGYAGYIDGGASQEEFYKFIKPILEKMDGTEELLVKLKREDLLRKQR
TFDNGIIPHQIHLGELHAILRRQGDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRLRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGGHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKGNSDKLIARKKDWDPKKYGGFNSPTVAYSVLVV
AKVEKGKSKKLKSVKELLGITIMERSSFEKNPIDFLEAKGYKEVKKDLIIKLPKYSLFE
LENGRKRMLASAGVLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTINRKQYNTTKEVLDATLIRQSITGLYETRIDLSQLGGD

PE6c-NRCH comprises the amino acid sequence:

(SEQ ID NO: 356)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRDK
PIREQAENIIHLFTLTNLGAPAAFKYFDTTINRKQYNTTKEVLDATLIRQSITGLYETRI
DLSQLGGDSGGSSGGSKRTADGSEFESPKKKRKVSGGSSGGSISSSKHTLSQMNKVS
NIVKEPELPDIYKEFKDITADTNTEKLPKPIKGLEFEVELTQENYRLPIRNYPLTPVKM
QAMNDEINQGLKGGIIRESKAINACPVIFVPRKEGTLRMVVDYRPLNKYVKPNVYPL
PLIEQLLAKIQGSTIFTKLDLKSAYHQIRVRKGDEHKLAFRCPRGVFEYLVMPYGIKT
APAHFQYFINTILGEAKESHVVCYMDDILIHSKSESEHVKHVKDVLQKLKNANLIINQ
AKCEFHQSQVKFLGYHISEKGLTPCQENIDKVLQWKQPKNQKELRQFLGQVNYLRK
FIPKTSQLTHPLNKLLKKDVRWKWTPTQTQAIENIKQCLVSPPVLRHFDFSKKILLET
DVSDVAVGAVLSQKHDDDKYYPVGYYSAKMSKAQLNYSVSDKEMLAIIKSLEHWR
HYLESTIEPFKILTDHRNLIGRITNESEPENKRLARWQLFLQDFNFEINYRPGSANHIAD
ALSRIVDETEPIPKDNEDNSINFVNQISIKRTADGSEFESPKKKRKVPAAKRVKLD.

The prime editor “PE6d-NRCH” comprises the reverse transcriptase of PE6d (or the non-truncated version of the MMLV reverse transcriptase variant of PE6d) and the NRCH-Cas9 protein. PE6d-NRCH comprises the amino acid sequence:

(SEQ ID NO: 357)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRDK
PIREQAENIIHLFTLTNLGAPAAFKYFDTTINRKQYNTTKEVLDATLIRQSITGLYETRI
DLSQLGGDSGGSSGGSKRTADGSEFESPKKKRKVSGGSSGGSTLNIEDEYRLHETSKE
PDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPNVPN
PYNLLSGLPPSHQWYTVLDLKDAFFCLRLHPTSQPLFAFEWRDPEMGISGQLTWTRL
PQGFKNSPTLFCEALHRDLADFRIQHPDLILLQYYDDLLLAATSELDCQQGTRALLQT
LGNLGYRASAKKAQICQKQVKYLGYLLKEGQRWLTEARKETVMGQPTPKTPRQLR
EFLGKAGFCRLFIPGFAEMAAPLYPLTKPGTLFNWGPDQQKAYQEIKQALLTAPALG
LPDLTKPFELFVDEKQGYAKGVLTQKLGPWRRPVAYLSKKLDPVAAGWPPCLRMV
AAIAVLTKDAGKLTMGQPLVILAPHAVEALVKQPPDRWLSNARMTHYQALLLDTD
RVQFGPVVALNPATLLPLPEEGLQHNCLDSGGSKRTADGSEFESPKKKRKVPAAKRV
KLD;

or the amino acid sequence (which comprises a C-terminally truncated reverse transcriptase domain):

(SEQ ID NO: 358)
MKRTADGSEFESPKKKRKVDKKYSIGLDIGTNSVGWAVITDEYKVPSKKFKVLGNT
DRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAIL
SARLSKSRKLENLIAQLPGEKKNGLFGNLIALSLGLTPNFKSNFDLAEDAKLQLSKDT
YDDDLDNLLAQIGDQYADLFLAAKNLSDAILLSDILRVNTEITKAPLSASMVKRYDE
HHQDLTLLKALVRQQLPEKYKEIFFDQSKNGYAGYIDGGASQEEFYKFIKPILEKMD
GTEELLVKLKREDLLRKQRTFDNGIIPHQIHLGELHAILRRQGDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRLRYTGWGRLSRKLINGIRD
KQSGKTILDFLKSDGFANRNFMQLIHDDSLTFKEDIQKAQVSGQGDSLHEHIANLAG
SPAIKKGILQTVKVVDELVKVMGGHKPENIVIEMARENQTTQKGQKNSRERMKRIEE
GIKELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKGNSDKLIARKKDWDP
KKYGGFNSPTVAYSVLVVAKVEKGKSKKLKSVKELLGITIMERSSFEKNPIDFLEAK
GYKEVKKDLIIKLPKYSLFELENGRKRMLASAGVLQKGNELALPSKYVNFLYLASHY
EKLKGSPEDNEQKQLFVEQHKHYLDEIIEQISEFSKRVILADANLDKVLSAYNKHRDK
PIREQAENIIHLFTLTNLGAPAAFKYFDTTINRKQYNTTKEVLDATLIRQSITGLYETRI
DLSQLGGDSGGSSGGSKRTADGSEFESPKKKRKVSGGSSGGSTLNIEDEYRLHETSKE
PDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPFVPNP
YNLLSGLPPSHQWYTVLDLKDAFFCLRLHPTSQPLFAFEWRDPEMGISGQLTWTRLP
QGFKNSPTLFNEALHRDLADFRIQHPDLILLQYMDDLLLAATSELDCQQGTRALLQT
LGNLGYRASAKKAQICQKQVKYLGYLLKEGQRWLTEARKETVMGQPTPKTPRQLR
EFLGKAGFCRLFIPGFAEMAAPLYPLTKPGTLFNWGPDQQKAYQEIKQALLTAPALG
LPDLTKPFELFVDEKQGYAKGVLTQKLGPWRRPVAYLSKKLDPVAAGWPPCLRMV
AAIAVLTKDAGKLTMGQPLVILAPHAVEALVKQPPDRWLSNARMTHYQALLLDTD
RVQFGPVVALNPATLLPLPEEGLQHNCLDILAEAHGTRPDLTDQPLPDADHTWYTDG
SSLLQEGQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLSIIHCPGHQK
GHSAEARGNRMADQAARKAAITETPDTSTLLIENSSPSGGSKRTADGSEFESPKKKR
KVPAAKRVKLD.

### PE7

The term “PE7” refers to the PE6 prime editors plus a second strand nicking guide RNA. For example, “PE7a” refers to the PE6a prime editor as provided herein, plus a second strand nicking guide RNA.

### PEmax

As used herein, “PEmax” refers to a prime editing composition comprising 1) a fusion protein comprising a Cas9 protein variant Cas9 (R221K N39K H840A) and a variant MMLV RT having the following structure: [bipartite NLS]-[Cas9 (R221K) (N394K) (H840A)]-[linker]-[MMLV_RT (D200N) (T330P) (L603W)]-[bipartite NLS]-[NLS] and 2) a desired PEgRNA, wherein the fusion protein (referred to as the PEmax protein) has the following amino acid sequence:

(SEQ ID NO: 350)
MKRTADGSEFESPKKKRKVDKKYSIGLDIGTNSVGWAVITDEYKVPSKKFKVLG
NTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPI
NASGVDAKAILSARLSKSRKLENLIAQLPGEKKNGLFGNLIALSLGLTPNFKSNF
DLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYID
GGASQEEFYKFIKPILEKMDGTEELLVKLKREDLLRKQRTFDNGSIPHQIHLGE
LHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEIS
GVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTIDRKRYT
STKEVLDATLIHQSITGLYETRIDLSQLGGDSGGSSGGSKRTADGSEFESPKKKR
KVSGGSSGGSTLNIEDEYRLHETSKEPDVSLGSTWLSDFPQAWAETGGMGLAVRQA
PLIIPLKATSTPVSIKQYPMSQEARLGIKPHIQRLLDQGILVPCQSPWNTPLLPVKKPGT
NDYRPVQDLREVNKRVEDIHPTVPNPYNLLSGLPPSHQWYTVLDLKDAFFCLRLHPT
SQPLFAFEWRDPEMGISGQLTWTRLPQGFKNSPTLFNEALHRDLADFRIQHPDLILLQ
YVDDLLLAATSELDCQQGTRALLQTLGNLGYRASAKKAQICQKQVKYLGYLLKEG
QRWLTEARKETVMGQPTPKTPRQLREFLGKAGFCRLFIPGFAEMAAPLYPLTKPGTL
FNWGPDQQKAYQEIKQALLTAPALGLPDLTKPFELFVDEKQGYAKGVLTQKLGPWR
RPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAKALPAGT
SAQRAELIALTQALKMAEGKKLNVYTDSRYAFATAHIHGEIYRRRGWLTSEGKEIKN
KDEILALLKALFLPKRLSIIHCPGHQKGHSAEARGNRMADQAARKAAITETPDTSTLL
IENSSPSGGSKRTADGSEFESPKKKRKVGSGPAAKRVKLD

KEY:
BIPARTITE SV40 NUCLEAR LOCALIZATION SEQUENCE (NLS),
CAS9(R221K N39K H840A)
SGGSx2-BIPARTITE SV40NLS-SGGSx2 LINKER
M-MLV reverse transcriptase (D200N T306K W313F T330P L603W)
Other linker sequence
BIPARTITE SV40NLS
Other linker sequence
c-Myc NLS

In some embodiments, a PEmax protein is a PE4max protein. In some embodiments, a PE4max protein comprises a Cas9 protein that recognizes a non-canonical PAM sequence (e.g., Cas9-NRCH). In some embodiments, PE4max-NRCH comprises the amino acid sequence:

(SEQ ID NO: 359)
MKRTADGSEFESPKKKRKVDKKYSIGLDIGTNSVGWAVITDEYKVPSKKFK
VLGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEENPINASGVD
AKAILSARLSKSRKLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMVK
RYDEHHQDLTLLKALVRQQLPEKYKEIFFDQSKNGYAGYIDGGASQEEFYKFIKPILE
KMDGTEELLVKLKREDLLRKQRTFDNGIIPHQIHLGELHAILRRQGDFYPFLKDNREK
IEKILTFRIPYYVGPLARGNSRFAWMTRKSEETITPWNFEEVVDKGASAQSFIERMTN
FDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANL
AGSPAIKKGILQTVKVVDELVKVMGGHKPENIVIEMARENQTTQKGQKNSRERMKR
IEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGE
TGEIVWDKGRDFATVRKVLSMPQVNIVKKTEVQTGGFSKESILPKGNSDKLIARKKD
WDPKKYGGFNSPTVAYSVLVVAKVEKGKSKKLKSVKELLGITIMERSSFEKNPIDFL
EAKGYKEVKKDLIIKLPKYSLFELENGRKRMLASAGVLQKGNELALPSKYVNFLYLA
SHYEKLKGSPEDNEQKQLFVEQHKHYLDEIIEQISEFSKRVILADANLDKVLSAYNKH
RDKPIREQAENIIHLFTLTNLGAPAAFKYFDTTINRKQYNTTKEVLDATLIRQSITGLY
ETRIDLSQLGGDSGGSSGGSKRTADGSEFESPKKKRKVSGGSSGGSTLNIEDEYRLHE
TSKEPDVSLGSTWLSDFPQAWAETGGMGLAVRQAPLIIPLKATSTPVSIKQYPMSQE
ARLGIKPHIQRLLDQGILVPCQSPWNTPLLPVKKPGTNDYRPVQDLREVNKRVEDIHP
TVPNPYNLLSGLPPSHQWYTVLDLKDAFFCLRLHPTSQPLFAFEWRDPEMGISGQLT
WTRLPQGFKNSPTLFNEALHRDLADFRIQHPDLILLQYVDDLLLAATSELDCQQGTR
ALLQTLGNLGYRASAKKAQICQKQVKYLGYLLKEGQRWLTEARKETVMGQPTPKT
PRQLREFLGKAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLDPVAAGWPPC
LRMVAAIAVLTKDAGKLTMGQPLVILAPHAVEALVKQPPDRWLSNARMTHYQALL
LDTDRVQFGPVVALNPATLLPLPEEGLQHNCLDILAEAHGTRPDLTDQPLPDADHTW
YTDGSSLLQEGQRKAGAAVTTETEVIWAKALPAGTSAQRAELIALTQALKMAEGKK
LNVYTDSRYAFATAHIHGEIYRRRGWLTSEGKEIKNKDEILALLKALFLPKRLSIIHCP
GHQKGHSAEARGNRMADQAARKAAITETPDTSTLLIENSSPKRTADGSEFESPKKKR
KVPAAKRVKLD;

or the amino acid sequence (which comprises a C-terminally truncated reverse transcriptase domain):

PEmaxΔRNaseH-NRCH:

(SEQ ID NO: 360)
MKRTADGSEFESPKKKRKVDKKYSIGLDIGTNSVGWAVITDEYKVPSKKFKV
LGNTDRHSIKKNLIGALLFDSGETAEATRLKRTARRRYTRRKNRICYLQEIFSNEMAK
VDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDA
KAILSARLSKSRKLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMVKR
YDEHHQDLTLLKALVRQQLPEKYKEIFFDQSKNGYAGYIDGGASQEEFYKFIKPILEK
MDGTEELLVKLKREDLLRKQRTFDNGIIPHQIHLGELHAILRRQGDFYPFLKDNREKI
EKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANL
AGSPAIKKGILQTVKVVDELVKVMGGHKPENIVIEMARENQTTQKGQKNSRERMKR
IEEGIKELGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGE
TGEIVWDKGRDFATVRKVLSMPQVNIVKKTEVQTGGFSKESILPKGNSDKLIARKKD
WDPKKYGGFNSPTVAYSVLVVAKVEKGKSKKLKSVKELLGITIMERSSFEKNPIDFL
EAKGYKEVKKDLIIKLPKYSLFELENGRKRMLASAGVLQKGNELALPSKYVNFLYLA
SHYEKLKGSPEDNEQKQLFVEQHKHYLDEIIEQISEFSKRVILADANLDKVLSAYNKH
RDKPIREQAENIIHLFTLTNLGAPAAFKYFDTTINRKQYNTTKEVLDATLIRQSITGLY
ETRIDLSQLGGDSGGSSGGSKRTADGSEFESPKKKRKVSGGSSGGSTLNIEDEYRLHE
TSKEPDVSLGSTWLSDFPQAWAETGGMGLAVRQAPLIIPLKATSTPVSIKQYPMSQE
ARLGIKPHIQRLLDQGILVPCQSPWNTPLLPVKKPGTNDYRPVQDLREVNKRVEDIHP
TVPNPYNLLSGLPPSHQWYTVLDLKDAFFCLRLHPTSQPLFAFEWRDPEMGISGQLT
WTRLPQGFKNSPTLFNEALHRDLADFRIQHPDLILLQYVDDLLLAATSELDCQQGTR
ALLQTLGNLGYRASAKKAQICQKQVKYLGYLLKEGQRWLTEARKETVMGQPTPKT
PRQLREFLGKAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLDPVAAGWPPC
LRMVAAIAVLTKDAGKLTMGQPLVILAPHAVEALVKQPPDRWLSNARMTHYQALL
LDTDRVQFGPVVALNPATLLPLPEEGLQHNCLDKRTADGSEFESPKKKRKVPAAKR
VKLD

In some embodiments, the prime editor is PE2, PE3, PE4, PE5, or a variant thereof (including PE2max, PE3max, PE4max, and PE5max). In certain embodiments, the prime editor is PE4max-NRCH, or a variant thereof. In some embodiments, the prime editor is a PE6 prime editor, or a variant thereof. In certain embodiments, the prime editor is PE6b-NRCH, PE6c-NRCH, PE6d-NRCH, or a variant thereof.

Additional prime editors known in the art may also be used in the present disclosure. Such prime editors include, but are not limited to, the following:

### Other Prime Editors

A prime editor also includes any modified prime editor that is available to the public, e.g., as published in scientific literature or in patent literature.

Prime editors and/or pegRNAs are described in the following literature and may be implemented in the disclosed methods and compositions. The content of each reference is incorporated herein by reference.

| | Date | | | | Issue | Start | End |
|---|---|---|---|---|---|---|---|
| Title | Published | Source Title | Author/s | Volume | Number | Page | Page |
| Search-and-replace | 209 | Nature | Anzalone, A. V., | 576 | | 149 | 157 |
| genome editing | | | Randolph, P. B., | | | | |
| without double-strand | | | Davis, J. R. et al. | | | | |
| breaks or donor DNA | | | | | | | |
| Prime genome editing | Mar. 16, 2020 | Nature | Qiupeng Lin; Yuan | 38 | 5 | 582 | 585 |
| in rice and wheat | | Biotechnology | Zong; Chenxiao | | | | |
| | | | Xue; Shengxing | | | | |
| | | | Wang; Shuai Jin; | | | | |
| | | | Zixu Zhu; | | | | |
| | | | Yanpeng Wang; | | | | |
| | | | Andrew V. | | | | |
| | | | Anzalone; Aditya | | | | |
| | | | Raguram; Jordan | | | | |
| | | | L. Doman; David | | | | |
| | | | R. Liu; Caixia Gao | | | | |
| Author Correction: | Jan. 25, 2024 | Nature | Martina Fiumara; | 42 | 6 | 986 | 986 |
| Genotoxic effects of | | Biotechnology | Samuele Ferrari; | | | | |
| base and prime editing | | | Attya Omer-Javed; | | | | |
| in human | | | Stefano Beretta; | | | | |
| hematopoietic stem | | | Luisa Albano; | | | | |
| cells. | | | Daniele Canarutto; | | | | |
| | | | Angelica Varesi; | | | | |
| | | | Chiara Gaddoni; | | | | |
| | | | Chiara Brombin; | | | | |
| | | | Federica Cugnata; | | | | |
| | | | Erika Zonari; | | | | |
| | | | Matteo Maria | | | | |
| | | | Naldini; Matteo | | | | |
| | | | Barcella; Bernhard | | | | |
| | | | Gentner; Ivan | | | | |
| | | | Merelli; Luigi | | | | |
| | | | Naldini | | | | |
| Cryo-EM structure of | Jul. 17, 2024 | Nature | Iris Marchal | 42 | 7 | 1027 | 1027 |
| a prime editor | | Biotechnology | | | | | |
| complex in multiple | | | | | | | |
| states. | | | | | | | |
| CRISPR prime editing | Apr. 29, 2021 | Nature | Karl Petri; Weiting | 40 | 2 | 1 | 5 |
| with ribonucleoprotein | | Biotechnology | Zhang; Junyan Ma; | | | | |
| complexes in zebrafish | | | Andrea Schmidts; | | | | |
| and primary human | | | Hyunho Lee; Joy | | | | |
| cells | | | E. Horng; Daniel | | | | |
| | | | Y. Kim; Ibrahim C. | | | | |
| | | | Kurt; Kendell | | | | |
| | | | Clement; Jonathan | | | | |
| | | | Y. Hsu; Luca | | | | |
| | | | Pinello; Marcela V. | | | | |
| | | | Maus; J. Keith | | | | |
| | | | Joung; Jing-Ruey | | | | |
| | | | J. Yeh | | | | |
| Prime editing deal | Apr. 17, 2024 | Nature | Carrie Arnold | 42 | 4 | 542 | 544 |
| flurry to nail down | | Biotechnology | | | | | |
| patent rights. | | | | | | | |
| FDA clears prime | May 17, 2024 | Nature | | 42 | 5 | 691 | 691 |
| editors for testing in | | Biotechnology | | | | | |
| humans. | | | | | | | |
| High-efficiency prime | Mar. 25, 2021 | Nature | Qiupeng Lin; | 39 | 8 | 923 | 927 |
| editing with | | biotechnology | Shuai Jin; Yuan | | | | |
| optimized, paired | | | Zong; Hong Yu; | | | | |
| pegRNAs in plants. | | | Zixu Zhu; | | | | |
| | | | Guanwen Liu; | | | | |
| | | | Liquan Kou; | | | | |
| | | | Yanpeng Wang; | | | | |
| | | | Jin-Long Qiu; | | | | |
| | | | Jiayang Li; Caixia | | | | |
| | | | Gao | | | | |
| Predicting prime | Jan. 16, 2023 | Nature | Nicolas Mathis; | 41 | 8 | 1151 | 1159 |
| editing efficiency and | | biotechnology | Ahmed Allam; | | | | |
| product purity by deep | | | Lucas Kissling; | | | | |
| learning. | | | Kim Fabiano | | | | |
| | | | Marquart; Lukas | | | | |
| | | | Schmidheini; | | | | |
| | | | Cristina Solari; | | | | |
| | | | Zsolt Balázs; | | | | |
| | | | Michael | | | | |
| | | | Krauthammer; | | | | |
| | | | Gerald Schwank | | | | |
| Author Correction: | Feb. 8, 2024 | Nature | Hui Kwon Kim; | 42 | 3 | 529 | 529 |
| Predicting the | | biotechnology | Goosang Yu; | | | | |
| efficiency of prime | | | Jinman Park; | | | | |
| editing guide RNAs in | | | Seonwoo Min; | | | | |
| human cells. | | | Sungtae Lee; | | | | |
| | | | Sungroh Yoon; | | | | |
| | | | Hyongbum Henry | | | | |
| | | | Kim | | | | |
| Genomic and | Mar. 14, 2022 | The CRISPR | Runze Gao; Zhi- | 5 | 2 | 276 | 293 |
| Transcriptomic | | journal | Can Fu; Xiangyang | | | | |
| Analyses of Prime | | | Li; Ying Wang; Jia | | | | |
| Editing Guide RNA- | | | Wei; Guangye Li; | | | | |
| Independent Off- | | | Lijie Wang; Jing | | | | |
| Target Effects by | | | Wu; Xingxu | | | | |
| Prime Editors. | | | Huang; Li Yang; | | | | |
| | | | Jia Chen | | | | |
| A prime editor mouse | May 11, 2023 | Nature | Zackery A Ely; | 42 | 3 | 424 | |
| to model a broad | | biotechnology | Nicolas Mathey- | | | | |
| spectrum of somatic | | | Andrews; Santiago | | | | |
| mutations in vivo. | | | Naranjo; Samuel I | | | | |
| | | | Gould; Kim L | | | | |
| | | | Mercer; Gregory A | | | | |
| | | | Newby; Christina | | | | |
| | | | M Cabana; | | | | |
| | | | William M | | | | |
| | | | Rideout; Grissel | | | | |
| | | | Cervantes | | | | |
| | | | Jaramillo; Jennifer | | | | |
| | | | M Khirallah; Katie | | | | |
| | | | Holland; Peyton B | | | | |
| | | | Randolph; William | | | | |
| | | | A Freed-Pastor; | | | | |
| | | | Jessie R Davis; | | | | |
| | | | Zachary Kulstad; | | | | |
| | | | Peter M K | | | | |
| | | | Westcott; Lin Lin; | | | | |
| | | | Andrew V | | | | |
| | | | Anzalone; Brendan | | | | |
| | | | L Horton; Nimisha | | | | |
| | | | B Pattada; Sean- | | | | |
| | | | Luc Shanahan; | | | | |
| | | | Zhongfeng Ye; | | | | |
| | | | Stefani Spranger; | | | | |
| | | | Qiaobing Xu; | | | | |
| | | | Francisco J | | | | |
| | | | Sánchez-Rivera; | | | | |
| | | | David R Liu; Tyler | | | | |
| | | | Jacks | | | | |
| Author Correction: An | Apr. 8, 2022 | Nature | Yuan Zong; Yijing | 40 | 9 | 1412 | 1412 |
| engineered prime | | biotechnology | Liu; Chenxiao | | | | |
| editor with enhanced | | | Xue; Boshu Li; | | | | |
| editing efficiency in | | | Xiangyang Li; | | | | |
| plants. | | | Yanpeng Wang; Ji | | | | |
| | | | Li; Guanwen Liu; | | | | |
| | | | Xingxu Huang; | | | | |
| | | | Xiaofeng Cao; | | | | |
| | | | Caixia Gao | | | | |
| Predicting the | Sep. 21, 2020 | Nature | Hui Kwon Kim; | 39 | 2 | 198 | 206 |
| efficiency of prime | | biotechnology | Goosang Yu; | | | | |
| editing guide RNAs in | | | Jinman Park; | | | | |
| human cells. | | | Seonwoo Min; | | | | |
| | | | Sungtae Lee; | | | | |
| | | | Sungroh Yoon; | | | | |
| | | | Hyongbum Kim | | | | |
| Author Correction: | May 13, 2021 | Nature | Karl Petri; Weiting | 40 | 2 | 1 | 1 |
| CRISPR prime editing | | biotechnology | Zhang; Junyan Ma; | | | | |
| with ribonucleoprotein | | | Andrea Schmidts; | | | | |
| complexes in zebrafish | | | Hyunho Lee; Joy | | | | |
| and primary human | | | E. Horng; Daniel | | | | |
| cells. | | | Y. Kim; Ibrahim C. | | | | |
| | | | Kurt; Kendell | | | | |
| | | | Clement; Jonathan | | | | |
| | | | Y. Hsu; Luca | | | | |
| | | | Pinello; Marcela V. | | | | |
| | | | Maus; J. Keith | | | | |
| | | | Joung; Jing-Ruey | | | | |
| | | | J. Yeh | | | | |
| Insertion of the | Feb. 7, 2022 | The CRISPR | Guillaume | 5 | 1 | 109 | 122 |
| Icelandic Mutation | | journal | Tremblay; Joël | | | | |
| (A673T) by Prime | | | Rousseau; Cedric | | | | |
| Editing: A Potential | | | Happi Mbakam; | | | | |
| Preventive Treatment | | | Jacques P | | | | |
| for Familial and | | | Tremblay | | | | |
| Sporadic Alzheimer's | | | | | | | |
| Disease. | | | | | | | |
| Gene editing enters | Nov. 7, 2019 | Nature | Cormac Sheridan | | | | |
| ‘prime’ time | | biotechnology | | | | | |
| Machine learning | Jun. 21, 2024 | Nature | Nicolas Mathis; | | | | |
| prediction of prime | | biotechnology | Ahmed Allam; | | | | |
| editing efficiency | | | András Tálas; | | | | |
| across diverse | | | Lucas Kissling; | | | | |
| chromatin contexts. | | | Elena Benvenuto; | | | | |
| | | | Lukas | | | | |
| | | | Schmidheini; | | | | |
| | | | Ruben Schep; | | | | |
| | | | Tanav | | | | |
| | | | Damodharan; Zsolt | | | | |
| | | | Balázs; Sharan | | | | |
| | | | Janjuha; Eleonora I | | | | |
| | | | Ioannidi; Desirée | | | | |
| | | | Böck; Bas van | | | | |
| | | | Steensel; Michael | | | | |
| | | | Krauthammer; | | | | |
| | | | Gerald Schwank | | | | |
| An engineered prime | Mar. 24, 2022 | Nature | Yuan Zong; Yijing | 40 | 9 | 1394 | 1402 |
| editor with enhanced | | biotechnology | Liu; Chenxiao | | | | |
| editing efficiency in | | | Xue; Boshu Li; | | | | |
| plants. | | | Xiangyang Li; | | | | |
| | | | Yanpeng Wang; Ji | | | | |
| | | | Li; Guanwen Liu; | | | | |
| | | | Xingxu Huang; | | | | |
| | | | Xiaofeng Cao; | | | | |
| | | | Caixia Gao | | | | |
| Genome-wide | Apr. 15, 2021 | Nature | Shuai Jin; Qiupeng | 39 | 10 | 1292 | 1299 |
| specificity of prime | | biotechnology | Lin; Yingfeng Luo; | | | | |
| editors in plants. | | | Zixu Zhu; | | | | |
| | | | Guanwen Liu; | | | | |
| | | | Yunjia Li; Kunling | | | | |
| | | | Chen; Jin-Long | | | | |
| | | | Qiu; Caixia Gao | | | | |
| Prime Editing in | Dec. 1, 2022 | The CRISPR | Dawei Wang; | 5 | 6 | 746 | 768 |
| Mammals: The Next | | journal | Xiude Fan; | | | | |
| Generation of | | | Mengzhu Li; | | | | |
| Precision Genome | | | Tianbo Liu; Peng | | | | |
| Editing. | | | Lu; Guangxin | | | | |
| | | | Wang; Yuan Li; | | | | |
| | | | JunMing Han; | | | | |
| | | | JiaJun Zhao | | | | |
| Genome editing with | Jun. 22, 2020 | Nature | Andrew V. | 38 | 7 | 824 | 844 |
| CRISPR-Cas | | biotechnology | Anzalone; Luke W. | | | | |
| nucleases, base | | | Koblan; David R. | | | | |
| editors, transposases | | | Liu | | | | |
| and prime editors | | | | | | | |
| Rapid generation of | Sep. 30, 2024 | Nature | Xinlin Lei; Anhui | | | | |
| long, chemically | | biotechnology | Huang; Didi Chen; | | | | |
| modified pegRNAs for | | | Xuebin Wang; | | | | |
| prime editing. | | | Ruijin Ji; Jinlin | | | | |
| | | | Wang; Yizhou | | | | |
| | | | Zhang; Yuming | | | | |
| | | | Zhang; Shuhan Lu; | | | | |
| | | | Kun Zhang; | | | | |
| | | | Qiubing Chen; | | | | |
| | | | Ying Zhang; Hao | | | | |
| | | | Yin | | | | |
| Predicting Mutations | Jun. 20, 2023 | The CRISPR | Juliane Weller; | 6 | 4 | 325 | 338 |
| Generated by Cas9, | | journal | Ananth Pallaseni; | | | | |
| Base Editing, and | | | Jonas Koeppel; | | | | |
| Prime Editing in | | | Leopold Parts | | | | |
| Mammalian Cells. | | | | | | | |
| Prediction of prime | Feb. 16, 2023 | Nature | Jonas Koeppel; | 41 | 10 | 1446 | 1456 |
| editing insertion | | biotechnology | Juliane Weller; | | | | |
| efficiencies using | | | Elin Madli Peets; | | | | |
| sequence features and | | | Ananth Pallaseni; | | | | |
| DNA repair | | | Ivan Kuzmin; Uku | | | | |
| determinants. | | | Raudvere; Hedi | | | | |
| | | | Peterson; Fabio | | | | |
| | | | Giuseppe | | | | |
| | | | Liberante; Leopold | | | | |
| | | | Parts | | | | |
| Genotoxicity concerns | Sep. 7, 2023 | Nature | | 42 | 6 | 850 | 851 |
| for base and prime | | biotechnology | | | | | |
| editors in | | | | | | | |
| hematopoietic stem | | | | | | | |
| cells. | | | | | | | |
| Engineered CRISPR | Sep. 26, 2022 | Nature | Julian Grünewald; | 41 | 3 | 337 | 343 |
| prime editors with | | biotechnology | Bret R Miller; | | | | |
| compact, untethered | | | Regan N Szalay; | | | | |
| reverse transcriptases. | | | Peter K | | | | |
| | | | Cabeceiras; | | | | |
| | | | Christopher J | | | | |
| | | | Woodilla; Eliza | | | | |
| | | | Jane B Holtz; Karl | | | | |
| | | | Petri; J Keith | | | | |
| | | | Joung | | | | |
| Deletion and | Oct. 14, 2021 | Nature | Tingting Jiang; | 40 | 2 | 1 | 8 |
| replacement of long | | biotechnology | Xiao-Ou Zhang; | | | | |
| genomic sequences | | | Zhiping Weng; | | | | |
| using prime editing. | | | Wen Xue | | | | |
| Programmable large | Dec. 9, 2021 | Nature | Andrew V. | 40 | 5 | 731 | 740 |
| DNA deletion, | | biotechnology | Anzalone; Xin D. | | | | |
| replacement, | | | Gao; Christopher J. | | | | |
| integration, and | | | Podracky; Andrew | | | | |
| inversion with twin | | | T. Nelson; Luke | | | | |
| prime editing and site- | | | W. Koblan; Aditya | | | | |
| specific recombinases | | | Raguram; Jonathan | | | | |
| | | | M. Levy; Jaron A. | | | | |
| | | | M. Mercer; David | | | | |
| | | | R. Liu | | | | |
| Genotoxic effects of | Sep. 7, 2023 | Nature | Martina Fiumara; | 42 | 6 | 877 | 891 |
| base and prime editing | | biotechnology | Samuele Ferrari; | | | | |
| in human | | | Attya Omer-Javed; | | | | |
| hematopoietic stem | | | Stefano Beretta; | | | | |
| cells. | | | Luisa Albano; | | | | |
| | | | Daniele Canarutto; | | | | |
| | | | Angelica Varesi; | | | | |
| | | | Chiara Gaddoni; | | | | |
| | | | Chiara Brombin; | | | | |
| | | | Federica Cugnata; | | | | |
| | | | Erika Zonari; | | | | |
| | | | Matteo Maria | | | | |
| | | | Naldini; Matteo | | | | |
| | | | Barcella; Bernhard | | | | |
| | | | Gentner; Ivan | | | | |
| | | | Merelli; Luigi | | | | |
| | | | Naldini | | | | |
| Engineered pegRNAs | Oct. 4, 2021 | Nature | James W. Nelson; | 40 | 3 | 1 | 9 |
| improve prime editing | | biotechnology | Peyton B. | | | | |
| efficiency | | | Randolph; Simon | | | | |
| | | | P. Shen; Kelcee A. | | | | |
| | | | Everette; Peter J. | | | | |
| | | | Chen; Andrew V. | | | | |
| | | | Anzalone; Meirui | | | | |
| | | | An; Gregory A. | | | | |
| | | | Newby; Jonathan | | | | |
| | | | C. Chen; Alvin | | | | |
| | | | Hsu; David R. Liu | | | | |
| Saturation variant | Feb. 21, 2022 | Nature | Steven Erwood; | 40 | 6 | 885 | 895 |
| interpretation using | | biotechnology | Teija M I Bily; | | | | |
| CRISPR prime | | | Jason Lequyer; | | | | |
| editing. | | | Joyce Yan; Nitya | | | | |
| | | | Gulati; Reid A | | | | |
| | | | Brewer; Liangchi | | | | |
| | | | Zhou; Laurence | | | | |
| | | | Pelletier; Evgueni | | | | |
| | | | A Ivakine; Ronald | | | | |
| | | | D Cohn | | | | |
| Prime editing. | Apr. 17, 2024 | Nature | | 42 | 4 | 570 | 570 |
| | | biotechnology | | | | | |
| Increasing | Sep. 25, 2024 | Nature | Pengpeng Liu; | | | | |
| intracellular dNTP | | biotechnology | Karthikeyan | | | | |
| levels improves prime | | | Ponnienselvan; | | | | |
| editing efficiency. | | | Thomas Nyalile; | | | | |
| | | | Sarah Oikemus; | | | | |
| | | | Anya T Joynt; | | | | |
| | | | Sukanya Iyer; | | | | |
| | | | Karen Kelly; | | | | |
| | | | Dongsheng Guo; | | | | |
| | | | Pyae P Kyawe; | | | | |
| | | | Emma | | | | |
| | | | Vanderleeden; | | | | |
| | | | Sambra D Redick; | | | | |
| | | | Lei Huang; | | | | |
| | | | Zexiang Chen; | | | | |
| | | | Jeong Min Lee; | | | | |
| | | | Celia A Schiffer; | | | | |
| | | | David M Harlan; | | | | |
| | | | Jennifer P Wang; | | | | |
| | | | Charles P | | | | |
| | | | Emerson; Nathan | | | | |
| | | | D Lawson; | | | | |
| | | | Jonathan K Watts; | | | | |
| | | | Erik J Sontheimer; | | | | |
| | | | Jeremy Luban; | | | | |
| | | | Scot A Wolfe | | | | |
| Author Correction: | Dec. 8, 2021 | Nature | James W Nelson; | 40 | 3 | 432 | 432 |
| Engineered pegRNAs | | biotechnology | Peyton B | | | | |
| improve prime editing | | | Randolph; Simon P | | | | |
| efficiency. | | | Shen; Kelcee A | | | | |
| | | | Everette; Peter J | | | | |
| | | | Chen; Andrew V | | | | |
| | | | Anzalone; Meirui | | | | |
| | | | An; Gregory A | | | | |
| | | | Newby; Jonathan | | | | |
| | | | C Chen; Alvin | | | | |
| | | | Hsu; David R Liu | | | | |
| Author Correction: | Feb. 8, 2024 | Nature | Ronghong Liang; | | | | |
| Prime editing using | | biotechnology | Zixin He; Kevin | | | | |
| CRISPR-Cas12a and | | | Tianmeng Zhao; | | | | |
| circular RNAs in | | | Haocheng Zhu; | | | | |
| human cells. | | | Jiacheng Hu; | | | | |
| | | | Guanwen Liu; | | | | |
| | | | Qiang Gao; | | | | |
| | | | Meiyan Liu; Rui | | | | |
| | | | Zhang; Jin-Long | | | | |
| | | | Qiu; Caixia Gao | | | | |
| A split prime editor | Apr. 4, 2022 | Nature | Bin Liu; Xiaolong | 40 | 9 | 1388 | 1393 |
| with untethered | | biotechnology | Dong; Haoyang | | | | |
| reverse transcriptase | | | Cheng; Chunwei | | | | |
| and circular RNA | | | Zheng; Zexiang | | | | |
| template. | | | Chen; Tomás C | | | | |
| | | | Rodríguez; Shun- | | | | |
| | | | Qing Liang; Wen | | | | |
| | | | Xue; Erik J | | | | |
| | | | Sontheimer | | | | |
| Generation of Human | Feb. 1, 2024 | The CRISPR | Lori L | 7 | 1 | 53 | 67 |
| Isogenic Induced | | journal | Bonnycastle; Amy | | | | |
| Pluripotent Stem Cell | | | J Swift; Erin C | | | | |
| Lines with CRISPR | | | Mansell; Angela | | | | |
| Prime Editing. | | | Lee; Elizabeth | | | | |
| | | | Winnicki; | | | | |
| | | | Elizabeth S Li; | | | | |
| | | | Catherine C | | | | |
| | | | Robertson; | | | | |
| | | | Victoria A | | | | |
| | | | Parsons; Trung | | | | |
| | | | Huynh; Chad | | | | |
| | | | Krilow; Karen L | | | | |
| | | | Mohlke; Michael R | | | | |
| | | | Erdos; Narisu | | | | |
| | | | Narisu; Francis S | | | | |
| | | | Collins | | | | |
| Prime Editing: Making | Oct. 1, 2020 | The CRISPR | Svetlana A | 3 | 5 | 319 | 321 |
| the Move to Prime | | journal | Smirnikhina | | | | |
| Time. | | | | | | | |
| Precise genomic | Oct. 14, 2021 | Nature | Junhong Choi; Wei | 40 | 2 | 1 | 9 |
| deletions using paired | | biotechnology | Chen; Chase C. | | | | |
| prime editing. | | | Suiter; Choli Lee; | | | | |
| | | | Florence M. | | | | |
| | | | Chardon; Wei | | | | |
| | | | Yang; Anh Leith; | | | | |
| | | | Riza M. Daza; | | | | |
| | | | Beth Martin; Jay | | | | |
| | | | Shendure | | | | |
| Efficient prime editing | May 4, 2023 | Nature | Jessie R Davis; | 42 | 2 | 253 | 264 |
| in mouse brain, liver | | biotechnology | Samagya | | | | |
| and heart with dual | | | Banskota; Jonathan | | | | |
| AAVs. | | | M Levy; Gregory | | | | |
| | | | A Newby; Xiao | | | | |
| | | | Wang; Andrew V | | | | |
| | | | Anzalone; Andrew | | | | |
| | | | T Nelson; Peter J | | | | |
| | | | Chen; Andrew D | | | | |
| | | | Hennes; Meirui | | | | |
| | | | An; Heejin Roh; | | | | |
| | | | Peyton B | | | | |
| | | | Randolph; Kiran | | | | |
| Efficient prime editing | Feb. 6, 2024 | Nature | Musunuru; David | | | | |
| in two-cell mouse | | biotechnology | R Liu | | | | |
| embryos using | | | Rebecca P Kim- | | | | |
| PEmbryo. | | | Yip; Ryan | | | | |
| | | | McNulty; Bradley | | | | |
| | | | Joyce; Antonio | | | | |
| | | | Mollica; Peter J | | | | |
| | | | Chen; Purnima | | | | |
| | | | Ravisankar; | | | | |
| | | | Benjamin K Law; | | | | |
| | | | David R Liu; Jared | | | | |
| | | | E Toettcher; | | | | |
| | | | Evgueni A Ivakine; | | | | |
| | | | Eszter Posfai; Britt | | | | |
| | | | Adamson | | | | |
| Correction to: A | Apr. 1, 2024 | The CRISPR | | 7 | 2 | 132 | 132 |
| Fluorescent Reporter | | journal | | | | | |
| Mouse for In Vivo | | | | | | | |
| Assessment of | | | | | | | |
| Genome Editing with | | | | | | | |
| Diverse Cas Nucleases | | | | | | | |
| and Prime Editors by | | | | | | | |
| Chen et al. The | | | | | | | |
| CRISPR Journal, | | | | | | | |
| 2023; 6(6): 570-582; | | | | | | | |
| DOI: | | | | | | | |
| 10.1089/crispr.2023.0 | | | | | | | |
| 048. | | | | | | | |
| An optimized toolkit | Jan. 29, 2024 | Nature | Ryan M Marks; | 42 | 2 | 187 | 189 |
| for prime editing. | | biotechnology | Ori Scott; Evgueni | | | | |
| | | | A Ivakine; Ronald | | | | |
| | | | D Cohn | | | | |
| Publisher Correction: | Aug. 12, 2024 | Nature | Nicolas Mathis; | | | | |
| Machine learning | | biotechnology | Ahmed Allam; | | | | |
| prediction of prime | | | András Tálas; | | | | |
| editing efficiency | | | Lucas Kissling; | | | | |
| across diverse | | | Elena Benvenuto; | | | | |
| chromatin contexts. | | | Lukas | | | | |
| | | | Schmidheini; | | | | |
| | | | Ruben Schep; | | | | |
| | | | Tanav | | | | |
| | | | Damodharan; Zsolt | | | | |
| | | | Bálazs; Sharan | | | | |
| | | | Janjuha; Eleonora I | | | | |
| | | | Ioannidi; Desirée | | | | |
| | | | Böck; Bas van | | | | |
| | | | Steensel; Michael | | | | |
| | | | Krauthammer; | | | | |
| | | | Gerald Schwank | | | | |
| Prime editing using | Jan. 10, 2024 | Nature | Ronghong Liang; | | | | |
| CRISPR-Cas12a and | | biotechnology | Zixin He; Kevin | | | | |
| circular RNAs in | | | Tianmeng Zhao; | | | | |
| human cells. | | | Haocheng Zhu; | | | | |
| | | | Jiacheng Hu; | | | | |
| | | | Guanwen Liu; | | | | |
| | | | Qiang Gao; | | | | |
| | | | Meiyan Liu; Rui | | | | |
| | | | Zhang; Jin-Long | | | | |
| | | | Qiu; Caixia Gao | | | | |
| Enhancing prime | May 28, 2024 | Nature | Sebastien | | | | |
| editing in | | biotechnology | Levesque; Andrea | | | | |
| hematopoietic stem | | | Cosentino; | | | | |
| and progenitor cells by | | | Archana Verma; | | | | |
| modulating nucleotide | | | Pietro Genovese; | | | | |
| metabolism. | | | Daniel E Bauer | | | | |
| High-throughput | Mar. 12, 2024 | Nature | Samuel I Gould; | | | | |
| evaluation of genetic | | biotechnology | Alexandra N | | | | |
| variants with prime | | | Wuest; Kexin | | | | |
| editing sensor | | | Dong; Grace A | | | | |
| libraries. | | | Johnson; Alvin | | | | |
| | | | Hsu; Varun K | | | | |
| | | | Narendra; Ondine | | | | |
| | | | Atwa; Stuart S | | | | |
| | | | Levine; David R | | | | |
| | | | Liu; Francisco J | | | | |
| | | | Sanchez Rivera | | | | |
| Prime Editing of | Aug. 7, 2024 | The CRISPR | Gaoen Ma; Hui Qi; | 7 | 4 | 188 | 196 |
| Vascular Endothelial | | Journal | Hongwei Deng; | | | | |
| Growth Factor | | | Lijun Dong; Qing | | | | |
| Receptor 2 Attenuates | | | Zhang; Junkai Ma; | | | | |
| Angiogenesis In Vitro. | | | Yanhui Yang; | | | | |
| | | | Xiaohe Yan; | | | | |
| | | | Yajian Duan; | | | | |
| | | | Hetian Lei | | | | |
| Engineered virus-like | Jan. 8, 2024 | Nature | Meirui An; Aditya | | | | |
| particles for transient | | biotechnology | Raguram; Samuel | | | | |
| delivery of prime | | | W Du; Samagya | | | | |
| editor | | | Banskota; Jessie R | | | | |
| ribonucleoprotein | | | Davis; Gregory A | | | | |
| complexes in vivo. | | | Newby; Paul Z | | | | |
| | | | Chen; Krzysztof | | | | |
| | | | Palczewski; David | | | | |
| | | | R Liu | | | | |
| A Fluorescent | Dec. 1, 2023 | The CRISPR | Zexiang Chen; | 6 | 6 | 570 | 582 |
| Reporter Mouse for In | | journal | Suet-Yan Kwan; | | | | |
| Vivo Assessment of | | | Aamir Mir; Max | | | | |
| Genome Editing with | | | Hazeltine; | | | | |
| Diverse Cas Nucleases | | | Minwook Shin; | | | | |
| and Prime Editors. | | | Shun-Qing Liang; | | | | |
| | | | Io Long Chan; | | | | |
| | | | Karen Kelly; | | | | |
| | | | Krishna S Ghanta; | | | | |
| | | | Nicholas Gaston; | | | | |
| | | | Yueying Cao; Jun | | | | |
| | | | Xie; Guangping | | | | |
| | | | Gao; Wen Xue; | | | | |
| | | | Erik J Sontheimer; | | | | |
| | | | Jonathan K Watts | | | | |

In addition, prime editors contemplated herein may include any improved version of a prime editor and/or pegRNA, for example, those described in the following references (each of which is incorporated by reference): (1) Truong et al., “Exonuclease-enhanced prime editors,” Nature Methods, Feb. 1, 2024, 21:455-464 (which describes an exonuclease-recruiting prime editor called Exo-PE which facilitates removal of the 5′ end endogenous flap); (2) Pengpeng Liu et al., “Improved prime editors enable pathogenic allele correction and cancer modelling in adult mice,” Nature Communications, Apr. 9, 2021, 12, 2121 (2021) (which describes a NLS-optimized SpCas9-based prime editor named PE2* that improves genome editing efficiency in both fluorescent reporter cells and at endogenous loci in cultured cell lines); (3) Ferreira da Silva, J., Oliveira, G. P., Arasa-Verge, E. A. et al., Prime editing efficiency and fidelity are enhanced in the absence of mismatch repair. Nat Commun 13, 760 (2022) (which describes cell-based work showing that the ablation of mismatch repair (MMR) affords a 2-17 fold increase in PE efficiency in modified cells); (4) Chen PJ, Hussmann J A, Yan J, Knipping F, Ravisankar P, Chen P F, Chen C, Nelson J W, Newby G A, Sahin M, Osborn M J, Weissman J S, Adamson B, Liu D R. Enhanced prime editing systems by manipulating cellular determinants of editing outcomes. Cell. 2021 Oct. 28; 184 (22): 5635-5652 (which developed PE4 and PE5 prime editing systems in which transient expression of an engineered MMR-inhibiting protein enhances the efficiency of substitution, small insertion, and small deletion prime edits by an average 7.7-fold and 2.0-fold compared to PE2 and PE3 systems, respectively, while improving edit/indel ratios by 3.4-fold in MMR-proficient cell types); (5) Yan, J., Oyler-Castrillo, P., Ravisankar, P. et al. Improving prime editing with an endogenous small RNA-binding protein. Nature 628, 639-647 (2024) (which reports the use of the small RNA-binding exonuclease protection factor La to enhance prime editing); (6) Liu, Y., Yang, G., Huang, S. et al. Enhancing prime editing by Csy4-mediated processing of pegRNA. Cell Res 31, 1134-1136 (2021) (which reports on several modifications to pegRNAs and PE to modify editing efficiency); (7) Qian, Y., Wang, D., Niu, W. et al. Development of a highly efficient prime editor system in mice and rabbits. Cell. Mol. Life Sci. 80, 346 (2023) (which reports on the development of a prime editor variant referred to as ePE3max which contains various modifications to the pegRNA and PE); (8) Zhang, G., Liu, Y., Huang, S. et al. Enhancement of prime editing via xrRNA motif-joined pegRNA. Nat Commun 13, 1856 (2022) (which describes an engineered PE called “xrPE” that is formed by appending a viral exoribonuclease-resistant RNA motif (xrRNA) to the 3′-extended portion of pegRNAs for their increased resistance against degradation); (9) Chauhan, Vikash P. et al. “Engineered prime editors with minimal genomic errors.” bioRxiv (2024): n. pag. (which describes an engineered prime editor called “vPE” which engineers the Cas9 nickase to reposition prime editor nicks to favor the installation of the 3′ new strands); and (10) Liu, Bin et al. “A split prime editor with untethered reverse transcriptase and circular RNA template.” Nature Biotechnology 40 (2022): 1388-1393 (which describes a split PE (sPE) in which the Cas9 nickase (nCas9) remains untethered from the reverse transcriptase (RT) showed similar efficiencies in installing precise edits as the parental unsplit PE3 and no increase in insertion-deletion (indel) byproducts).

Prime editors and/or pegRNA are also described in the following patent literature, each of which is incorporated herein by reference and which may be utilized in accordance with the present disclosure.

| | | Earliest | | | |
|---|---|---|---|---|---|
| | Publication | Priority | | | |
| Display Key | Date | Date | Title | Applicants | Inventors |
| WO 2020/191248 | Sep. 24, 2020 | Mar. 19, 2019 | METHOD AND | BROAD INST | LIU DAVID;; |
| | | | COMPOSITIONS | INC;; HARVARD | ANZALONE ANDREW;; |
| | | | FOR EDITING | COLLEGE | NELSON JAMES |
| | | | NUCLEOTIDE | | |
| | | | SEQUENCES | | |
| WO 2021/072328 | Apr. 15, 2021 | Oct. 10, 2019 | METHODS AND | BROAD INST | LIU DAVID;; |
| A1 | | | COMPOSITIONS | INC;; HARVARD | ANZALONE ANDREW;; |
| | | | FOR PRIME | COLLEGE | NELSON JAMES;; |
| | | | EDITING RNA | | CHEN PETER |
| WO 2021/092204 | May 14, 2021 | Nov. 5, 2019 | METHODS AND | SPOTLIGHT | TAMBE AKSHAY;; |
| A1 | | | COMPOSITIONS | THERAPEUTICS | JAYARAM HARIHARAN;; |
| | | | FOR NUCLEIC | | STRUTT STEVEN |
| | | | ACID-GUIDED | | |
| | | | NUCLEASE CELL | | |
| | | | TARGETING | | |
| | | | SCREEN | | |
| WO 2021/165508 | Aug. 26, 2021 | Feb. 21, 2020 | PRIME EDITING | BIOGEMMAFR | NOGUÉ FABIEN;; |
| A1 | | | TECHNOLOGY | | PERROUD PIERRE-FRANçOIS;; |
| | | | FOR PLANT | | GUYON-DEBAST ANOUCHKA;; |
| | | | GENOME | | PAUL WYATT;; |
| | | | ENGINEERING | | PICHON JEAN-PHILIPPE |
| WO 2021/215827 | Oct. 28, 2021 | Apr. 21, 2020 | GENOME EDITING | INST BASIC | KIM JIN-SOO;; |
| A1 | | | USING CAS9 OR | SCIENCE | LEE HYUNJI;; |
| | | | CAS9 VARIANT | | KANG BEUM-CHANG |
| WO 2021/215897 | Oct. 28, 2021 | Apr. 24, 2020 | GENOME EDITION | INST BASIC | KIM JIN-SOO;; |
| A1 | | | USING CAS9 OR | SCIENCE | LIM KAYEONG;; |
| | | | CAS9 VARIANT | | LEE JAESUK |
| WO 2022/025623 | Feb. 3, 2022 | Jul. 29, 2020 | SYSTEM AND | UNIV YONSEI | KIM HYONG BUM;; |
| A1 | | | METHOD FOR | IACF | KIM HUI KWON;; |
| | | | PRIME EDITING | | YU GOO SANG |
| | | | EFFICIENCY | | |
| | | | PREDICTION | | |
| | | | USING DEEP | | |
| | | | LEARNING | | |
| WO 2022/032085 | Feb. 10, 2022 | Aug. 7, 2020 | TARGETED | JACKSON LAB | CHENG ALBERT;; |
| A1 | | | SEQUENCE | | JILLETTE NATHANIEL |
| | | | INSERTION | | |
| | | | COMPOSITIONS | | |
| | | | AND METHODS | | |
| WO 2022/067130 | Mar. 31, 2022 | Sep. 24, 2020 | PRIME EDITING | BROAD INST | LIU DAVIDR;; |
| A2 | | | GUIDE RNAS, | INC;; HARVARD | NELSON JAMES WILLIAM;; |
| | | | COMPOSITIONS | COLLEGE | RANDOLPH PEYTON BARKSDALE;; |
| | | | THEREOF, AND | | ANZALONE ANDREW VITO;; |
| | | | METHODS OF | | SHEN SIMON;; |
| | | | USING THE SAME | | EVERETTE KELCEE;; |
| | | | | | CHEN PETER J |
| WO 2022/065689 | Mar. 31, 2022 | Sep. 24, 2020 | PRIME EDITING- | UNIV KOREA | KIM KYOUNGMI;; |
| A1 | | | BASED GENE | RES & BUS | PARK SOO JI;; |
| | | | EDITING | FOUND;; SEOUL | JEONG TAE-YEONG;; |
| | | | COMPOSITION | NAT UNIV | SHIN SEUNG KYUN;; |
| | | | WITH ENHANCED | R&DB | SEONG JE KYUNG |
| | | | EDITING | FOUNDATION;; | |
| | | | EFFICIENCY AND | KOREA | |
| | | | USE THEREOF | MOUSE | |
| | | | | PHENOTYPING | |
| | | | | CENTER | |
| WO 2022/071745 | Apr. 7, 2022 | Sep. 29, 2020 | PRIME EDITING | INST BASIC | KIM JIN-SOO;; |
| A1 | | | USING HIV | SCIENCE | PARK CHUL-YONG |
| | | | REVERSE | | |
| | | | TRANSCRIPTASE | | |
| | | | AND CAS9 OR | | |
| | | | VARIANT | | |
| | | | THEREOF | | |
| WO 2022/098765 | May 12, 2022 | Nov. 3, 2020 | SPLIT PRIME | UNIV | PEREZ-PINERA PABLO;; |
| A1 | | | EDITING | ILLINOIS | WOODS WENDY;; |
| | | | PLATFORMS | | WINTER JACKSON;; |
| | | | | | GAPINSKE MICHAEL |
| WO 2022/098885 | May 12, 2022 | Nov. 5, 2020 | PRECISE GENOME | UNIV | SHENDURE JAY ASHOK;; |
| A1 | | | DELETION AND | WASHINGTON | CHEN WEI;; |
| | | | REPLACEMENT | | CHOI JUNHONG |
| | | | METHOD BASED | | |
| | | | ON PRIME | | |
| | | | EDITING | | |
| WO 2022/100662 | May 19, 2022 | Nov. 12, 2020 | GENOMIC EDITING | UNIV | CHEN JIA;; |
| A1 | | | OF IMPROVED | SHANGHAI | YANG BEI;; |
| | | | EFFICIENCY AND | TECHNOLOGY | YANG LI;; |
| | | | ACCURACY | | LI XIAOSA;; |
| | | | | | WANG XIAO;; |
| | | | | | ZHAO WENWEN;; |
| | | | | | ZHOU LINA;; |
| | | | | | LI JIFANG |
| WO 2022/114815 | Jun. 2, 2022 | Nov. 25, 2020 | COMPOSITION | UNIV YONSEI | KIM HYONG BUM;; |
| A1 | | | FOR PRIME | IACF | JANG HYE WON |
| | | | EDITING | | |
| | | | COMPRISING | | |
| | | | TRANS-SPLICING | | |
| | | | ADENO- | | |
| | | | ASSOCIATED | | |
| | | | VIRUS VECTOR | | |
| WO 2022/149166 | Jul. 14, 2022 | Jan. 8, 2021 | A COCKTAIL | CHRISTIAN | THANGAVEL SARAVANABHAVAN;; |
| A1 | | | FORMULATION | MEDICAL | SRIVASTAVA ALOK;; |
| | | | FOR SELECTIVE | COLLEGE; | MAREPALLY SRUJAN |
| | | | ENRICHMENT OF | INST FOR | |
| | | | GENE-MODIFIED | STEM CELL | |
| | | | CELLS | SCIENCE AND | |
| | | | | REGENERATIVE | |
| | | | | MEDICINE | |
| WO 2022/150790 | Jul. 14, 2022 | Jan. 11, 2021 | PRIME EDITOR | BROAD INST | LIU DAVID;; |
| A2 | | | VARIANTS, | INC;; HARVARD | CHEN PETER;; |
| | | | CONSTRUCTS, | COLLEGE;; UNIV | ADAMSON BRITTANY;; |
| | | | AND METHODS | PRINCETON;; | HUSSMANN JEFFREY |
| | | | FOR ENHANCING | UNIV | |
| | | | PRIME EDITING | CALIFORNIA;; | |
| | | | EFFICIENCY AND | LIU DAVID R;; | |
| | | | PRECISION | CHEN PETER J | |
| WO 2022/169235 | Aug. 11, 2022 | Feb. 4, 2021 | PRIME EDITING | UNIV YONSEI | KIM HYONGBUM;; |
| A1 | | | COMPOSITION | IACF | SONG MYUNGJAE;; |
| | | | WITH IMPROVED | | LIM JUNG MIN |
| | | | EDITING | | |
| | | | EFFICIENCY | | |
| WO 2022/170058 | Aug. 11, 2022 | Feb. 5, 2021 | PRIME EDITOR | UNIV | LIANG SHUN-QING;; |
| A1 | | | SYSTEM FOR IN | MASSACHUSETTS | LIU PENGPENG;; |
| | | | VIVO GENOME | | XUE WEN;; |
| | | | EDITING | | WOLFE SCOT |
| WO 2022/174829 | Aug. 25, 2022 | Feb. 19, 2021 | EDITING OF | UNIV WUHAN | ZHANG YING;; |
| A1 | | | DOUBLE- | | SHI YAJING |
| | | | STRANDED DNA | | |
| | | | WITH RELAXED | | |
| | | | PAM | | |
| | | | REQUIREMENT | | |
| | | | FIELD OF THE | | |
| | | | DISCLOSURE | | |
| WO 2022/203905 | Sep. 29, 2022 | Mar. 24, 2021 | PRIME EDITING- | UNIV | JIANG TINGTING;; |
| A1 | | | BASED | MASSACHUSETTS | ZHANG XIAO-OU;; |
| | | | SIMULTANEOUS | | WENG ZHIPING;; |
| | | | GENOMIC | | XUE WEN |
| | | | DELETION AND | | |
| | | | INSERTION | | |
| WO 2022/204476 | Sep. 29, 2022 | Mar. 26, 2021 | NUCLEOTIDE | UNIV TEXAS | OLSON ERIC;; |
| A1 | | | EDITING TO | | CHEMELLO FRANCESCO;; |
| | | | REFRAME DMD | | BASSEL-DUBY RHONDA |
| | | | TRANSCRIPTS BY | | |
| | | | BASE EDITING | | |
| | | | AND PRIME | | |
| | | | EDITING | | |
| WO 2022/212926 | Oct. 6, 2022 | Apr. 1, 2021 | METHODS AND | PRIME | WILSON CHRISTOPHER;; |
| A1 | | | COMPOSITIONS | MEDICINE | ANZALONE ANDREW V;; |
| | | | FOR EDITING | INC | LEVY JONATHAN M |
| | | | NUCLEOTIDE | | |
| | | | SEQUENCES | | |
| WO 2022/206352 | Oct. 6, 2022 | Apr. 2, 2021 | PRIME EDITING | UNIV | LIU YAO;; |
| A1 | | | TOOL, FUSION | SHANGHAI | YANG GUANG;; |
| | | | RNA, AND USE | TECHNOLOGY | LI GUANGLEI;; |
| | | | THEREOF | | HUANG XINGXU |
| WO 2022/234051 | Nov. 10, 2022 | May 6, 2021 | SPLIT PRIME | UNIV ZURICH | SCHWANK GERALD;; |
| A1 | | | EDITING ENZYME | | VILLIGER LUKAS;; |
| | | | | | BÖCK DESIREE |
| WO 2022/256714 | Dec. 8, 2022 | Jun. 3, 2021 | GENOME EDITING | PRIME | LEVY JONATHAN M;; |
| A2 | | | COMPOSITIONS | MEDICINE | YEH WEI HSI;; |
| | | | AND METHODS | INC | CHANG AARON NAKWON; |
| | | | FOR TREATMENT | | STILLER JOHN |
| | | | OF WILSON'S | | |
| | | | DISEASE | | |
| WO 2023/283092 | Jan. 12, 2023 | Jul. 6, 2021 | COMPOSITIONS | PRIME | REES HOLLY A;; |
| A1 | | | AND METHODS | MEDICINE | PACKER MICHAEL;; |
| | | | FOR EFFICIENT | INC | BARRERA LUIS;; |
| | | | GENOME EDITING | | SLAYMAKER IAN |
| WO 2023/283246 | Jan. 12, 2023 | Jul. 9, 2021 | MODULAR PRIME | UNIV | LIU BIN;; |
| A1 | | | EDITOR SYSTEMS | MASSACHUSETTS | SONTHEIMER ERIK;; |
| | | | FOR GENOME | | XUE WEN;; |
| | | | ENGINEERING | | DONG XIAOLONG |
| WO 2023/288332 | Jan. 19, 2023 | Jul. 16, 2021 | GENOME EDITING | PRIME | LEVY JONATHAN M;; |
| A2 | | | COMPOSITIONS | MEDICINE | YEH WEI HSI;; |
| | | | AND METHODS | INC | CHANG AARON NAKWON |
| | | | FOR TREATMENT | | |
| | | | OF WILSON'S | | |
| | | | DISEASE | | |
| WO 2023/004439 | Mar. 2, 2023 | Jul. 23, 2021 | GENOME EDITING | PRIME | GORI JENNIFER L;; |
| A3 | | | COMPOSITIONS | MEDICINE | WATERMAN DAVID; |
| | | | AND METHODS | INC | HEATH JACK;; |
| | | | FOR TREATMENT | | ANZALONE ANDREW V |
| | | | OF CHRONIC | | |
| | | | GRANULOMATOUS | | |
| | | | DISEASE | | |
| WO 2023/015318 | Feb. 9, 2023 | Aug. 5, 2021 | GENOME EDITING | PRIME | ANZALONE ANDREW V;; |
| A2 | | | COMPOSITIONS | MEDICINE | CHANG AARON NAKWON;; |
| | | | AND METHODS | INC | XI LINGHE;; |
| | | | FOR TREATMENT | | HARJANTO DEWI |
| | | | OF CYSTIC | | |
| | | | FIBROSIS | | |
| WO 2023/015014 | Feb. 9, 2023 | Aug. 5, 2021 | GENOME EDITING | PRIME | HARVEY TYLER;; |
| A1 | | | COMPOSITIONS | MEDICINE | ZHENG YU;; |
| | | | AND METHODS | INC | KOSHELEVA EKATERINA;; |
| | | | FOR TREATMENT | | SUN CHICHENG;; |
| | | | OF MYOTONIC | | TANG SPENCER |
| | | | DYSTROPHY | | |
| WO 2023/015309 | Mar. 16, 2023 | Aug. 6, 2021 | IMPROVED PRIME | BROAD INST | LIU DAVID;; |
| A3 | | | EDITORS AND | INC;; HARVARD | CHEN PETER;; |
| | | | METHODS OF USE | COLLEGE | DOMAN JORDAN;; |
| | | | | | PANDEY SMRITI;; |
| | | | | | NEUGEBAUER MONICA |
| WO 2023/028180 | Aug. 24, 2023 | Aug. 24, 2021 | GENOME EDITING | PRIME | YEH WEI HSI;; |
| A3 | | | COMPOSITIONS | MEDICINE | HARJANTO DEWI |
| | | | AND METHODS | INC | |
| | | | FOR TREATMENT | | |
| | | | OF RETINOPATHY | | |
| WO 2023/034931 | Mar. 9, 2023 | Sep. 2, 2021 | MULTIPLEX, | UNIV | CHEN WEI;; |
| A1 | | | TEMPORALLY | WASHINGTON | SHENDURE JAY;; |
| | | | RESOLVED | | CHOI JUNHONG |
| | | | MOLECULAR | | |
| | | | SIGNAL | | |
| | | | RECORDER AND | | |
| | | | RELATED | | |
| | | | METHODS | | |
| WO 2023/039586 | Mar. 16, 2023 | Sep. 10, 2021 | GUIDE RNAS WITH | AGILENT | RYAN DANIEL E;; |
| A1 | | | CHEMICAL | TECHNOLOGIES | DELLINGER DOUGLAS J;; |
| | | | MODIFICATION | INC | KAISER ROBERT |
| | | | FOR PRIME | | |
| | | | EDITING | | |
| WO 2023/039508 | Mar. 16, 2023 | Sep. 13, 2021 | IMPROVED PRIME | SIGMA | KANG QIAOHUA |
| A1 | | | EDITING SYSTEM | ALDRICH CO | |
| | | | EFFICIENCY WITH | LLC | |
| | | | CIS-ACTING | | |
| | | | REGULATORY | | |
| | | | ELEMENTS | | |
| WO 2023/049931 | Mar. 30, 2023 | Sep. 27, 2021 | METHODS AND | UNIV | QUINN PETER M J;; |
| A1 | | | SYSTEMS FOR | COLUMBIA;; | LOPES DA COSTA BRUNA;; |
| | | | MODIFYING THE | BROAD INST | TSANG STEPHEN H;; |
| | | | CRUMBS | INC;; HARVARD | LIU DAVID R |
| | | | HOMOLOGUE-1 | COLLEGE | |
| | | | (CRB1) GENE | | |
| WO 2023/070110 | Apr. 27, 2023 | Oct. 21, 2021 | GENOME EDITING | PRIME | YEH WEI |
| A2 | | | COMPOSITIONS | MEDICINE | |
| | | | AND METHODS | INC | |
| | | | FOR TREATMENT | | |
| | | | OF RETINITIS | | |
| | | | PIGMENTOSA | | |
| WO 2023/070062 | May 25, 2023 | Oct. 21, 2021 | GENOME EDITING | PRIME | YEH WEI HSI |
| A3 | | | COMPOSITIONS | MEDICINE | |
| | | | AND METHODS | INC | |
| | | | FOR TREATMENT | | |
| | | | OF USHER | | |
| | | | SYNDROME TYPE | | |
| | | | 3 | | |
| WO 2023/076898 | May 4, 2023 | Oct. 25, 2021 | METHODS AND | BROAD INST | LIU DAVID;; |
| A1 | | | COMPOSITIONS | INC;; HARVARD | ANZALONE ANDREW;; |
| | | | FOR EDITING A | COLLEGE | PODRACKY CHRISTOPHER;; |
| | | | GENOME WITH | | GAO XIN |
| | | | PRIME EDITING | | |
| | | | AND A | | |
| | | | RECOMBINASE | | |
| WO 2023/081787 | May 11, 2023 | Nov. 4, 2021 | GENOME EDITING | PRIME | GORI JENNIFER L;; |
| A2 | | | COMPOSITIONS | MEDICINE | WATERMAN DAVID;; |
| | | | AND METHODS | INC | HARJANTO DEWI |
| | | | FOR TREATMENT | | |
| | | | OF FANCONI | | |
| | | | ANEMIA | | |
| WO 2023/081426 | May 11, 2023 | Nov. 5, 2021 | GENOME EDITING | PRIME | HARVEY TYLER;; |
| A1 | | | COMPOSITIONS | MEDICINE | ZHENG YU;; |
| | | | AND METHODS | INC | CHEN PETER;; |
| | | | FOR TREATMENT | | KOSHELEVA EKATERINA |
| | | | OF FRIEDREICH'S | | ALEXEEVNA;; |
| | | | ATAXIA | | HEFFERNAN JULIA;; |
| | | | | | FASCHING LIANA |
| WO 2023/086389 | May 19, 2023 | Nov. 9, 2021 | GENOME EDITING | PRIME | HARVEY TYLER;; |
| A1 | | | COMPOSITIONS | MEDICINE | ZHENG YU |
| | | | AND METHODS | INC | |
| | | | FOR TREATMENT | | |
| | | | OF AMYOTROPHIC | | |
| | | | LATERAL | | |
| | | | SCLEROSIS | | |
| WO 2023/086842 | May 19, 2023 | Nov. 9, 2021 | GENOME EDITING | PRIME | HARVEY TYLER;; |
| A1 | | | COMPOSITIONS | MEDICINE | ZHENG YU |
| | | | AND METHODS | INC | |
| | | | FOR TREATMENT | | |
| | | | OF FUCHS | | |
| | | | ENDOTHELIAL | | |
| | | | CORNEAL | | |
| | | | DYSTROPHY | | |
| WO 2023/086558 | May 19, 2023 | Nov. 11, 2021 | GENOME EDITING | PRIME | HARVEY TYLER;; |
| A1 | | | COMPOSITIONS | MEDICINE | ZHENG YU;; |
| | | | AND METHODS | INC | SUN CHICHENG |
| | | | FOR TREATMENT | | |
| | | | OF FRAGILE X | | |
| | | | SYNDROME | | |
| WO 2023/096992 | Jun. 1, 2023 | Nov. 23, 2021 | GENOME EDITING | PRIME | HART CHRISTOPHER;; |
| A1 | | | COMPOSITIONS | MEDICINE | LEVY JONATHAN |
| | | | AND METHODS | INC;; HART | |
| | | | FOR TREATMENT | CHRISTOPHER | |
| | | | OF GLYCOGEN | AUSTIN | |
| | | | STORAGE DISEASE | | |
| | | | TYPE 1B | | |
| WO 2023/096847 | Jun. 1, 2023 | Nov. 24, 2021 | METHODS AND | PRIME | WATERMAN DAVID;; |
| A2 | | | COMPOSITIONS | MEDICINE | GE PEI;; |
| | | | FOR INHIBITING | INC | KANDIMALLA EKAMBAR |
| | | | MISMATCH | | |
| | | | REPAIR | | |
| WO 2023/096977 | Jun. 1, 2023 | Nov. 24, 2021 | MODIFIED PRIME | PRIME | ANZALONE ANDREW;; |
| A2 | | | EDITING GUIDE | MEDICINE INC;; | STILLER JOHN;; |
| | | | RNAS | ANZALONE | WILEY DAVID |
| | | | | ANDREW V;; | |
| | | | | STILLER JOHN;; | |
| | | | | WILEY DAVID | |
| WO 2023/114992 | Jun. 22, 2023 | Dec. 17, 2021 | PROGRAMMABLE | MASSACHUSETTS | ABUDAYYEH OMAR;; |
| A1 | | | INSERTION | INST | GOOTENBERG JONATHAN;; |
| | | | APPROACHES VIA | TECHNOLOGY | VILLIGER LUKAS;; |
| | | | REVERSE | | JIANG KAIYI |
| | | | TRANSCRIPTASE | | |
| | | | RECRUITMENT | | |
| WO 2023/129095 | Jul. 6, 2023 | Dec. 31, 2021 | CRISPR-PE | T C USKUDAR | AKGUL GORKEM;; |
| A1 | | | SYSTEM FOR | UNIV | AKYAR ALI DOSTCAN;; |
| | | | RETINOL | | YOLVER BATUHAN;; |
| | | | DEHYDROGENASE | | TASTAN CIHAN |
| | | | 12 (RDH12) GENE | | |
| | | | MUTATIONS FOR | | |
| | | | USE IN THE | | |
| | | | TREATMENT OF | | |
| | | | RETINITIS | | |
| | | | PIGMENTOSA (RP) | | |
| | | | DISEASE | | |
| WO 2023/140409 | Jul. 27, 2023 | Jan. 20, 2022 | COMPOSITION | MKBIOTECH | KIM MIN KYU;; |
| A1 | | | FOR TREATING | CO LTD;; IAC | PARK KANG SUN;; |
| | | | CANINE HIP | IN NAT UNIV | KIM DONG ERN;; |
| | | | DYSPLASIA USING | CHUNGNAM | JI KUK BIN;; |
| | | | PRIME EDITOR | | LEE JI HYE;; |
| | | | | | KIM EUN YOUNG;; |
| | | | | | PARK YOUN BAE;; |
| | | | | | KIL TAE YOUNG |
| WO 2023/141734 | Aug. 3, 2023 | Jan. 25, 2022 | MODIFIED PRIME | NORTHWEST | WANG XIAOLONG |
| A1 | | | EDITING SYSTEM | A & F UNIV | |
| | | | AND USE | | |
| | | | THEREOF | | |
| WO 2023/154011 | Aug. 17, 2023 | Feb. 8, 2022 | COMPOSITIONS | NAT UNIV | YU HAOJIE |
| A2 | | | AND METHODS | SINGAPORE | |
| | | | FOR GENOME | | |
| | | | EDITING | | |
| WO 2023/153811 | Aug. 17, 2023 | Feb. 8, 2022 | METHOD FOR | TOOLGEN INC | LEE JEONGJOON;; |
| A1 | | | PREDICTING OFF- | | KWON JEONGHUN;; |
| | | | TARGET WHICH | | KIM MINYOUNG;; |
| | | | CAN OCCUR IN | | JO ANNA;; |
| | | | PROCESS OF | | KIM YOUNG HO |
| | | | EDITING GENOME | | |
| | | | BY USING PRIME | | |
| | | | EDITING SYSTEM | | |
| WO 2023/192655 | Dec. 28, 2023 | Apr. 1, 2022 | METHODS AND | PRIME | WILSON CHRISTOPHER;; |
| A3 | | | COMPOSITIONS | MEDICINE | ANZALONE ANDREW V |
| | | | FOR EDITING | INC | |
| | | | NUCLEOTIDE | | |
| | | | SEQUENCES | | |
| WO 2023/205687 | Oct. 26, 2023 | Apr. 20, 2022 | IMPROVED PRIME | BROAD INST | LIU DAVID;; |
| A1 | | | EDITING | INC;; HARVARD | SOUSA ALEXANDER;; |
| | | | METHODS AND | COLLEGE | DOMAN JORDAN |
| | | | COMPOSITIONS | | |
| WO 2023/212594 | Nov. 2, 2023 | Apr. 26, 2022 | SINGLE pegRNA- | UNIV | ZHENG CHUNWEI;; |
| A2 | | | MEDIATED LARGE | MASSACHUSETTS | XUE WEN;; |
| | | | INSERTIONS | | SONTHEIMER ERIK;; |
| | | | | | LIU BIN;; |
| | | | | | DONG XIAOLONG |
| WO 2023/220732 | Nov. 16, 2023 | May 13, 2022 | METHODS AND | UNIV | TSAI YI-TING;; |
| A1 | | | SYSTEMS FOR | COLUMBIA | CARUSO SALVATORE M;; |
| | | | CORRECTING | | LOPES DA COSTA BRUNA;; |
| | | | MUTATIONS IN | | TSANG STEPHEN H;; |
| | | | PRPH2 | | QUINN PETER M J |
| WO 2023/235501 | Dec. 7, 2023 | Jun. 2, 2022 | HIGH FIDELITY | UNIV | SONTHEIMER ERIK;; |
| A1 | | | NUCLEOTIDE | MASSACHUSETTS | XUE WEN;; |
| | | | POLYMERASE | | LIU BIN;; |
| | | | CHIMERIC PRIME | | DONG XIAOLONG |
| | | | EDITOR SYSTEMS | | |
| WO 2024/026415 | Feb. 1, 2024 | Jul. 27, 2022 | COMPOSITIONS, | UNIV | QUINN PETER M J;; |
| A1 | | | SYSTEMS, AND | COLUMBIA | TSAI YI-TING;; |
| | | | METHODS FOR | | LOPES DA COSTA BRUNA;; |
| | | | PRIME EDITING | | TSANG STEPHEN H |
| WO 2024/028917 | Feb. 8, 2024 | Aug. 5, 2022 | GENOME EDITING | UNIV DEGLI | QUATTRONE ALESSANDRO;; |
| A1 | | | OF THE KOZAK | STUDI DI | AMBROSINI CHIARA;; |
| | | | SEQUENCE FOR | TRENTO | CERESETO ANNA;; |
| | | | TREATING | | PETRIS GIANLUCA;; |
| | | | DISEASES | | DESTEFANIS ELIANA;; |
| | | | | | RAOSS ALBERTO |
| WO 2024/042489 | Feb. 29, 2024 | Aug. 25, 2022 | CHEMICAL | LIFEEDIT | COYLE MICHAEL;; |
| A1 | | | MODIFICATION OF | THERAPEUTICS | CRAWLEY ALEXANDRA BRINER;; |
| | | | GUIDE RNAS WITH | INC | ELICH TEDD D;; |
| | | | LOCKED NUCLEIC | | KELSO DREW;; |
| | | | ACID FOR RNA | | YU-KEMP HUI-CHIA;; |
| | | | GUIDED | | WILEY DAVID |
| | | | NUCLEASE- | | |
| | | | MEDIATED GENE | | |
| | | | EDITING | | |
| WO 2024/053921 | Mar. 14, 2024 | Sep. 7, 2022 | METHOD AND | IND ACADEMICCO | KIM HYONGBUM HENRY;; |
| A1 | | | DEVICE FOR | OPERATION | YOO GOO SANG;; |
| | | | PREDICTING | FOUNDATION | KIM HUI KWON;; |
| | | | PRIME EDITING | YONSEIUNIVERSITY | PARK JINMAN |
| | | | EFFICIENCY OF | | |
| | | | VARIOUS PRIME | | |
| | | | EDITORS IN | | |
| | | | DIFFERENT CELL | | |
| | | | TYPES | | |
| WO 2024/060461 | Mar. 28, 2024 | Sep. 20, 2022 | DOUBLE-PLASMID | ZJU | BAO ZEHUA;; |
| A1 | | | SYSTEM | HANGZHOU | SHI XIAOXIAO;; |
| | | | SUITABLE FOR | GLOBAL | XIE WEIYU;; |
| | | | YEAST PRIME | SCIENTIFIC | DENG LEI;; |
| | | | EDITING, USE, | AND TECH | CAI ZHENKUN |
| | | | AND METHOD FOR | INNOVATION | |
| | | | YEAST PRIME | CENTER | |
| | | | EDITING | | |
| WO 2024/077267 | Apr. 11, 2024 | Oct. 7, 2022 | PRIME EDITING | BROAD INST | LIU DAVID;; |
| A1 | | | METHODS AND | INC;; HARVARD | MATUSZEK ZANETA;; |
| | | | COMPOSITIONS | COLLEGE | ARBAB MANDANA |
| | | | FOR TREATING | | |
| | | | TRIPLET REPEAT | | |
| | | | DISORDERS | | |
| WO 2024/083135 | Apr. 25, 2024 | Oct. 17, 2022 | ISCB | HUIDAGENE | ZHANG HAINAN;; |
| A1 | | | POLYPEPTIDES | THERAPEUTICS | ZHOU YINGSI |
| | | | AND USES | SINGAPORE | |
| | | | THEREOF | PTE LTD;; | |
| | | | | HUIDAGENE | |
| | | | | THERAPEUTICS | |
| | | | | CO LTD | |
| WO 2024/095245 | Jul. 18, 2024 | Nov. 4, 2022 | EVOLVED | LIFEEDIT | COYLEMICHAEL; |
| A9 | | | ADENINE | THERAPEUTICS | CRAWLEY ALEXANDRA BRINER;; |
| | | | DEAMINASES AND | INC | ELICH TEDD D;; |
| | | | RNA-GUIDED | | FAHAM SALEM;; |
| | | | NUCLEASE | | KELSO DREW;; |
| | | | FUSION PROTEINS | | OLIVER ADRIAN PICKAR;; |
| | | | WITH INTERNAL | | RIBEIRO LUCAS FERREIRA;; |
| | | | INSERTION SITES | | RICKELS RYAN;; |
| | | | AND METHODS OF | | COMPTON SARAH |
| | | | USE | | |
| WO 2024/112876 | May 30, 2024 | Nov. 23, 2022 | SPLIT SYNTHESIS | PRIME | KANDIMALLA |
| A2 | | | OF LONG RNAS | MEDICINE | EKAMBARESWARA RAO;; |
| | | | | INC | KOTIKAM VENUBABU;; |
| | | | | | SETH MEETU;; |
| | | | | | ZHOU CONG;; |
| | | | | | DOYLE ANDREW;; |
| | | | | | WILEY DAVID;; |
| | | | | | HUSSMANN JEFFREY;; |
| | | | | | LEVY JONATHAN;; |
| | | | | | ALEXANDER SETH;; |
| | | | | | ANZALONE ANDREW;; |
| | | | | | KUMBHARE ROHAN;; |
| | | | | | PUTTA MALLIKARJUNA |
| WO 2024/138087 | Aug. 8, 2024 | Dec. 23, 2022 | METHODS AND | BROAD INST | LIU DAVID R;; |
| A3 | | | COMPOSITIONS | INC;; HARVARD | CHEN PETER J;; |
| | | | FOR MODULATING | COLLEGE | GAO XIN |
| | | | CELLULAR FACTORS | | |
| | | | TO INCREASE PRIME | | |
| | | | EDITING | | |
| | | | EFFICIENCIES | | |
| WO 2024/148313 | Sep. 19, 2024 | Jan. 5, 2023 | GENOME EDITING | PRIME | PODRACKY CHRISTOPHER J;; |
| A3 | | | COMPOSITIONS | MEDICINE | POMEROY EMILY;; |
| | | | AND METHODS OF | INC | BLOCH NOAH B;; |
| | | | USE | | ANZALONE ANDREW V |
| WO 2024/155741 | Jul. 25, 2024 | Jan. 18, 2023 | PRIME EDITING- | BROAD INST | LIU DAVID;; |
| A1 | | | MEDIATED | INC;; HARVARD | RAGURAM ADITYA;; |
| | | | READTHROUGH | COLLEGE | ERWOOD STEVEN;; |
| | | | OF PREMATURE | | PIERCE SARAH;; |
| | | | TERMINATION | | OYE OLUKEYEDE |
| | | | CODONS (PERT) | | |
| WO 2024/163679 | Aug. 8, 2024 | Feb. 1, 2023 | GENOME EDITING | PRIME | XI LINGHE;; |
| A1 | | | COMPOSITIONS | MEDICINE | STILLER JOHN;; |
| | | | AND METHODS | INC | HARJANTO DEWI |
| | | | FOR TREATMENT | | |
| | | | OF CYSTIC | | |
| | | | FIBROSIS | | |
| WO 2024/168116 | Aug. 15, 2024 | Feb. 8, 2023 | GENOME EDITING | PRIME | STILLER JOHN BENJAMIN |
| A1 | | | COMPOSITIONS | MEDICINE | |
| | | | AND METHODS | INC | |
| | | | FOR TREATMENT | | |
| | | | OF MYOTONIC | | |
| | | | DYSTROPHY | | |
| WO 2024/168147 | Aug. 15, 2024 | Feb. 9, 2023 | EVOLVED | BROAD INST | LIU DAVID;; |
| A2 | | | RECOMBINASES | INC;; HARVARD | PANDEY SMRITI;; |
| | | | FOR EDITING A | COLLEGE | GAO XIN |
| | | | GENOME IN | | |
| | | | COMBINATION | | |
| | | | WITH PRIME | | |
| | | | EDITING | | |
| WO 2024/165060 | Aug. 15, 2024 | Feb. 9, 2023 | CULTIVATION | INST | YE JIAN;; |
| A1 | | | METHOD FOR | MICROBIOLOGY | ZHAO PINGZHI;; |
| | | | ANTI-YELLOW- | CAS | SUN YANWEI;; |
| | | | SHOOT-DISEASE | | YANG HUAN |
| | | | PLANT AND USE | | |
| | | | THEREOF | | |
| WO 2024/170866 | Aug. 22, 2024 | Feb. 16, 2023 | METHODS FOR | GENOME RES | PARTS LEOPOLD;; |
| A1 | | | MODIFYING A | LTD;; BROAD | KOEPPEL JONAS;; |
| | | | CELL GENOME | INST | ELLIS TOM;; |
| | | | | INC;; HARVARD | FERREIRA RAPHAEL;; |
| | | | | COLLEGE | CHURCH GEORGE MCDONALD |
| WO 2024/178144 | Aug. 29, 2024 | Feb. 22, 2023 | METHODS AND | PRIME | WILSON CHRISTOPHER;; |
| A1 | | | COMPOSITIONS | MEDICINE | ANZALONE ANDREW V |
| | | | FOR EDITING | INC | |
| | | | NUCLEOTIDE | | |
| | | | SEQUENCES | | |
| WO 2024/187173 | Sep. 12, 2024 | Mar. 9, 2023 | HIGH | UNIV | SHEN YIN;; |
| A1 | | | THROUGHPUT | CALIFORNIA | REN XINGJIE;; |
| | | | PRIME EDITING | | YANG HAN |
| | | | SCREENS | | |
| | | | IDENTIFY | | |
| | | | FUNCTIONAL DNA | | |
| | | | VARIANTS IN THE | | |
| | | | HUMAN GENOME | | |
| WO 2024/206125 | Oct. 3, 2024 | Mar. 24, 2023 | USE OF PRIME | THE BROAD | LIU DAVID;; |
| A1 | | | EDITING FOR | INST INC;; | EVERETTE KELCEE; |
| | | | TREATING SICKLE | PRESIDENT | NEWBY GREGORY;; |
| | | | CELL DISEASE | AND | YEN JONATHAN;; |
| | | | | FELLOWS OF | WEISS MITCHELL |
| | | | | HARVARD | |
| | | | | COLLEGE;; ST | |
| | | | | JUDE | |
| | | | | CHILDRENS | |
| | | | | RES | |
| | | | | HOSPITAL | |
| | | | | INC | |
| WO 2024/215712 | Oct. 17, 2024 | Apr. 11, 2023 | METHODS FOR | UNIV OF | LI XIAOYI;; |
| A2 | | | IDENTIFYING | WASHINGTON | SHENDURE JAY ASHOK;; |
| | | | EPIGENETIC | | CHEN WEI;; |
| | | | FACTORS | | CHOI JUNHONG |
| | | | INFLUENCING | | |
| | | | GENE EDITING | | |
| | | | AND | | |
| | | | MODULATING | | |
| | | | GENE EDITING | | |
| | | | OUTCOME VIA | | |
| | | | EPIGENETIC | | |
| | | | MODULATION | | |
| WO 2024/220135 | Oct. 24, 2024 | Apr. 17, 2023 | PRIME EDITING | UNIVERSITY | WOLFE, Scot;; |
| A1 | | | SYSTEMS HAVING | OF | WATTS, Jonathan;; |
| | | | PEGRNA WITH | MASSACHUSE | LIU, Pengpeng;; |
| | | | REDUCED AUTO- | TTS | PONNIENSELVAN, Karthikeyan;; |
| | | | INHIBITORY | | KEENER, David;; |
| | | | INTERACTION | | DEVI, Gitali |

### Methods of Treatment and Uses

In another aspect, the present disclosure provides methods for correcting a pathogenic mutation in the CDKL5 gene by prime editing, including, but not limited to, the correction of a c.1412delA mutation in the CDKL5 gene by prime editing, and therefore treating CDKL5 deficiency disorder.

The methods of the present disclosure use a prime editor in combination with a pegRNA (and optionally an ngRNA) to carry out prime editing to directly correct mutations in the CDKL5 gene that cause CDKL5 deficiency disorder. In one aspect, the present disclosure provides methods of correcting a 1412delA mutation in a CDKL5 gene by prime editing comprising contacting a target DNA sequence comprising the CDKL5 gene with a prime editor and a prime editing guide RNA (pegRNA), wherein the pegRNA comprises a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6). In certain embodiments, the pegRNA comprises the sequence of any of the pegRNAs provided in Tables 1, 2, and 4 herein, or a sequence at least 80%, at least 85%, at least 90%, at least 95%, at least 96%, at least 97%, at least 98%, or at least 99% identical to any of the pegRNAs provided in Tables 1, 2, and 4 herein.

In some embodiments, the contacting further comprises using a nicking guide RNA (ngRNA). In some embodiments, the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13). In certain embodiments, the ngRNA comprises the sequence of any one of any one of the ngRNAs provided in Table 3 herein, or a sequence at least 80%, at least 85%, at least 90%, at least 95%, at least 96%, at least 97%, at least 98%, or at least 99% identical to any one of the ngRNAs provided in Table 3 herein.

In some embodiments, the prime editor comprises (i) a napDNAbp domain, and (ii) a polymerase domain. In certain embodiments, the napDNAbp domain has a nickase activity. In some embodiments, the napDNAbp domain is a Cas9 protein or a variant thereof (e.g., Cas9-NRCH). In certain embodiments, the napDNAbp domain is a Cas9 nickase (nCas9). In certain embodiments, the napDNAbp domain comprises a sequence of any one of the napDNAbps provided herein, or an amino acid sequence at least 80%, at least 85%, at least 90%, at least 95%, at least 96%, at least 97%, at least 98%, or at least 99% identical to any one of the napDNAbps provided herein. In some embodiments, the polymerase domain is a reverse transcriptase domain. In certain embodiments, the polymerase domain comprises a sequence of any one of the polymerases provided herein, or an amino acid sequence at least 80%, at least 85%, at least 90%, at least 95%, at least 96%, at least 97%, at least 98%, or at least 99% identical to any one of the polymerases provided herein.

In some embodiments, the prime editor is PE2, PE3, PE4, PE5, or a variant thereof (including PE2max, PE3max, PE4max, and PE5max). In certain embodiments, the prime editor is PE4max-NRCH, or a variant thereof. In some embodiments, the prime editor is a PE6 prime editor, or a variant thereof. In certain embodiments, the prime editor is PE6b-NRCH, PE6c-NRCH, PE6d-NRCH, or a variant thereof.

In some embodiments, the method is performed in vitro. In some embodiments, the method is performed in vivo. In some embodiments, the method is performed in a subject. In certain embodiments, the subject is a human. In some embodiments, the method is a method of treating CDKL5 deficiency disorder in the subject. In certain embodiments, the method achieves greater than 20%, greater than 25%, greater than 30%, greater than 35%, or greater than 40% correction of the 1412delA mutation. In certain embodiments, the method results in introduction of less than 2%, less than 3%, less than 4%, or less than 5% indel byproducts in the target DNA sequence.

In another aspect, the present disclosure provides methods for correcting a c.826-1 G-to-A point mutation in a CDKL5 gene by prime editing. In some embodiments, the method comprises contacting a target DNA sequence comprising the CDKL5 gene with a prime editor and a prime editing guide RNA (pegRNA), wherein the pegRNA comprises a spacer sequence of GCTGTCAAGTATCTGTCAGCT (SEQ ID NO: 7), or a spacer sequence at least 80%, at least 85%, at least 90%, at least 95%, at least 96%, at least 97%, at least 98%, or at least 99% identical to GCTGTCAAGTATCTGTCAGCT (SEQ ID NO: 7), or a spacer sequence comprising one, two, three, four, or five mutations, insertions, or deletions relative to GCTGTCAAGTATCTGTCAGCT (SEQ ID NO: 7). In certain embodiments, the pegRNA comprises the sequence GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence at least 80%, at least 85%, at least 90%, at least 95%, at least 96%, at least 97%, at least 98%, or at least 99% identical to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence comprising one, two, three, four, or five mutations, insertions, or deletions relative to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39). In some embodiments, the method further comprises contacting the target DNA sequence with a nicking guide RNA. In certain embodiments, the nicking guide RNA comprises the sequence GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence at least 80%, at least 85%, at least 90%, at least 95%, at least 96%, at least 97%, at least 98%, or at least 99% identical to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence comprising one, two, three, four, or five mutations, insertions, or deletions relative to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17).

In another aspect, the present disclosure provides methods for correcting a cluster of mutations in exon 8 of the CDKL5 gene by prime editing. In some embodiments, the method comprises contacting a target DNA sequence comprising the CDKL5 gene with a prime editor and a prime editing guide RNA (pegRNA), wherein the pegRNA comprises a spacer sequence of CAGAGTACGTTGCCACCAGA (SEQ ID NO: 8), or a spacer sequence at least 80%, at least 85%, at least 90%, at least 95%, at least 96%, at least 97%, at least 98%, or at least 99% identical to CAGAGTACGTTGCCACCAGA (SEQ ID NO: 8), or a spacer sequence comprising one, two, three, four, or five mutations, insertions, or deletions relative to CAGAGTACGTTGCCACCAGA (SEQ ID NO: 8). In certain embodiments, the pegRNA comprises the sequence CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence at least 80%, at least 85%, at least 90%, at least 95%, at least 96%, at least 97%, at least 98%, or at least 99% identical to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence comprising one, two, three, four, or five mutations, insertions, or deletions relative to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41). In some embodiments, the method further comprises contacting the target DNA sequence with a nicking guide RNA. In certain embodiments, the nicking guide RNA comprises the sequence GTGGAAATGTCATTCTATTTT (SEQ ID NO: 18), or a sequence at least 80%, at least 85%, at least 90%, at least 95%, at least 96%, at least 97%, at least 98%, or at least 99% identical to GTGGAAATGTCATTCTATTTT (SEQ ID NO: 18), or a sequence comprising one, two, three, four, or five mutations, insertions, or deletions relative to GTGGAAATGTCATTCTATTTT (SEQ ID NO: 18).

In another aspect, the present disclosure provides methods for treating CDKL5 deficiency disorder (CDD) by prime editing using the prime editor PE6b-NRCH, PE6C-NRCH, or PE6d-NRCH. In some embodiments, the method comprises contacting a target DNA sequence comprising a CDKL5 gene comprising a pathogenic mutation with a prime editor, or a nucleotide sequence encoding a prime editor, and a pegRNA, or a nucleotide sequence encoding a prime editor, wherein the prime editor comprises an amino acid sequence of any one of SEQ ID NOs: 355-357, or an amino acid sequence at least 80%, at least 85%, at least 90%, at least 95%, at least 96%, at least 97%, at least 98%, or at least 99% identical to an amino acid sequence of any one of SEQ ID NOs: 355-357. In some embodiments, the pegRNA is capable of correcting a c.1412delA mutation in the CDKL5 gene. In some embodiments, the pegRNA is any of the pegRNAs described herein.

In another aspect, the present disclosure provides for the use of any of the pegRNAs, compositions, polynucleotides, vectors, AAV particles, VLPs, or pharmaceutical compositions provided herein in the treatment of CDKL5 deficiency disorder.

In another aspect, the present disclosure provides for the use of any of the pegRNAs, compositions, polynucleotides, vectors, AAV particles, VLPs, or pharmaceutical compositions provided herein in in the manufacture of a medicament for the treatment of CDKL5 deficiency disorder.

### Complexes, Polynucleotides, Vectors, Cells, and Kits

The present disclosure provides, in some aspects, complexes comprising any of the pegRNAs or ngRNAs disclosed herein bound to any of the prime editors disclosed herein (e.g., PE6b-NRCH, PE6c-NRCH, and PE6d-NRCH). The present disclosure also provides, in some aspects, polynucleotides and vectors encoding any of the pegRNAs, ngRNAs, prime editors, complexes, systems, and/or compositions described herein. In some aspects, the present disclosure provides polynucleotides and vectors encoding a pegRNA and a prime editor as disclosed herein. In some embodiments, the polynucleotides and vectors provided herein comprise DNA (e.g., plasmid DNA or viral DNA). In some embodiments, the polynucleotides and vectors provided herein comprise RNA (e.g., mRNA or viral RNA).

Cells that may contain any of the pegRNAs, ngRNAs, prime editors, complexes, systems, and/or compositions described herein are also provided by the present disclosure. The methods described herein may be used to deliver a pegRNA and prime editor (and optionally an ngRNA) into a eukaryotic cell (e.g., a mammalian cell, such as a human cell, for example a human nerve cell). In some embodiments, the cell is in vitro (e.g., a cultured cell). In some embodiments, the cell is in vivo (e.g., in a subject, such as a human subject). In some embodiments, the cell is ex vivo (e.g., isolated from a subject and may be administered back to the same or a different subject).

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In some embodiments, a cell transfected with one or more vectors described herein is used to establish a new cell line comprising one or more vector-derived sequences. In some embodiments, a cell transiently transfected with the components of a prime editing system as described herein (such as by transient transfection of one or more vectors, or transfection with RNA), and modified through the activity of a prime editing complex, is used to establish a new cell line comprising cells containing the modification but lacking any other exogenous sequence.

The pegRNAs, ngRNAs, prime editors, systems, complexes, compositions, polynucleotides, and/or vectors described herein may also be assembled into kits. In some embodiments, the kit comprises polynucleotides for expression of the pegRNAs, ngRNAs, prime editors, complexes, systems, and/or compositions described herein. In some embodiments, the kit comprises appropriate pegRNAs or nucleic acid vectors for the expression of such pegRNAs to target the Cas9 protein of a prime editor to a desired target sequence, e.g., in CDKL5. In some embodiments, the pegRNAs in the kit are useful for correcting a c.1412delA mutation in a CDKL5 gene.

The kits described herein may include one or more containers housing components for performing the methods described herein, and optionally instructions for use. Any of the kits described herein may further comprise components needed for performing the prime editing methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### EXAMPLES

### Example 1. Prime Editing for CDD

### Creation of a Mouse Cell Line:

An epegRNA and nicking guide capable of installing the c.1412delA mutation in an N2a cell line were designed. Synthetic guide sequences were ordered from an oligonucleotide synthesis company, and 90 μmol of synthetic epegRNA and 60 μmol of synthetic nicking sgRNA were electroporated into 2×10⁵ wildtype N2a cells with 1000 ng each of in vitro transcribed PEmax mRNA and MLH1dn mRNA. The cells were allowed to grow for three days before being diluted to 1 cell per well of a 96 well plate. After growing up single cell colonies for two weeks, colonies were sequenced, and growth of heterozygous cell lines with the c.1412delA mutation and without indels was continued for use in further experiments.

### Creation of a Human Cell Line:

16 epegRNAs and 4 nicking guides capable of installing the c.1412delA mutation in a HEK293T cell line were designed and screened. After the most efficient PE5max editing strategy was determined, approximately 3×10⁴ cells were transfected and allowed to grow for three days before being diluted into a single cell per well of a 96 well plate. After growing single cell colonies for two weeks, colonies were sequenced, and growth of heterozygous and homozygous cell lines with the c.1412delA mutation and without indels was continued for use in further experiments.

Cloning of epegRNAs:

An isothermal assembly method was used to clone epegRNAs.

Cloning of PEmax, PEmaxΔRNaseH and PE6b-d with SpCas9-NRCH:

PCR primers were designed to amplify SpCas9-NRCH and to amplify the backbone, or the reverse transcriptase, antibiotic resistance cassette, and other non-Cas features of the editing plasmids. Gibson assembly protocol was then used to clone plasmids with SpCas9-NRCH and the desired reverse transcriptase from each prime editor.

Transfections to Correct c.1412delA in the N2a and HEK293T Disease Cell Lines:

All transfections were completed with Lipofectamine 2000 and included 60 ng of epegRNA, 200 ng of prime editor, 22 ng of nicking guide, and 100 ng of MLH1dn.

All pegRNAs were encoded using the promoter sequence

(SEQ ID NO: 403)
TACGTGACGTAGAAAGTAATAATTTCTTGGGTAGTTTGCAGTTTTAAAATTATGTTTTAA
AATGGACTATCATATGCTTACCGTAACTTGAAAGTATTTCGATTTCTTGGCTTTATATATC
TTGTGGAAAGGACGAAACACC.

Tested pegRNAs and gRNAs

pegRNAs and nicking gRNAs of the following sequences were tested:

TABLE 1
epegRNAs for SpCas9-NRCH in N2a mouse model cell line (All sequences are shown 5′ to 3′)
(All pegRNAs encode a +2 G > T seed edit and the +6T correction (corresponding to the A
that needs inserting to correct the A deletion on the opposite strand))

| | | | | | | Sequence | |
|---|---|---|---|---|---|---|---|
| | | | | | | Identifier | |
| | | | | | | for Full | |
| | | | | | | epegRNA | |
| | | | | | Term- | (without | |
| Spacer | Scaffold | Template | PBS | 3′ motif | inator | terminator) | Notes |
| GCCGGGAGGACTGG | GTTTTAGAG | CTATAT | TTCC | CGCGGTTCTA | TTTT | 42 | PBS 9 RTT |
| GGAATGG (SEQ ID | CTAGAAATA | TGACA | CCAG | TCTAGTTACG | TT | | 14 |
| NO: 1) | GCAAGTTAA | CAA | T | CGTTAAACCA | | | |
| | AATAAGGCT | (SEQ ID | | ACTAGAA | | | |
| | AGTCCGTTA | NO: 404) | | (SEQ ID NO: | | | |
| | TCAACTTGA | | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 43 | PBS 9 RTT |
| GGAATGG (SEQ ID | CTAGAAATA | TTGACA | CCAG | TCTAGTTACG | TT | | 15 |
| NO: 1) | GCAAGTTAA | CAA | T | CGTTAAACCA | | | |
| | AATAAGGCT | (SEQ ID | | ACTAGAA | | | |
| | AGTCCGTTA | NO: 405) | | (SEQ ID NO: | | | |
| | TCAACTTGA | | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 44 | PBS 9 RTT |
| GGAATGG (SEQ ID | CTAGAAATA | ATTGAC | CCAG | TCTAGTTACG | TT | | 16 |
| NO: 1) | GCAAGTTAA | ACAA | T | CGTTAAACCA | | | |
| | AATAAGGCT | (SEQ ID | | ACTAGAA | | | |
| | AGTCCGTTA | NO: 406) | | (SEQ ID NO: | | | |
| | TCAACTTGA | | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 45 | PBS 9 RTT |
| GGAATGG (SEQ ID | CTAGAAATA | TATTGA | CCAG | TCTAGTTACG | TT | | 17 |
| NO: 1) | GCAAGTTAA | CACAA | T | CGTTAAACCA | | | |
| | AATAAGGCT | (SEQ ID | | ACTAGAA | | | |
| | AGTCCGTTA | NO: 407) | | (SEQ ID NO: | | | |
| | TCAACTTGA | | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 46 | PBS 9 RTT |
| GGAATGG (SEQ ID | CTAGAAATA | TATATT | CCAG | TCTAGTTACG | TT | | 18 |
| NO: 1) | GCAAGTTAA | GACAC | T | CGTTAAACCA | | | |
| | AATAAGGCT | AA (SEQ | | ACTAGAA | | | |
| | AGTCCGTTA | ID NO: | | (SEQ ID NO: | | | |
| | TCAACTTGA | 408) | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 47 | PBS 9 RTT |
| GGAATGG (SEQ ID | CTAGAAATA | CTATAT | CCAG | TCTAGTTACG | TT | | 19 |
| NO: 1) | GCAAGTTAA | TGACA | T | CGTTAAACCA | | | |
| | AATAAGGCT | CAA | | ACTAGAA | | | |
| | AGTCCGTTA | (SEQ ID | | (SEQ ID NO: | | | |
| | TCAACTTGA | NO: 409) | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 48 | PBS 9 RTT |
| GGAATGG (SEQ ID | CTAGAAATA | GCTATA | CCAG | TCTAGTTACG | TT | | 20 |
| NO: 1) | GCAAGTTAA | TTGACA | | CGTTAAACCA | | | |
| | AATAAGGCT | CAA | | ACTAGAA | | | |
| | AGTCCGTTA | (SEQ ID | | (SEQ ID NO: | | | |
| | TCAACTTGA | NO: 410) | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 49 | PBS 9 RTT |
| GGAATGG (SEQ ID | CTAGAAATA | AGCTAT | CCAG | TCTAGTTACG | TT | | 21 |
| NO: 1) | GCAAGTTAA | ATTGAC | T | CGTTAAACCA | | | |
| | AATAAGGCT | ACAA | | ACTAGAA | | | |
| | AGTCCGTTA | (SEQ ID | | (SEQ ID NO: | | | |
| | TCAACTTGA | NO: 411) | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 50 | PBS 10 |
| GGAATGG (SEQ ID | CTAGAAATA | TGACA | CCAG | TCTAGTTACG | TT | | RTT 14 |
| NO: 1) | GCAAGTTAA | CAA | TC | CGTTAAACCA | | | |
| | AATAAGGCT | (SEQ ID | (SEQ | ACTAGAA | | | |
| | AGTCCGTTA | NO: 404) | ID NO: | (SEQ ID NO: | | | |
| | TCAACTTGA | | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 51 | PBS 10 |
| GGAATGG (SEQ ID | CTAGAAATA | TTGACA | CCAG | TCTAGTTACG | TT | | RTT 15 |
| NO: 1) | GCAAGTTAA | CAA | TC | CGTTAAACCA | | | |
| | AATAAGGCT | (SEQ ID | (SEQ | ACTAGAA | | | |
| | AGTCCGTTA | NO: 405) | ID NO: | (SEQ ID NO: | | | |
| | TCAACTTGA | | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 52 | PBS 10 |
| GGAATGG (SEQ ID | CTAGAAATA | ATTGAC | CCAG | TCTAGTTACG | TT | | RTT 16 |
| NO: 1) | GCAAGTTAA | ACAA | TC | CGTTAAACCA | | | |
| | AATAAGGCT | (SEQ ID | (SEQ | ACTAGAA | | | |
| | AGTCCGTTA | NO: 406) | ID NO: | (SEQ ID NO: | | | |
| | TCAACTTGA | | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 53 | PBS 10 |
| GGAATGG (SEQ ID | CTAGAAATA | TATTGA | CCAG | TCTAGTTACG | TT | | RTT 17 |
| NO: 1) | GCAAGTTAA | CACAA | TC | CGTTAAACCA | | | |
| | AATAAGGCT | (SEQ ID | (SEQ | ACTAGAA | | | |
| | AGTCCGTTA | NO: 407) | ID NO: | (SEQ ID NO: | | | |
| | TCAACTTGA | | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 54 | PBS 10 |
| GGAATGG (SEQ ID | CTAGAAATA | TATATT | CCAG | TCTAGTTACG | TT | | RTT 18 |
| NO: 1) | GCAAGTTAA | GACAC | TC | CGTTAAACCA | | | |
| | AATAAGGCT | AA (SEQ | (SEQ | ACTAGAA | | | |
| | AGTCCGTTA | ID NO: | ID NO: | (SEQ ID NO: | | | |
| | TCAACTTGA | 408) | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 55 | PBS 10 |
| GGAATGG (SEQ ID | CTAGAAATA | CTATAT | CCAG | TCTAGTTACG | TT | | RTT 19 |
| NO: 1) | GCAAGTTAA | TGACA | TC | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 409) | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 56 | PBS 10 |
| GGAATGG (SEQ ID | CTAGAAATA | GCTATA | CCAG | TCTAGTTACG | TT | | RTT 20 |
| NO: 1) | GCAAGTTAA | TTGACA | TC | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 410) | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 57 | PBS 10 |
| GGAATGG (SEQ ID | CTAGAAATA | AGCTAT | CCAG | TCTAGTTACG | TT | | RTT 21 |
| NO: 1) | GCAAGTTAA | ATTGAC | TC | CGTTAAACCA | | | |
| | AATAAGGCT | ACAA | (SEQ | ACTAGAA | | | |
| | AGTCCGTTA | (SEQ ID | ID NO: | (SEQ ID NO: | | | |
| | TCAACTTGA | NO: 411) | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 58 | PBS 11 |
| GGAATGG (SEQ ID | CTAGAAATA | TGACA | CCAG | TCTAGTTACG | TT | | RTT 14 |
| NO: 1) | GCAAGTTAA | CAA | TCC | CGTTAAACCA | | | |
| | AATAAGGCT | (SEQ ID | (SEQ | ACTAGAA | | | |
| | AGTCCGTTA | NO: 404) | ID NO: | (SEQ ID NO: | | | |
| | TCAACTTGA | | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | CCAG | CGCGGTTCTA | TTTT | 59 | PBS 11 |
| GGAATGG (SEQ ID | CTAGAAATA | TTGACA | TTCC | TCTAGTTACG | TT | | RTT 15 |
| NO: 1) | GCAAGTTAA | CAA | TCC | CGTTAAACCA | | | |
| | AATAAGGCT | (SEQ ID | (SEQ | ACTAGAA | | | |
| | AGTCCGTTA | NO: 405) | ID NO: | (SEQ ID NO: | | | |
| | TCAACTTGA | | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 60 | PBS 11 |
| GGAATGG (SEQ ID | CTAGAAATA | ATTGAC | CCAG | TCTAGTTACG | TT | | RTT 16 |
| NO: 1) | GCAAGTTAA | ACAA | TCC | CGTTAAACCA | | | |
| | AATAAGGCT | (SEQ ID | (SEQ | ACTAGAA | | | |
| | AGTCCGTTA | NO: 406) | ID NO: | (SEQ ID NO: | | | |
| | TCAACTTGA | | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 61 | PBS 11 |
| GGAATGG (SEQ ID | CTAGAAATA | TATTGA | CCAG | TCTAGTTACG | TT | | RTT 17 |
| NO: 1) | GCAAGTTAA | CACAA | TCC | CGTTAAACCA | | | |
| | AATAAGGCT | (SEQ ID | (SEQ | ACTAGAA | | | |
| | AGTCCGTTA | NO: 407) | ID NO: | (SEQ ID NO: | | | |
| | TCAACTTGA | | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 62 | PBS 11 |
| GGAATGG (SEQ ID | CTAGAAATA | TATATT | CCAG | TCTAGTTACG | TT | | RTT 18 |
| NO: 1) | GCAAGTTAA | GACAC | TCC | CGTTAAACCA | | | |
| | AATAAGGCT | AA (SEQ | (SEQ | ACTAGAA | | | |
| | AGTCCGTTA | ID NO: | ID NO: | (SEQ ID NO: | | | |
| | TCAACTTGA | 408) | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 63 | PBS 11 |
| GGAATGG (SEQ ID | CTAGAAATA | CTATAT | CCAG | TCTAGTTACG | TT | | RTT 19 |
| NO: 1) | GCAAGTTAA | TGACA | TCC | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 409) | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 64 | PBS 11 |
| GGAATGG (SEQ ID | CTAGAAATA | GCTATA | CCAG | TCTAGTTACG | TT | | RTT 20 |
| NO: 1) | GCAAGTTAA | TTGACA | TCC | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 410) | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 65 | PBS 11 |
| GGAATGG (SEQ ID | CTAGAAATA | AGCTAT | CCAG | TCTAGTTACG | TT | | RTT 21 |
| NO: 1) | GCAAGTTAA | ATTGAC | TCC | CGTTAAACCA | | | |
| | AATAAGGCT | ACAA | (SEQ | ACTAGAA | | | |
| | AGTCCGTTA | (SEQ ID | ID NO: | (SEQ ID NO: | | | |
| | TCAACTTGA | NO: 411) | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 66 | PBS 12 |
| GGAATGG (SEQ ID | CTAGAAATA | TGACA | CCAG | TCTAGTTACG | TT | | RTT 14 |
| NO: 1) | GCAAGTTAA | CAA | TCCT | CGTTAAACCA | | | |
| | AATAAGGCT | (SEQ ID | (SEQ | ACTAGAA | | | |
| | AGTCCGTTA | NO: 404) | ID NO: | (SEQ ID NO: | | | |
| | TCAACTTGA | | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 67 | PBS 12 |
| GGAATGG (SEQ ID | CTAGAAATA | TTGACA | CCAG | TCTAGTTACG | TT | | RTT 15 |
| NO: 1) | GCAAGTTAA | CAA | TCCT | CGTTAAACCA | | | |
| | AATAAGGCT | (SEQ ID | (SEQ | ACTAGAA | | | |
| | AGTCCGTTA | NO: 405) | ID NO: | (SEQ ID NO: | | | |
| | TCAACTTGA | | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 68 | PBS 12 |
| GGAATGG (SEQ ID | CTAGAAATA | ATTGAC | CCAG | TCTAGTTACG | TT | | RTT 16 |
| NO: 1) | GCAAGTTAA | ACAA | TCCT | CGTTAAACCA | | | |
| | AATAAGGCT | (SEQ ID | (SEQ | ACTAGAA | | | |
| | AGTCCGTTA | NO: 406) | ID NO: | (SEQ ID NO: | | | |
| | TCAACTTGA | | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 69 | PBS 12 |
| GGAATGG (SEQ ID | CTAGAAATA | TATTGA | CCAG | TCTAGTTACG | TT | | RTT 17 |
| NO: 1) | GCAAGTTAA | CACAA | TCCT | CGTTAAACCA | | | |
| | AATAAGGCT | (SEQ ID | (SEQ | ACTAGAA | | | |
| | AGTCCGTTA | NO: 407) | ID NO: | (SEQ ID NO: | | | |
| | TCAACTTGA | | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 70 | PBS 12 |
| GGAATGG (SEQ ID | CTAGAAATA | TATATT | CCAG | TCTAGTTACG | TT | | RTT 18 |
| NO: 1) | GCAAGTTAA | GACAC | TCCT | CGTTAAACCA | | | |
| | AATAAGGCT | AA (SEQ | (SEQ | ACTAGAA | | | |
| | AGTCCGTTA | ID NO: | ID NO: | (SEQ ID NO: | | | |
| | TCAACTTGA | 408) | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 71 | PBS 12 |
| GGAATGG (SEQ ID | CTAGAAATA | CTATAT | CCAG | TCTAGTTACG | TT | | RTT 19 |
| NO: 1) | GCAAGTTAA | TGACA | TCCT | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 409) | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 72 | PBS 12 |
| GGAATGG (SEQ ID | CTAGAAATA | GCTATA | CCAG | TCTAGTTACG | TT | | RTT 20 |
| NO: 1) | GCAAGTTAA | TTGACA | TCCT | CGTTAAACCA | | | |
| | AATAAGGCT | CAA | (SEQ | ACTAGAA | | | |
| | AGTCCGTTA | (SEQ ID | ID NO: | (SEQ ID NO: | | | |
| | TCAACTTGA | NO: 410) | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 73 | PBS 12 |
| GGAATGG (SEQ ID | CTAGAAATA | AGCTAT | CCAG | TCTAGTTACG | TT | | RTT 21 |
| NO: 1) | GCAAGTTAA | ATTGAC | TCCT | CGTTAAACCA | | | |
| | AATAAGGCT | ACAA | (SEQ | ACTAGAA | | | |
| | AGTCCGTTA | (SEQ ID | ID NO: | (SEQ ID NO: | | | |
| | TCAACTTGA | NO: 411) | 31) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 74 | PBS 13 |
| GGAATGG (SEQ ID | CTAGAAATA | TGACA | CCAG | TCTAGTTACG | TT | | RTT 14 |
| NO: 1) | GCAAGTTAA | CAA | TCCT | CGTTAAACCA | | | |
| | AATAAGGCT | (SEQ ID | C (SEQ | ACTAGAA | | | |
| | AGTCCGTTA | NO: 404) | ID NO: | (SEQ ID NO: | | | |
| | TCAACTTGA | | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 75 | PBS 13 |
| GGAATGG (SEQ ID | CTAGAAATA | TTGACA | CCAG | TCTAGTTACG | TT | | RTT 15 |
| NO: 1) | GCAAGTTAA | CAA | TCCT | CGTTAAACCA | | | |
| | AATAAGGCT | (SEQ ID | C (SEQ | ACTAGAA | | | |
| | AGTCCGTTA | NO: 405) | ID NO: | (SEQ ID NO: | | | |
| | TCAACTTGA | | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 76 | PBS 13 |
| GGAATGG (SEQ ID | CTAGAAATA | ATTGAC | CCAG | TCTAGTTACG | TT | | RTT 16 |
| NO: 1) | GCAAGTTAA | ACAA | TCCT | CGTTAAACCA | | | |
| | AATAAGGCT | (SEQ ID | C (SEQ | ACTAGAA | | | |
| | AGTCCGTTA | NO: 406) | ID NO: | (SEQ ID NO: | | | |
| | TCAACTTGA | | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 77 | PBS 13 |
| GGAATGG (SEQ ID | CTAGAAATA | TATTGA | CCAG | TCTAGTTACG | TT | | RTT 17 |
| NO: 1) | GCAAGTTAA | CACAA | TCCT | CGTTAAACCA | | | |
| | AATAAGGCT | (SEQ ID | C (SEQ | ACTAGAA | | | |
| | AGTCCGTTA | NO: 407) | ID NO: | (SEQ ID NO: | | | |
| | TCAACTTGA | | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 78 | PBS 13 |
| GGAATGG (SEQ ID | CTAGAAATA | TATATT | CCAG | TCTAGTTACG | TT | | RTT 18 |
| NO: 1) | GCAAGTTAA | GACAC | TCCT | CGTTAAACCA | | | |
| | AATAAGGCT | AA (SEQ | C (SEQ | ACTAGAA | | | |
| | AGTCCGTTA | 408) | ID NO: | (SEQ ID NO: | | | |
| | TCAACTTGA | ID NO: | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 79 | PBS 13 |
| GGAATGG (SEQ ID | CTAGAAATA | CTATAT | CCAG | TCTAGTTACG | TT | | RTT 19 |
| NO: 1) | GCAAGTTAA | TGACA | TCCT | CGTTAAACCA | | | |
| | AATAAGGCT | CAA | C (SEQ | ACTAGAA | | | |
| | AGTCCGTTA | (SEQ ID | ID NO: | (SEQ ID NO: | | | |
| | TCAACTTGA | NO: 409) | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 80 | PBS 13 |
| GGAATGG (SEQ ID | CTAGAAATA | GCTATA | CCAG | TCTAGTTACG | TT | | RTT 20 |
| NO: 1) | GCAAGTTAA | TTGACA | TCCT | CGTTAAACCA | | | |
| | AATAAGGCT | CAA | C (SEQ | ACTAGAA | | | |
| | AGTCCGTTA | (SEQ ID | ID NO: | (SEQ ID NO: | | | |
| | TCAACTTGA | NO: 410) | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 81 | PBS 13 |
| GGAATGG (SEQ ID | CTAGAAATA | AGCTAT | CCAG | TCTAGTTACG | TT | | RTT 21 |
| NO: 1) | GCAAGTTAA | ATTGAC | TCCT | CGTTAAACCA | | | |
| | AATAAGGCT | ACAA | C (SEQ | ACTAGAA | | | |
| | AGTCCGTTA | (SEQ ID | ID NO: | (SEQ ID NO: | | | |
| | TCAACTTGA | NO: 411) | 32) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 82 | PBS 14 |
| GGAATGG (SEQ ID | CTAGAAATA | TGACA | CCAG | TCTAGTTACG | TT | | RTT 14 |
| NO: 1) | GCAAGTTAA | CAA | TCCT | CGTTAAACCA | | | |
| | AATAAGGCT | (SEQ ID | CC | ACTAGAA | | | |
| | AGTCCGTTA | NO: 404) | (SEQ | (SEQ ID NO: | | | |
| | TCAACTTGA | | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 83 | PBS 14 |
| GGAATGG (SEQ ID | CTAGAAATA | TTGACA | CCAG | TCTAGTTACG | TT | | RTT 15 |
| NO: 1) | GCAAGTTAA | CAA | TCCT | CGTTAAACCA | | | |
| | AATAAGGCT | (SEQ ID | CC | ACTAGAA | | | |
| | AGTCCGTTA | NO: 405) | (SEQ | (SEQ ID NO: | | | |
| | TCAACTTGA | | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 84 | PBS 14 |
| GGAATGG (SEQ ID | CTAGAAATA | ATTGAC | CCAG | TCTAGTTACG | TT | | RTT 16 |
| NO: 1) | GCAAGTTAA | ACAA | TCCT | CGTTAAACCA | | | |
| | AATAAGGCT | (SEQ ID | CC | ACTAGAA | | | |
| | AGTCCGTTA | NO: 406) | (SEQ | (SEQ ID NO: | | | |
| | TCAACTTGA | | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 85 | PBS 14 |
| GGAATGG (SEQ ID | CTAGAAATA | TATTGA | CCAG | TCTAGTTACG | TT | | RTT 17 |
| NO: 1) | GCAAGTTAA | CACAA | TCCT | CGTTAAACCA | | | |
| | AATAAGGCT | (SEQ ID | CC | ACTAGAA | | | |
| | AGTCCGTTA | NO: 407) | (SEQ | (SEQ ID NO: | | | |
| | TCAACTTGA | | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 86 | PBS 14 |
| GGAATGG (SEQ ID | CTAGAAATA | TATATT | CCAG | TCTAGTTACG | TT | | RTT 18 |
| NO: 1) | GCAAGTTAA | GACAC | TCCT | CGTTAAACCA | | | |
| | AATAAGGCT | AA (SEQ | CC | ACTAGAA | | | |
| | AGTCCGTTA | ID NO: | (SEQ | (SEQ ID NO: | | | |
| | TCAACTTGA | 408) | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 87 | PBS 14 |
| GGAATGG (SEQ ID | CTAGAAATA | CTATAT | CCAG | TCTAGTTACG | TT | | RTT 19 |
| NO: 1) | GCAAGTTAA | TGACA | TCCT | CGTTAAACCA | | | |
| | AATAAGGCT | CAA | CC | ACTAGAA | | | |
| | AGTCCGTTA | (SEQ ID | (SEQ | (SEQ ID NO: | | | |
| | TCAACTTGA | NO: 409) | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 88 | PBS 14 |
| GGAATGG (SEQ ID | CTAGAAATA | GCTATA | CCAG | TCTAGTTACG | TT | | RTT 20 |
| NO: 1) | GCAAGTTAA | TTGACA | TCCT | CGTTAAACCA | | | |
| | AATAAGGCT | CAA | CC | ACTAGAA | | | |
| | AGTCCGTTA | (SEQ ID | (SEQ | (SEQ ID NO: | | | |
| | TCAACTTGA | NO: 410) | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 89 | PBS 14 |
| GGAATGG (SEQ ID | CTAGAAATA | AGCTAT | CCAG | TCTAGTTACG | TT | | RTT 21 |
| NO: 1) | GCAAGTTAA | ATTGAC | TCCT | CGTTAAACCA | | | |
| | AATAAGGCT | ACAA | CC | ACTAGAA | | | |
| | AGTCCGTTA | (SEQ ID | (SEQ | (SEQ ID NO: | | | |
| | TCAACTTGA | NO: 411) | ID NO: | 14) | | | |
| | AAAAGTGG | | 33) | | | | |
| | CACCGAGTC | | | | | | |
| | GGTGC (SEQ | | | | | | |
| | ID NO: 576) | | | | | | |
| GCCGGGAGGACTGG | GTTTTAGAG | CTATAT | TTCC | CGCGGTTCTA | TTTT | 90 | PBS 15 |
| GGAATGG (SEQ ID | CTAGAAATA | TGACA | CCAG | TCTAGTTACG | TT | | RTT 14 |
| NO: 1) | GCAAGTTAA | CAA | TCCT | CGTTAAACCA | | | |
| | AATAAGGCT | (SEQ ID | CCC | ACTAGAA | | | |
| | AGTCCGTTA | NO: 404) | (SEQ | (SEQ ID NO: | | | |
| | TCAACTTGA | | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 91 | PBS 15 |
| GGAATGG (SEQ ID | CTAGAAATA | TTGACA | CCAG | TCTAGTTACG | TT | | RTT 15 |
| NO: 1) | GCAAGTTAA | CAA | TCCT | CGTTAAACCA | | | |
| | AATAAGGCT | (SEQ ID | CCC | ACTAGAA | | | |
| | AGTCCGTTA | NO: 405) | (SEQ | (SEQ ID NO: | | | |
| | TCAACTTGA | | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | AGCTAT | TTCC | CGCGGTTCTA | TTTT | 92 | PBS 15 |
| GGAATGG (SEQ ID | CTAGAAATA | ATTGAC | CCAG | TCTAGTTACG | TT | | RTT 16 |
| NO: 1) | GCAAGTTAA | ACAA | TCCT | CGTTAAACCA | | | |
| | AATAAGGCT | (SEQ ID | CCC | ACTAGAA | | | |
| | AGTCCGTTA | NO: 406) | (SEQ | (SEQ ID NO: | | | |
| | TCAACTTGA | | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | CAGCTA | TTCC | CGCGGTTCTA | TTTT | 93 | PBS 15 |
| GGAATGG (SEQ ID | CTAGAAATA | TATTGA | CCAG | TCTAGTTACG | TT | | RTT 17 |
| NO: 1) | GCAAGTTAA | CACAA | TCCT | CGTTAAACCA | | | |
| | AATAAGGCT | (SEQ ID | CCC | ACTAGAA | | | |
| | AGTCCGTTA | NO: 407) | (SEQ | (SEQ ID NO: | | | |
| | TCAACTTGA | | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | ACAGC | TTCC | CGCGGTTCTA | TTTT | 94 | PBS 15 |
| GGAATGG (SEQ ID | CTAGAAATA | TATATT | CCAG | TCTAGTTACG | TT | | RTT 18 |
| NO: 1) | GCAAGTTAA | GACAC | TCCT | CGTTAAACCA | | | |
| | AATAAGGCT | AA (SEQ | CCC | ACTAGAA | | | |
| | AGTCCGTTA | ID NO: | (SEQ | (SEQ ID NO: | | | |
| | TCAACTTGA | 408) | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | CACAG | TTCC | CGCGGTTCTA | TTTT | 95 | PBS 15 |
| GGAATGG (SEQ ID | CTAGAAATA | CTATAT | CCAG | TCTAGTTACG | TT | | RTT 19 |
| NO: 1) | GCAAGTTAA | TGACA | TCCT | CGTTAAACCA | | | |
| | AATAAGGCT | CAA | CCC | ACTAGAA | | | |
| | AGTCCGTTA | (SEQ ID | (SEQ | (SEQ ID NO: | | | |
| | TCAACTTGA | NO: 409) | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | GCACA | TTCC | CGCGGTTCTA | TTTT | 96 | PBS 15 |
| GGAATGG (SEQ ID | CTAGAAATA | GCTATA | CCAG | TCTAGTTACG | TT | | RTT 20 |
| NO: 1) | GCAAGTTAA | TTGACA | TCCT | CGTTAAACCA | | | |
| | AATAAGGCT | CAA | CCC | ACTAGAA | | | |
| | AGTCCGTTA | (SEQ ID | (SEQ | (SEQ ID NO: | | | |
| | TCAACTTGA | NO: 410) | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | GGCAC | TTCC | CGCGGTTCTA | TTTT | 97 | PBS 15 |
| GGAATGG (SEQ ID | CTAGAAATA | AGCTAT | CCAG | TCTAGTTACG | TT | | RTT 21 |
| NO: 1) | GCAAGTTAA | ATTGAC | TCCT | CGTTAAACCA | | | |
| | AATAAGGCT | ACAA | CCC | ACTAGAA | | | |
| | AGTCCGTTA | (SEQ ID | (SEQ | (SEQ ID NO: | | | |
| | TCAACTTGA | NO: 411) | ID NO: | 14) | | | |
| | AAAAGTGG | | 34) | | | | |
| | CACCGAGTC | | | | | | |
| | GGTGC (SEQ | | | | | | |
| | ID NO: 576) | | | | | | |
| GCCGGGAGGACTGG | GTTTTAGAG | GCTATA | TTCC | CGCGGTTCTA | TTTT | 59 | |
| GGAATGG (SEQ ID | CTAGAAATA | TTGACA | CCAG | TCTAGTTACG | TTT | | |
| NO: 1) | GCAAGTTAA | CAA | TCC | CGTTAAACCA | | | |
| | AATAAGGCT | (SEQ ID | (SEQ | ACTAGAA | | | |
| | AGTCCGTTA | NO: 405) | ID NO: | (SEQ ID NO: | | | |
| | TCAACTTGA | | 30) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTAAGAG | GCTATA | TTCC | CGCGGTTCTA | TTTT | 98 | |
| GGAATGG (SEQ ID | CTAAGCTGG | TTGACA | CCAG | TCTAGTTACG | TTT | | |
| NO: 1) | AAACAGCAT | CAA | TCC | CGTTAAACCA | | | |
| | AGCAAGTTT | (SEQ ID | (SEQ | ACTAGAA | | | |
| | AAATAAGGC | NO: 405) | ID NO: | (SEQ ID NO: | | | |
| | TAGTCCGTT | | 30) | 14) | | | |
| | ATCAACTTG | | | | | | |
| | AAAAAGTG | | | | | | |
| | GCACCGAGT | | | | | | |
| | CGGTGC | | | | | | |
| | (SEQ ID NO: | | | | | | |
| | 578) | | | | | | |
| GCCGGGAGGACTGG | GTTTTAGAG | GCTATA | TTCC | CGCGGTTCTA | TTTT | 43 | |
| GGAATGG (SEQ ID | CTAGAAATA | TTGACA | CCAG | TCTAGTTACG | TTT | | |
| NO: 1) | GCAAGTTAA | CAA | T | CGTTAAACCA | | | |
| | AATAAGGCT | (SEQ ID | | ACTAGAA | | | |
| | AGTCCGTTA | NO: 405) | | (SEQ ID NO: | | | |
| | TCAACTTGA | | | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTAAGAG | GCTATA | TTCC | CGCGGTTCTA | TTTT | 99 | |
| GGAATGG (SEQ ID | CTAAGCTGG | TTGACA | CCAG | TCTAGTTACG | TTT | | |
| NO: 1) | AAACAGCAT | CAA | T | CGTTAAACCA | | | |
| | AGCAAGTTT | (SEQ ID | | ACTAGAA | | | |
| | AAATAAGGC | NO: 405) | | (SEQ ID NO: | | | |
| | TAGTCCGTT | | | 14) | | | |
| | ATCAACTTG | | | | | | |
| | AAAAAGTG | | | | | | |
| | GCACCGAGT | | | | | | |
| | CGGTGC | | | | | | |
| | (SEQ ID NO: | | | | | | |
| | 578) | | | | | | |
| GCCGGGAGGACTGG | GTTTTAGAG | GCTATA | TTCC | CGCGGTTCTA | TTTT | 51 | |
| GGAATGG (SEQ ID | CTAGAAATA | TTGACA | CCAG | TCTAGTTACG | TTT | | |
| NO: 1) | GCAAGTTAA | CAA | TC | CGTTAAACCA | | | |
| | AATAAGGCT | (SEQ ID | (SEQ | ACTAGAA | | | |
| | AGTCCGTTA | NO: 405) | ID NO: | (SEQ ID NO: | | | |
| | TCAACTTGA | | 29) | 14) | | | |
| | AAAAGTGG | | | | | | |
| | CACCGAGTC | | | | | | |
| | GGTGC (SEQ | | | | | | |
| | ID NO: 576) | | | | | | |
| GCCGGGAGGACTGG | GTTTAAGAG | GCTATA | TTCC | CGCGGTTCTA | TTTT | 100 | |
| GGAATGG (SEQ ID | CTAAGCTGG | TTGACA | CCAG | TCTAGTTACG | TTT | | |
| NO: 1) | AAACAGCAT | CAA | TC | CGTTAAACCA | | | |
| | AGCAAGTTT | (SEQ ID | (SEQ | ACTAGAA | | | |
| | AAATAAGGC | NO: 405) | ID NO: | (SEQ ID NO: | | | |
| | TAGTCCGTT | | 29) | 14) | | | |
| | ATCAACTTG | | | | | | |
| | AAAAAGTG | | | | | | |
| | GCACCGAGT | | | | | | |
| | CGGTGC | | | | | | |
| | (SEQ ID NO: | | | | | | |
| | 578) | | | | | | |
| GCCAGATCTCAGCA | GTTTTAGAG | | | | TTTT | 101 | |
| GAACAGG (SEQ ID | CTAGAAATA | | | | TTT | | |
| NO: 2) | GCAAGTTAA | | | | | | |
| | AATAAGGCT | | | | | | |
| | AGTCCGTTA | | | | | | |
| | TCAACTTGA | | | | | | |
| | AAAAGTGG | | | | | | |
| | CACCGAGTC | | | | | | |
| | GGTGC (SEQ | | | | | | |
| | ID NO: 576) | | | | | | |
| GCATTCATGGAAAG | GTTTTAGAG | | | | TTTT | 102 | |
| CTCCCAG (SEQ ID | CTAGAAATA | | | | TTT | | |
| NO: 3) | GCAAGTTAA | | | | | | |
| | AATAAGGCT | | | | | | |
| | AGTCCGTTA | | | | | | |
| | TCAACTTGA | | | | | | |
| | AAAAGTGG | | | | | | |
| | CACCGAGTC | | | | | | |
| | GGTGC (SEQ | | | | | | |
| | ID NO: 576) | | | | | | |
| GCCAGAGCAAAGCT | GTTTTAGAG | | | | TTTT | 103 | |
| GGGACCC (SEQ ID | CTAGAAATA | | | | TTT | | |
| NO: 4) | GCAAGTTAA | | | | | | |
| | AATAAGGCT | | | | | | |
| | AGTCCGTTA | | | | | | |
| | TCAACTTGA | | | | | | |
| | AAAAGTGG | | | | | | |
| | CACCGAGTC | | | | | | |
| | GGTGC (SEQ | | | | | | |
| | ID NO: 576) | | | | | | |

TABLE 2
epegRNAs for SpCas9-NRCH in HEK293T model cell line (All sequences are
shown 5′ to 3′)

| | | Reverse | | | | Sequence | |
|---|---|---|---|---|---|---|---|
| | | Trans- | Primer- | | U6 RNA | Identifier | |
| | | cription | binding | Structured | polymerase | for Full | |
| | Guide RNA | Template | site | 3′ | trans- | epegRNA | |
| epegRNA spacer | scaffold | or RTT | or PBS | tevopreQ1 | criptional | (without | |
| sequence | sequence | (variable | (variable | motif | terminator | term- | |
| (20-21 bp): | (76 bp): | length): | length): | (37 bp): | (7 bp) | inator) | notes |
| GCAGAGTCGGC | GTTTTAGAG | GAATTG | TAGCTATGC | CGCGGTT | TTTTTTT | 104 | correction |
| ATAGCTATAT | CTAGAAATA | TGTCAA | | CTATCTA | | | with +2 |
| (SEQ ID NO: 5) | GCAAGTTAA | GA (SEQ | | GTTACGC | | | seed, PBS |
| | AATAAGGCT | ID NO: | | GTTAAAC | | | 9 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 105 | correction |
| ATAGCTATAT | CTAGAAATA | GTGTCA | | CTATCTA | | | with +2 |
| (SEQ ID NO: 5) | GCAAGTTAA | AGA | | GTTACGC | | | seed, PBS |
| | AATAAGGCT | (SEQ ID | | GTTAAAC | | | 9 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 106 | correction |
| ATAGCTATAT | CTAGAAATA | TTGTGT | | CTATCTA | | | with +2 |
| (SEQ ID NO: 5) | GCAAGTTAA | CAAGA | | GTTACGC | | | seed, PBS |
| | AATAAGGCT | (SEQ ID | | GTTAAAC | | | 9 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 107 | correction |
| ATAGCTATAT | CTAGAAATA | ATTGTG | | CTATCTA | | | with +2 |
| (SEQ ID NO: 5) | GCAAGTTAA | TCAAG | | GTTACGC | | | seed, PBS |
| | AATAAGGCT | A (SEQ | | GTTAAAC | | | 9 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 108 | correction |
| ATAGCTATAT | CTAGAAATA | AATTGT | | CTATCTA | | | with +2 |
| (SEQ ID NO: 5) | GCAAGTTAA | GTCAA | | GTTACGC | | | seed, PBS |
| | AATAAGGCT | GA (SEQ | | GTTAAAC | | | 9 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 109 | correction |
| ATAGCTATAT | CTAGAAATA | GAATTG | | CTATCTA | | | with +2 |
| (SEQ ID NO: 5) | GCAAGTTAA | TGTCAA | | GTTACGC | | | seed, PBS |
| | AATAAGGCT | GA (SEQ | | GTTAAAC | | | 9 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 110 | correction |
| ATAGCTATAT | CTAGAAATA | GGAATT | | CTATCTA | | | with +2 |
| (SEQ ID NO: 5) | GCAAGTTAA | GTGTCA | | GTTACGC | | | seed, PBS |
| | AATAAGGCT | AGA | | GTTAAAC | | | 9 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 111 | correction |
| ATAGCTATAT | CTAGAAATA | GGGAA | | CTATCTA | | | with +2 |
| (SEQ ID NO: 5) | GCAAGTTAA | TTGTGT | | GTTACGC | | | seed, PBS |
| | AATAAGGCT | CAAGA | | GTTAAAC | | | 9 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTTT | 112 | correction |
| ATAGCTATAT | CTAGAAATA | TGTCAA | C (SEQ ID | CTATCTA | | | with +2 |
| (SEQ ID NO: 5) | GCAAGTTAA | GA (SEQ | NO: 35) | GTTACGC | | | seed, PBS |
| | AATAAGGCT | ID NO: | | GTTAAAC | | | 10 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 113 | correction |
| ATAGCTATAT | CTAGAAATA | GTGTCA | C (SEQ ID | CTATCTA | | | with +2 |
| (SEQ ID NO: 5) | GCAAGTTAA | AGA | NO: 35) | GTTACGC | | | seed, PBS |
| | AATAAGGCT | (SEQ ID | | GTTAAAC | | | 10 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 114 | correction |
| ATAGCTATAT | CTAGAAATA | TTGTGT | C (SEQ ID | CTATCTA | | | with +2 |
| (SEQ ID NO: 5) | GCAAGTTAA | CAAGA | NO: 35) | GTTACGC | | | seed, PBS |
| | AATAAGGCT | (SEQ ID | | GTTAAAC | | | 10 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 115 | correction |
| ATAGCTATAT | CTAGAAATA | ATTGTG | C (SEQ ID | CTATCTA | | | with +2 |
| (SEQ ID NO: 5) | GCAAGTTAA | TCAAG | NO: 35) | GTTACGC | | | seed, PBS |
| | AATAAGGCT | A (SEQ | | GTTAAAC | | | 10 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 116 | correction |
| ATAGCTATAT | CTAGAAATA | AATTGT | C (SEQ ID | CTATCTA | | | with +2 |
| (SEQ ID NO: 5) | GCAAGTTAA | GTCAA | NO: 35) | GTTACGC | | | seed, PBS |
| | AATAAGGCT | GA (SEQ | | GTTAAAC | | | 10 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 117 | correction |
| ATAGCTATAT | CTAGAAATA | GAATTG | C (SEQ ID | CTATCTA | | | with +2 |
| (SEQ ID NO: 5) | GCAAGTTAA | TGTCAA | NO: 35) | GTTACGC | | | seed, PBS |
| | AATAAGGCT | GA (SEQ | | GTTAAAC | | | 10 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 118 | correction |
| ATAGCTATAT | CTAGAAATA | GGAATT | C (SEQ ID | CTATCTA | | | with +2 |
| (SEQ ID NO: 5) | GCAAGTTAA | GTGTCA | NO: 35) | GTTACGC | | | seed, PBS |
| | AATAAGGCT | AGA | | GTTAAAC | | | 10 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 119 | correction |
| ATAGCTATAT | CTAGAAATA | GGGAA | C (SEQ ID | CTATCTA | | | with +2 |
| (SEQ ID NO: 5) | GCAAGTTAA | TTGTGT | NO: 35) | GTTACGC | | | seed, PBS |
| | AATAAGGCT | CAAGA | | GTTAAAC | | | 10 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTTT | 120 | correction |
| ATAGCTATAT | CTAGAAATA | TGTCAA | CG (SEQ ID | CTATCTA | | | with +2 |
| (SEQ ID NO: 5) | GCAAGTTAA | GA (SEQ | NO: 36) | GTTACGC | | | seed, PBS |
| | AATAAGGCT | ID NO: | | GTTAAAC | | | 11 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 121 | correction |
| ATAGCTATAT | CTAGAAATA | GTGTCA | CG (SEQ ID | CTATCTA | | | with +2 |
| (SEQ ID NO: 5) | GCAAGTTAA | AGA | NO: 36) | GTTACGC | | | seed, PBS |
| | AATAAGGCT | (SEQ ID | | GTTAAAC | | | 11 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 122 | correction |
| ATAGCTATAT | CTAGAAATA | TTGTGT | CG (SEQ ID | CTATCTA | | | with +2 |
| (SEQ ID NO: 5) | GCAAGTTAA | CAAGA | NO: 36) | GTTACGC | | | seed, PBS |
| | AATAAGGCT | (SEQ ID | | GTTAAAC | | | 11 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 123 | correction |
| ATAGCTATAT | CTAGAAATA | ATTGTG | CG (SEQ ID | CTATCTA | | | with +2 |
| (SEQ ID NO: 5) | GCAAGTTAA | TCAAG | NO: 36) | GTTACGC | | | seed, PBS |
| | AATAAGGCT | A (SEQ | | GTTAAAC | | | 11 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 124 | correction |
| ATAGCTATAT | CTAGAAATA | AATTGT | CG (SEQ ID | CTATCTA | | | with +2 |
| (SEQ ID NO: 5) | GCAAGTTAA | GTCAA | NO: 36) | GTTACGC | | | seed, PBS |
| | AATAAGGCT | GA (SEQ | | GTTAAAC | | | 11 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 125 | correction |
| ATAGCTATAT | CTAGAAATA | GAATTG | CG (SEQ ID | CTATCTA | | | with +2 |
| (SEQ ID NO: 5) | GCAAGTTAA | TGTCAA | NO: 36) | GTTACGC | | | seed, PBS |
| | AATAAGGCT | GA (SEQ | | GTTAAAC | | | 11 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 126 | correction |
| ATAGCTATAT | CTAGAAATA | GGAATT | CG (SEQ ID | CTATCTA | | | with +2 |
| (SEQ ID NO: 5) | GCAAGTTAA | GTGTCA | NO: 36) | GTTACGC | | | seed, PBS |
| | AATAAGGCT | AGA | | GTTAAAC | | | 11 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 127 | correction |
| ATAGCTATAT | CTAGAAATA | GGGAA | CG (SEQ ID | CTATCTA | | | with +2 |
| (SEQ ID NO: 5) | GCAAGTTAA | TTGTGT | NO: 36) | GTTACGC | | | seed, PBS |
| | AATAAGGCT | CAAGA | | GTTAAAC | | | 11 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTTT | 128 | correction |
| ATAGCTATAT | CTAGAAATA | TGTCAA | CGA (SEQ ID | CTATCTA | | | with +2 |
| (SEQ ID NO: 5) | GCAAGTTAA | GA (SEQ | NO: 37) | GTTACGC | | | seed, PBS |
| | AATAAGGCT | ID NO: | | GTTAAAC | | | 12 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 129 | correction |
| ATAGCTATAT | CTAGAAATA | GTGTCA | CGA (SEQ ID | CTATCTA | | | with +2 |
| (SEQ ID NO: 5) | GCAAGTTAA | AGA | NO: 37) | GTTACGC | | | seed, PBS |
| | AATAAGGCT | (SEQ ID | | GTTAAAC | | | 12 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 130 | correction |
| ATAGCTATAT | CTAGAAATA | TTGTGT | CGA (SEQ ID | CTATCTA | | | with +2 |
| (SEQ ID NO: 5) | GCAAGTTAA | CAAGA | NO: 37) | GTTACGC | | | seed, PBS |
| | AATAAGGCT | (SEQ ID | | GTTAAAC | | | 12 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 131 | correction |
| ATAGCTATAT | CTAGAAATA | ATTGTG | CGA (SEQ ID | CTATCTA | | | with +2 |
| (SEQ ID NO: 5) | GCAAGTTAA | TCAAG | NO: 37) | GTTACGC | | | seed, PBS |
| | AATAAGGCT | A (SEQ | | GTTAAAC | | | 12 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 132 | correction |
| ATAGCTATAT | CTAGAAATA | AATTGT | CGA (SEQ ID | CTATCTA | | | with +2 |
| (SEQ ID NO: 5) | GCAAGTTAA | GTCAA | NO: 37) | GTTACGC | | | seed, PBS |
| | AATAAGGCT | GA (SEQ | | GTTAAAC | | | 12 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 133 | correction |
| ATAGCTATAT | CTAGAAATA | GAATTG | CGA (SEQ ID | CTATCTA | | | with +2 |
| (SEQ ID NO: 5) | GCAAGTTAA | TGTCAA | NO: 37) | GTTACGC | | | seed, PBS |
| | AATAAGGCT | GA (SEQ | | GTTAAAC | | | 12 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 134 | correction |
| ATAGCTATAT | CTAGAAATA | GGAATT | CGA (SEQ ID | CTATCTA | | | with +2 |
| (SEQ ID NO: 5) | GCAAGTTAA | GTGTCA | NO: 37) | GTTACGC | | | seed, PBS |
| | AATAAGGCT | AGA | NO: 37) | GTTAAAC | | | 12 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 135 | correction |
| ATAGCTATAT | CTAGAAATA | GGGAA | CGA (SEQ ID | CTATCTA | | | with +2 |
| (SEQ ID NO: 5) | GCAAGTTAA | TTGTGT | | GTTACGC | | | seed, PBS |
| | AATAAGGCT | CAAGA | | GTTAAAC | | | 12 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTTT | 136 | correction |
| ATAGCTATAT | CTAGAAATA | TGTCAA | CGAC (SEQ | CTATCTA | | | with +2 |
| (SEQ ID NO: 5) | GCAAGTTAA | GA (SEQ | ID NO: 38) | GTTACGC | | | seed, PBS |
| | AATAAGGCT | ID NO: | | GTTAAAC | | | 13 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 137 | correction |
| ATAGCTATAT | CTAGAAATA | GTGTCA | CGAC (SEQ | CTATCTA | | | with +2 |
| (SEQ ID NO: 5) | GCAAGTTAA | AGA | ID NO: 38) | GTTACGC | | | seed, PBS |
| | AATAAGGCT | (SEQ ID | | GTTAAAC | | | 13 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 138 | correction |
| ATAGCTATAT | CTAGAAATA | TTGTGT | CGAC (SEQ | CTATCTA | | | with +2 |
| (SEQ ID NO: 5) | GCAAGTTAA | CAAGA | ID NO: 38) | GTTACGC | | | seed, PBS |
| | AATAAGGCT | (SEQ ID | | GTTAAAC | | | 13 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 139 | correction |
| ATAGCTATAT | CTAGAAATA | ATTGTG | CGAC (SEQ | CTATCTA | | | with +2 |
| (SEQ ID NO: 5) | GCAAGTTAA | TCAAG | ID NO: 38) | GTTACGC | | | seed, PBS |
| | AATAAGGCT | A (SEQ | | GTTAAAC | | | 13 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 140 | correction |
| ATAGCTATAT | CTAGAAATA | AATTGT | CGAC (SEQ | CTATCTA | | | with +2 |
| (SEQ ID NO: 5) | GCAAGTTAA | GTCAA | ID NO: 38) | GTTACGC | | | seed, PBS |
| | AATAAGGCT | GA (SEQ | | GTTAAAC | | | 13 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 141 | correction |
| ATAGCTATAT | CTAGAAATA | GAATTG | CGAC (SEQ | CTATCTA | | | with +2 |
| (SEQ ID NO: 5) | GCAAGTTAA | TGTCAA | ID NO: 38) | GTTACGC | | | seed, PBS |
| | AATAAGGCT | GA (SEQ | | GTTAAAC | | | 13 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 142 | correction |
| ATAGCTATAT | CTAGAAATA | GGAATT | CGAC (SEQ | CTATCTA | | | with +2 |
| (SEQ ID NO: 5) | GCAAGTTAA | GTGTCA | ID NO: 38) | GTTACGC | | | seed, PBS |
| | AATAAGGCT | AGA | | GTTAAAC | | | 13 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 143 | correction |
| ATAGCTATAT | CTAGAAATA | GGGAA | CGAC (SEQ | CTATCTA | | | with +2 |
| (SEQ ID NO: 5) | GCAAGTTAA | TTGTGT | ID NO: 38) | GTTACGC | | | seed, PBS |
| | AATAAGGCT | CAAGA | | GTTAAAC | | | 13 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTTT | 144 | correction |
| ATAGCTATAT | CTAGAAATA | TGTCAA | CGACT (SEQ | CTATCTA | | | with +2 |
| (SEQ ID NO: 5) | GCAAGTTAA | GA (SEQ | ID NO: 6) | GTTACGC | | | seed, PBS |
| | AATAAGGCT | ID NO: | | GTTAAAC | | | 14 RTT 14 |
| | AGTCCGTTA | 412) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGAATT | TAGCTATGC | CGCGGTT | TTTTTTT | 145 | correction |
| ATAGCTATAT | CTAGAAATA | GTGTCA | CGACT (SEQ | CTATCTA | | | with +2 |
| (SEQ ID NO: 5) | GCAAGTTAA | AGA | ID NO: 6) | GTTACGC | | | seed, PBS |
| | AATAAGGCT | (SEQ ID | | GTTAAAC | | | 14 RTT 15 |
| | AGTCCGTTA | NO: 413) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGAA | TAGCTATGC | CGCGGTT | TTTTTTT | 146 | correction |
| ATAGCTATAT | CTAGAAATA | TTGTGT | CGACT (SEQ | CTATCTA | | | with +2 |
| (SEQ ID NO: 5) | GCAAGTTAA | CAAGA | ID NO: 6) | GTTACGC | | | seed, PBS |
| | AATAAGGCT | (SEQ ID | | GTTAAAC | | | 14 RTT 16 |
| | AGTCCGTTA | NO: 414) | | CAACTAG | | | |
| | TCAACTTGA | | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GGGGA | TAGCTATGC | CGCGGTT | TTTTTTT | 147 | correction |
| ATAGCTATAT | CTAGAAATA | ATTGTG | CGACT (SEQ | CTATCTA | | | with +2 |
| (SEQ ID NO: 5) | GCAAGTTAA | TCAAG | ID NO: 6) | GTTACGC | | | seed, PBS |
| | AATAAGGCT | A (SEQ | | GTTAAAC | | | 14 RTT 17 |
| | AGTCCGTTA | ID NO: | | CAACTAG | | | |
| | TCAACTTGA | 415) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | TGGGG | TAGCTATGC | CGCGGTT | TTTTTTT | 148 | correction |
| ATAGCTATAT | CTAGAAATA | AATTGT | CGACT (SEQ | CTATCTA | | | with +2 |
| (SEQ ID NO: 5) | GCAAGTTAA | GTCAA | ID NO: 6) | GTTACGC | | | seed, PBS |
| | AATAAGGCT | GA (SEQ | | GTTAAAC | | | 14 RTT 18 |
| | AGTCCGTTA | ID NO: | | CAACTAG | | | |
| | TCAACTTGA | 416) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | CTGGG | TAGCTATGC | CGCGGTT | TTTTTTT | 149 | correction |
| ATAGCTATAT | CTAGAAATA | GAATTG | CGACT (SEQ | CTATCTA | | | with +2 |
| (SEQ ID NO: 5) | GCAAGTTAA | TGTCAA | ID NO: 6) | GTTACGC | | | seed, PBS |
| | AATAAGGCT | GA (SEQ | | GTTAAAC | | | 14 RTT 19 |
| | AGTCCGTTA | ID NO: | | CAACTAG | | | |
| | TCAACTTGA | 417) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | ACTGG | TAGCTATGC | CGCGGTT | TTTTTTT | 150 | correction |
| ATAGCTATAT | CTAGAAATA | GGAATT | CGACT (SEQ | CTATCTA | | | with +2 |
| (SEQ ID NO: 5) | GCAAGTTAA | GTGTCA | ID NO: 6) | GTTACGC | | | seed, PBS |
| | AATAAGGCT | AGA | | GTTAAAC | | | 14 RTT 20 |
| | AGTCCGTTA | (SEQ ID | | CAACTAG | | | |
| | TCAACTTGA | NO: 418) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GACTG | TAGCTATGC | CGCGGTT | TTTTTTT | 151 | correction |
| ATAGCTATAT | CTAGAAATA | GGGAA | CGACT (SEQ | CTATCTA | | | with +2 |
| (SEQ ID NO: 5) | GCAAGTTAA | TTGTGT | ID NO: 6) | GTTACGC | | | seed, PBS |
| | AATAAGGCT | CAAGA | | GTTAAAC | | | 14 RTT 21 |
| | AGTCCGTTA | (SEQ ID | | CAACTAG | | | |
| | TCAACTTGA | NO: 419) | | AA (SEQ | | | |
| | AAAAGTGG | | | ID NO: 14) | | | |
| | CACCGAGTC | | | | | | |
| | GGTGC (SEQ | | | | | | |
| | ID NO: 576) | | | | | | |
| GCAGAGTCGGC | GTTTTAGAG | GAATTG | TAGCTATGC | CGCGGTT | TTTTTT | 152 | correction |
| ATAGCTATAT | CTAGAAATA | TGTGA | | CTATCTA | | | with +2 |
| (SEQ ID NO: 5) | GCAAGTTAA | GGA | | GTTACGC | | | seed, |
| | AATAAGGCT | SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 14 |
| GCAGAGTCGGC | GTTTTAGAG | GGAATT | TAGCTATGC | CGCGGTT | TTTTTT | 153 | correction |
| ATAGCTATAT | CTAGAAATA | GTGTG | | CTATCTA | | | with +2 |
| (SEQ ID NO: 5) | GCAAGTTAA | AGGA | | GTTACGC | | | seed, |
| | AATAAGGCT | (SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 15 |
| GCAGAGTCGGC | GTTTTAGAG | GGGAA | TAGCTATGC | CGCGGTT | TTTTTT | 154 | correction |
| ATAGCTATAT | CTAGAAATA | TTGTGT | | CTATCTA | | | with +2 |
| (SEQ ID NO: 5) | GCAAGTTAA | GAGGA | | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 16 |
| GCAGAGTCGGC | GTTTTAGAG | GGGGA | TAGCTATGC | CGCGGTT | TTTTTT | 155 | correction |
| ATAGCTATAT | CTAGAAATA | ATTGTG | | CTATCTA | | | with +2 |
| (SEQ ID NO: 5) | GCAAGTTAA | TGAGG | | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 17 |
| GCAGAGTCGGC | GTTTTAGAG | TGGGG | TAGCTATGC | CGCGGTT | TTTTTT | 156 | correction |
| ATAGCTATAT | CTAGAAATA | AATTGT | | CTATCTA | | | with +2 |
| (SEQ ID NO: 5) | GCAAGTTAA | GTGAG | | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 18 |
| GCAGAGTCGGC | GTTTTAGAG | CTGGG | TAGCTATGC | CGCGGTT | TTTTTT | 157 | correction |
| ATAGCTATAT | CTAGAAATA | GAATTG | | CTATCTA | | | with +2 |
| (SEQ ID NO: 5) | GCAAGTTAA | TGTGA | | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 19 |
| GCAGAGTCGGC | GTTTTAGAG | ACTGG | TAGCTATGC | CGCGGTT | TTTTTT | 158 | correction |
| ATAGCTATAT | CTAGAAATA | GGAATT | | CTATCTA | | | with +2 |
| (SEQ ID NO: 5) | GCAAGTTAA | GTGTG | | GTTACGC | | | seed, |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 20 |
| GCAGAGTCGGC | GTTTTAGAG | GACTG | TAGCTATGC | CGCGGTT | TTTTTT | 159 | correction |
| ATAGCTATAT | CTAGAAATA | GGGAA | | CTATCTA | | | with +2 |
| (SEQ ID NO: 5) | GCAAGTTAA | TTGTGT | | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 9 |
| | ID NO: 576) | | | | | | RTT 21 |
| GCAGAGTCGGC | GTTTTAGAG | GAATTG | TAGCTATGC | CGCGGTT | TTTTTT | 160 | correction |
| ATAGCTATAT | CTAGAAATA | TGTGA | C (SEQ ID | CTATCTA | | | with +2 |
| (SEQ ID NO: 5) | GCAAGTTAA | GGA | NO: 35) | GTTACGC | | | seed, |
| | AATAAGGCT | (SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 14 |
| GCAGAGTCGGC | GTTTTAGAG | GGAATT | TAGCTATGC | CGCGGTT | TTTTTT | 161 | correction |
| ATAGCTATAT | CTAGAAATA | GTGTG | C (SEQ ID | CTATCTA | | | with +2 |
| (SEQ ID NO: 5) | GCAAGTTAA | AGGA | NO: 35) | GTTACGC | | | seed, |
| | AATAAGGCT | (SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 15 |
| GCAGAGTCGGC | GTTTTAGAG | GGGAA | TAGCTATGC | CGCGGTT | TTTTTT | 162 | correction |
| ATAGCTATAT | CTAGAAATA | TTGTGT | C (SEQ ID | CTATCTA | | | with +2 |
| (SEQ ID NO: 5) | GCAAGTTAA | GAGGA | NO: 35) | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 16 |
| GCAGAGTCGGC | GTTTTAGAG | GGGGA | TAGCTATGC | CGCGGTT | TTTTTT | 163 | correction |
| ATAGCTATAT | CTAGAAATA | ATTGTG | C (SEQ ID | CTATCTA | | | with +2 |
| (SEQ ID NO: 5) | GCAAGTTAA | TGAGG | NO: 35) | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 17 |
| GCAGAGTCGGC | GTTTTAGAG | TGGGG | TAGCTATGC | CGCGGTT | TTTTTT | 164 | correction |
| ATAGCTATAT | CTAGAAATA | AATTGT | C (SEQ ID | CTATCTA | | | with +2 |
| (SEQ ID NO: 5) | GCAAGTTAA | GTGAG | NO: 35) | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 18 |
| GCAGAGTCGGC | GTTTTAGAG | CTGGG | TAGCTATGC | CGCGGTT | TTTTTT | 165 | correction |
| ATAGCTATAT | CTAGAAATA | GAATTG | C (SEQ ID | CTATCTA | | | with +2 |
| (SEQ ID NO: 5) | GCAAGTTAA | TGTGA | NO: 35) | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 19 |
| GCAGAGTCGGC | GTTTTAGAG | ACTGG | TAGCTATGC | CGCGGTT | TTTTTT | 166 | correction |
| ATAGCTATAT | CTAGAAATA | GGAATT | C (SEQ ID | CTATCTA | | | with +2 |
| (SEQ ID NO: 5) | GCAAGTTAA | GTGTG | NO: 35) | GTTACGC | | | seed, |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 20 |
| GCAGAGTCGGC | GTTTTAGAG | GACTG | TAGCTATGC | CGCGGTT | TTTTTT | 167 | correction |
| ATAGCTATAT | CTAGAAATA | GGGAA | C (SEQ ID | CTATCTA | | | with +2 |
| (SEQ ID NO: 5) | GCAAGTTAA | TTGTGT | NO: 35) | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 10 |
| | ID NO: 576) | | | | | | RTT 21 |
| GCAGAGTCGGC | GTTTTAGAG | GAATTG | TAGCTATGC | CGCGGTT | TTTTTT | 168 | correction |
| ATAGCTATAT | CTAGAAATA | TGTGA | CG (SEQ ID | CTATCTA | | | with +2 |
| (SEQ ID NO: 5) | GCAAGTTAA | GGA | NO: 36) | GTTACGC | | | seed, |
| | AATAAGGCT | (SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 14 |
| GCAGAGTCGGC | GTTTTAGAG | GGAATT | TAGCTATGC | CGCGGTT | TTTTTT | 169 | correction |
| ATAGCTATAT | CTAGAAATA | GTGTG | CG (SEQ ID | CTATCTA | | | with +2 |
| (SEQ ID NO: 5) | GCAAGTTAA | AGGA | NO: 36) | GTTACGC | | | seed, |
| | AATAAGGCT | (SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 15 |
| GCAGAGTCGGC | GTTTTAGAG | GGGAA | TAGCTATGC | CGCGGTT | TTTTTT | 170 | correction |
| ATAGCTATAT | CTAGAAATA | TTGTGT | CG (SEQ ID | CTATCTA | | | with +2 |
| (SEQ ID NO: 5) | GCAAGTTAA | GAGGA | NO: 36) | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 16 |
| GCAGAGTCGGC | GTTTTAGAG | GGGGA | TAGCTATGC | CGCGGTT | TTTTTT | 171 | correction |
| ATAGCTATAT | CTAGAAATA | ATTGTG | CG (SEQ ID | CTATCTA | | | with +2 |
| (SEQ ID NO: 5) | GCAAGTTAA | TGAGG | NO: 36) | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 17 |
| GCAGAGTCGGC | GTTTTAGAG | TGGGG | TAGCTATGC | CGCGGTT | TTTTTT | 172 | correction |
| ATAGCTATAT | CTAGAAATA | AATTGT | CG (SEQ ID | CTATCTA | | | with +2 |
| (SEQ ID NO: 5) | GCAAGTTAA | GTGAG | NO: 36) | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G>C), |
| | GGTGC (SEQ | | | | | | PBS 11 |
| | ID NO: 576) | | | | | | RTT 18 |
| GCAGAGTCGGC | GTTTTAGAG | CTGGG | TAGCTATGC | CGCGGTT | TTTTTT | 173 | correction |
| ATAGCTATAT | CTAGAAATA | GAATTG | CG (SEQ ID | CTATCTA | | | with +2 |
| (SEQ ID NO: 5) | GCAAGTTAA | TGTGA | NO: 36) | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 19 |
| GCAGAGTCGGC | GTTTTAGAG | ACTGG | TAGCTATGC | CGCGGTT | TTTTTT | 174 | correction |
| ATAGCTATAT | CTAGAAATA | GGAATT | CG (SEQ ID | CTATCTA | | | with +2 |
| (SEQ ID NO: 5) | GCAAGTTAA | GTGTG | NO: 36) | GTTACGC | | | seed, |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 20 |
| GCAGAGTCGGC | GTTTTAGAG | GACTG | TAGCTATGC | CGCGGTT | TTTTTT | 175 | correction |
| ATAGCTATAT | CTAGAAATA | GGGAA | CG (SEQ ID | CTATCTA | | | with +2 |
| (SEQ ID NO: 5) | GCAAGTTAA | TTGTGT | NO: 36) | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 11 |
| | ID NO: 576) | | | | | | RTT 21 |
| GCAGAGTCGGC | GTTTTAGAG | GAATTG | TAGCTATGC | CGCGGTT | TTTTTT | 176 | correction |
| ATAGCTATAT | CTAGAAATA | TGTGA | CGA (SEQ ID | CTATCTA | | | with +2 |
| (SEQ ID NO: 5) | GCAAGTTAA | GGA | NO: 37) | GTTACGC | | | seed, |
| | AATAAGGCT | (SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 14 |
| GCAGAGTCGGC | GTTTTAGAG | GGAATT | TAGCTATGC | CGCGGTT | TTTTTT | 177 | correction |
| ATAGCTATAT | CTAGAAATA | GTGTG | CGA (SEQ ID | CTATCTA | | | with +2 |
| (SEQ ID NO: 5) | GCAAGTTAA | AGGA | NO: 37) | GTTACGC | | | seed, |
| | AATAAGGCT | (SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 15 |
| GCAGAGTCGGC | GTTTTAGAG | GGGAA | TAGCTATGC | CGCGGTT | TTTTTT | 178 | correction |
| ATAGCTATAT | CTAGAAATA | TTGTGT | CGA (SEQ ID | CTATCTA | | | with +2 |
| (SEQ ID NO: 5) | GCAAGTTAA | GAGGA | NO: 37) | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 16 |
| GCAGAGTCGGC | GTTTTAGAG | GGGGA | TAGCTATGC | CGCGGTT | TTTTTT | 179 | correction |
| ATAGCTATAT | CTAGAAATA | ATTGTG | CGA (SEQ ID | CTATCTA | | | with +2 |
| (SEQ ID NO: 5) | GCAAGTTAA | TGAGG | NO: 37) | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 17 |
| GCAGAGTCGGC | GTTTTAGAG | TGGGG | TAGCTATGC | CGCGGTT | TTTTTT | 180 | correction |
| ATAGCTATAT | CTAGAAATA | AATTGT | CGA (SEQ ID | CTATCTA | | | with +2 |
| (SEQ ID NO: 5) | GCAAGTTAA | GTGAG | NO: 37) | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 18 |
| GCAGAGTCGGC | GTTTTAGAG | CTGGG | TAGCTATGC | CGCGGTT | TTTTTT | 181 | correction |
| ATAGCTATAT | CTAGAAATA | GAATTG | CGA (SEQ ID | CTATCTA | | | with +2 |
| (SEQ ID NO: 5) | GCAAGTTAA | TGTGA | NO: 37) | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 19 |
| GCAGAGTCGGC | GTTTTAGAG | ACTGG | TAGCTATGC | CGCGGTT | TTTTTT | 182 | correction |
| ATAGCTATAT | CTAGAAATA | GGAATT | CGA (SEQ ID | CTATCTA | | | with +2 |
| (SEQ ID NO: 5) | GCAAGTTAA | GTGTG | NO: 37) | GTTACGC | | | seed. |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 20 |
| GCAGAGTCGGC | GTTTTAGAG | GACTG | TAGCTATGC | CGCGGTT | TTTTTT | 183 | correction |
| ATAGCTATAT | CTAGAAATA | GGGAA | CGA (SEQ ID | CTATCTA | | | with +2 |
| (SEQ ID NO: 5) | GCAAGTTAA | TTGTGT | NO: 37) | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 12 |
| | ID NO: 576) | | | | | | RTT 21 |
| GCAGAGTCGGC | GTTTTAGAG | GAATTG | TAGCTATGC | CGCGGTT | TTTTTT | 184 | correction |
| ATAGCTATAT | CTAGAAATA | TGTGA | CGAC (SEQ | CTATCTA | | | with +2 |
| (SEQ ID NO: 5) | GCAAGTTAA | GGA | ID NO: 38) | GTTACGC | | | seed, |
| | AATAAGGCT | (SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 14 |
| GCAGAGTCGGC | GTTTTAGAG | GGAATT | TAGCTATGC | CGCGGTT | TTTTTT | 185 | correction |
| ATAGCTATAT | CTAGAAATA | GTGTG | CGAC (SEQ | CTATCTA | | | with +2 |
| (SEQ ID NO: 5) | GCAAGTTAA | AGGA | ID NO: 38) | GTTACGC | | | seed, |
| | AATAAGGCT | SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 15 |
| GCAGAGTCGGC | GTTTTAGAG | GGGAA | TAGCTATGC | CGCGGTT | TTTTTT | 186 | correction |
| ATAGCTATAT | CTAGAAATA | TTGTGT | CGAC (SEQ | CTATCTA | | | with +2 |
| (SEQ ID NO: 5) | GCAAGTTAA | GAGGA | ID NO: 38) | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 16 |
| GCAGAGTCGGC | GTTTTAGAG | GGGGA | TAGCTATGC | CGCGGTT | TTTTTT | 187 | correction |
| ATAGCTATAT | CTAGAAATA | ATTGTG | CGAC (SEQ | CTATCTA | | | with +2 |
| (SEQ ID NO: 5) | GCAAGTTAA | TGAGG | ID NO: 38) | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 17 |
| GCAGAGTCGGC | GTTTTAGAG | TGGGG | TAGCTATGC | CGCGGTT | TTTTTT | 188 | correction |
| ATAGCTATAT | CTAGAAATA | AATTGT | CGAC (SEQ | CTATCTA | | | with +2 |
| (SEQ ID NO: 5) | GCAAGTTAA | GTGAG | ID NO: 38) | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 18 |
| GCAGAGTCGGC | GTTTTAGAG | CTGGG | TAGCTATGC | CGCGGTT | TTTTTT | 189 | correction |
| ATAGCTATAT | CTAGAAATA | GAATTG | CGAC (SEQ | CTATCTA | | | with +2 |
| (SEQ ID NO: 5) | GCAAGTTAA | TGTGA | ID NO: 38) | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 19 |
| GCAGAGTCGGC | GTTTTAGAG | ACTGG | TAGCTATGC | CGCGGTT | TTTTTT | 190 | correction |
| ATAGCTATAT | CTAGAAATA | GGAATT | CGAC (SEQ | CTATCTA | | | with +2 |
| (SEQ ID NO: 5) | GCAAGTTAA | GTGTG | ID NO: 38) | GTTACGC | | | seed, |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 20 |
| GCAGAGTCGGC | GTTTTAGAG | GACTG | TAGCTATGC | CGCGGTT | TTTTTT | 191 | correction |
| ATAGCTATAT | CTAGAAATA | GGGAA | CGAC (SEQ | CTATCTA | | | with +2 |
| (SEQ ID NO: 5) | GCAAGTTAA | TTGTGT | ID NO: 38) | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 13 |
| | ID NO: 576) | | | | | | RTT 21 |
| GCAGAGTCGGC | GTTTTAGAG | GAATTG | TAGCTATGC | CGCGGTT | TTTTTT | 192 | correction |
| ATAGCTATAT | CTAGAAATA | TGTGA | CGACT (SEQ | CTATCTA | | | with +2 |
| (SEQ ID NO: 5) | GCAAGTTAA | GGA | ID NO: 6) | GTTACGC | | | seed, |
| | AATAAGGCT | (SEQ ID | | GTTAAAC | | | recoded |
| | AGTCCGTTA | NO: 564) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 14 |
| GCAGAGTCGGC | GTTTTAGAG | GGAATT | TAGCTATGC | CGCGGTT | TTTTTT | 193 | correction |
| ATAGCTATAT | CTAGAAATA | GTGTG | CGACT (SEQ | CTATCTA | | | with +2 |
| (SEQ ID NO: 5) | GCAAGTTAA | AGGA | ID NO: 6) | GTTACGC | | | seed, |
| | AATAAGGCT | (SEQ ID | | GTTAAAC | | | recoded |
| | AGTCCGTTA | NO: 565) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 15 |
| GCAGAGTCGGC | GTTTTAGAG | GGGAA | TAGCTATGC | CGCGGTT | TTTTTT | 194 | correction |
| ATAGCTATAT | CTAGAAATA | TTGTGT | CGACT (SEQ | CTATCTA | | | with +2 |
| (SEQ ID NO: 5) | GCAAGTTAA | GAGGA | ID NO: 6) | GTTACGC | | | seed, |
| | AATAAGGCT | (SEQ ID | | GTTAAAC | | | recoded |
| | AGTCCGTTA | NO: 566) | | CAACTAG | | | Leucine |
| | TCAACTTGA | | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 16 |
| GCAGAGTCGGC | GTTTTAGAG | GGGGA | TAGCTATGC | CGCGGTT | TTTTTT | 195 | correction |
| ATAGCTATAT | CTAGAAATA | ATTGTG | CGACT (SEQ | CTATCTA | | | with +2 |
| (SEQ ID NO: 5) | GCAAGTTAA | TGAGG | ID NO: 6) | GTTACGC | | | seed, |
| | AATAAGGCT | A (SEQ | | GTTAAAC | | | recoded |
| | AGTCCGTTA | ID NO: | | CAACTAG | | | Leucine |
| | TCAACTTGA | 567) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 17 |
| GCAGAGTCGGC | GTTTTAGAG | TGGGG | TAGCTATGC | CGCGGTT | TTTTTT | 196 | correction |
| ATAGCTATAT | CTAGAAATA | AATTGT | CGACT (SEQ | CTATCTA | | | with +2 |
| (SEQ ID NO: 5) | GCAAGTTAA | GTGAG | ID NO: 6) | GTTACGC | | | seed, |
| | AATAAGGCT | GA (SEQ | | GTTAAAC | | | recoded |
| | AGTCCGTTA | ID NO: | | CAACTAG | | | Leucine |
| | TCAACTTGA | 568) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 18 |
| GCAGAGTCGGC | GTTTTAGAG | CTGGG | TAGCTATGC | CGCGGTT | TTTTTT | 197 | correction |
| ATAGCTATAT | CTAGAAATA | GAATTG | CGACT (SEQ | CTATCTA | | | with +2 |
| (SEQ ID NO: 5) | GCAAGTTAA | TGTGA | ID NO: 6) | GTTACGC | | | seed, |
| | AATAAGGCT | GGA | | GTTAAAC | | | recoded |
| | AGTCCGTTA | (SEQ ID | | CAACTAG | | | Leucine |
| | TCAACTTGA | NO: 569) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 19 |
| GCAGAGTCGGC | GTTTTAGAG | ACTGG | TAGCTATGC | CGCGGTT | TTTTTT | 198 | correction |
| ATAGCTATAT | CTAGAAATA | GGAATT | CGACT (SEQ | CTATCTA | | | with +2 |
| (SEQ ID NO: 5) | GCAAGTTAA | GTGTG | ID NO: 6) | GTTACGC | | | seed, |
| | AATAAGGCT | AGGA | | GTTAAAC | | | recoded |
| | AGTCCGTTA | (SEQ ID | | CAACTAG | | | Leucine |
| | TCAACTTGA | NO: 570) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 20 |
| GCAGAGTCGGC | GTTTTAGAG | GACTG | TAGCTATGC | CGCGGTT | TTTTTT | 199 | correction |
| ATAGCTATAT | CTAGAAATA | GGGAA | CGACT (SEQ | CTATCTA | | | with +2 |
| (SEQ ID NO: 5) | GCAAGTTAA | TTGTGT | ID NO: 6) | GTTACGC | | | seed, |
| | AATAAGGCT | GAGGA | | GTTAAAC | | | recoded |
| | AGTCCGTTA | (SEQ ID | | CAACTAG | | | Leucine |
| | TCAACTTGA | NO: 571) | | AA (SEQ | | | (+3 T > C |
| | AAAAGTGG | | | ID NO: 14) | | | and +5 |
| | CACCGAGTC | | | | | | G > C), |
| | GGTGC (SEQ | | | | | | PBS 14 |
| | ID NO: 576) | | | | | | RTT 21 |

TABLE 3
Nicking guide RNAs (All sequences are shown 5′ to 3′)

| spacer | scaffold | terminator | notes |
|---|---|---|---|
| GCTGGGGAATTGT | GTTTAAGAGCTAA | TTTTTT | NRCH 3b Nick for |
| GTCAAGAT (SEQ | GCTGGAAACAGCA | | correction with +2 |
| ID NO: 10) | TAGCAAGTTTAAA | | seed |
| | TAAGGCTAGTCCG | | |
| | TTATCAACTTGAA | | |
| | AAAGTGGCACCGA | | |
| | GTCGGTGC (SEQ | | |
| | ID NO: 578) | | |
| GGCCTTGGTCCTG | GTTTAAGAGCTAA | TTTTTT | General NRCH NG |
| TAGGAGG (SEQ ID | GCTGGAAACAGCA | | |
| NO: 11) | TAGCAAGTTTAAA | | |
| | TAAGGCTAGTCCG | | |
| | TTATCAACTTGAA | | |
| | AAAGTGGCACCGA | | |
| | GTCGGTGC (SEQ | | |
| | ID NO: 578) | | |
| GTTGCTCACAGAC | GTTTAAGAGCTAA | TTTTTT | General NRCH NG |
| TTGGAGT (SEQ ID | GCTGGAAACAGCA | | |
| NO: 12) | TAGCAAGTTTAAA | | |
| | TAAGGCTAGTCCG | | |
| | TTATCAACTTGAA | | |
| | AAAGTGGCACCGA | | |
| | GTCGGTGC (SEQ | | |
| | ID NO: 578) | | |
| GCTGGGGAATTGT | GTTTAAGAGCTAA | TTTTTT | NRCH 3b Nick for |
| GTGAGGAT (SEQ | GCTGGAAACAGCA | | Leucine recoded |
| ID NO: 13) | TAGCAAGTTTAAA | | correction |
| | TAAGGCTAGTCCG | | |
| | TTATCAACTTGAA | | |
| | AAAGTGGCACCGA | | |
| | GTCGGTGC (SEQ | | |
| | ID NO: 578) | | |

### Example 2. Prime Editing Strategies for the Correction of CDKL5 Deficiency Disorder Mutations

CDKL5 deficiency disorder (CDD) is an X-linked, neurological disorder caused by pathogenic mutations in the cyclin-dependent kinase-like 5 (CDKL5) gene¹⁷³. CDKL5 is a serine/threonine kinase with key roles in brain development and function¹⁷⁴ including dendritic microtubule regulation¹⁷⁵ and maintenance of dendritic spine morphologyl^{176,177}. Though originally thought of as an early-onset form of Rett syndrome¹⁷⁸, CDD is now considered a distinct disorder^{179,180} with hundreds of causative mutations in the CDKL5 gene identified in patients^{178,181-191}. While most mutations that cause the disorder are spontaneous de novo mutations, at least one case of a maternally-inherited pathogenic variant of CDKL5 has been described¹⁹². Pathogenic mutations in CDD typically result in haploinsufficiency either through full or partial loss of CDKL5 catalytic activity^{193,194}, though at least one example of a hypermorphic mutation has been described¹⁹⁵, and some lesser-studied CDD mutations may behave similarly to dominant negative mutations found via functional and biochemical assays^{176,196,197}.

Symptoms of CDD begin in early postnatal life, correlating with peak CDKL5 expression^{173,176,196}. A major clinical manifestation of CDD is infantile-onset seizures which are treatment-resistant, however patients also experience developmental delay, intellectual disability, extreme difficulty sleeping, visual impairment, gastrointestinal issues, and respiratory difficulties, among other symptoms^{173,194}. Treatment for CDD is limited to symptom management. Symptom management options include broad-range anti-seizure medications (ASMs) like levetiracetam, the only FDA-approved ASM specifically for CDD called ganaxolone, ketogenic diet, and non-FDA approved cannabidiol derivatives^{198,199}. Currently, there are no curative therapies available for CDD, though investigational disease-modifying therapeutics have been pursued with varying effectiveness^{198,199}. Current investigational therapeutic strategies include epigenetic reactivation of CDKL5 expression²⁰⁰ and a gene therapy to deliver a functional copy of the CDKL5 protein to the brain²⁰¹. Though each of these strategies have unique advantages, none of them directly address the underlying genetic mutations that cause CDD. In contrast, development of a genome editing therapeutic has the potential to be curative by directly correcting pathogenic CDKL5 mutations.

Because CDD mutations can be caused by a suite of genetic changes such as deletions, duplications, and transition or transversion mutations^{178,181-191,195}, development of a genome editing strategy for CDD must utilize a genome editing tool capable of performing precise changes of any variation. Traditional CRISPR nuclease-mediated genome editing results in uncontrolled mixtures of insertion and deletion (indel) alleles^{5,10}, a strategy best suited for disruption of a locus, but not the correction of specific insertions, deletions, or other small mutations that cause CDD. Additionally, nuclease-based strategies require double-strand DNA breaks (DSBs), which can result in p53 activation^{41,43} and undesirable chromosomal abnormalities like large deletions or chromothripsis^{37,42,202}.

In contrast, prime editing can encode deletions, insertions, point mutations, and any combination thereof across hundreds of base pairs at a targeted locus in living cells without requiring a DSB intermediate^{3,86,89,157-160}, making it an optimal choice for the development of corrective genome editing strategies for CDD. As presented herein, three uniquely prime editable (i.e., difficult or impossible to correct precisely with base editing^{53,54} or Cas9 nuclease editing) pathogenic mutations in CDKL5 were focused on, namely a single nucleotide deletion (c.1412delA), a splice acceptor variant surrounded by additional adenines (c.826-1 G>A), and the installation of clustering pathogenic mutations in the eighth exon of CDKL5¹⁸¹. The studies presented herein demonstrate that prime editing can efficiently correct multiple classes of pathogenic CDKL5 variants.

Reported herein is the development of prime editing strategies for all three regions of interest with high on-target efficiency and with minimal indel by-products. The pathogenic allele harboring the equivalent c.1412delA mutation was specifically targeted in murine CDKL5 with correction efficiencies up to 40% by utilizing a bespoke prime editor comprised of a SpCas9-NRCH DNA targeting domain⁶⁰ and PE6 variants (PE6b-d) with evolved and/or engineered reverse transcriptase (RT) domains⁸⁸.

A previously reported c.1412delA correction strategy⁸⁶ was optimized, and 42.3% and 36.2% desired correction was achieved with high specificity in patient primary fibroblasts using PE3max and PE6c⁸⁸ respectively. Finally, the applicability of prime editing to various other pathogenic mutations in CDKL5, including correction of c.826-1 G>A at an average of 8.8% and installing a series of seven pathogenic mutations or seed edits for pathogenic mutations in exon 8, a known hotspot for CDKL5 mutations¹⁸¹, is demonstrated at nearly 30% efficiency. Taken together, these results show that prime editing systems enable the efficient and precise correction of multiple types of pathogenic CDKL5 mutations, even simultaneously with a single prime editing guide RNA (pegRNA), and without requiring DSBs.

### Results

Optimizing Prime Editing to Correct c.1412delA in Murine Cdk15

Nuclease-based editing strategies for the correction of specific nucleotides could result in more indel alleles than alleles with the desired correction^{26,30-32}. This editing strategy is of limited therapeutic benefit for c.1412delA as it is located in the catalytic domain where both in-frame and out-of-frame mutations can manifest in disease¹⁹⁹. Indel alleles created by nuclease editing are predictable but uncontrollable¹⁸, making it hard to specify a particular indel allele as the desired outcome. Though base editors are more precise and cause fewer indel alleles than nuclease-mediated editing strategies, deletions like c.1412delA are not addressable with base editing technology. Base editors enable efficient A.T-to-G.C editing⁵⁴ or C.G-to-T.A⁵³ editing and vice versa, but base editors cannot revert a deletion mutation.

c.1412delA represents a uniquely prime-editable pathogenic variant of CDKL5. A pegRNA was previously optimized to correct c.1412delA via electroporation of in vitro transcribed PE mRNA and synthetic pegRNA into patient-derived induced pluripotent stem cells (iPSCs)⁸⁶. It was sought to further improve this editing strategy with the use of engineered pegRNAs⁸⁹ and a suite of newly evolved and engineered PE6s⁸⁸ at both mouse and human CDKL5.

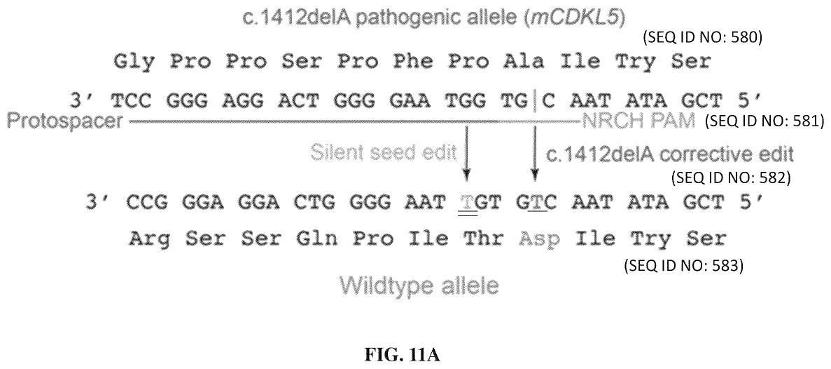

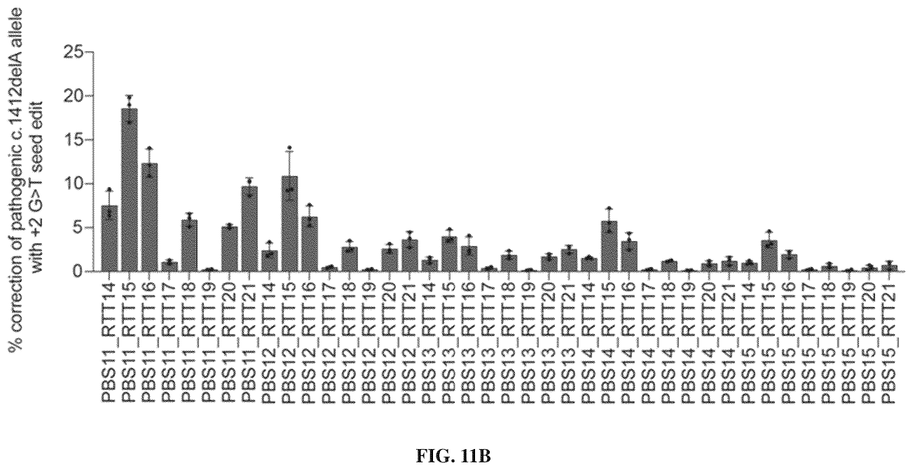

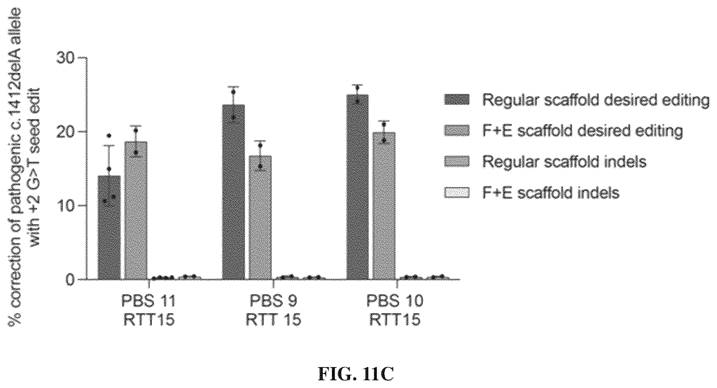

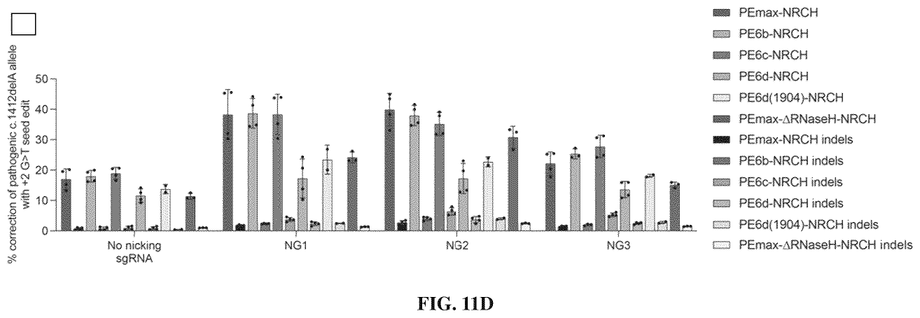

Because the mouse model for the c.1412delA mutation is not humanized, a prime editing strategy to correct the equivalent c.1412delA mutation in the murine Cdkl5 gene was first developed to enable future in vivo studies. The c.1412delA mutation occurred in a female patient, so the patient still has one healthy CDKL5 gene intact. Consequently, it was reasoned that an optimal genome editing strategy would preferentially target the disease allele while bypassing the healthy allele. To this end, an allele-specific prime editing strategy that incorporates recent prime editing advances was developed. Engineered pegRNAs (epegRNAs) that included a structured 3′ motif that protects the reverse transcriptase template (RTT) from endogenous exonuclease degradation⁸⁹ were designed and screened. A dominant negative version of MLH1 (MLH1dn) was included to downregulate mismatch repair (MMR) as inhibition of MMR leads to improved prime editing outcomes^{86,87}. PE6 variants⁸⁸, or next-generation prime editors with evolved and/or rationally designed mutations in the Cas9 or reverse transcriptase domains, were also utilized. In order to specifically target the disease allele, the SpCas9-NRCH⁶⁰ DNA-targeting domain was used to create bespoke PEmax-NRCH and PE6-NRCH variants that recognize NRCH protospacer adjacent motifs (PAMs), where N is any nucleotide, R is adenine or guanine, and H is any nucleotide except guanine. The pathogenic c.1412delA mutation allows the creation of an NRCH PAM (TGCA), and correction of the mutation disrupts the NRCH PAM (TGTCA) (FIG. 11A).

First, a monoclonal heterozygous murine Neuro-2a (N2a) cell line was created with 50% of alleles harboring the murine equivalent of c.1412delA. All combinations of epegRNAs with primer binding site (PBS) lengths of 11-15 nt and RTT lengths of 14-21 nt were then designed and screened for efficient correction of the equivalent c.1412delA mutation in Cdkl5 (FIG. 11B). In addition to correcting the pathogenic mutation (+6 T insertion), the epegRNAs were also designed to incorporate a silent mutation in the spacer region (seed edit, +2 G>T) as this kind of edit has been shown to improve editing outcomes⁸⁶. The cells were then transfected with a PE4max strategy constituting plasmid DNA for PEmax-NRCH, MLH1dn, and each epegRNA.

Genomic DNA was collected from transfected cells without enriching for edited cells 3 days post-transfection, and on-target editing was quantified with high-throughput sequencing (HTS). The most efficient desired editing was observed in the 50% of targetable alleles containing the pathogenic mutation was 18.6±1.5% desired editing without indels using an epegRNA with a PBS of 11 nt and an RTT of 15 nt (FIG. 11B).

To further optimize allele-specific correction, epegRNAs were designed with shorter PBS lengths of 9 nt and 10 nt while keeping the RTT template the same length (15 nt). EpegRNAs were also designed with the same PBS and RTT parameters, but with a “flip and extension” (F+E) scaffold203 to determine whether the F+E scaffold could further improve prime editing as previously demonstrated when used with epegRNAs at other loci⁸⁹. The F+E guide RNA scaffold mutates the fourth base pair of the direct repeat in the standard scaffold from U•A to A•U and extends the direct repeat by five base pairs, removing a potential pol III terminator and improving Cas9 binding respectively²⁰³. Using a PE4max strategy, plasmid DNA was transfected for PEmax-NRCH, MLH1dn, and each of the newly designed epegRNAs into heterozygous N2a cells (FIG. 11C). Amongst the epegRNAs designed with the standard guide RNA scaffold, an increase in desired editing was observed when using a PBS length of 9 nt or 10nt (23.7±2.4% and 25±1.3% respectively, FIG. 11C). Furthermore, no increase in desired editing outcomes was observed when using the F+E scaffold for the shortened PBS epegRNAs.

Because it achieved the highest level of on-target editing, the epegRNA with a standard scaffold and a PBS length of 10 nt was used for all further studies. Taken together, the epegRNA optimization efforts reveal that development of an allele-specific correction strategy for the murine equivalent of c.1412delA in CDKL5 is achievable with PEmax-NRCH and does not require an engineered guide RNA scaffold for efficient editing. Next, it was sought to optimize the prime editing system used in the correction strategy presented herein.

Next-generation prime editor systems, the PE6 variants, have shown equal or improved editing efficiency compared to PEmax while taking advantage of size-minimizing evolution and engineering efforts⁸⁸. In particular, editing strategies were developed that included PE6b, whose reverse transcriptase (RT) was evolved from a yeast RT, PE6c, which was derived from PE6b with the addition of rationally engineered RT mutations, and PE6d, an RNaseH-truncated version of the M-MLV RT utilized in PEmax with the addition of evolved and rationally engineered mutations⁸⁸.

To determine if the PE6 variants were applicable to the allele-specific editing strategy for the murine equivalent of c.1412delA in CDKL5, NRCH variants PE6b-NRCH, PE6c-NRCH, and PE6d-NRCH were created. PE6d (1904)-NRCH, a variant of PE6d-NRCH only with mutations T128F and V223M, was also created, and PEmax-ΔRNaseH-NRCH, an RNaseH-truncated version of PEmax-NRCH with no other evolved or engineered mutations, was additionally created. The optimized epegRNA, MLH1dn, and each prime editor was transfected via plasmid DNA transfection, and allele-specific correction was quantified with HTS (FIG. 11D). It was found that PEmax-NRCH (17±3.4%), PE6b-NRCH (18±2%), and PE6c-NRCH (19±2%) had comparable levels of desired editing, all of which were higher levels of editing than that achieved by PE6d-NRCH (11.6±2.2%), PE6d (1904)-NRCH (13.8±1.5%), and PEmax-ΔRNaseH-NRCH (11.4±1%) (FIG. 11D). These results suggest that PE6 variants, particularly PE6b and PE6c, are amenable to the NRCH-based allele-specific prime editing strategy.

To further optimize the allele-specific prime editing strategy, three different nicking single guide RNAs (sgRNAs) were screened with opposite strand nick-to-nick distances of 87 nt (NG1), 63 nt (NG2), or 47 nt (NG3). Consistent with the previous prime editor screen, it was observed that PE5max-NRCH, PE6b-NRCH, and PE6c-NRCH out-performed PE6d-NRCH, PE6d (1901)-NRCH, and PE5max-ΔRNaseH-NRCH across all nicking sgRNAs (FIG. 11D).

It was found that for each prime editing system, the desired editing efficiencies were lowest when using NG3 and highest with NG1 and NG2 (FIG. 11D). For all screened prime editors, indel formation was minimally increased when paired with NG2, so it was determined that NG1 was the most therapeutically-relevant nicking sgRNA. While the desired editing efficiency for PE5max-NRCH (38.3±8.3%), PE6b-NRCH (38.7±4.9%), and PE6c-NRCH (38.3±6.7%) were similar on average when using NG1, average indel rates of 3.7±0.7% with PE6c-NRCH were 2× and 1.5× higher than average indel rates with PE5max-NRCH (1.8±0.2%) and PE6b-NRCH (2.4±0.1%), respectively (FIG. 11D). As such, it was determined that both PE5max-NRCH and PE6b-NRCH with NG1 were optimal systems to use in future studies for in vivo correction of c.1412delA in Cdkl5. Overall, these results establish an allele-specific correction strategy for c.1412delA in Cdkl5 with PEmax or PE6 variants, an optimized epegRNA, and optimized nicking sgRNA placement.

Optimizing Prime Editing to Correct c.1412delA in Human CDKL5

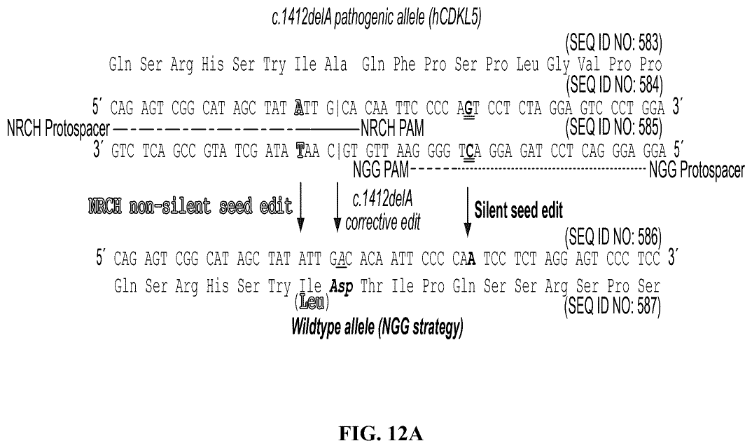

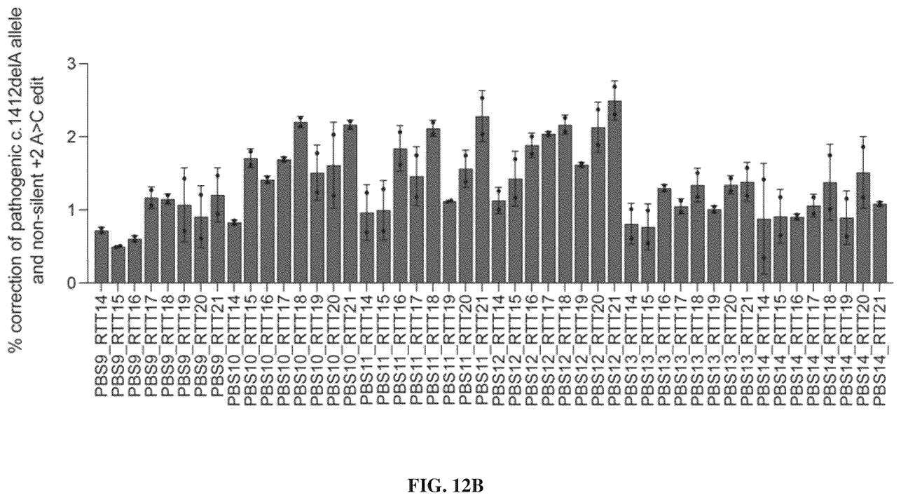

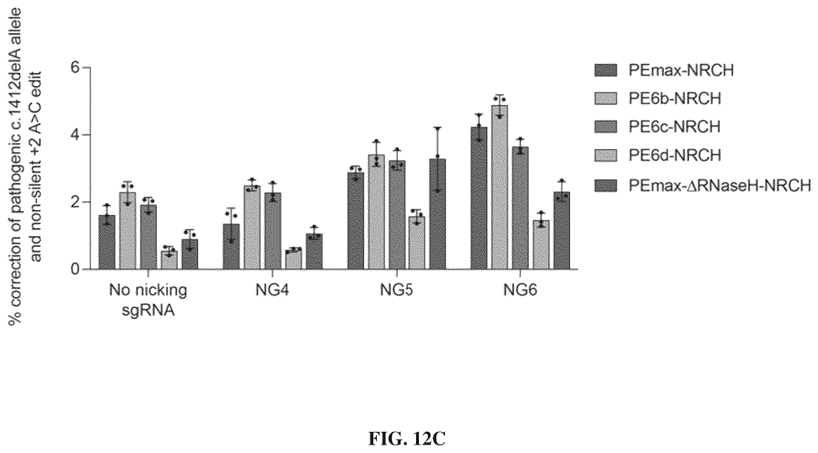

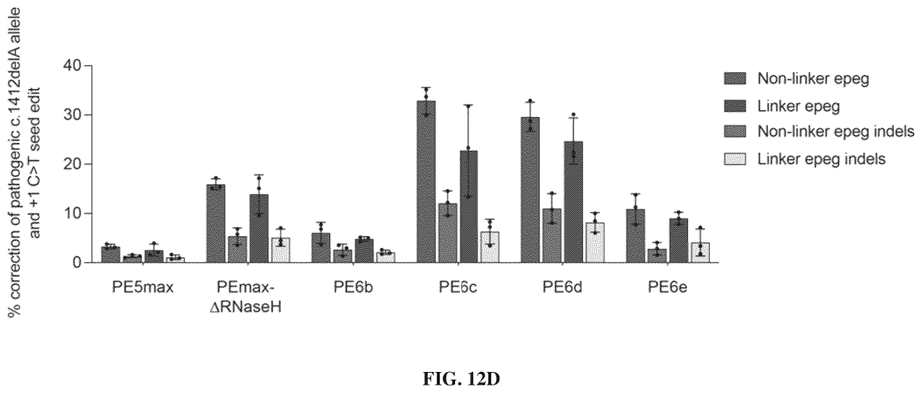

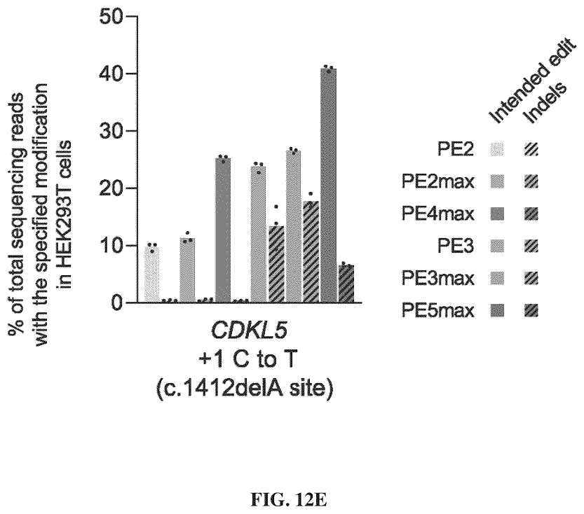

Next, it was sought to optimize a prime editing strategy to correct the pathogenic c.1412delA mutation in human CDKL5. Monoclonal homozygous HEK293T cell lines harboring the mutation of interest were created. A corrective strategy similar to the allele-specific editing strategy was then developed by designing epegRNAs to be used with NRCH variants of the prime editors (FIG. 12A). Of all the 80+epegRNAs screened, the epegRNAs that exhibited detectable editing with NRCH editors had a non-silent seed edit encoding I470L (FIGS. 12A-12B). Furthermore, additional PE and nicking sgRNA screening with the optimal I470L-encoding epegRNA achieved 4.9% editing with the most optimal conditions utilizing PE6b-NRCH and a nicking sgRNA with a nick-to-nick distance of 76 nt (NG6) (FIG. 12C). This data suggests that development of a prime editing strategy with an SpCas9-NRCH DNA targeting domain for human CDKL5 requires a unique set of optimizations that cannot be directly inferred from development of an analogous strategy in murine Cdkl5.

Correction of c.1412delA in CDKL5 has been previously optimized for PEmax (which utilizes an SpCas9 binding domain that recognizes the canonical NGG PAM) and a pegRNA encoding a silent seed edit (+1 C>T) in addition to the corrective edit (+14 T insertion, FIG. 12A)⁸⁶. Given that epegRNAs are the state-of-the-art guide RNA for prime editors^{85,89}, the pegRNA was converted to an epegRNA by adding a structured 3′ trimmed evopreQ1 motif⁸⁹. In order to determine if an optional 8 nt linker designed to minimize interactions between the protective 3′ motif and the RTT⁸⁹ would improve editing efficiencies over not including the linker, epegRNAs were designed both with and without the linker to test in a head-to-head comparison (FIG. 12D). Plasmid DNA was transfected for either the linker-containing or non-linker epegRNA along with the previously optimized nicking sgRNA86, MLH1dn, and PEmax constituting a PE5max editing strategy. PE5max desired editing was observed with the non-linker epegRNA at 3.3±0.5%, which minimally outperformed the desired editing achieved with the linker-containing epegRNA at 2.6±1.2% (FIG. 12D). These results suggested that the presence of the optional 8 nt linker does not improve prime editing outcomes at the desired locus when using PEmax.

To fully optimize the prime editing strategy and determine whether the linker-containing epegRNA might improve editing with other PE systems, desired editing was compared across both epegRNAs when used with the previously optimized nicking sgRNA86, MLH1dn, and one of six prime editor variants-PEmax, PEmax-ΔRNaseH, PE6b, PE6c, PE6d, and PE6e, a PE6 variant with a Cas9 domain harboring evolved and engineered mutations⁸⁸.

Across all PE systems, it was found that the non-linker epegRNA achieved higher desired editing than the linker-containing epegRNA (FIG. 12D). In particular, it was observed that using the non-linker epegRNA with PE6c resulted in the highest level of correction at 32.9±2.7% desired editing with 12±2.5% indels while PE6c with the linker-containing epegRNA resulted in 22.8±9.2% desired editing with 6.3±2.5% indels. PE6d editing outcomes were modestly decreased with 29.6±3% desired correction with the non-linker epegRNA and 24.7±4.7% desired correction with the linker-containing epegRNA (FIG. 12D). In contrast to the highly efficient PE6b-NRCH editing optimized for murine Cdkl5 (FIG. 11D), PE6b desired editing was among the lowest for human CDKL5 at just 6±2.1% for the non-linker epegRNA (FIG. 12D). Because it achieved the highest on-target editing, PE6c is the optimal candidate for future studies in therapeutically relevant models such as neurons or cortical organoids differentiated from patient-derived iPSCs.

c.1412delA Correction in Patient Primary Fibroblasts

Next, it was determined whether the PE6c strategy could correct the c.1412delA mutation in primary patient cells. Efficient editing via electroporation of in vitro transcribed PE mRNA and synthetic pegRNA has been previously demonstrated in patient-derived iPSCs up to 17% with PE3 or 34% with PE5⁸⁶. To determine if c.1412delA correction with PE6c or PEmax was efficient in primary patient cells, PE6c or PEmax mRNA was electroporated with synthetic non-linker-containing epegRNA and nicking sgRNA into patient fibroblasts isolated from a female patient heterozygous for the pathogenic mutation.

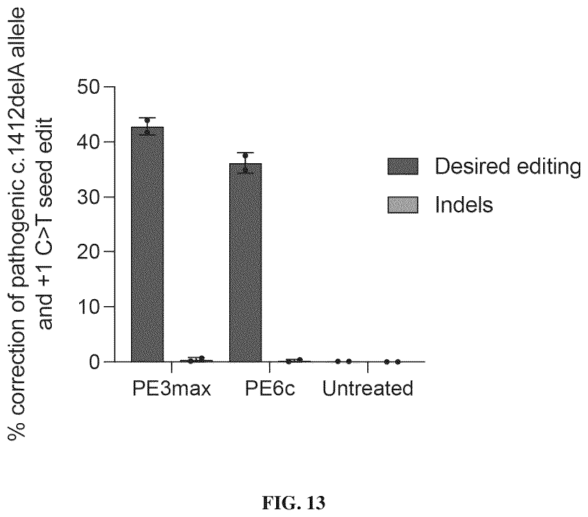

These electroporations yielded 36.2% correction of editable alleles and 1.9% indel products with PE6c, and 42.8% correction of editable alleles with 1.6% indel products with PE3max (FIG. 13). Notably, this level of correction did not require the addition of MLH1dn.

Given PE6c's outsized c.1412delA correction compared to PE5max in HEK293T cells homozygous for c.1412delA (FIG. 12D), it was anticipated that PE6c would outperform PE3max in primary patient cells. Additionally, the HEK293T experiments utilized MLH1dn for a PE6c and PE5max comparison, thus PE6c editing outcomes may benefit more from the presence of MLH1dn than PEmax.

### Genome-Wide Off-Target Editing Analysis in Patient Primary Fibroblasts

After optimizing the prime editing strategy for corrective editing of c.1412delA in CDKL5 and quantifying its efficacy in patient primary fibroblasts, it was next sought to quantify any off-target editing. Prime editing has widely been reported to have substantially lower levels of off-target editing compared to other CRISPR-based gene editing methods^{79,97-105}, reflecting the multiple steps at which an off-target sequence can be rejected without modification³. To assess off-target prime editing outcomes from the PEmax and PE6c correction strategies in patient primary fibroblasts, Cas-OFFinder, a computational method for nominating potential off-target loci¹²⁸, was used. The Cas-OFFinder algorithm was used to nominate off-target loci with up to three mismatches to the target protospacer sequences of the epegRNA and the nicking sgRNA. For the epegRNA, 15 off-target loci were nominated (Peg OT1-15). Just three off-target loci were nominated for the nicking sgRNA (Nick OT1-3). In both cases, all nominated off-target loci had exactly three mismatches.

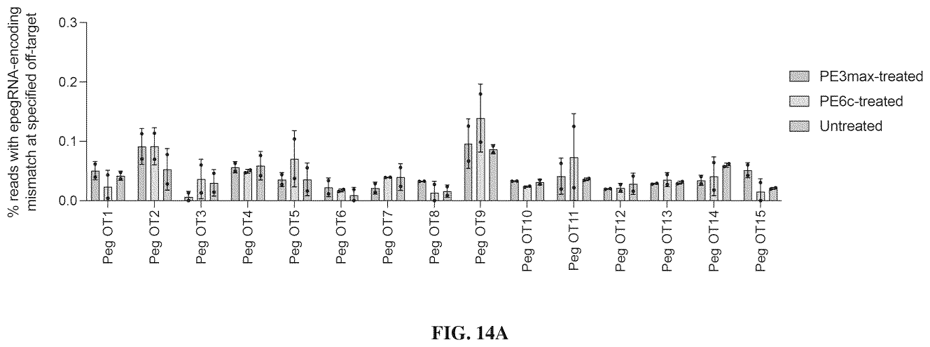

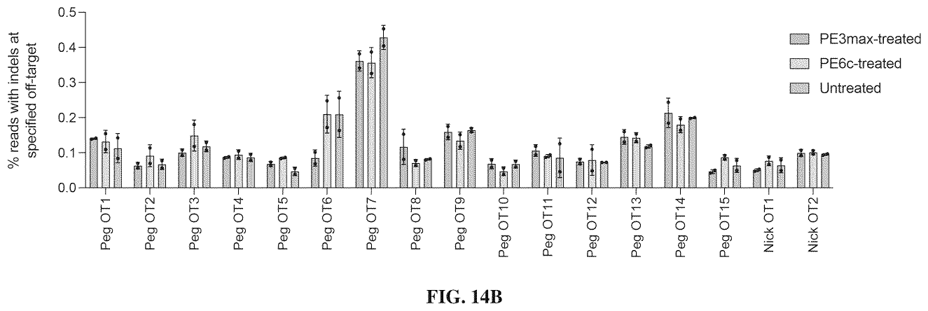

HTS was used to quantify off-target editing at each of the nominated loci. For the epegRNA off-targets, the mutation frequency was quantified at the position of the first substitution that would be encoded by the epegRNA RTT as this position is the most likely nucleotide to be changed during prime editing^{3,79}. Since the probability of an incorrect base call for the HTS method is 1 in 1000 bases²⁰⁴, a threshold of 0.1% was established in the analysis for bona fide epegRNA-dependent off-target substitution. It was observed that epegRNA-dependent off-target substitutions in PE3max-treated or PE6c-treated fibroblasts were largely similar to that of untreated fibroblasts (FIG. 14A). Only at Peg OT9 was any editing above the 0.1% threshold observed. PE6c-treated fibroblasts accumulated an average of 0.13±0.06% substitutions at Peg OT9, a range that overlaps with the observed substitution rate for the untreated samples at the same locus (0.09±0.01%). The initial analysis in primary patient fibroblasts is consistent with previous reports that prime editing leads to minimal pegRNA-dependent off-target editing^{79,97-105}.

The indel frequency at each of the epegRNA nominated sites was also quantified. It was found that off-target indel formation in PE3max-treated or PE6c-treated fibroblasts was similar to that of untreated fibroblasts at nearly every site (FIG. 14B). Though off-target indel formation at Peg OT5 for PE6c-treated samples was minimally increased compared to untreated samples, the rate of indel formation was quite low (0.08%). Overall, these results are consistent with previous reports of low PE-mediated off-target editing^{79,97-105}.

To quantify off-target editing at Cas-OFFinder-nominated sites for the nicking sgRNA, the indel frequency was quantified at two of the three nominated sites via HTS (FIG. 14B). Editing at Nick OT3 could not be quantified as it is located on the Y chromosome and the fibroblasts were derived from a female patient. Compared to untreated control fibroblasts, there was no nicking sgRNA-dependent indel formation at either of the remaining 2 nominated loci in treated fibroblasts (FIG. 14B). Collectively, these results suggest that PE3max-edited and PE6c-edited c.1412delA fibroblasts experience minimal PE-mediated off-target editing, and the prime editing strategy could be used to correct c.1412delA with minimal detectable off-target editing in the human genome.

### Corrective Prime Editing of Alternative Pathogenic CDKL5 Mutations

There are hundreds of pathogenic mutations known to cause CDD^{178,181-192}. Because of this, it was sought to apply prime editing strategies to other loci of interest in CDKL5. One locus of interest is a pathogenic mutation at a splice site acceptor, c.826-1 G>A. This CDKL5 variant is uniquely prime-editable given the short-comings of nuclease-based editing and the presence of multiple other adenines in the editing window that could result in bystander A>G editing if the locus were targeted by a base editor.

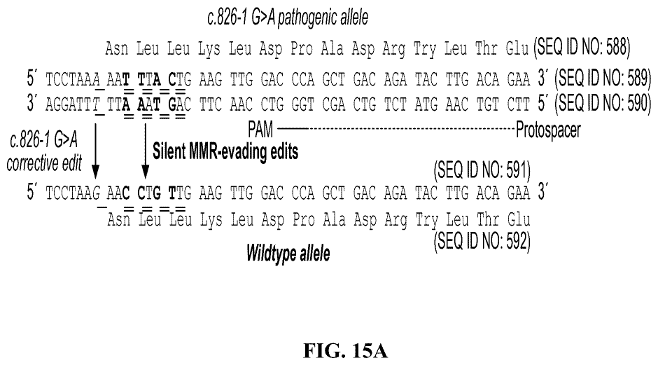

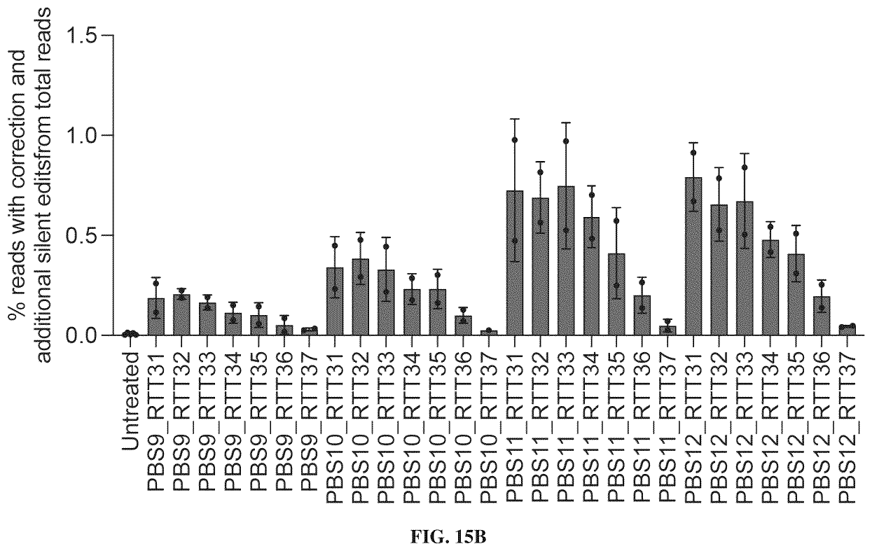

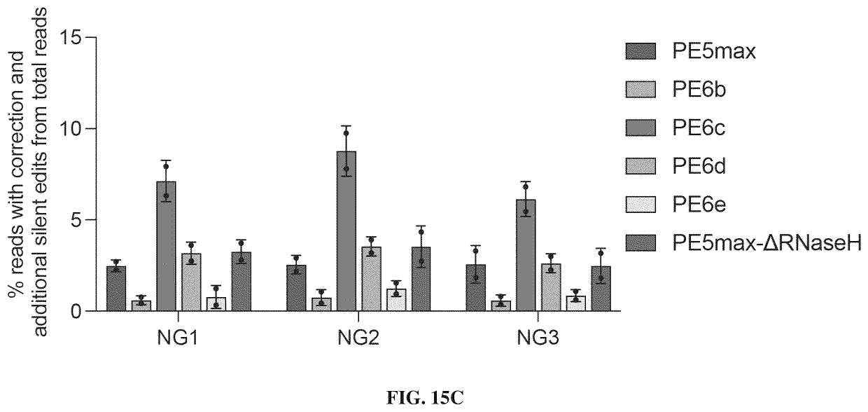

In order to begin initial optimizations for a corrective c.826-1 G>A prime editing strategy, two heterozygous HEK293T cell lines were first created harboring the pathogenic mutation about 30% of alleles. After establishing the cell lines, epegRNAs were then designed and screened that encode the desired corrective edit (+24 A>G) and multiple silent mutations near the desired corrective edit (+17-21 TTTAC>CCTGT) (FIG. 15A). These benign mutations help the prime editing intermediate to naturally evade MMR, a DNA repair process known to decrease desired prime editing outcomes^{86,87}. An epegRNA screen was conducted by transfecting PEmax-ΔRNaseH, MHL1dn, and various epegRNAs into the model cell lines (FIG. 15A). While editing efficiencies were low, several epegRNAs were observed with desired editing percentages above 0.7% across all alleles. The epegRNA with PBS and RTT lengths of 11 and 33 nt respectively were elected to be used, reaching an average of 0.75±0.32% editing for additional optimization.

Next, to determine the most optimal prime editor for the corrective strategy (FIG. 15C), the most optimal nicking sgRNA position with 3 nicking sgRNAs in total (NG1, NG2, NG3, FIG. 15C) was also screened. NG3 was designed for edit-specific complementary nicking in which nicking does not occur until after the desired edit has been installed. This edit-specific nicking sgRNA design leads to minimal indel formation³. Unlike NG1 and NG2, NG3 utilizes an NGA PAM instead of the canonical NGG PAM because SpCas9 can have residual catalytic activity on NGA PAMs^{44,205}. It was observed that PE6c resulted in the highest correction efficiency with up to 9.8% desired editing when paired with NG2 (FIG. 15C). Average indel formation was minimal for all samples, with indel formation for all nicking guides occurring at less than 0.11% in all samples. Therefore, corrective editing with PE6c and NG2 currently represents the strongest prime editing strategy for further studies. Overall, these data suggest that the c.826-1 G>A, like c.1412delA, is amenable to prime editing correction, most notably with PE6c.

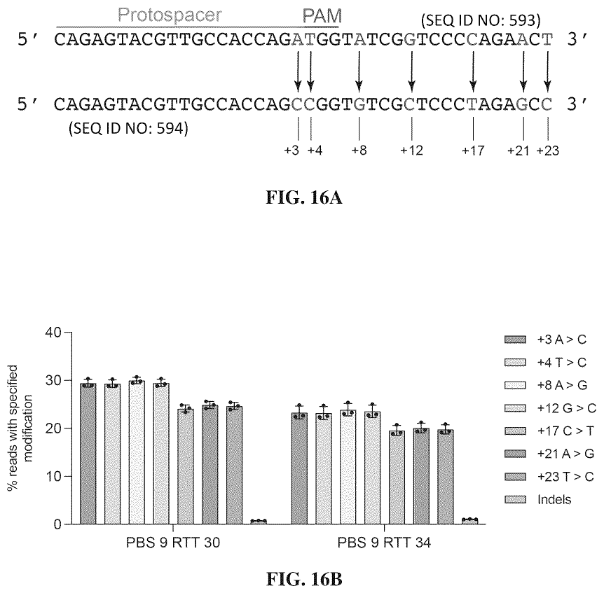

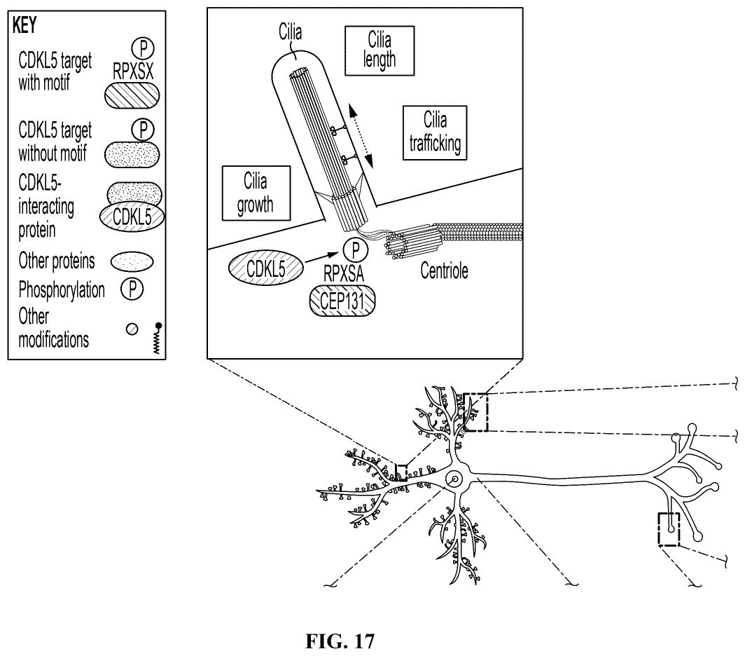

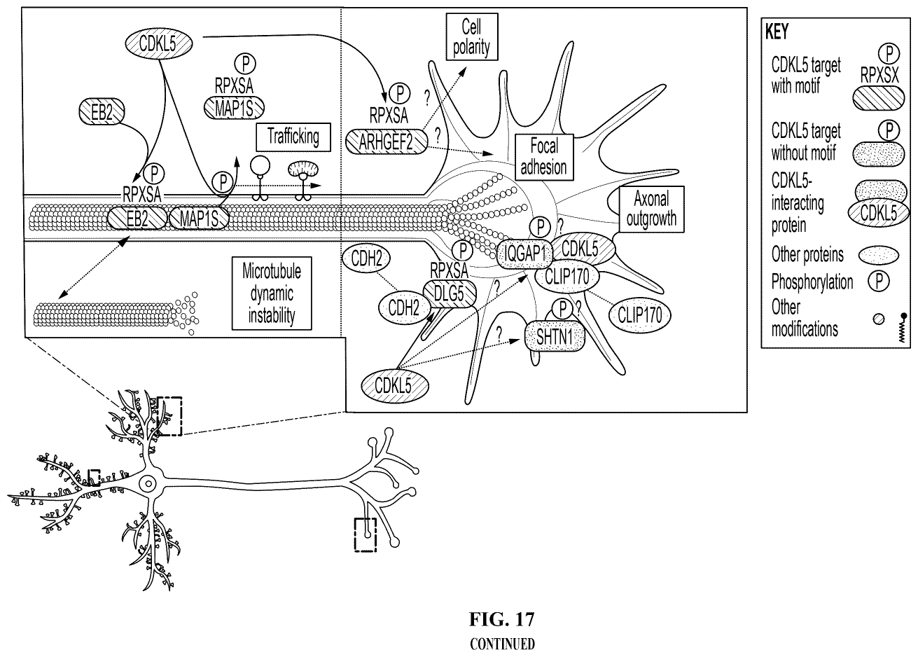

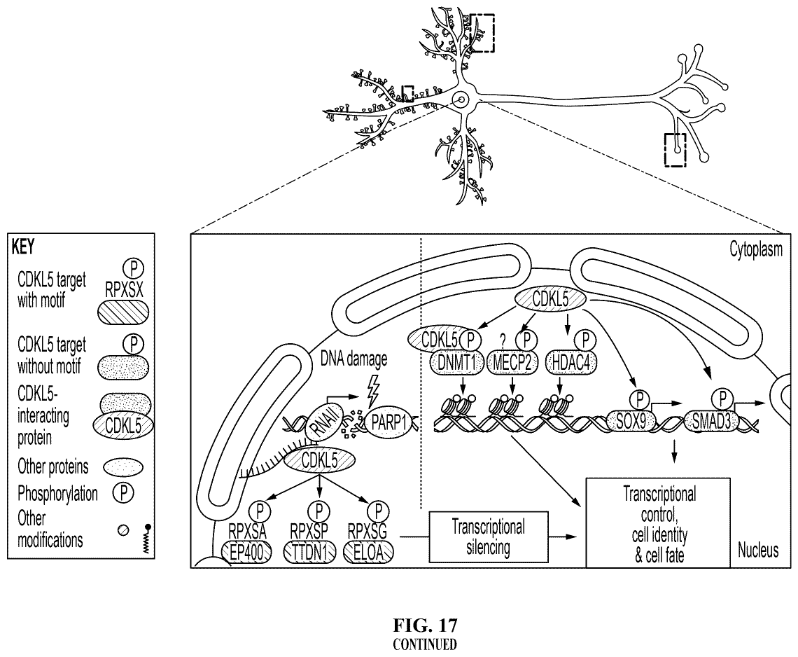

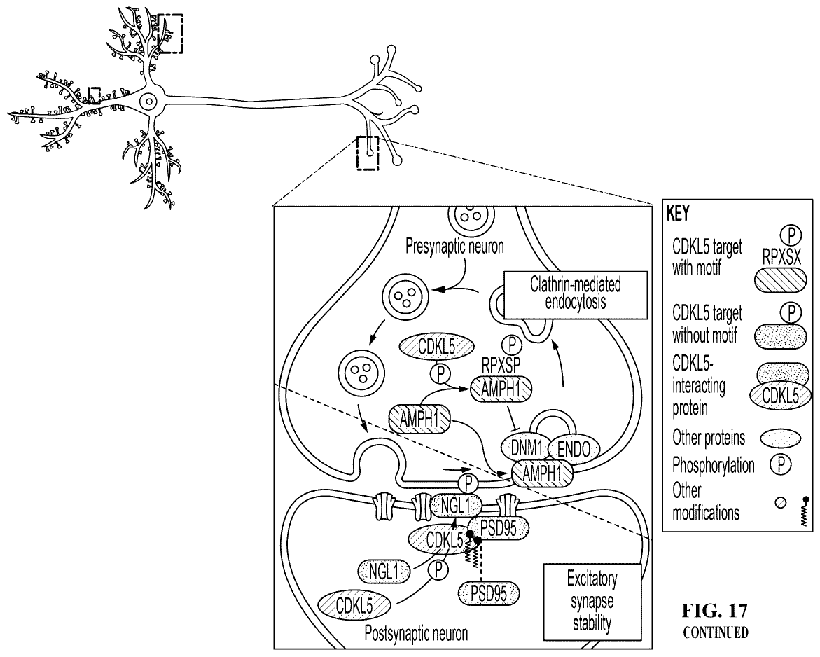

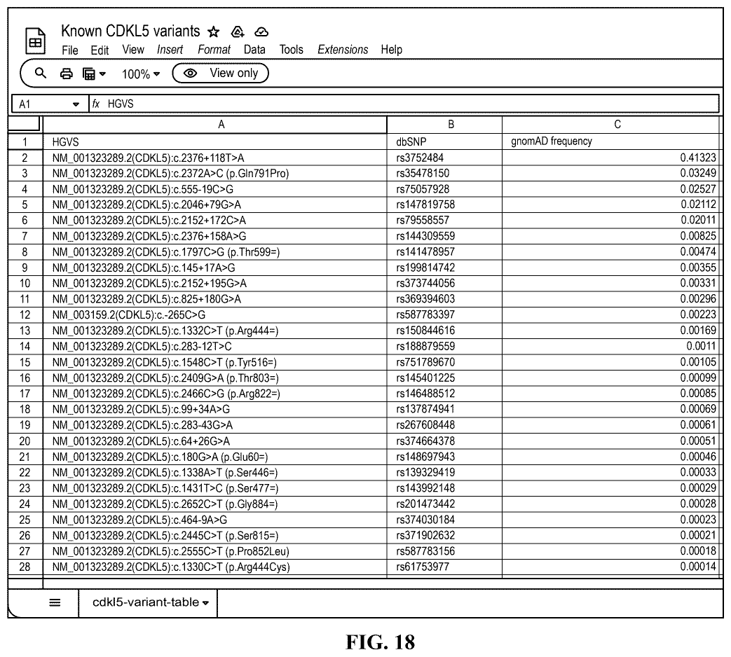

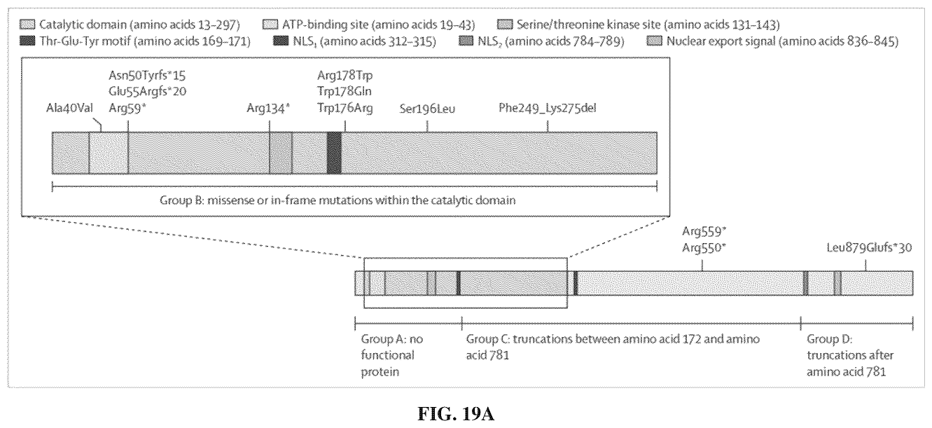

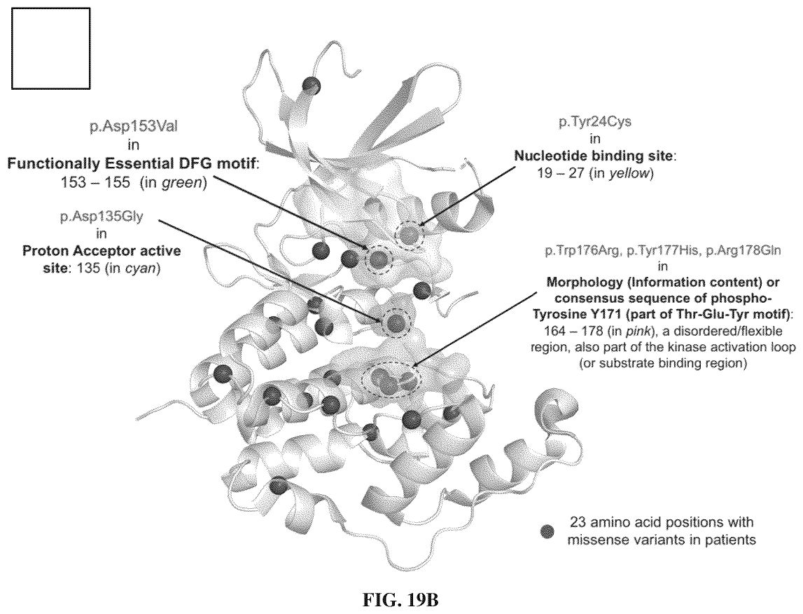

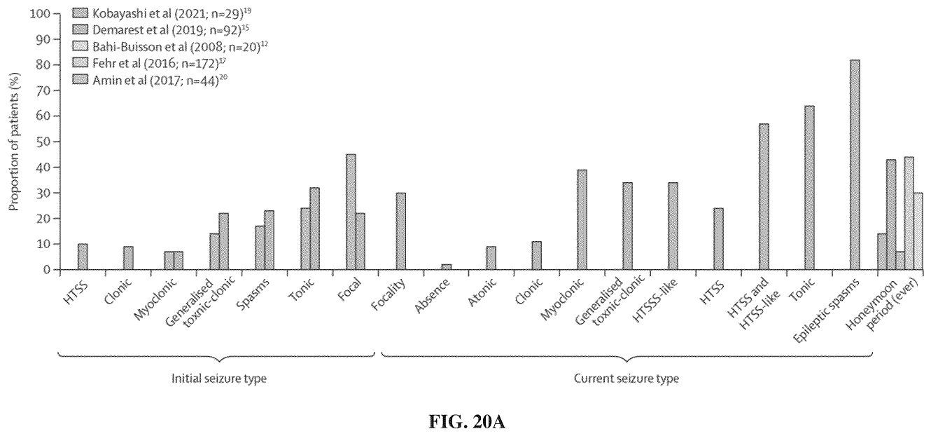

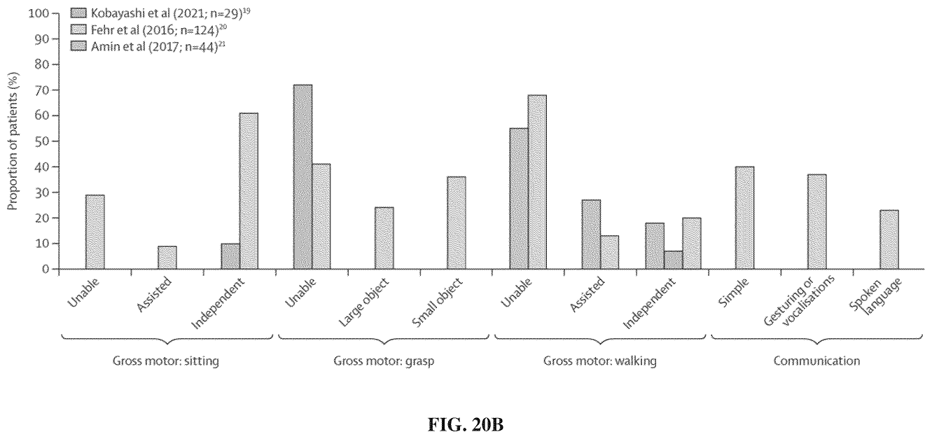

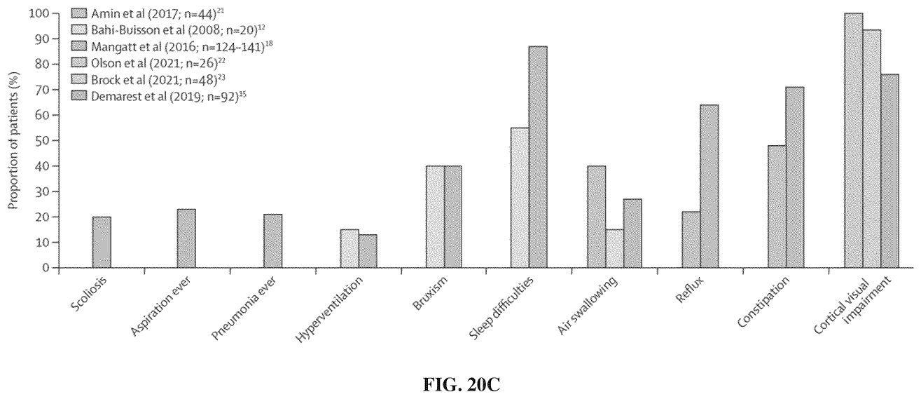

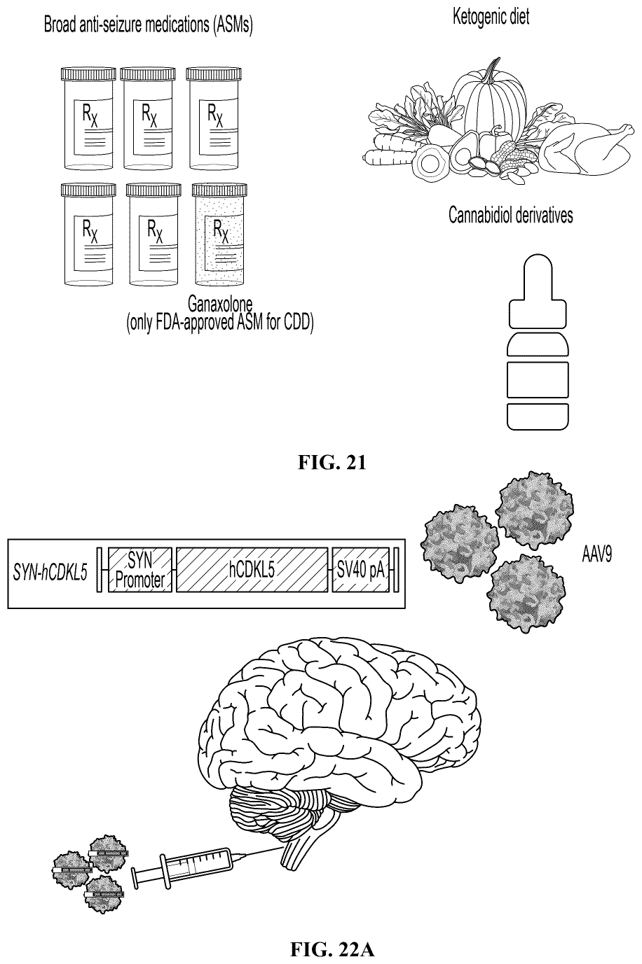

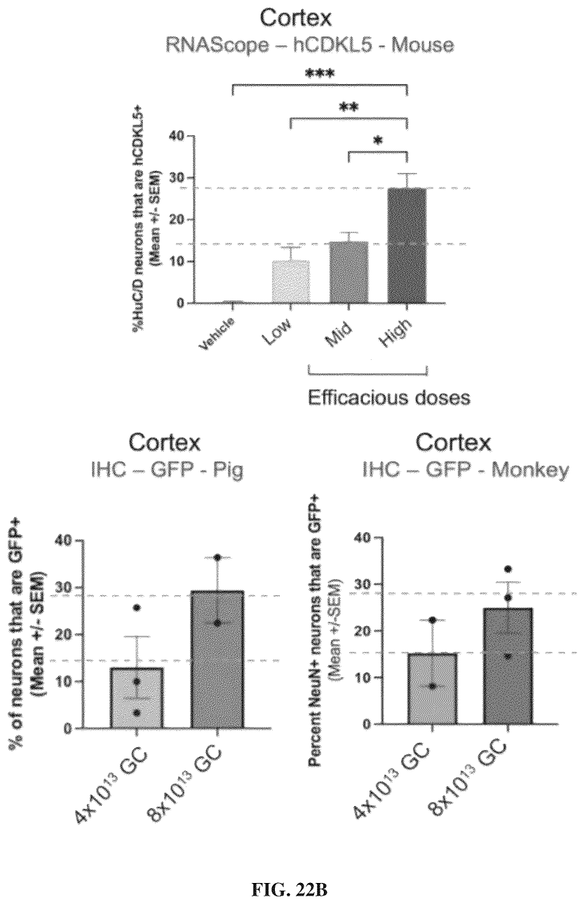

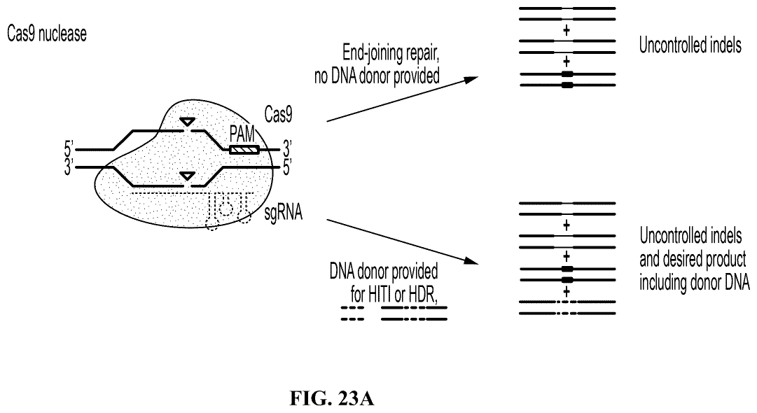

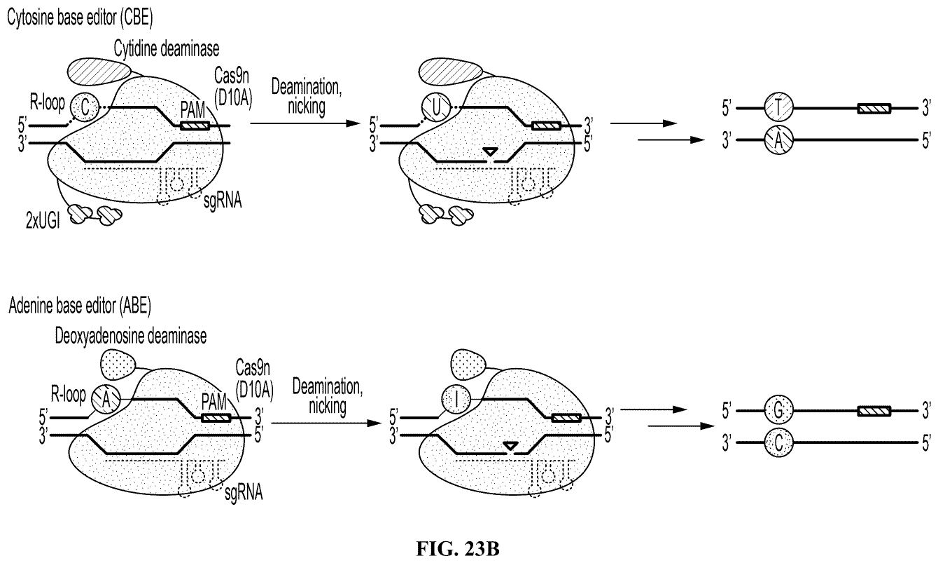

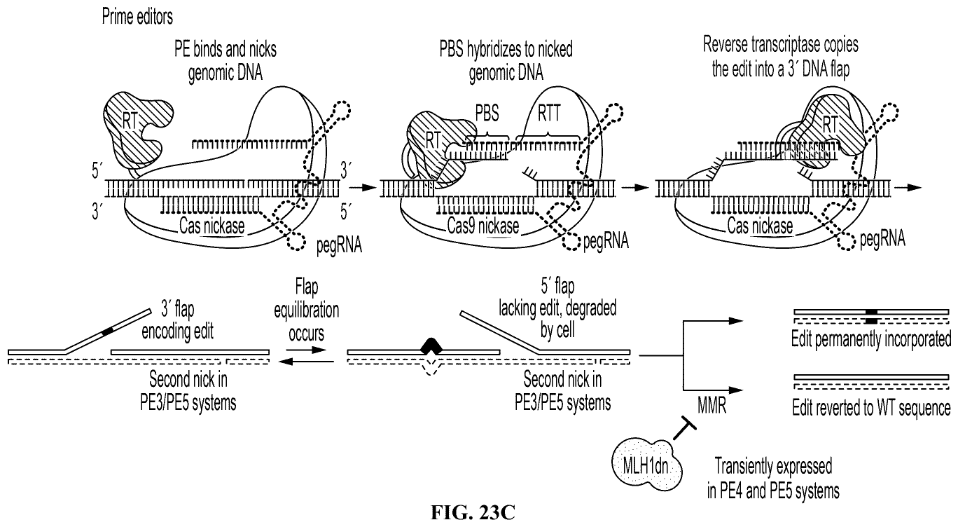

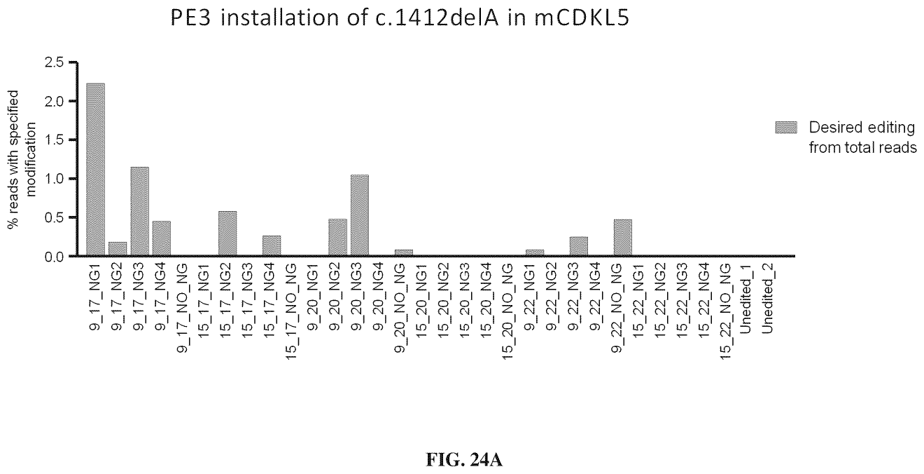

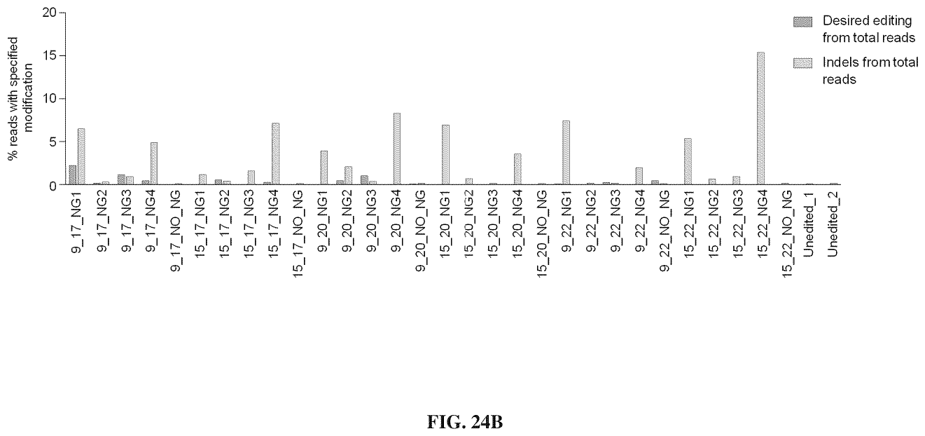

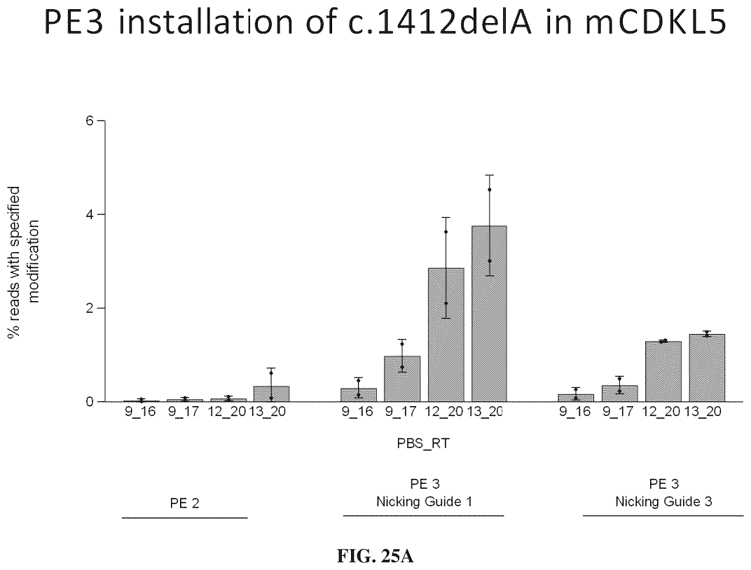

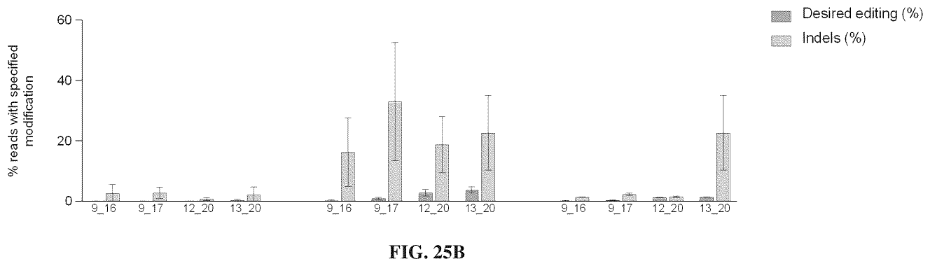

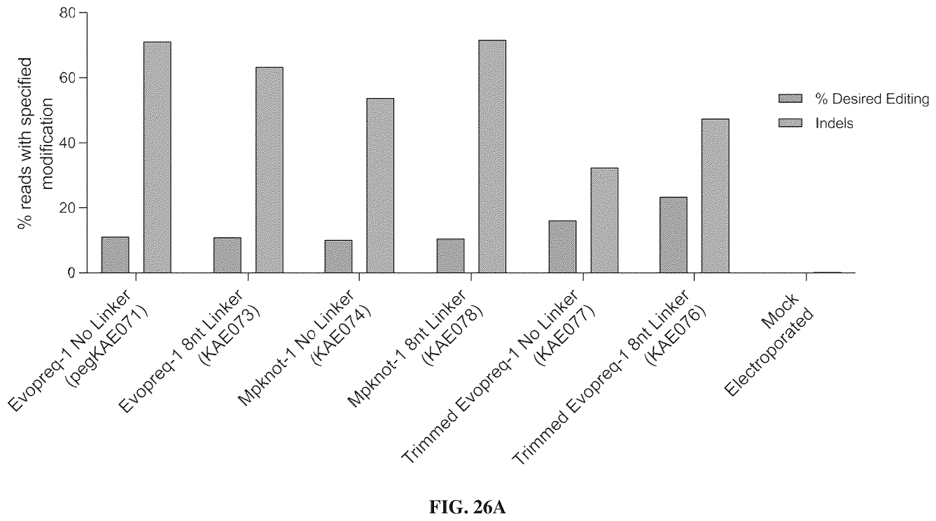

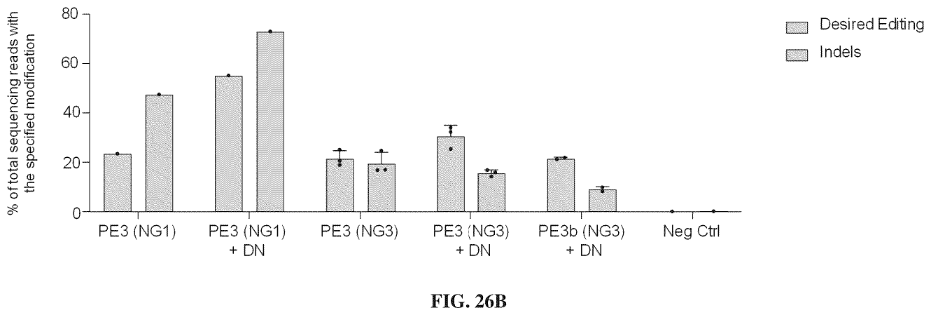

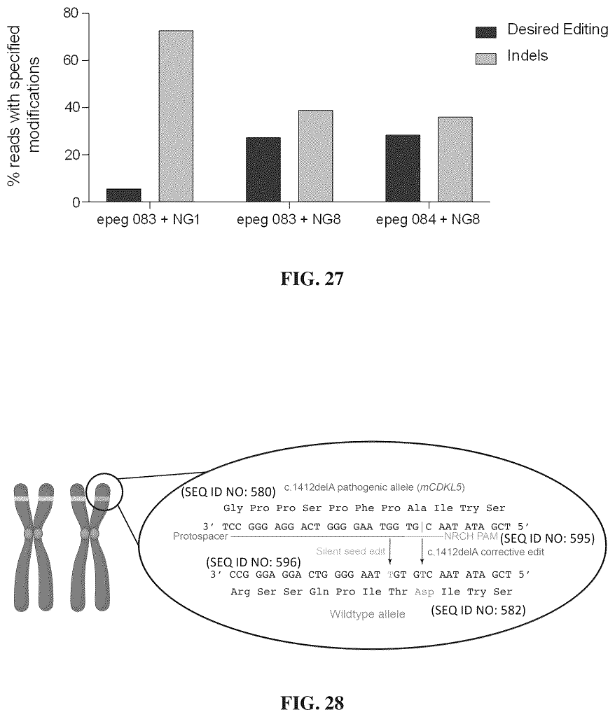

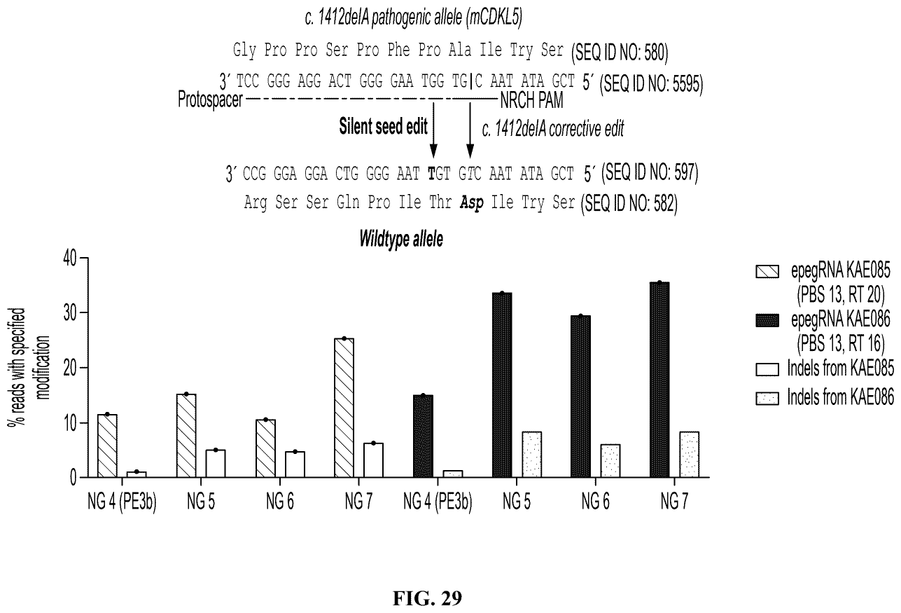

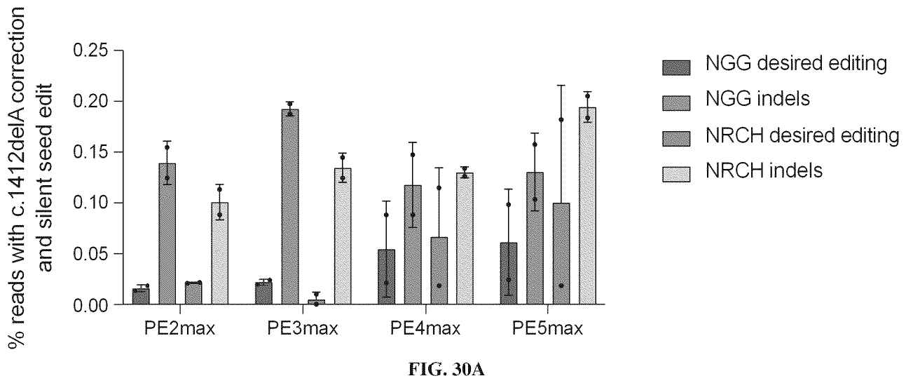

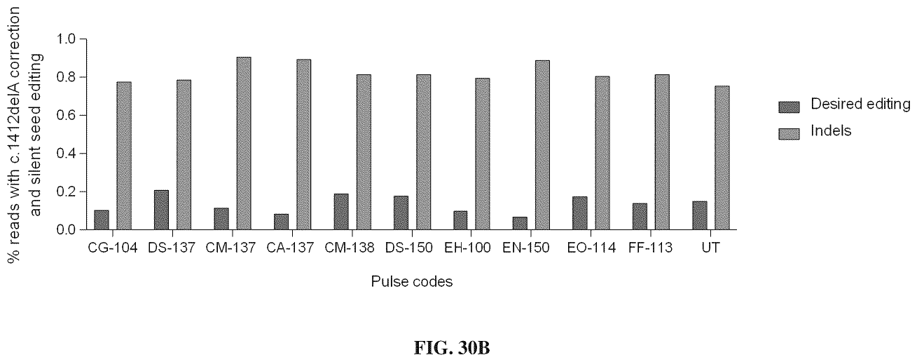

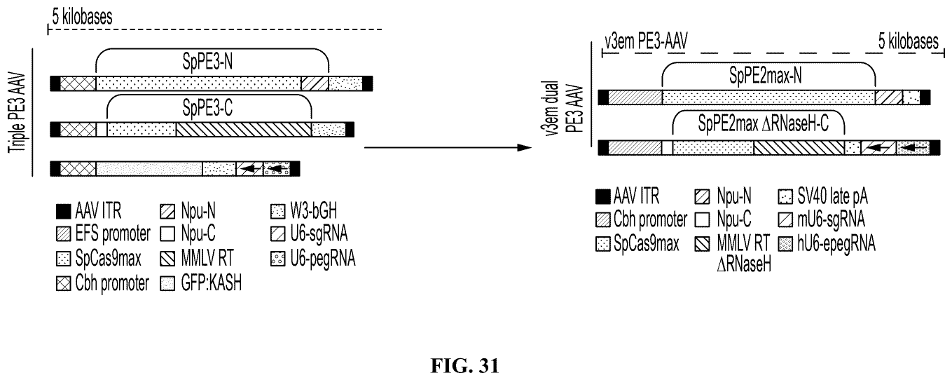

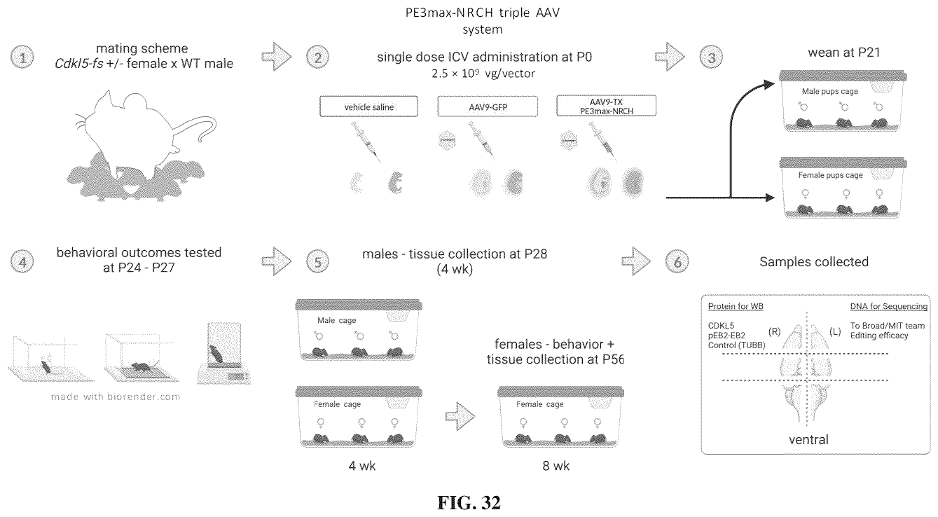

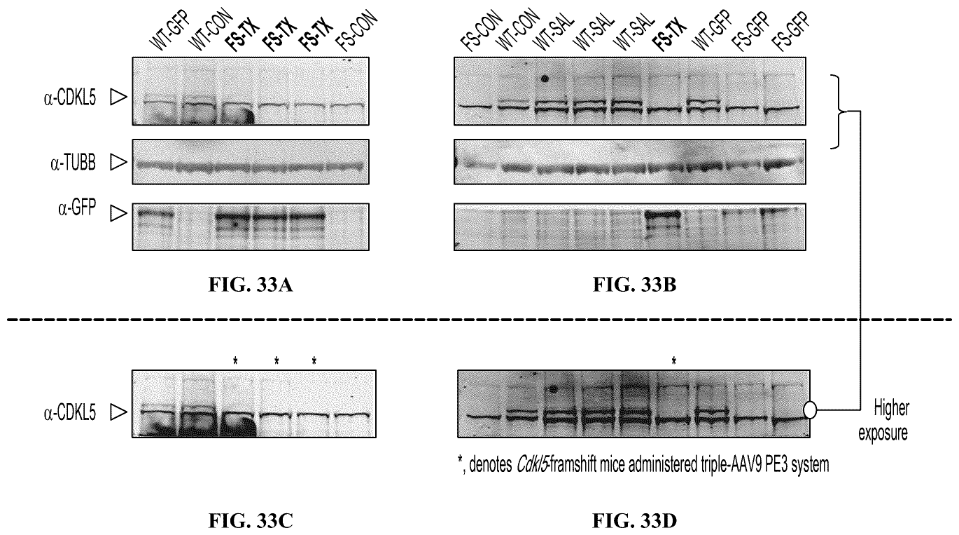

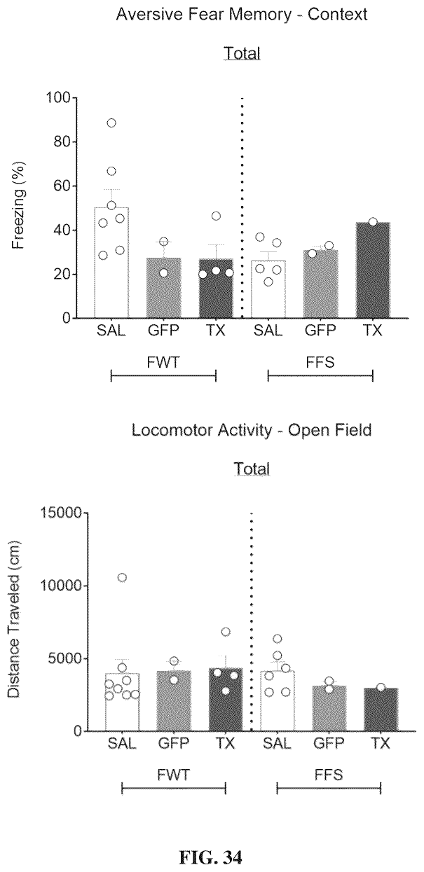

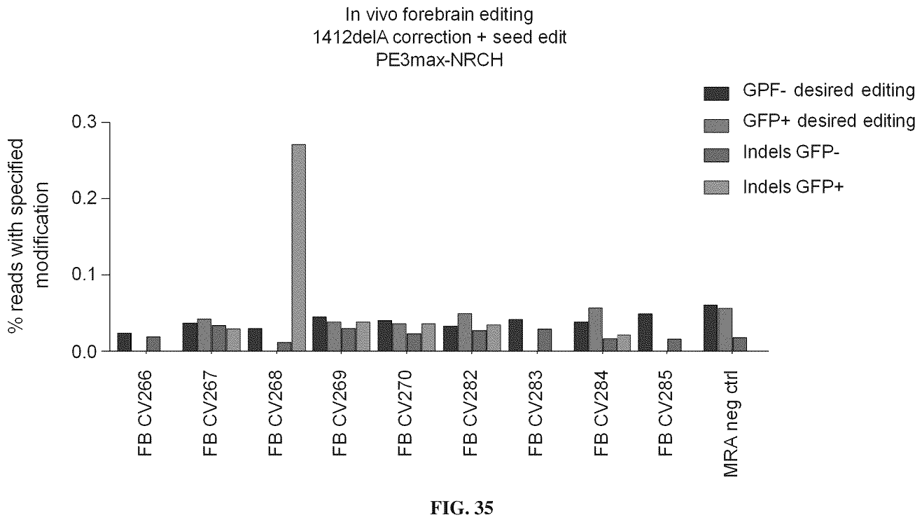

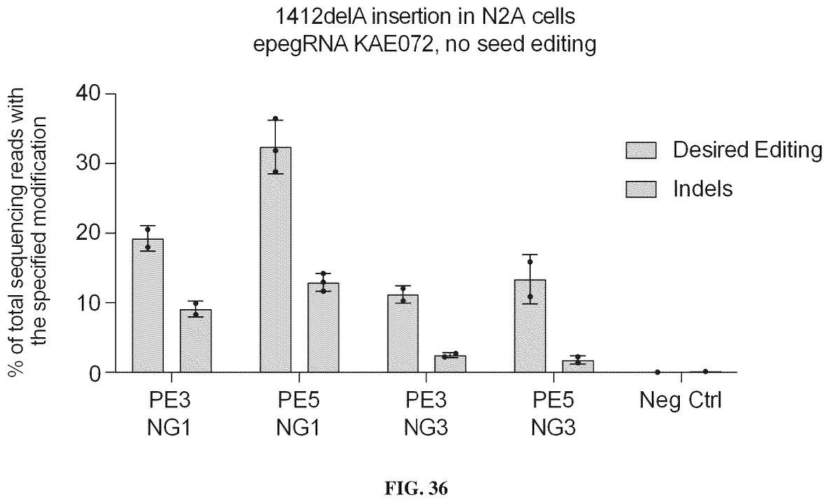

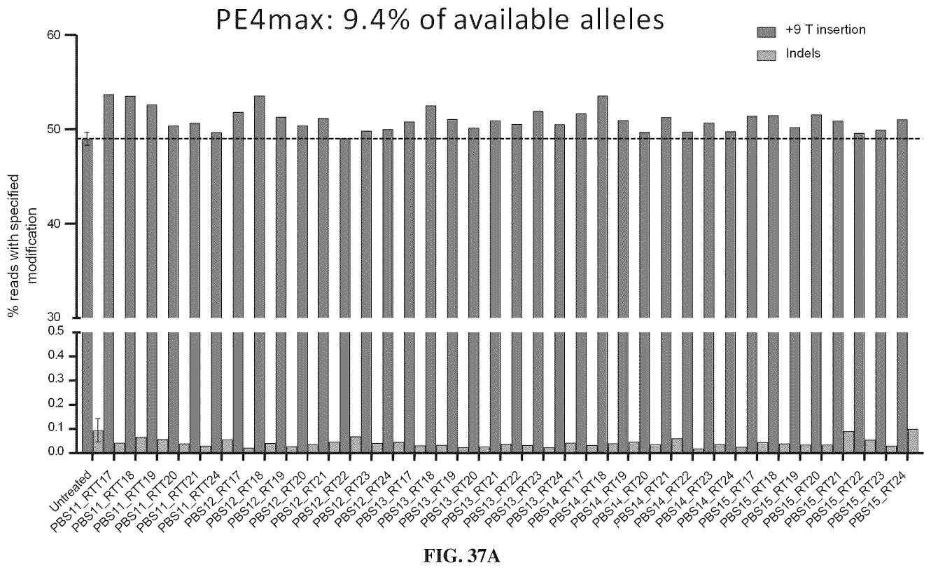

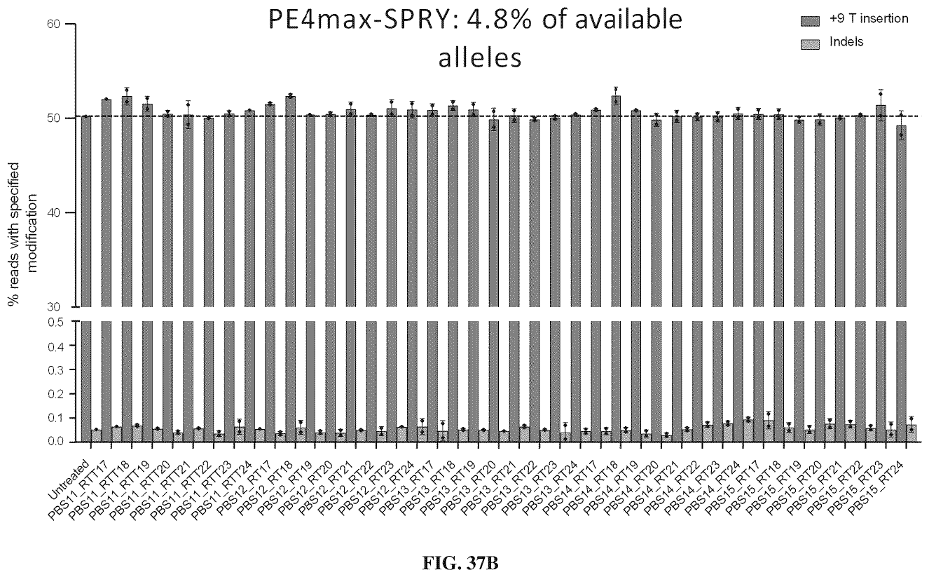

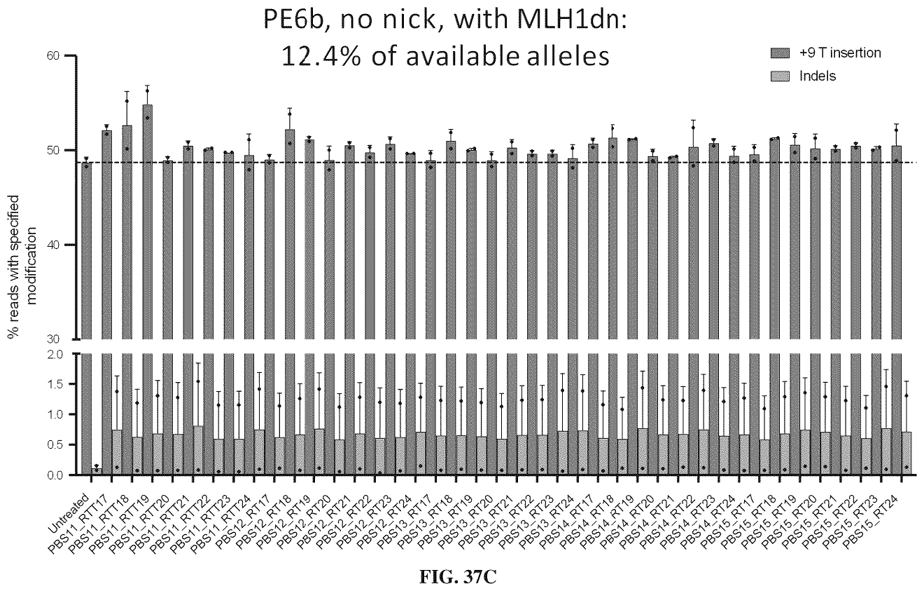

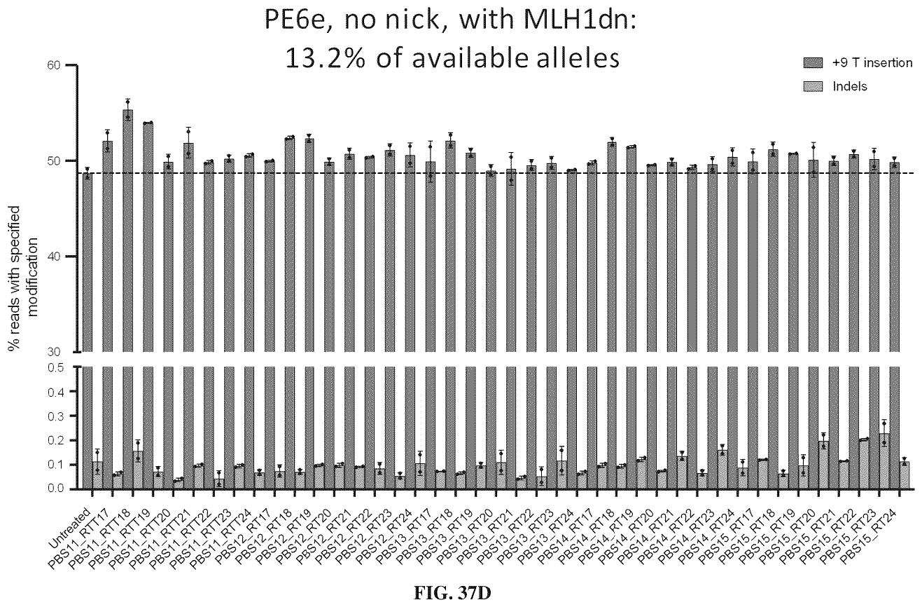

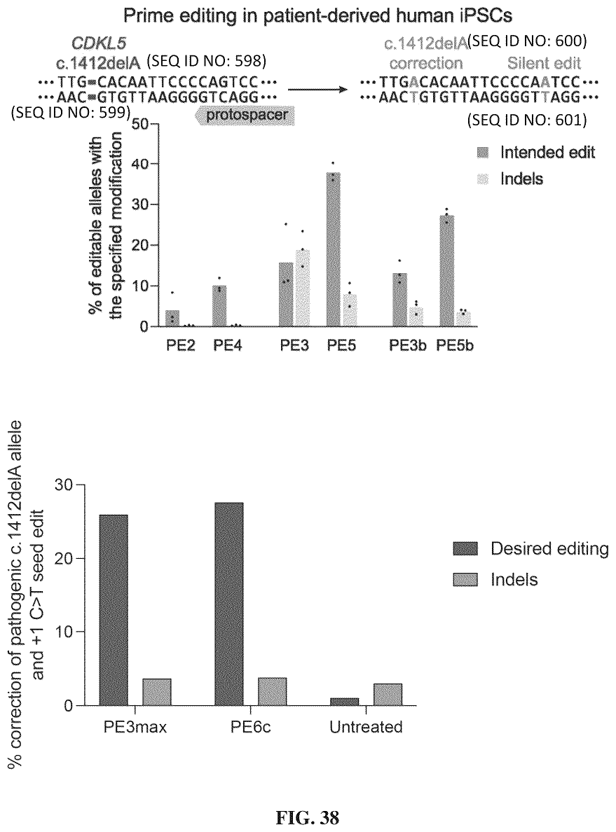

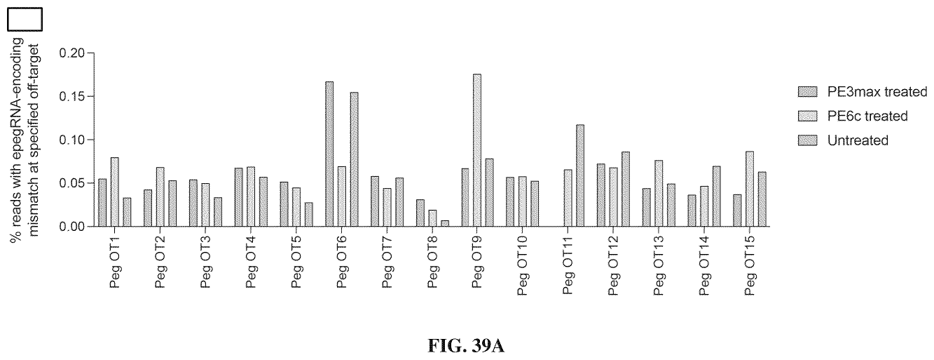

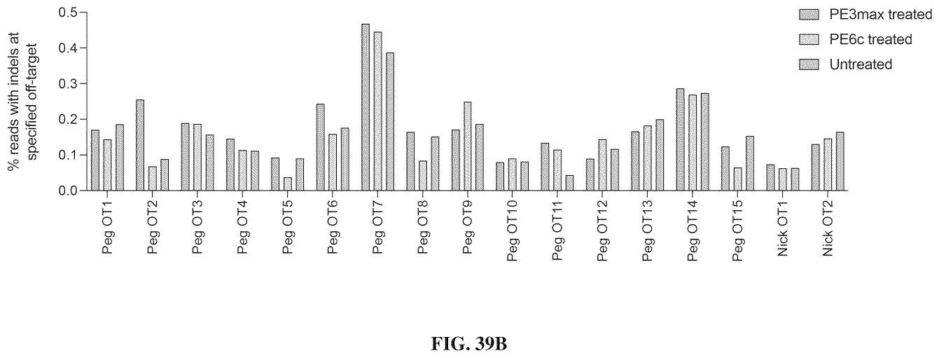

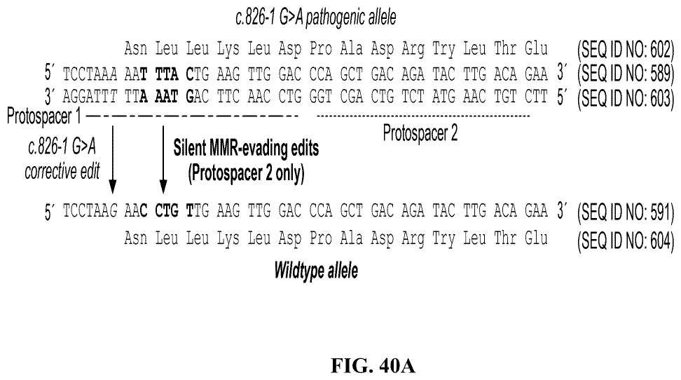

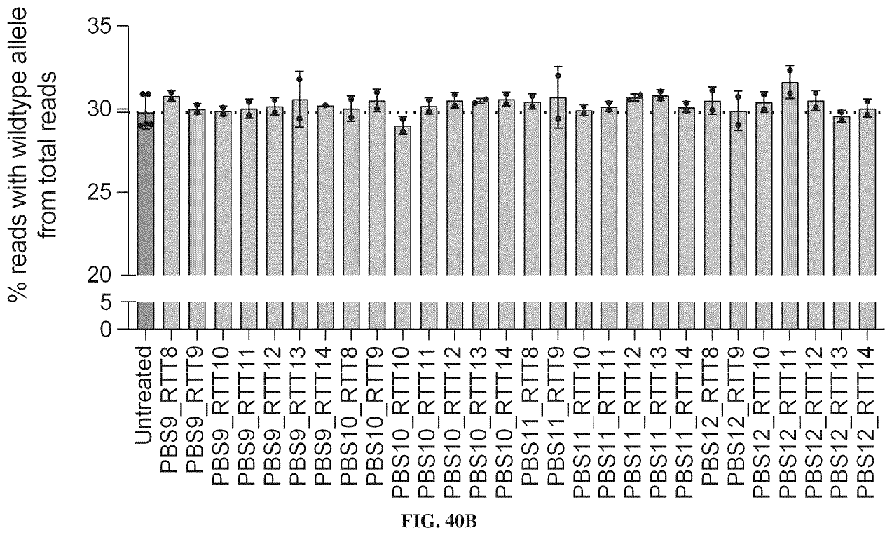

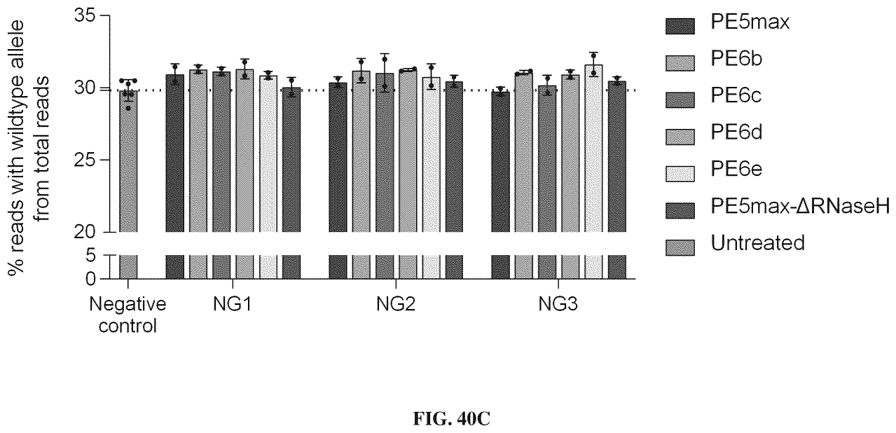

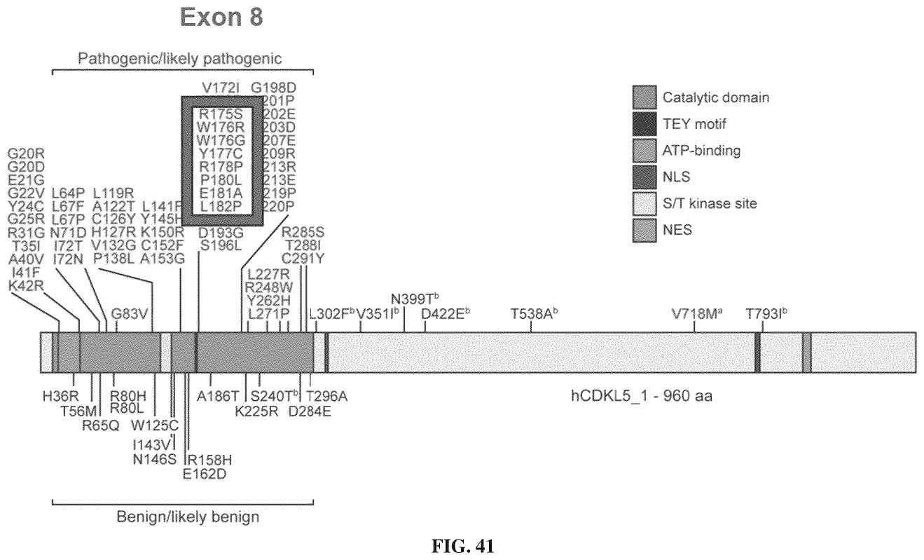

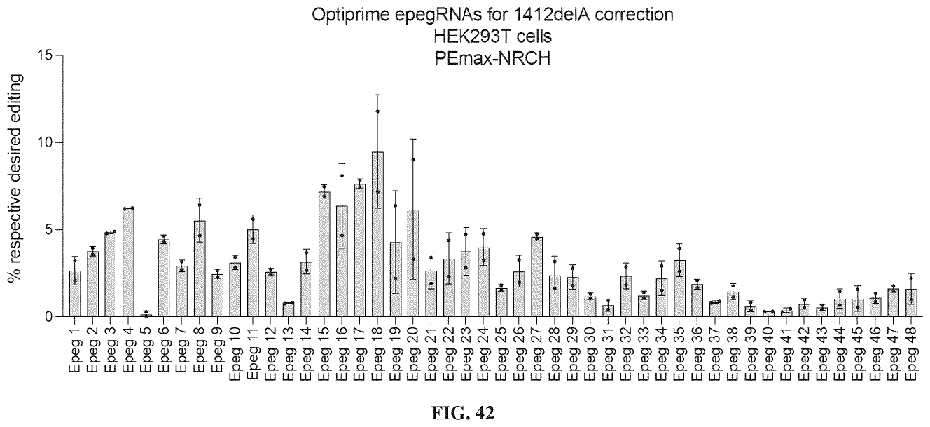

Most patients with CDD have unique pathogenic mutations, and these mutations occur throughout the CDKL5 gene, but pathogenic missense occur almost exclusively the catalytic domain of CDKL5^{181,187,199}. The ability to use a single epegRNA to correct pathogenic mutations for multiple patients is ideal. This strategy would allow for the development of a single composition of matter and a single clinical trial for a group of patients as opposed to the development of multiple compositions of matter for each individual patient, and consequently multiple clinical trials. Thus, exon 8 was chosen as a model site for demonstrating the ability of a single epegRNA to modify multiple bases in a region like the catalytic domain where multiple pathogenic mutations cluster. As a proof-of-principle, two epegRNAs were designed to install five known pathogenic mutations (p.R175S (+3 A>C), p.W176G (+4 T>C), p.Y177C (+8 A>G), p.P180L (+17 C>T), and p.L182P (+23 T>C)) 181,191 and two additional MMR evading mutations (+12 G>C and +21 A>G) in exon 8 (FIG. 16A). These epegRNAs were not fully optimized for PBS and RTT lengths. The epegRNAs were transfected into wildtype HEK293T cells with PEmax and a +50 nicking sgRNA, and editing efficiency of all seven edits being incorporated into the target locus at once was quantified. It was found that on average, the first four desired modifications were installed at an editing efficiency of 29.4%, and all seven modifications were installed at 25.4% editing efficiency (FIG. 16B). The decreased editing efficiency for the final three modifications suggests potential degradation of the newly synthesized DNA strand by endogenous exonucleases before resolution of editing via DNA repair. Having a longer RTT that extends more bases past the last desired edit could remediate this issue. These findings suggest the potential for a therapeutic strategy to correct multiple pathogenic CDD mutations across several amino acids with a single epegRNA.

### DISCUSSION

The lack of approved disease-modifying therapeutics for CDD highlights the urgent unmet medical need of CDD patients and their caregivers. While a gene therapy that would deliver a functional copy of CDKL5 is being developed, this therapeutic strategy may be intractable for patients with dominant negative mutations²⁰⁶. Additionally, the dosage sensitivity of many genetic epilepsy disorders, including CDD where overexpression of CDKL5 has been reported to manifest in disease²⁰⁷, limits the applicability of a gene replacement therapy since endogenous transcriptional control of the target gene would be lost^{206,208}. The most physiological approach to treating CDD would be to revert the disease alleles back to wildtype.

PEmax-NRCH and PE6-NRCH strategies capable of enabling 38% allele-specific correction of c.1412delA in heterozygous murine N2a cells were developed. Though development of a PE-NRCH correction strategy in human CDKL5 was not initially successful without the addition of a non-silent seed edit, nearly 40% correction of the c.1412delA allele in patient primary fibroblasts was demonstrated with PE6c and a previously reported pegRNA converted to an epegRNA. Initial off-target editing analyses with Cas-OFFinder-nominated loci revealed that the prime editing strategy was highly-specific. Initial experiments for the correction of c.826-1 G>A resulted in up to 9.8% prime editing with PE6c, and prime editing at a mutational hotspot in exon 8 resulted in 25.4% installation of seven additional CDKL5 mutations or relevant seed edits. These results demonstrate that the development of prime editing strategies, including those with the recently described PE6 variants, lead to robust levels of editing across the CDKL5 gene.

Another important question in the development of CDD therapeutics is timing of drug administration. Since CDD is early-onset, and clinical symptoms occur within the first few months of life, there have historically been concerns about the therapeutic window at which a patient must be treated²⁰⁸. A recent study has shown that restoration of CDKL5 expression in adult mice rescued CDD-associated phenotypes¹⁷⁷. These findings suggest that the ability to treat CDD beyond early infancy may be possible.

### Methods

### Culture of Cell Lines and Primary Patient Fibroblasts

HEK293T cells and Neuro-2a (N2a) cells were cultured in Dulbecco's Modified Eagle Medium (DMEM) plus GlutaMAX (Thermo Fisher Scientific) supplemented with 10% (v/v) fetal bovine serum (FBS) (Thermo Fisher Scientific). Primary fibroblasts were obtained from a patient sample²¹⁵ and cultured in DMEM supplemented with 20% fetal bovine serum.

Generation of Cell Lines with CDKL5 Mutations

For c.1412delA installation, 1.2-1.5×10⁴ N2a or HEK293T cells were transfected with 200 ng of PEmax, 60 ng of an appropriate epegRNA installing the desired mutation (c.1412delA in murine or human CDKL5), 22 ng of an appropriate nicking sgRNA, and 100 ng of MHL1dn. For c.826-1 G>A installation, 1.5×10⁴ HEK293T cells were transfected with 200 ng of CBE6b67 and 50 ng of sgRNA. All DNA transfections were completed with 0.5 μl Lipofectamine 2000 (Thermo Fisher Scientific) and 5.75 μl OptiMEM combined with an appropriate DNA plasmid mixture.

Transfected cells were then diluted to approximately 1 cell/well and subsequently allowed to grow into single cell colonies for 7-14 days. Colonies were split evenly for HTS verification, and cell culture was continued. Sequence verified cells were expanded until a confluent T-75 flask could be established. From confluent flasks, several vials of 1×10⁶ cells were cryopreserved for future experiments, while 2×10⁵ cells were seeded for continued culture and corrective editing experiments.

Transfections for epegRNA, Nicking sgRNA, and Prime Editor Screens

1.5×10⁴ N2a or HEK293T cells per well in a 96-well tissue culture plate were seeded 1 day before transfection. All DNA transfections were completed with 0.5 μl Lipofectamine 2000 (Thermo Fisher Scientific) and 5.75 μl OptiMEM combined with an appropriate DNA plasmid mixture. The DNA plasmid mixture included 200 ng of prime editor plasmid, 60 ng of epegRNA plasmid, and in experiments where nicking guides or MLH1dn are also included, 22 ng of nicking sgRNA plasmid and/or 100 ng of MLH1dn plasmid were also transfected. After 3 days, genomic DNA was extracted from the cells and desired editing was measured via HTS.

Sequences of Relevant epegRNAs and Nicking sgRNAs:

Newly described guide RNA sequences are in Tables 4 and 5:

TABLE 4
Additional pegRNAs targeting CDKL5 c.1412delA used in Example 2:

| | | | | Sequence |
|---|---|---|---|---|
| | | | | Identifier |
| | | | | for Full |
| Spacer | Scaffold | Template | PBS | pegRNA |
| GAGAGTCG | GTTTCAGAGCTATGCTGGAAACAGCATAGC | GACTGGGGAAT | TAGCTA | 200 |
| GCATAGCT | AAGTTGAAATAAGGCTAGTCCGTTATCAAC | TGTGTCGATG | TGCCG | |
| ATAT (SEQ | TTGAAAAAGTGGCACCGAGTCGGTGC | (SEQ ID NO: 420) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTGGGGAAT | TAGCTA | 201 |
| GCATAGCT | AAGTTGAAATAAGGCTAGTCCGTTATCAAC | TGTGTCAATG | TGCCG | |
| ATAT (SEQ | TTGAAAAAGTGGCACCGAGTCGGTGC | (SEQ ID NO: 421) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTGGGGAAT | TAGCTA | 202 |
| GCATAGCT | AAGTTGAAATAAGGCTAGTCCGTTATCAAC | TGTGTCGATA | TGCCG | |
| ATAT (SEQ | TTGAAAAAGTGGCACCGAGTCGGTGC | (SEQ ID NO: 422) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTGGGGAAT | TAGCTA | 203 |
| GCATAGCT | AAGTTGAAATAAGGCTAGTCCGTTATCAAC | TGTGTCTATG | TGCCG | |
| ATAT (SEQ | TTGAAAAAGTGGCACCGAGTCGGTGC | (SEQ ID NO: 423) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTGGGGAAT | TAGCTA | 204 |
| GCATAGCT | AAGTTGAAATAAGGCTAGTCCGTTATCAAC | TGTGTCAATA | TGCCG | |
| ATAT (SEQ | TTGAAAAAGTGGCACCGAGTCGGTGC | (SEQ ID NO: 424) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTGGGGAAT | TAGCTA | 205 |
| GCATAGCT | AAGTTGAAATAAGGCTAGTCCGTTATCAAC | TGTGTCTATA | TGCCG | |
| ATAT (SEQ | TTGAAAAAGTGGCACCGAGTCGGTGC | (SEQ ID NO: 425) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GGACTGGGGAA | TAGCTA | 206 |
| GCATAGCT | AAGTTGAAATAAGGCTAGTCCGTTATCAAC | TTGTATCGATG | TGCCG | |
| ATAT (SEQ | TTGAAAAAGTGGCACCGAGTCGGTGC | (SEQ ID NO: 426) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GGACTGGGGAA | TAGCTA | 207 |
| GCATAGCT | AAGTTGAAATAAGGCTAGTCCGTTATCAAC | TTGTATCAATG | TGCCG | |
| ATAT (SEQ | TTGAAAAAGTGGCACCGAGTCGGTGC | (SEQ ID NO: 427) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GGACTGGGGAA | TAGCTA | 208 |
| GCATAGCT | AAGTTGAAATAAGGCTAGTCCGTTATCAAC | TTGTATCTATG | TGCCG | |
| ATAT (SEQ | TTGAAAAAGTGGCACCGAGTCGGTGC | (SEQ ID NO: 428) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | TAGAGGACTGG | TAGCTA | 209 |
| GCATAGCT | AAGTTGAAATAAGGCTAGTCCGTTATCAAC | GGAATCGTGTCA | TGCCG | |
| ATAT (SEQ | TTGAAAAAGTGGCACCGAGTCGGTGC | ATA | ACT | |
| ID NO: 9) | (SEQ ID NO: 577) | (SEQ ID NO: 429) | (SEQ ID | |
| | | | NO: 6) | |
| GAGAGTCG | GTTTCAGAGCTATGCTGGAAACAGCATAGC | GGACTGGGGAA | TAGCTA | 210 |
| GCATAGCT | AAGTTGAAATAAGGCTAGTCCGTTATCAAC | TTGTATCGATA | TGCCG | |
| ATAT (SEQ | TTGAAAAAGTGGCACCGAGTCGGTGC | (SEQ ID NO: 430) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | TAGAGGACTGG | TAGCTA | 211 |
| GCATAGCT | AAGTTGAAATAAGGCTAGTCCGTTATCAAC | GGAATCGTGTCG | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 431) | (SEQ ID | |
| | | | NO: 6) | |
| GAGAGTCG | GTTTCAGAGCTATGCTGGAAACAGCATAGC | GGACTGGGGAA | TAGCTA | 212 |
| GCATAGCT | AAGTTGAAATAAGGCTAGTCCGTTATCAAC | TTGTATCTATA | TGCCG | |
| ATAT (SEQ | TTGAAAAAGTGGCACCGAGTCGGTGC | (SEQ ID NO: 432) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | GACTCCTAGAG | TAGCTA | 213 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | CGTGTCAATA | ACT | |
| ID NO: 9) | (SEQ ID NO: 577) | (SEQ ID NO: 433) | (SEQ ID | |
| | | | NO: 6) | |
| GAGAGTCG | GTTTCAGAGCTATGCTGGAAACAGCATAGC | TCCTAGAGGACT | TAGCTA | 214 |
| GCATAGCT | AAGTTGAAATAAGGCTAGTCCGTTATCAAC | GGGGAATCGTAT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 434) | (SEQ ID | |
| | | | NO: 6) | |
| GAGAGTCG | GTTTCAGAGCTATGCTGGAAACAGCATAGC | GGACTGGGGAA | TAGCTA | 215 |
| GCATAGCT | AAGTTGAAATAAGGCTAGTCCGTTATCAAC | TTGTATCAATA | TGCCG | |
| ATAT (SEQ | TTGAAAAAGTGGCACCGAGTCGGTGC | (SEQ ID NO: 435) | ACT | |
| ID NO: 9) | (SEQ ID NO: 577) | | (SEQ ID | |
| | | | NO: 6) | |
| GAGAGTCG | GTTTCAGAGCTATGCTGGAAACAGCATAGC | TAGAGGACTGG | TGCCG | 216 |
| GCATAGCT | AAGTTGAAATAAGGCTAGTCCGTTATCAAC | GGAATAGTGTCA | TAGCTA | |
| ATAT (SEQ | TTGAAAAAGTGGCACCGAGTCGGTGC | ATA | ACT | |
| ID NO: 9) | (SEQ ID NO: 577) | (SEQ ID NO: | (SEQ ID | |
| | | 436) | NO: 6) | |
| GAGAGTCG | GTTTCAGAGCTATGCTGGAAACAGCATAGC | TAGAGGACTGG | TAGCTA | 217 |
| GCATAGCT | AAGTTGAAATAAGGCTAGTCCGTTATCAAC | GGAATCGTGTCG | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 437) | (SEQ ID | |
| | | | NO: 6) | |
| GAGAGTCG | GTTTCAGAGCTATGCTGGAAACAGCATAGC | TAGAGGACTGG | TAGCTA | 218 |
| GCATAGCT | AAGTTGAAATAAGGCTAGTCCGTTATCAAC | GGAATCGTGTCA | ACT | |
| ATAT (SEQ | TTGAAAAAGTGGCACCGAGTCGGTGC | ATG (SEQ ID NO: | TGCCG | |
| ID NO: 9) | (SEQ ID NO: 577) | 438) | (SEQ ID | |
| | | | NO: 6) | |
| GAGAGTCG | GTTTCAGAGCTATGCTGGAAACAGCATAGC | TAGAGGACTGG | TAGCTA | 219 |
| GCATAGCT | AAGTTGAAATAAGGCTAGTCCGTTATCAAC | GGAATCGTGTCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 439) | (SEQ ID | |
| | | | NO: 6) | |
| GAGAGTCG | GTTTCAGAGCTATGCTGGAAACAGCATAGC | TAGAGGACTGG | TAGCTA | 220 |
| GCATAGCT | AAGTTGAAATAAGGCTAGTCCGTTATCAAC | GGAATAGTATCA | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 440) | (SEQ ID | |
| | | | NO: 6) | |
| GAGAGTCG | GTTTCAGAGCTATGCTGGAAACAGCATAGC | TAGAGGACTGG | TAGCTA | 221 |
| GCATAGCT | AAGTTGAAATAAGGCTAGTCCGTTATCAAC | GGAATGGTGTC | TGCCG | |
| ATAT (SEQ | TTGAAAAAGTGGCACCGAGTCGGTGC | AATA (SEQ ID | ACT | |
| ID NO: 9) | (SEQ ID NO: 577) | NO: 441) | (SEQ ID | |
| | | | NO: 6) | |
| GAGAGTCG | GTTTCAGAGCTATGCTGGAAACAGCATAGC | CTCCTAGAGGA | TAGCTA | 222 |
| GCATAGCT | AAGTTGAAATAAGGCTAGTCCGTTATCAAC | CTGGGGGATCGT | TGCCG | |
| ATAT (SEQ | TTGAAAAAGTGGCACCGAGTCGGTGC | ATCAATA (SEQ | ACT | |
| ID NO: 9) | (SEQ ID NO: 577) | ID NO: 442) | (SEQ ID | |
| | | | NO: 6) | |
| GAGAGTCG | GTTTCAGAGCTATGCTGGAAACAGCATAGC | CTCCTAGAGGA | TAGCTA | 223 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCAATA (SEQ | ACT | |
| ID NO: 9) | (SEQ ID NO: 577) | ID NO: 443) | (SEQ ID | |
| | | | NO: 6) | |
| GAGAGTCG | GTTTCAGAGCTATGCTGGAAACAGCATAGC | CTCCTAGAGGA | TAGCTA | 224 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCAATG (SEQ | ACT | |
| ID NO: 9) | (SEQ ID NO: 577) | ID NO: 444) | (SEQ ID | |
| | | | NO: 6) | |
| GAGAGTCG | GTTTCAGAGCTATGCTGGAAACAGCATAGC | GACTCCTAGAG | TAGCTA | 225 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCAATG | ACT | |
| ID NO: 9) | (SEQ ID NO: 577) | (SEQ ID NO: 445) | (SEQ ID | |
| | | | NO: 6) | |
| GAGAGTCG | GTTTCAGAGCTATGCTGGAAACAGCATAGC | CTCCTAGAGGA | TGCCG | 226 |
| GCATAGCT | AAGTTGAAATAAGGCTAGTCCGTTATCAAC | CTGGGGGATCGT | TAGCTA | |
| ATAT (SEQ | TTGAAAAAGTGGCACCGAGTCGGTGC | ATCGATA (SEQ | ACT | |
| ID NO: 9) | (SEQ ID NO: 577) | ID NO: 446) | (SEQ ID | |
| | | | NO: 6) | |
| GAGAGTCG | GTTTCAGAGCTATGCTGGAAACAGCATAGC | TCCTAGAGGACT | TAGCTA | 227 |
| GCATAGCT | AAGTTGAAATAAGGCTAGTCCGTTATCAAC | GGGGAATCGTAT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 447) | (SEQ ID | |
| | | | NO: 6) | |
| GAGAGTCG | GTTTCAGAGCTATGCTGGAAACAGCATAGC | GACTCCTAGAG | TAGCTA | 228 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | GGTGTCAATG | ACT | |
| ID NO: 9) | (SEQ ID NO: 577) | (SEQ ID NO: 448) | (SEQ ID | |
| | | | NO: 6) | |
| GAGAGTCG | GTTTCAGAGCTATGCTGGAAACAGCATAGC | TCCTAGAGGACT | TAGCTA | 229 |
| GCATAGCT | AAGTTGAAATAAGGCTAGTCCGTTATCAAC | GGGGAATCGTAT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 449) | (SEQ ID | |
| | | | NO: 6) | |
| GAGAGTCG | GTTTCAGAGCTATGCTGGAAACAGCATAGC | TAGAGGACTGG | TAGCTA | 230 |
| GCATAGCT | AAGTTGAAATAAGGCTAGTCCGTTATCAAC | GGAATGGTATCA | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 450) | (SEQ ID | |
| | | | NO: 6) | |
| GAGAGTCG | GTTTCAGAGCTATGCTGGAAACAGCATAGC | TAGAGGACTGG | TAGCTA | 231 |
| GCATAGCT | AAGTTGAAATAAGGCTAGTCCGTTATCAAC | GGAATGGTGTC | TGCCG | |
| ATAT (SEQ | TTGAAAAAGTGGCACCGAGTCGGTGC | GATA (SEQ ID | ACT | |
| ID NO: 9) | (SEQ ID NO: 577) | NO: 451) | (SEQ ID | |
| | | | NO: 6) | |
| GAGAGTCG | GTTTCAGAGCTATGCTGGAAACAGCATAGC | TAGAGGACTGG | TAGCTA | 232 |
| GCATAGCT | AAGTTGAAATAAGGCTAGTCCGTTATCAAC | GGAATAGTGTCG | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 452) | (SEQ ID | |
| | | | NO: 6) | |
| GAGAGTCG | GTTTCAGAGCTATGCTGGAAACAGCATAGC | TAGAGGACTGG | TAGCTA | 233 |
| GCATAGCT | AAGTTGAAATAAGGCTAGTCCGTTATCAAC | GGAATGGTGTC | TGCCG | |
| ATAT (SEQ | TTGAAAAAGTGGCACCGAGTCGGTGC | AATG (SEQ ID | ACT | |
| ID NO: 9) | (SEQ ID NO: 577) | NO: 453) | (SEQ ID | |
| | | | NO: 6) | |
| GAGAGTCG | GTTTCAGAGCTATGCTGGAAACAGCATAGC | TAGAGGACTGG | TAGCTA | 234 |
| GCATAGCT | AAGTTGAAATAAGGCTAGTCCGTTATCAAC | GGAATAGTATCG | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 454) | (SEQ ID | |
| | | | NO: 6) | |
| GAGAGTCG | GTTTCAGAGCTATGCTGGAAACAGCATAGC | TAGAGGACTGG | TAGCTA | 235 |
| GCATAGCT | AAGTTGAAATAAGGCTAGTCCGTTATCAAC | GGAATAGTGTCA | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 455) | (SEQ ID | |
| | | | NO: 6) | |
| GAGAGTCG | GTTTCAGAGCTATGCTGGAAACAGCATAGC | TAGAGGACTGG | TAGCTA | 236 |
| GCATAGCT | AAGTTGAAATAAGGCTAGTCCGTTATCAAC | GGAATAGTATCA | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 456) | (SEQ ID | |
| | | | NO: 6) | |
| GAGAGTCG | GTTTCAGAGCTATGCTGGAAACAGCATAGC | CCTAGAGGACT | TAGCTA | 237 |
| GCATAGCT | AAGTTGAAATAAGGCTAGTCCGTTATCAAC | GGGGGATCGTG | TGCCG | |
| ATAT (SEQ | TTGAAAAAGTGGCACCGAGTCGGTGC | TCGATA (SEQ ID | ACT | |
| ID NO: 9) | (SEQ ID NO: 577) | NO: 457) | (SEQ ID | |
| | | | NO: 6) | |
| GAGAGTCG | GTTTCAGAGCTATGCTGGAAACAGCATAGC | TAGAGGACTGG | TAGCTA | 238 |
| GCATAGCT | AAGTTGAAATAAGGCTAGTCCGTTATCAAC | GGAATGGTGTC | TGCCG | |
| ATAT (SEQ | TTGAAAAAGTGGCACCGAGTCGGTGC | GATG (SEQ ID | ACT | |
| ID NO: 9) | (SEQ ID NO: 577) | NO: 458) | (SEQ ID | |
| | | | NO: 6) | |
| GAGAGTCG | GTTTCAGAGCTATGCTGGAAACAGCATAGC | TCCTAGAGGACT | TAGCTA | 239 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 459) | (SEQ ID | |
| | | | NO: 6) | |
| GAGAGTCG | GTTTCAGAGCTATGCTGGAAACAGCATAGC | TAGAGGACTGG | TAGCTA | 240 |
| GCATAGCT | AAGTTGAAATAAGGCTAGTCCGTTATCAAC | GGAATAGTATCG | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 460) | (SEQ ID | |
| | | | NO: 6) | |
| GAGAGTCG | GTTTCAGAGCTATGCTGGAAACAGCATAGC | TAGAGGACTGG | TAGCTA | 241 |
| GCATAGCT | AAGTTGAAATAAGGCTAGTCCGTTATCAAC | GGAATAGTGTCG | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 461) | (SEQ ID | |
| | | | NO: 6) | |
| GAGAGTCG | GTTTCAGAGCTATGCTGGAAACAGCATAGC | CTCCTAGAGGA | TAGCTA | 242 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCAATG (SEQ | ACT | |
| ID NO: 9) | (SEQ ID NO: 577) | ID NO: 462) | (SEQ ID | |
| | | | NO: 6) | |
| GAGAGTCG | GTTTCAGAGCTATGCTGGAAACAGCATAGC | TAGAGGACTGG | TAGCTA | 243 |
| GCATAGCT | AAGTTGAAATAAGGCTAGTCCGTTATCAAC | GGAATGGTATCG | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 463) | (SEQ ID | |
| | | | NO: 6) | |
| GAGAGTCG | GTTTCAGAGCTATGCTGGAAACAGCATAGC | CCTAGAGGACT | TAGCTA | 244 |
| GCATAGCT | AAGTTGAAATAAGGCTAGTCCGTTATCAAC | GGGGGATCGTG | TGCCG | |
| ATAT (SEQ | TTGAAAAAGTGGCACCGAGTCGGTGC | TCGATG (SEQ ID | ACT | |
| ID NO: 9) | (SEQ ID NO: 577) | NO: 464) | (SEQ ID | |
| | | | NO: 6) | |
| GAGAGTCG | GTTTCAGAGCTATGCTGGAAACAGCATAGC | GACTCCTAGAG | TAGCTA | 245 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | GGTGTCAATA | ACT | |
| ID NO: 9) | (SEQ ID NO: 577) | (SEQ ID NO: 465) | (SEQ ID | |
| | | | NO: 6) | |
| GAGAGTCG | GTTTCAGAGCTATGCTGGAAACAGCATAGC | CTCCTAGAGGA | TAGCTA | 246 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCAATA (SEQ | ACT | |
| ID NO: 9) | (SEQ ID NO: 577) | ID NO: 466) | (SEQ ID | |
| | | | NO: 6) | |
| GAGAGTCG | GTTTCAGAGCTATGCTGGAAACAGCATAGC | TAGAGGACTGG | TAGCTA | 247 |
| GCATAGCT | AAGTTGAAATAAGGCTAGTCCGTTATCAAC | GGAATGGTATCA | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 467) | (SEQ ID | |
| | | | NO: 6) | |
| GAGAGTCG | GTTTCAGAGCTATGCTGGAAACAGCATAGC | TCCTAGAGGACT | TAGCTA | 248 |
| GCATAGCT | AAGTTGAAATAAGGCTAGTCCGTTATCAAC | GGGGAATCGTAT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 468) | (SEQ ID | |
| | | | NO: 6) | |
| GAGAGTCG | GTTTCAGAGCTATGCTGGAAACAGCATAGC | TAGAGGACTGG | TAGCTA | 249 |
| GCATAGCT | AAGTTGAAATAAGGCTAGTCCGTTATCAAC | GGAATAGTATCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 469) | (SEQ ID | |
| | | | NO: 6) | |
| GAGAGTCG | GTTTCAGAGCTATGCTGGAAACAGCATAGC | GACTCCTAGAG | TAGCTA | 250 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | CGTGTCAATG | ACT | |
| ID NO: 9) | (SEQ ID NO: 577) | (SEQ ID NO: 470) | (SEQ ID | |
| | | | NO: 6) | |
| GAGAGTCG | GTTTCAGAGCTATGCTGGAAACAGCATAGC | TAGAGGACTGG | TAGCTA | 251 |
| GCATAGCT | AAGTTGAAATAAGGCTAGTCCGTTATCAAC | GGAATCGTGTCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 471) | (SEQ ID | |
| | | | NO: 6) | |
| GAGAGTCG | GTTTCAGAGCTATGCTGGAAACAGCATAGC | TAGAGGACTGG | TAGCTA | 252 |
| GCATAGCT | AAGTTGAAATAAGGCTAGTCCGTTATCAAC | GGAATGGTATCG | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 472) | (SEQ ID | |
| | | | NO: 6) | |
| GAGAGTCG | GTTTCAGAGCTATGCTGGAAACAGCATAGC | GACTCCTAGAG | TAGCTA | 253 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCAATA | ACT | |
| ID NO: 9) | (SEQ ID NO: 577) | (SEQ ID NO: 473) | (SEQ ID | |
| | | | NO: 6) | |
| GAGAGTCG | GTTTCAGAGCTATGCTGGAAACAGCATAGC | TAGAGGACTGG | TAGCTA | 254 |
| GCATAGCT | AAGTTGAAATAAGGCTAGTCCGTTATCAAC | GGAATGGTGTCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 474) | (SEQ ID | |
| | | | NO: 6) | |
| GAGAGTCG | GTTTCAGAGCTATGCTGGAAACAGCATAGC | TAGAGGACTGG | TAGCTA | 255 |
| GCATAGCT | AAGTTGAAATAAGGCTAGTCCGTTATCAAC | GGAATGGTATCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 475) | (SEQ ID | |
| | | | NO: 6) | |
| GAGAGTCG | GTTTCAGAGCTATGCTGGAAACAGCATAGC | TAGAGGACTGG | TAGCTA | 256 |
| GCATAGCT | AAGTTGAAATAAGGCTAGTCCGTTATCAAC | GGAATAGTGTCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 476) | (SEQ ID | |
| | | | NO: 6) | |
| GAGAGTCG | GTTTCAGAGCTATGCTGGAAACAGCATAGC | CCTAGAGGACT | TAGCTA | 257 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 477) | (SEQ ID | |
| | | | NO: 6) | |
| GAGAGTCG | GTTTCAGAGCTATGCTGGAAACAGCATAGC | TAGAGGACTGG | TAGCTA | 258 |
| GCATAGCT | AAGTTGAAATAAGGCTAGTCCGTTATCAAC | GGAATAGTGTCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 478) | (SEQ ID | |
| | | | NO: 6) | |
| GAGAGTCG | GTTTCAGAGCTATGCTGGAAACAGCATAGC | TCCTAGAGGACT | TAGCTA | 259 |
| GCATAGCT | AAGTTGAAATAAGGCTAGTCCGTTATCAAC | GGGGGATGGTG | TGCCG | |
| ATAT (SEQ | TTGAAAAAGTGGCACCGAGTCGGTGC | TCGATA (SEQ ID | ACT | |
| ID NO: 9) | (SEQ ID NO: 577) | NO: 479) | (SEQ ID | |
| | | | NO: 6) | |
| GAGAGTCG | GTTTCAGAGCTATGCTGGAAACAGCATAGC | TAGAGGACTGG | TAGCTA | 260 |
| GCATAGCT | AAGTTGAAATAAGGCTAGTCCGTTATCAAC | GGAATCGTATCT | TGCCG | |
| ATAT (SEQ | TTGAAAAAGTGGCACCGAGTCGGTGC | ATA (SEQ ID NO: | ACT | |
| ID NO: 9) | (SEQ ID NO: 577) | 480) | (SEQ ID | |
| | | | NO: 6) | |
| GAGAGTCG | GTTTCAGAGCTATGCTGGAAACAGCATAGC | CTCCTAGAGGA | TAGCTA | 261 |
| GCATAGCT | AAGTTGAAATAAGGCTAGTCCGTTATCAAC | CTGGGGGATCGT | TGCCG | |
| ATAT (SEQ | TTGAAAAAGTGGCACCGAGTCGGTGC | ATCGATG (SEQ | ACT | |
| ID NO: 9) | (SEQ ID NO: 577) | ID NO: 481) | (SEQ ID | |
| | | | NO: 6) | |
| GAGAGTCG | GTTTCAGAGCTATGCTGGAAACAGCATAGC | TCCTAGAGGACT | TAGCTA | 262 |
| GCATAGCT | AAGTTGAAATAAGGCTAGTCCGTTATCAAC | GGGGGATGGTG | TGCCG | |
| ATAT (SEQ | TTGAAAAAGTGGCACCGAGTCGGTGC | TCGATG (SEQ ID | ACT | |
| ID NO: 9) | (SEQ ID NO: 577) | NO: 482) | (SEQ ID | |
| | | | NO: 6) | |
| GAGAGTCG | GTTTCAGAGCTATGCTGGAAACAGCATAGC | TAGAGGACTGG | TAGCTA | 263 |
| GCATAGCT | AAGTTGAAATAAGGCTAGTCCGTTATCAAC | GGAATAGTATCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 483) | (SEQ ID | |
| | | | NO: 6 | |
| GAGAGTCG | GTTTCAGAGCTATGCTGGAAACAGCATAGC | TAGAGGACTGG | TAGCTA | 264 |
| GCATAGCT | AAGTTGAAATAAGGCTAGTCCGTTATCAAC | GGAATGGTGTCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 484) | (SEQ ID | |
| | | | NO: 6) | |
| GAGAGTCG | GTTTCAGAGCTATGCTGGAAACAGCATAGC\| | CTCCTAGAGGA | TAGCTA | 265 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCAATA (SEQ | ACT | |
| ID NO: 9) | (SEQ ID NO: 577) | ID NO: 485) | (SEQ ID | |
| | | | NO: 6) | |
| GAGAGTCG | GTTTCAGAGCTATGCTGGAAACAGCATAGC | GACTCCTAGAG | TAGCTA | 266 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCGATG | ACT | |
| ID NO: 9) | (SEQ ID NO: 577) | (SEQ ID NO: 486) | (SEQ ID | |
| | | | NO: 6) | |
| GAGAGTCG | GTTTCAGAGCTATGCTGGAAACAGCATAGC | TCCTAGAGGACT | TAGCTA | 267 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 487) | (SEQ ID | |
| | | | NO: 6) | |
| GAGAGTCG | GTTTCAGAGCTATGCTGGAAACAGCATAGC | CTCCTAGAGGA | TAGCTA | 268 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCGATA (SEQ | ACT | |
| ID NO: 9) | (SEQ ID NO: 577) | ID NO: 488) | (SEQ ID | |
| | | | NO: 6) | |
| GAGAGTCG | GTTTCAGAGCTATGCTGGAAACAGCATAGC | CTCCTAGAGGA | TAGCTA | 269 |
| GCATAGCT | AAGTTGAAATAAGGCTAGTCCGTTATCAAC | CTGGGGGATCGT | TGCCG | |
| ATAT (SEQ | TTGAAAAAGTGGCACCGAGTCGGTGC | ATCAATG (SEQ | ACT | |
| ID NO: 9) | (SEQ ID NO: 577) | ID NO: 489) | (SEQ ID | |
| | | | NO: 6) | |
| GAGAGTCG | GTTTCAGAGCTATGCTGGAAACAGCATAGC | CTCCTAGAGGA | TAGCTA | 270 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCAATG (SEQ | ACT | |
| ID NO: 9) | (SEQ ID NO: 577) | ID NO: 490) | (SEQ ID | |
| | | | NO: 6) | |
| GAGAGTCG | GTTTCAGAGCTATGCTGGAAACAGCATAGC | CTCCTAGAGGA | TAGCTA | 271 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCGATA (SEQ | ACT | |
| ID NO: 9) | (SEQ ID NO: 577) | ID NO: 491) | (SEQ ID | |
| | | | NO: 6) | |
| GAGAGTCG | GTTTCAGAGCTATGCTGGAAACAGCATAGC | GACTCCTAGAG | TAGCTA | 272 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCGATA | ACT | |
| ID NO: 9) | (SEQ ID NO: 577) | (SEQ ID NO: 492) | (SEQ ID | |
| | | | NO: 6) | |
| GAGAGTCG | GTTTCAGAGCTATGCTGGAAACAGCATAGC | CTCCTAGAGGA | TAGCTA | 273 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCGATG (SEQ | ACT | |
| ID NO: 9) | (SEQ ID NO: 577) | ID NO: 493) | (SEQ ID | |
| | | | NO: 6) | |
| GAGAGTCG | GTTTCAGAGCTATGCTGGAAACAGCATAGC | CTCCTAGAGGA | TAGCTA | 274 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCGATG (SEQ | ACT | |
| ID NO: 9) | (SEQ ID NO: 577) | ID NO: 494) | (SEQ ID | |
| | | | NO: 6) | |
| GAGAGTCG | GTTTCAGAGCTATGCTGGAAACAGCATAGC | TAGAGGACTGG | TAGCTA | 275 |
| GCATAGCT | AAGTTGAAATAAGGCTAGTCCGTTATCAAC | GGAATGGTATCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 495) | (SEQ ID | |
| | | | NO: 6) | |
| GAGAGTCG | GTTTCAGAGCTATGCTGGAAACAGCATAGC | TCCTAGAGGACT | TAGCTA | 276 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 496) | (SEQ ID | |
| | | | NO: 6) | |
| GAGAGTCG | GTTTCAGAGCTATGCTGGAAACAGCATAGC | CTCCTAGAGGA | TAGCTA | 277 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCGATA (SEQ | ACT | |
| ID NO: 9) | (SEQ ID NO: 577) | ID NO: 497) | (SEQ ID | |
| | | | NO: 6) | |
| GAGAGTCG | GTTTCAGAGCTATGCTGGAAACAGCATAGC | CTCCTAGAGGA | TAGCTA | 278 |
| GCATAGCT | AAGTTGAAATAAGGCTAGTCCGTTATCAAC | CTGGGGGATCGT | TGCCG | |
| ATAT (SEQ | TTGAAAAAGTGGCACCGAGTCGGTGC | ATCTATA (SEQ ID | ACT | |
| ID NO: 9) | (SEQ ID NO: 577) | NO: 498) | (SEQ ID | |
| | | | NO: 6) | |
| GAGAGTCG | GTTTCAGAGCTATGCTGGAAACAGCATAGC | CTCCTAGAGGA | TAGCTA | 279 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCGATG (SEQ | ACT | |
| ID NO: 9) | (SEQ ID NO: 577) | ID NO: 499) | (SEQ ID | |
| | | | NO: 6) | |
| GAGAGTCG | GTTTCAGAGCTATGCTGGAAACAGCATAGC | TAGAGGACTGG | TAGCTA | 280 |
| GCATAGCT | AAGTTGAAATAAGGCTAGTCCGTTATCAAC | GGAATCGTATCT | TGCCG | |
| ATAT (SEQ | TTGAAAAAGTGGCACCGAGTCGGTGC | ATG (SEQ ID NO: | ACT | |
| ID NO: 9) | (SEQ ID NO: 577) | 500) | (SEQ ID | |
| | | | NO: 6) | |
| GAGAGTCG | GTTTCAGAGCTATGCTGGAAACAGCATAGC | CCTAGAGGACT | TGCCG | 281 |
| GCATAGCT | AAGTTGAAATAAGGCTAGTCCGTTATCAAC | GGGGGATTGTGT | TAGCTA | |
| ATAT (SEQ | TTGAAAAAGTGGCACCGAGTCGGTGC | CAATG (SEQ ID | ACT | |
| ID NO: 9) | (SEQ ID NO: 577) | NO: 501) | (SEQ ID | |
| | | | NO: 6) | |
| GAGAGTCG | GTTTCAGAGCTATGCTGGAAACAGCATAGC | CCTAGAGGACT | TAGCTA | 282 |
| GCATAGCT | AAGTTGAAATAAGGCTAGTCCGTTATCAAC | GGGGTATGGTAT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 502) | (SEQ ID | |
| | | | NO: 6) | |
| GAGAGTCG | GTTTCAGAGCTATGCTGGAAACAGCATAGC | CCTAGAGGACT | TAGCTA | 283 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 503) | (SEQ ID | |
| | | | NO: 6) | |
| GAGAGTCG | GTTTCAGAGCTATGCTGGAAACAGCATAGC | CTCCTAGAGGA | TAGCTA | 284 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCTATA (SEQ | ACT | |
| ID NO: 9) | (SEQ ID NO: 577) | ID NO: 504) | (SEQ ID | |
| | | | NO: 6) | |
| GAGAGTCG | GTTTCAGAGCTATGCTGGAAACAGCATAGC | TCCTAGAGGACT | TAGCTA | 285 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 505) | (SEQ ID | |
| | | | NO: 6) | |
| GAGAGTCG | GTTTCAGAGCTATGCTGGAAACAGCATAGC | CTCCTAGAGGA | TAGCTA | 286 |
| GCATAGCT | AAGTTGAAATAAGGCTAGTCCGTTATCAAC | CTGGGGTATGGT | TGCCG | |
| ATAT (SEQ | TTGAAAAAGTGGCACCGAGTCGGTGC | ATCAATA (SEQ | ACT | |
| ID NO: 9) | (SEQ ID NO: 577) | ID NO: 506) | (SEQ ID | |
| | | | NO: 6) | |
| GAGAGTCG | GTTTCAGAGCTATGCTGGAAACAGCATAGC | GACTGGGGGAT | TAGCTA | 287 |
| GCATAGCT | AAGTTGAAATAAGGCTAGTCCGTTATCAAC | GACTCCTAGAG | TGCCG | |
| ATAT (SEQ | TTGAAAAAGTGGCACCGAGTCGGTGC | CGTGTCTATA | ACT | |
| ID NO: 9) | (SEQ ID NO: 577) | (SEQ ID NO: 507) | (SEQ ID | |
| | | | NO: 6) | |
| GAGAGTCG | GTTTCAGAGCTATGCTGGAAACAGCATAGC | CCTAGAGGACT | TAGCTA | 288 |
| GCATAGCT | AAGTTGAAATAAGGCTAGTCCGTTATCAAC | GGGGGATTGTGT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 508) | (SEQ ID | |
| | | | NO: 6) | |
| GAGAGTCG | GTTTCAGAGCTATGCTGGAAACAGCATAGC | CCTAGAGGACT | TAGCTA | 289 |
| GCATAGCT | AAGTTGAAATAAGGCTAGTCCGTTATCAAC | GGGGGATTGTGT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 509) | (SEQ ID | |
| | | | NO: 6) | |
| GAGAGTCG | GTTTCAGAGCTATGCTGGAAACAGCATAGC | GACTCCTAGAG | TAGCTA | 290 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | CGTGTCTATG | ACT | |
| ID NO: 9) | (SEQ ID NO: 577) | (SEQ ID NO: 510) | (SEQ ID | |
| | | | NO: 6) | |
| GAGAGTCG | GTTTCAGAGCTATGCTGGAAACAGCATAGC | CTCCTAGAGGA | TAGCTA | 291 |
| GCATAGCT | AAGTTGAAATAAGGCTAGTCCGTTATCAAC | CTGGGGGATGG | TGCCG | |
| ATAT (SEQ | TTGAAAAAGTGGCACCGAGTCGGTGC | TATCTATG (SEQ | ACT | |
| ID NO: 9) | (SEQ ID NO: 577) | ID NO: 511) | (SEQ ID | |
| | | | NO: 6) | |
| GAGAGTCG | GTTTCAGAGCTATGCTGGAAACAGCATAGC | CTCCTAGAGGA | TAGCTA | 292 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCTATA (SEQ ID | ACT | |
| ID NO: 9) | (SEQ ID NO: 577) | NO: 512) | (SEQ ID | |
| | | | NO: 6) | |
| GAGAGTCG | GTTTCAGAGCTATGCTGGAAACAGCATAGC | CCTAGAGGACT | TAGCTA | 293 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 513) | (SEQ ID | |
| | | | NO: 6) | |
| GAGAGTCG | GTTTCAGAGCTATGCTGGAAACAGCATAGC | TCCTAGAGGACT | TAGCTA | 294 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 514) | (SEQ ID | |
| | | | NO: 6) | |
| GAGAGTCG | GTTTCAGAGCTATGCTGGAAACAGCATAGC | CCTAGAGGACT | TAGCTA | 295 |
| GCATAGCT | AAGTTGAAATAAGGCTAGTCCGTTATCAAC | GGGGGATTGTGT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 515) | (SEQ ID | |
| | | | NO: 6) | |
| GAGAGTCG | GTTTCAGAGCTATGCTGGAAACAGCATAGC | GACTCCTAGAG | TAGCTA | 296 |
| GCATAGCT | AAGTTGAAATAAGGCTAGTCCGTTATCAAC | GACTGGGGTATA | TGCCG | |
| ATAT (SEQ | TTGAAAAAGTGGCACCGAGTCGGTGC | GTATCAATA | ACT | |
| ID NO: 9) | (SEQ ID NO: 577) | (SEQ ID NO: 516) | (SEQ ID | |
| | | | NO: 6) | |
| GAGAGTCG | GTTTCAGAGCTATGCTGGAAACAGCATAGC | TCCTAGAGGACT | TAGCTA | 297 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 517) | (SEQ ID | |
| | | | NO: 6) | |
| GAGAGTCG | GTTTCAGAGCTATGCTGGAAACAGCATAGC | CCTAGAGGACT | TAGCTA | 298 |
| GCATAGCT | AAGTTGAAATAAGGCTAGTCCGTTATCAAC | GGGGTATGGTAT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 518) | (SEQ ID | |
| | | | NO: 6) | |
| GAGAGTCG | GTTTCAGAGCTATGCTGGAAACAGCATAGC | GACTCCTAGAG | TAGCTA | 299 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | GGTGTCTATG | ACT | |
| ID NO: 9) | (SEQ ID NO: 577) | (SEQ ID NO: 519) | (SEQ ID | |
| | | | NO: 6) | |
| GAGAGTCG | GTTTCAGAGCTATGCTGGAAACAGCATAGC | CTCCTAGAGGA | TAGCTA | 300 |
| GCATAGCT | AAGTTGAAATAAGGCTAGTCCGTTATCAAC | CTGGGGGATCGT | TGCCG | |
| ATAT (SEQ | TTGAAAAAGTGGCACCGAGTCGGTGC | ATCTATG (SEQ | ACT | |
| ID NO: 9) | (SEQ ID NO: 577) | ID NO: 520) | (SEQ ID | |
| | | | NO: 6) | |
| GAGAGTCG | GTTTCAGAGCTATGCTGGAAACAGCATAGC | GACTCCTAGAG | TAGCTA | 301 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | GGTGTCTATA | ACT | |
| ID NO: 9) | (SEQ ID NO: 577) | (SEQ ID NO: 521) | (SEQ ID | |
| | | | NO: 6) | |
| GAGAGTCG | GTTTCAGAGCTATGCTGGAAACAGCATAGC | CTCCTAGAGGA | TAGCTA | 302 |
| GCATAGCT | AAGTTGAAATAAGGCTAGTCCGTTATCAAC | CTGGGGGATAGT | TGCCG | |
| ATAT (SEQ | TTGAAAAAGTGGCACCGAGTCGGTGC | ATCTATG (SEQ | ACT | |
| ID NO: 9) | (SEQ ID NO: 577) | ID NO: 522) | (SEQ ID | |
| | | | NO: 6) | |
| GAGAGTCG | GTTTCAGAGCTATGCTGGAAACAGCATAGC | TCCTAGAGGACT | TAGCTA | 303 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 523) | (SEQ ID | |
| | | | NO: 6) | |
| GAGAGTCG | GTTTCAGAGCTATGCTGGAAACAGCATAGC | GACTCCTAGAG | TAGCTA | 304 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCTATG | ACT | |
| ID NO: 9) | (SEQ ID NO: 577) | (SEQ ID NO: 524) | (SEQ ID | |
| | | | NO: 6) | |
| GAGAGTCG | GTTTCAGAGCTATGCTGGAAACAGCATAGC | GACTCCTAGAG | TAGCTA | 305 |
| GCATAGCT | AAGTTGAAATAAGGCTAGTCCGTTATCAAC | GACTGGGGTATA | TGCCG | |
| ATAT (SEQ | TTGAAAAAGTGGCACCGAGTCGGTGC | GTATCAATG | ACT | |
| ID NO: 9) | (SEQ ID NO: 577) | (SEQ ID NO: 525) | (SEQ ID | |
| | | | NO: 6) | |
| GAGAGTCG | GTTTCAGAGCTATGCTGGAAACAGCATAGC | TCCTAGAGGACT | TAGCTA | 306 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 526) | (SEQ ID | |
| | | | NO: 6) | |
| GAGAGTCG | GTTTCAGAGCTATGCTGGAAACAGCATAGC | GACTCCTAGAG | TAGCTA | 307 |
| GCATAGCT | AAGTTGAAATAAGGCTAGTCCGTTATCAAC | GACTGGGGGAT | TGCCG | |
| ATAT (SEQ | TTGAAAAAGTGGCACCGAGTCGGTGC | AGTGTCTATA | ACT | |
| ID NO: 9) | (SEQ ID NO: 577) | (SEQ ID NO: 527) | (SEQ ID | |
| | | | NO: 6) | |
| GAGAGTCG | GTTTCAGAGCTATGCTGGAAACAGCATAGC | CCTAGAGGACT | TAGCTA | 308 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 528) | (SEQ ID | |
| | | | NO: 6) | |
| GAGAGTCG | GTTTCAGAGCTATGCTGGAAACAGCATAGC | CTCCTAGAGGA | TAGCTA | 309 |
| GCATAGCT | AAGTTGAAATAAGGCTAGTCCGTTATCAAC | CTGGGGTATGGT | TGCCG | |
| ATAT (SEQ | TTGAAAAAGTGGCACCGAGTCGGTGC | ATCAATG (SEQ | ACT | |
| ID NO: 9) | (SEQ ID NO: 577) | ID NO: 529) | (SEQ ID | |
| | | | NO: 6) | |
| GAGAGTCG | \|GTTTCAGAGCTATGCTGGAAACAGCATAGC | CTCCTAGAGGA | TAGCTA | 310 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCTATA (SEQ ID | ACT | |
| ID NO: 9) | (SEQ ID NO: 577) | NO: 530) | (SEQ ID | |
| | | | NO: 6) | |
| GAGAGTCG | GTTTCAGAGCTATGCTGGAAACAGCATAGC | GACTCCTAGAG | TAGCTA | 311 |
| GCATAGCT | AAGTTGAAATAAGGCTAGTCCGTTATCAAC | GACTGGGGTATA | TGCCG | |
| ATAT (SEQ | TTGAAAAAGTGGCACCGAGTCGGTGC | GTATCGATA | ACT | |
| ID NO: 9) | (SEQ ID NO: 577) | (SEQ ID NO: 531) | (SEQ ID | |
| | | | NO: 6) | |
| GAGAGTCG | GTTTCAGAGCTATGCTGGAAACAGCATAGC | TCCTAGAGGACT | TAGCTA | 312 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 532) | (SEQ ID | |
| | | | NO: 6) | |
| GAGAGTCG | GTTTCAGAGCTATGCTGGAAACAGCATAGC | CCTAGAGGACT | TAGCTA | 313 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CAATA (SEQ ID | ACT | |
| ID NO: 9) | (SEQ ID NO: 577) | NO: 533) | (SEQ ID | |
| | | | NO: 6) | |
| GAGAGTCG | GTTTCAGAGCTATGCTGGAAACAGCATAGC | TCCTAGAGGACT | TAGCTA | 314 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 534) | (SEQ ID | |
| | | | NO: 6) | |
| GAGAGTCG | GTTTCAGAGCTATGCTGGAAACAGCATAGC | CTCCTAGAGGA | TAGCTA | 315 |
| GCATAGCT | AAGTTGAAATAAGGCTAGTCCGTTATCAAC | CTGGGGGATTGT | TGCCG | |
| ATAT (SEQ | TTGAAAAAGTGGCACCGAGTCGGTGC | ATCTATG (SEQ | ACT | |
| ID NO: 9) | (SEQ ID NO: 577) | ID NO: 535) | (SEQ ID | |
| | | | NO: 6) | |
| GAGAGTCG | GTTTCAGAGCTATGCTGGAAACAGCATAGC | GACTCCTAGAG | TAGCTA | 316 |
| GCATAGCT | AAGTTGAAATAAGGCTAGTCCGTTATCAAC | GACTGGGGTATG | TGCCG | |
| ATAT (SEQ | TTGAAAAAGTGGCACCGAGTCGGTGC | GTGTCAATA | ACT | |
| ID NO: 9) | (SEQ ID NO: 577) | (SEQ ID NO: 536) | (SEQ ID | |
| | | | NO: 6) | |
| GAGAGTCG | GTTTCAGAGCTATGCTGGAAACAGCATAGC | TCCTAGAGGACT | TAGCTA | 317 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 537) | (SEQ ID | |
| | | | NO: 6) | |
| GAGAGTCG | GTTTCAGAGCTATGCTGGAAACAGCATAGC | CCTAGAGGACT | TAGCTA | 318 |
| GCATAGCT | AAGTTGAAATAAGGCTAGTCCGTTATCAAC | GGGGGATTGTGT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 538) | (SEQ ID | |
| | | | NO: 6) | |
| GAGAGTCG | GTTTCAGAGCTATGCTGGAAACAGCATAGC | GACTCCTAGAG | TAGCTA | 319 |
| GCATAGCT | AAGTTGAAATAAGGCTAGTCCGTTATCAAC | GACTGGGGTATA | TGCCG | |
| ATAT (SEQ | TTGAAAAAGTGGCACCGAGTCGGTGC | GTATCGATG | ACT | |
| ID NO: 9) | (SEQ ID NO: 577) | (SEQ ID NO: 539) | (SEQ ID | |
| | | | NO: 6) | |
| GAGAGTCG | GTTTCAGAGCTATGCTGGAAACAGCATAGC | CCTAGAGGACT | TAGCTA | 320 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CGATA (SEQ ID | ACT | |
| ID NO: 9) | (SEQ ID NO: 577) | NO: 540) | (SEQ ID | |
| | | | NO: 6) | |
| GAGAGTCG | GTTTCAGAGCTATGCTGGAAACAGCATAGC | CCTAGAGGACT | TGCCG | 321 |
| GCATAGCT | AAGTTGAAATAAGGCTAGTCCGTTATCAAC | GGGGGATTGTGT | TAGCTA | |
| ATAT (SEQ | TTGAAAAAGTGGCACCGAGTCGGTGC | CTATA (SEQ ID | ACT | |
| ID NO: 9) | (SEQ ID NO: 577) | NO: 541) | (SEQ ID | |
| | | | NO: 6) | |
| GAGAGTCG | GTTTCAGAGCTATGCTGGAAACAGCATAGC | CCTAGAGGACT | TAGCTA | 322 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CAATG (SEQ ID | ACT | |
| ID NO: 9) | (SEQ ID NO: 577) | NO: 542) | (SEQ ID | |
| | | | NO: 6) | |
| GAGAGTCG | GTTTCAGAGCTATGCTGGAAACAGCATAGC | TCCTAGAGGACT | TAGCTA | 323 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 543) | (SEQ ID | |
| | | | NO: 6) | |
| GAGAGTCG | GTTTCAGAGCTATGCTGGAAACAGCATAGC | GACTCCTAGAG | TAGCTA | 324 |
| GCATAGCT | AAGTTGAAATAAGGCTAGTCCGTTATCAAC | GACTGGGGTATG | TGCCG | |
| ATAT (SEQ | TTGAAAAAGTGGCACCGAGTCGGTGC | GTGTCAATG | ACT | |
| ID NO: 9) | (SEQ ID NO: 577) | (SEQ ID NO: 544) | (SEQ ID | |
| | | | NO: 6) | |
| GAGAGTCG | GTTTCAGAGCTATGCTGGAAACAGCATAGC | CTCCTAGAGGA | TAGCTA | 325 |
| GCATAGCT | AAGTTGAAATAAGGCTAGTCCGTTATCAAC | CTGGGGTATGGT | TGCCG | |
| ATAT (SEQ | TTGAAAAAGTGGCACCGAGTCGGTGC | GTCGATA (SEQ | ACT | |
| ID NO: 9) | (SEQ ID NO: 577) | ID NO: 545) | (SEQ ID | |
| | | | NO: 6) | |
| GAGAGTCG | GTTTCAGAGCTATGCTGGAAACAGCATAGC | CCTAGAGGACT | TAGCTA | 326 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CGATG (SEQ ID | ACT | |
| ID NO: 9) | (SEQ ID NO: 577) | NO: 546) | (SEQ ID | |
| | | | NO: 6) | |
| GAGAGTCG | GTTTCAGAGCTATGCTGGAAACAGCATAGC | TCCTAGAGGACT | TAGCTA | 327 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 547) | (SEQ ID | |
| | | | NO: 6) | |
| GAGAGTCG | GTTTCAGAGCTATGCTGGAAACAGCATAGC | TCCTAGAGGACT | TAGCTA | 328 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 548) | (SEQ ID | |
| | | | NO: 6) | |
| GAGAGTCG | GTTTCAGAGCTATGCTGGAAACAGCATAGC | CTCCTAGAGGA | TAGCTA | 329 |
| GCATAGCT | AAGTTGAAATAAGGCTAGTCCGTTATCAAC | CTGGGGTATGGT | TGCCG | |
| ATAT (SEQ | TTGAAAAAGTGGCACCGAGTCGGTGC | GTCGATG (SEQ | ACT | |
| ID NO: 9) | (SEQ ID NO: 577) | ID NO: 549) | (SEQ ID | |
| | | | NO: 6) | |
| GAGAGTCG | GTTTCAGAGCTATGCTGGAAACAGCATAGC | TCCTAGAGGACT | TAGCTA | 330 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 550) | (SEQ ID | |
| | | | NO: 6) | |
| GAGAGTCG | GTTTCAGAGCTATGCTGGAAACAGCATAGC | CCTAGAGGACT | TAGCTA | 331 |
| GCATAGCT | AAGTTGAAATAAGGCTAGTCCGTTATCAAC | GGGGTATGGTAT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 551) | (SEQ ID | |
| | | | NO: 6) | |
| GAGAGTCG | GTTTCAGAGCTATGCTGGAAACAGCATAGC | TCCTAGAGGACT | TAGCTA | 332 |
| GCATAGCT | AAGTTGAAATAAGGCTAGTCCGTTATCAAC | GGGGTATAGTGT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 552) | (SEQ ID | |
| | | | NO: 6) | |
| GAGAGTCG | GTTTCAGAGCTATGCTGGAAACAGCATAGC | TCCTAGAGGACT | TAGCTA | 333 |
| GCATAGCT | AAGTTGAAATAAGGCTAGTCCGTTATCAAC | GGGGTATCGTGT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 553) | (SEQ ID | |
| | | | NO: 6) | |
| GAGAGTCG | GTTTCAGAGCTATGCTGGAAACAGCATAGC | CCTAGAGGACT | TAGCTA | 334 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 554) | (SEQ ID | |
| | | | NO: 6) | |
| GAGAGTCG | GTTTCAGAGCTATGCTGGAAACAGCATAGC | CCTAGAGGACT | TAGCTA | 335 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 555) | (SEQ ID | |
| | | | NO: 6) | |
| GAGAGTCG | GTTTCAGAGCTATGCTGGAAACAGCATAGC | CCTAGAGGACT | TAGCTA | 336 |
| GCATAGCT | AAGTTGAAATAAGGCTAGTCCGTTATCAAC | GGGGTATTGTGT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 556) | (SEQ ID | |
| | | | NO: 6) | |
| GAGAGTCG | GTTTCAGAGCTATGCTGGAAACAGCATAGC | CTCCTAGAGGA | TAGCTA | 337 |
| GCATAGCT | AAGTTGAAATAAGGCTAGTCCGTTATCAAC | CTGGGGTATAGT | TGCCG | |
| ATAT (SEQ | TTGAAAAAGTGGCACCGAGTCGGTGC | ATCTATA (SEQ ID | ACT | |
| ID NO: 9) | (SEQ ID NO: 577) | NO: 557) | (SEQ ID | |
| | | | NO: 6) | |
| GAGAGTCG | GTTTCAGAGCTATGCTGGAAACAGCATAGC | CCTAGAGGACT | TAGCTA | 338 |
| GCATAGCT | AAGTTGAAATAAGGCTAGTCCGTTATCAAC | GGGGTATGGTGT | TGCCG | |
| ATAT (SEQ | TTGAAAAAGTGGCACCGAGTCGGTGC | CTATA (SEQ ID | ACT | |
| ID NO: 9) | (SEQ ID NO: 577) | NO: 558) | (SEQ ID | |
| | | | NO: 6) | |
| GAGAGTCG | GTTTCAGAGCTATGCTGGAAACAGCATAGC | TCCTAGAGGACT | TAGCTA | 339 |
| GCATAGCT | AAGTTGAAATAAGGCTAGTCCGTTATCAAC | GGGGTATGGTAT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 559) | (SEQ ID | |
| | | | NO: 6) | |
| GAGAGTCG | GTTTCAGAGCTATGCTGGAAACAGCATAGC | CCTAGAGGACT | TAGCTA | 340 |
| GCATAGCT | AAGTTGAAATAAGGCTAGTCCGTTATCAAC | GGGGTATTGTAT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 560) | (SEQ ID | |
| | | | NO: 6) | |
| GAGAGTCG | GTTTCAGAGCTATGCTGGAAACAGCATAGC | CCTAGAGGACT | TAGCTA | 341 |
| GCATAGCT | AAGTTGAAATAAGGCTAGTCCGTTATCAAC | GGGGTATGGTGT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 561) | (SEQ ID | |
| | | | NO: 6) | |
| GAGAGTCG | GTTTCAGAGCTATGCTGGAAACAGCATAGC | CTCCTAGAGGA | TAGCTA | 342 |
| GCATAGCT | AAGTTGAAATAAGGCTAGTCCGTTATCAAC | CTGGGGTATAGT | TGCCG | |
| ATAT (SEQ | TTGAAAAAGTGGCACCGAGTCGGTGC | ATCTATG (SEQ | ACT | |
| ID NO: 9) | (SEQ ID NO: 577) | ID NO: 562) | (SEQ ID | |
| | | | NO: 6) | |
| GAGAGTCG | GTTTCAGAGCTATGCTGGAAACAGCATAGC | TCCTAGAGGACT | TAGCTA | 343 |
| GCATAGCT | AAGTTGAAATAAGGCTAGTCCGTTATCAAC | GGGGTATCGTAT | TGCCG | |
| ATAT (SEQ | TTGAAAAAGTGGCACCGAGTCGGTGC | CTATG (SEQ ID | ACT | |
| ID NO: 9) | (SEQ ID NO: 577) | NO: 563) | (SEQ ID | |
| | | | NO: 6) | |

TABLE 5
EpegRNA and nicking sgRNA sequences for described mutations

| Site | epegRNA sequence |
|---|---|
| c.1412delA | GCCGGGAGGACTGGGGAATGGGTTTTAGAGCTAGAAATAGCAAGTTAA |
| correction in | AATAAGGCTAGTCCGTTATCAACTTGAAAAAGTGGCACCGAGTCGGTGC |
| mCDKL5 (PBS | GCTATATTGACACAATTCCCCAGTCCGCGGTTCTATCTAGTTACGCGTTA |
| 10 RTT 15) | AACCAACTAGAATTTTTTT (SEQ ID NO: 344) |
| c.1412delA | GAGGGACTCCTAGAGGACTGGTTTTAGAGCTAGAAATAGCAAGTTAAA |
| correction in | ATAAGGCTAGTCCGTTATCAACTTGAAAAAGTGGCACCGAGTCGGTGCA |
| hCDKL5 (NGG | TATTGACACAATTCCCCAATCCTCTAGGAGTCCGCGGTTCTATCTAGTTA |
| PAM, no linker) | CGCGTTAAACCAACTAGAATTTTTTT (SEQ ID NO: 345) |
| c.826-1 G > A | GCTGTCAAGTATCTGTCAGCTGTTTTAGAGCTAGAAATAGCAAGTTAAA |
| correction | ATAAGGCTAGTCCGTTATCAACTTGAAAAAGTGGCACCGAGTCGGTGCA |
| | TTTCCTAAGAACCTGTTGAAGTTGGACCCAGCTGACAGATACTCGCGGT |
| | TCTATCTAGTTACGCGTTAAACCAACTAGAATTTTTTT (SEQ ID NO: 39) |
| Exon 8 cluster | CAGAGTACGTTGCCACCAGAGTTTTAGAGCTAGAAATAGCAAGTTAAAA |
| mutations (PBS | TAAGGCTAGTCCGTTATCAACTTGAAAAAGTGGCACCGAGTCGGTGCAA |
| 9 RTT 30) | GTAAGGGCTCTAGGGAGCGACACCGGCTGGTGGCAACCGCGGTTCTATC |
| | TAGTTACGCGTTAAACCAACTAGAATTTTTTT (SEQ ID NO: 40) |
| Exon 8 cluster | CAGAGTACGTTGCCACCAGAGTTTTAGAGCTAGAAATAGCAAGTTAAAA |
| mutations (PBS | TAAGGCTAGTCCGTTATCAACTTGAAAAAGTGGCACCGAGTCGGTGCAC |
| 9 RTT 34) | CCAAGTAAGGGCTCTAGGGAGCGACACCGGCTGGTGGCAACCGCGGTT |
| | CTATCTAGTTACGCGTTAAACCAACTAGAATTTTTTT (SEQ ID NO: 41) |
| Site | Nicking sgRNA sequence |
| c.826-1 G > A | GATTCTTAGGAAATAAAATTT (SEQ ID NO: 19) |
| installation | |
| mCDKL5 NG1 (NRCH) | GCCAGATCTCAGCAGAACAGG (SEQ ID NO: 2) |
| mCDKL5 NG2 (NRCH) | GCATTCATGGAAAGCTCCCAG (SEQ ID NO: 3) |
| mCDKL5 NG3 (NRCH) | GCCAGAGCAAAGCTGGGACCC (SEQ ID NO: 4) |
| c.826-1 G > A NG1 | GATTTCAAACCCAGAGACTTC (SEQ ID NO: 15) |
| c.826-1 G > A NG2 | GTTCTGGATCGTTCTCCTTCA (SEQ ID NO: 16) |
| c.826-1 G > A NG3 | GTACTGAAGTTGGACCCAGCT (SEQ ID NO: 17) |
| Exon 8 +50 | GTGGAAATGTCATTCTATTTT (SEQ ID NO: 18) |

Plasmids were cloned via current best practices⁸⁵. Corrective pegRNA (GAGGGACTCCTAGAGGACTGGTTTTAGAGCTAGAAATAGCAAGTTAAAATAAG GCTAGTCCGTTATCAACTTGAAAAAGTGGCACCGAGTCGGTGCATATTGACACAA TTCCCCAATCCTCTAGGAGTCTTT SEQ ID NO: 346) and nicking sgRNA (GCAGAACCGCCACTCATTCA SEQ ID NO: 20) for CDKL5 were previously described⁸⁶. The epegRNA, described in Table 5, includes the 3′ tevo-preQ1 motif⁸⁹.

### In Vitro Transcription of PEmax and PE6c

In vitro transcribed PEmax and PE6c mRNA were ordered from GenScript with full substitution of uridine for N1-methylpseudouridine. The final mRNA products were diluted in 1 mM Sodium Citrate (pH 6.5) at a concentration of 0.5 mg/ml.

### Synthetic Guide RNAs

Synthetic pegRNA was ordered from Integrated DNA Technologies. Each epeg contained 2′-O-methyl modifications at the first and last three nucleotides and phosphorothioate linkages between the three first and last nucleotides. Synthetic nicking sgRNAs were ordered from Synthego and included 2′-O-methyl modifications at the first and last three nucleotides and phosphorothioate bonds between the first three and last two bases.

### Electroporation of Patient Fibroblasts

Electroporations were performed with the Lonza 4D Nucleofector. 2.5×10⁵ fibroblasts per condition were electroporated with the SE Cell Line X Kit S (Lonza, V4XC) according to manufacturer instructions using pulse code CM-130. Electroporations included 2000 ng of either PEmax or PE6c mRNA, 180 μmol of synthetic pegRNA, and 120 μmol of synthetic nicking sgRNA. Cells underwent media exchange on day 3 post-transfection and genomic DNA was collected 6 days post-transfection.

### HTS

Genomic DNA (gDNA) was extracted from N2a cells, HEK293T cells, and primary fibroblasts using a lysis buffer (10 mM Tris-HCl, pH 8.0, 0.05% SDS, 25 μg ml⁻¹ proteinase K (Qaigen)) followed by incubation at 37° C. for an hour and heat inactivation at 80° C. for 30 minutes. Primers for the amplification of c.1412delA in Cdkl5 were forward:

(SEQ ID NO: 361)
ACACTCTTTCCCTACACGACGCTCTTCCGATCTNNNNGGCTGGTGGGAGA
GTTCAAG
and
reverse:
(SEQ ID NO: 362)
TGGAGTTCAGACGTGTGCTCTTCCGATCTGGTCCAGGCACAAAGTACCTC.

Primers for the amplification of c.1412delA in CDKL5 were forward:

(SEQ ID NO: 363)
ACACTCTTTCCCTACACGACGCTCTTCCGATCTNNNNCTTCAGAAGGCCC
AGGGACAAAG
and
reverse:
(SEQ ID NO: 364)
TGGAGTTCAGACGTGTGCTCTTCCGATCTCTGGTACTGGGCTCCGCAATT
T.

Primers for the amplification of c.826-1 G>A in CDKL5 were forward:

(SEQ ID NO: 365)
ACACTCTTTCCCTACACGACGCTCTTCCGATCTNNNNGTGTTTACTTGAT
ATTCTGCAATGACTG
and
reverse:
(SEQ ID NO: 366)
TGGAGTTCAGACGTGTGCTCTTCCGATCTCCAACACTGCATCTGTATTTC
CTTAAAAG.

Primers for the amplification of mutations in exon 8 were forward:

(SEQ ID NO: 367)
ACACTCTTTCCCTACACGACGCTCTTCCGATCTNNNNAGCCCATGCGAGA
ACAGTCA
and
reverse:
(SEQ ID NO: 368)
TGGAGTTCAGACGTGTGCTCTTCCGATCTTGACAATAGAATCAGCAGATG
TGGA.

Subsequent HTS was performed via Crispresso2. Briefly, fastq files were aligned and editing frequency was quantified in Crispresso2 in batch mode with a window with spanning at least 10nt past each nick site as previously described⁷⁹.

### Cas-OFFinder Off-Target Nomination, Amplicon Sequencing, and Analysis

Computational nomination of potential off-target sites with three or fewer mismatches to the epegRNA or nicking sgRNA protospacers of the c.1412delA prime editing strategy were nominated using Cas-OFFinder¹²⁸. HTS sequencing primers for each site were designed as follows:

TABLE 6
HTS sequences for Cas-OFFinder-nominated loci

| Off- | | Fwd SEQ | | Rvs SEQ |
|---|---|---|---|---|
| target | Forward primer | ID NO: | Reverse primer | ID NO: |
| Epeg | ACACTCTTTCCCTACACGACGCTCT | 369 | TGGAGTTCAGACGTGTGCTCTTCCG | 386 |
| OT1 | TCCGATCTNNNN | | ATCT CACTGATACAGTGTCCAGGAC | |
| | GTTTGAATGGAGCCGCCTG | | | |
| Epeg | ACACTCTTTCCCTACACGACGCTCT | 370 | TGGAGTTCAGACGTGTGCTCTTCCG | 387 |
| OT2 | TCCGATCTNNNN | | ATCT GGCCAGCCTCACACCC | |
| | CCCTGGTGGGTGCATACC | | | |
| Epeg | ACACTCTTTCCCTACACGACGCTCT | 371 | TGGAGTTCAGACGTGTGCTCTTCCG | 388 |
| OT3 | TCCGATCTNNNN | | ATCT GTGGGAGGCAGAACAGGC | |
| | CTCCACCCAGTTCCGGAG | | | |
| Epeg | ACACTCTTTCCCTACACGACGCTCT | 372 | TGGAGTTCAGACGTGTGCTCTTCCG | 389 |
| OT4 | TCCGATCTNNNN | | ATCT GTTCTCTCCTTGCTGGTCAC | |
| | CTGAAATGGGATGGACTTGTGTTG | | | |
| Epeg | ACACTCTTTCCCTACACGACGCTCT | 373 | TGGAGTTCAGACGTGTGCTCTTCCG | 390 |
| OT5 | TCCGATCTNNNN | | ATCT CATCTCAAGCAGGTGTGATCC | |
| | GCTGTGTGGATGCCTGTGC | | | |
| Epeg | ACACTCTTTCCCTACACGACGCTCT | 374 | TGGAGTTCAGACGTGTGCTCTTCCG | 391 |
| OT6 | TCCGATCTNNNN | | ATCT CTCCATATCTCAGGCTGAAGG | |
| | GCCTTTAGTCTGGCTCTCAG | | | |
| Epeg | ACACTCTTTCCCTACACGACGCTCT | 375 | TGGAGTTCAGACGTGTGCTCTTCCG | 392 |
| OT7 | TCCGATCTNNNN | | ATCT CTCTGCCCTATCCCCTCTG | |
| | GTGTCTCTGCTAGGCCAATC | | | |
| Epeg | ACACTCTTTCCCTACACGACGCTCT | 376 | TGGAGTTCAGACGTGTGCTCTTCCG | 393 |
| OT8 | TCCGATCTNNNN | | ATCT CTGTGTCCATTCTGTCCCTG | |
| | CACAGGCCCAGAGTGAATC | | | |
| Epeg | ACACTCTTTCCCTACACGACGCTCT | 377 | TGGAGTTCAGACGTGTGCTCTTCCG | 394 |
| OT9 | TCCGATCTNNNN | | ATCT GAAGTCCTGCATCACTGACC | |
| | CAGCCAGGACTGGAATGCG | | | |
| Epeg | ACACTCTTTCCCTACACGACGCTCT | 378 | TGGAGTTCAGACGTGTGCTCTTCCG | 395 |
| OT10 | TCCGATCTNNNN | | ATCT CCCTTCCCTCTCACTCCG | |
| | GCACTTGCCAGACCTATGAC | | | |
| Epeg | ACACTCTTTCCCTACACGACGCTCT | 379 | TGGAGTTCAGACGTGTGCTCTTCCG | 396 |
| OT11 | TCCGATCTNNNN | | ATCT GGGACCGACCACGACTC | |
| | GATGAGTGGCGGAGGGAC | | | |
| Epeg | ACACTCTTTCCCTACACGACGCTCT | 380 | TGGAGTTCAGACGTGTGCTCTTCCG | 397 |
| OT12 | TCCGATCTNNNN | | ATCT GTTATCATGGCCCTGAGCAAC | |
| | CACAGTTCCTACCTGACCC | | | |
| Epeg | ACACTCTTTCCCTACACGACGCTCT | 381 | TGGAGTTCAGACGTGTGCTCTTCCG | 398 |
| OT13 | TCCGATCTNNNN | | ATCT | |
| | CAATACTCCAGAGCCTACTTCC | | CTTCTAAGAAGCGAGCTGCATAG | |
| Epeg | ACACTCTTTCCCTACACGACGCTCT | 382 | TGGAGTTCAGACGTGTGCTCTTCCG | 399 |
| OT14 | TCCGATCTNNNN | | ATCT CTTCACTTTGGTGAGGCAGTC | |
| | CCAGGGATTGGCCATCCAG | | | |
| Epeg | ACACTCTTTCCCTACACGACGCTCT | 383 | TGGAGTTCAGACGTGTGCTCTTCCG | 400 |
| OT15 | TCCGATCTNNNN | | ATCT | |
| | GCATGCTCTTGACACATGCC | | GCTACACCACAGCATGTTTGTTTC | |
| Nick | ACACTCTTTCCCTACACGACGCTCT | 384 | TGGAGTTCAGACGTGTGCTCTTCCG | 401 |
| OT1 | TCCGATCTNNNN | | ATCT | |
| | CTCCTCAAAGACCACTGAATTTGC | | CAGGGAAAGAAGAAAGGGACAAAG | |
| Nick | ACACTCTTTCCCTACACGACGCTCT | 385 | TGGAGTTCAGACGTGTGCTCTTCCG | 402 |
| OT2 | TCCGATCTNNNN | | ATCT CACTGTCCAGACTGTATGCC | |
| | TTACACAGCAGCTAGAAGAGTG | | | |

Nick OT 3 was not amplified as it is located in the Y chromosome and the pathogenic mutation is only known to occur in a female patient. After amplification, off-target sites were analyzed via Crispresso2 as described above.

### REFERENCES

1. Landrum, M. J. et al. ClinVar: public archive of interpretations of clinically relevant variants. Nucleic Acids Res. 44, D862-868 (2016).
2. FDA News Release. FDA Approves First Gene Therapies to Treat Patients with Sickle Cell Disease. (2023).
3. Anzalone, A. V. et al. Search-and-replace genome editing without double-strand breaks or donor DNA. Nature (2019) doi: 10.1038/s41586-019-1711-4.
4. Wiedenheft, B., Sternberg, S. H. & Doudna, J. A. RNA-guided genetic silencing systems in bacteria and archaea. Nature 482, 331-338 (2012).
5. Cong, L. et al. Multiplex Genome Engineering Using CRISPR/Cas Systems. Science 339, 819-823 (2013).
6. Mali, P. et al. RNA-Guided Human Genome Engineering via Cas9. Science 339, 823-826 (2013).
7. Makarova, K. S. et al. Evolutionary classification of CRISPR-Cas systems: a burst of class 2 and derived variants. Nat. Rev. Microbiol. 18, 67-83 (2020).
8. Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
9. Kato, K. et al. RNA-triggered protein cleavage and cell growth arrest by the type III-E CRISPR nuclease-protease. Science 378, 882-889 (2022).
10. Jinek, M. et al. A Programmable Dual-RNA-Guided DNA Endonuclease in Adaptive Bacterial Immunity. Science 337, 816-821 (2012).
11. Ran, F. A. et al. Genome engineering using the CRISPR-Cas9 system. Nat. Protoc. 8, 2281-2308 (2013).
12. Deltcheva, E. et al. CRISPR RNA maturation by trans-encoded small RNA and host factor RNase III. Nature 471, 602-607 (2011).
13. van Gent, D. C. & van der Burg, M. Non-homologous end-joining, a sticky affair. Oncogene 26, 7731-7740 (2007).
14. Lieber, M. R. The mechanism of double-strand DNA break repair by the nonhomologous DNA end-joining pathway. Annu. Rev. Biochem. 79, 181-211 (2010).
15. San Filippo, J., Sung, P. & Klein, H. Mechanism of eukaryotic homologous recombination. Annu. Rev. Biochem. 77, 229-257 (2008).
16. Sfeir, A. & Symington, L. S. Microhomology-Mediated End Joining: A Back-up Survival Mechanism or Dedicated Pathway? Trends Biochem. Sci. 40, 701-714 (2015).
17. Seol, J.-H., Shim, E. Y. & Lee, S. E. Microhomology-mediated end joining: Good, bad and ugly. Mutat. Res. 809, 81-87 (2018).
18. Shen, M. W. et al. Predictable and precise template-free CRISPR editing of pathogenic variants. Nature 563, 646-651 (2018).
19. Newby, G. A. et al. Base editing of haematopoietic stem cells rescues sickle cell disease in mice. Nature 595, 295-302 (2021).
20. Traxler, E. A. et al. A genome-editing strategy to treat β-hemoglobinopathies that recapitulates a mutation associated with a benign genetic condition. Nat. Med. 22, 987-990 (2016).
21. Métais, J.-Y. et al. Genome editing of HBG1 and HBG2 to induce fetal hemoglobin. Blood Adv. 3, 3379-3392 (2019).
22. Mayuranathan, T. et al. Potent and uniform fetal hemoglobin induction via base editing. Nat. Genet. 55, 1210-1220 (2023).
23. Saleh-Gohari, N. & Helleday, T. Conservative homologous recombination preferentially repairs DNA double-strand breaks in the S phase of the cell cycle in human cells. Nucleic Acids Res. 32, 3683-3688 (2004).
24. Sakamoto, Y. et al. Mitotic cells can repair DNA double-strand breaks via a homology-directed pathway. J. Radiat. Res. (Tokyo) 62, 25-33 (2021).
25. Suzuki, K. et al. In vivo genome editing via CRISPR/Cas9 mediated homology-independent targeted integration. Nature 540, 144-149 (2016).
26. Wilkinson, A. C. et al. Cas9-AAV6 gene correction of beta-globin in autologous HSCs improves sickle cell disease erythropoiesis in mice. Nat. Commun. 12, 686 (2021).
27. Lattanzi, A. et al. Development of β-globin gene correction in human hematopoietic stem cells as a potential durable treatment for sickle cell disease. Sci. Transl. Med. 13, eabf2444 (2021).
28. Cromer, M. K. et al. Gene replacement of a-globin with β-globin restores hemoglobin balance in B-thalassemia-derived hematopoietic stem and progenitor cells. Nat. Med. 27, 677-687 (2021).
29. Genovese, P. et al. Targeted genome editing in human repopulating haematopoietic stem cells. Nature 510, 235-240 (2014).
30. Hale, M. et al. Homology-Directed Recombination for Enhanced Engineering of Chimeric Antigen Receptor T Cells. Mol. Ther. Methods Clin. Dev. 4, 192-203 (2017).
31. Vaidyanathan, S. et al. High-Efficiency, Selection-free Gene Repair in Airway Stem Cells from Cystic Fibrosis Patients Rescues CFTR Function in Differentiated Epithelia. Cell Stem Cell 26, 161-171.e4 (2020).
32. Srifa, W. et al. Cas9-AAV6-engineered human mesenchymal stromal cells improved cutaneous wound healing in diabetic mice. Nat. Commun. 11, 2470 (2020).
33. Magis, W. et al. High-level correction of the sickle mutation is amplified in vivo during erythroid differentiation. iScience 25, 104374 (2022).
34. Romero, Z. et al. Editing the Sickle Cell Disease Mutation in Human Hematopoietic Stem Cells: Comparison of Endonucleases and Homologous Donor Templates. Mol. Ther. 27, 1389-1406 (2019).
35. Stephanie Yao. Graphite Bio Announces Voluntary Pause of Phase 1/2 CEDAR Study of nulabeglogene autogedtemcel (nula-cel) for Sickle Cell Disease [Press Release]. (2023).
36. Newby, G. A. & Liu, D. R. In vivo somatic cell base editing and prime editing. Mol. Ther. 29, 3107-3124 (2021).
37. Leibowitz, M. L. et al. Chromothripsis as an on-target consequence of CRISPR-Cas9 genome editing. Nat. Genet. 53, 895-905 (2021).
38. Zuccaro, M. V. et al. Allele-Specific Chromosome Removal after Cas9 Cleavage in Human Embryos. Cell 183, 1650-1664.e15 (2020).
39. Jiang, J. et al. Induction of site-specific chromosomal translocations in embryonic stem cells by CRISPR/Cas9. Sci. Rep. 6, 21918 (2016).
40. Enache, O. M. et al. Cas9 activates the p53 pathway and selects for p53-inactivating mutations. Nat. Genet. 52, 662-668 (2020).
41. Ihry, R. J. et al. p53 inhibits CRISPR-Cas9 engineering in human pluripotent stem cells. Nat. Med. 24, 939-946 (2018).
42. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
43. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
44. Kleinstiver, B. P. et al. Engineered CRISPR-Cas9 nucleases with altered PAM specificities. Nature 523, 481-485 (2015).
45. Hu, J. H. et al. Evolved Cas9 variants with broad PAM compatibility and high DNA specificity. Nature 556, 57-63 (2018).
46. Vakulskas, C. A. et al. A high-fidelity Cas9 mutant delivered as a ribonucleoprotein complex enables efficient gene editing in human hematopoietic stem and progenitor cells. Nat. Med. 24, 1216-1224 (2018).
47. Lee, J. K. et al. Directed evolution of CRISPR-Cas9 to increase its specificity. Nat. Commun. 9, 3048 (2018).
48. Casini, A. et al. A highly specific SpCas9 variant is identified by in vivo screening in yeast. Nat. Biotechnol. 36, 265-271 (2018).
49. Kulcsár, P. I. et al. Crossing enhanced and high fidelity SpCas9 nucleases to optimize specificity and cleavage. Genome Biol. 18, 190 (2017).
50. Chen, J. S. et al. Enhanced proofreading governs CRISPR-Cas9 targeting accuracy. Nature 550, 407-410 (2017).
51. Slaymaker, I. M. et al. Rationally engineered Cas9 nucleases with improved specificity. Science 351, 84-88 (2016).
52. Anzalone, A. V., Koblan, L. W. & Liu, D. R. Genome editing with CRISPR-Cas nucleases, base editors, transposases and prime editors. Nat. Biotechnol. 38, 824-844 (2020).
53. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
54. Gaudelli, N. M. et al. Programmable base editing of A.T to G.C in genomic DNA without DNA cleavage. Nature 551, 464-471 (2017).
55. Gaudelli, N. M. et al. Directed evolution of adenine base editors with increased activity and therapeutic application. Nat. Biotechnol. 38, 892-900 (2020).
56. Rees, H. A. & Liu, D. R. Base editing: precision chemistry on the genome and transcriptome of living cells. Nat. Rev. Genet. 19, 770-788 (2018).
57. Kim, Y. B. et al. Increasing the genome-targeting scope and precision of base editing with engineered Cas9-cytidine deaminase fusions. Nat. Biotechnol. 35, 371-376 (2017).
58. Li, X. et al. Base editing with a Cpf1-cytidine deaminase fusion. Nat. Biotechnol. 36, 324-327 (2018).
59. Liu, Z. et al. Efficient and high-fidelity base editor with expanded PAM compatibility for cytidine dinucleotide. Sci. China Life Sci. 64, 1355-1367 (2021).
60. Miller, S. M. et al. Continuous evolution of SpCas9 variants compatible with non-G PAMs. Nat. Biotechnol. (2020) doi: 10.1038/s41587-020-0412-8.
61. Huang, T. P. et al. High-throughput continuous evolution of compact Cas9 variants targeting single-nucleotide-pyrimidine PAMs. Nat. Biotechnol. 41, 96-107 (2023).
62. Nishimasu, H. et al. Engineered CRISPR-Cas9 nuclease with expanded targeting space. Science 361, 1259-1262 (2018).
63. Nakagawa, R. et al. Engineered Campylobacter jejuni Cas9 variant with enhanced activity and broader targeting range. Commun. Biol. 5, 211 (2022).
64. Huang, T. P. et al. Circularly permuted and PAM-modified Cas9 variants broaden the targeting scope of base editors. Nat. Biotechnol. 37, 626-631 (2019).
65. Richter, M. F. et al. Phage-assisted evolution of an adenine base editor with improved Cas domain compatibility and activity. Nat. Biotechnol. (2020) doi: 10.1038/s41587-020-0453-z.
66. Neugebauer, M. E. et al. Evolution of an adenine base editor into a small, efficient cytosine base editor with low off-target activity. Nat. Biotechnol. 41, 673-685 (2023).
67. Zhang, E., Neugebauer, M. E., Krasnow, N. A. & Liu, D. R. Phage-assisted evolution of highly active cytosine base editors with enhanced selectivity and minimal sequence context preference. Nat. Commun. 15, 1697 (2024).
68. Grünewald, J. et al. CRISPR DNA base editors with reduced RNA off-target and self-editing activities. Nat. Biotechnol. 37, 1041-1048 (2019).
69. Burdett, T. & Nuseibeh, S. Changing trends in the development of AAV-based gene therapies: a meta-analysis of past and present therapies. Gene Ther. 30, 323-335 (2023).
70. Dong, J. Y., Fan, P. D. & Frizzell, R. A. Quantitative analysis of the packaging capacity of recombinant adeno-associated virus. Hum. Gene Ther. 7, 2101-2112 (1996).
71. Wu, Z., Yang, H. & Colosi, P. Effect of genome size on AAV vector packaging. Mol. Ther. J. Am. Soc. Gene Ther. 18, 80-86 (2010).
72. Levy, J. M. et al. Cytosine and adenine base editing of the brain, liver, retina, heart and skeletal muscle of mice via adeno-associated viruses. Nat. Biomed. Eng. 4, 97-110 (2020).
73. Davis, J. R. et al. Efficient in vivo base editing via single adeno-associated viruses with size-optimized genomes encoding compact adenine base editors. Nat. Biomed. Eng. 6, 1272-1283 (2022).
74. Karen O'Hanlon Cohrt. Clinical Trial Roundup: Base Editors in the Clinic. crisprmedicinenews.com/news/clinical-trial-roundup-base-editors-in-the-clinic/.
75. VERVE-101: CRISPR-Based Gene Editing Therapy Shows Promise in Reducing LDL-C and PCSK9 Levels in Patients With HeFH. https://www.acc.org/Latest-in-Cardiology/Articles/2023/11/08/20/14/sun-445 μm-heart1-aha-2023 (2023).
76. Chiesa, R. et al. Base-Edited CAR7 T Cells for Relapsed T-Cell Acute Lymphoblastic Leukemia. N. Engl. J. Med. 389, 899-910 (2023).
77. BEACON: A Study Evaluating the Safety and Efficacy of BEAM-101 in Patients With Severe Sickle Cell Disease (BEACON).
78. Beam Therapeutics Highlights Progress Across Base Editing Portfolio and Outlines 2024 Anticipated Milestones. (2024).
79. Everette, K. A. et al. Ex vivo prime editing of patient haematopoietic stem cells rescues sickle-cell disease phenotypes after engraftment in mice. Nat. Biomed. Eng. 7, 616-628 (2023).
80. Yeh, W.-H., Chiang, H., Rees, H. A., Edge, A. S. B. & Liu, D. R. In vivo base editing of post-mitotic sensory cells. Nat. Commun. 9, 2184 (2018).
81. Doman, J. L., Raguram, A., Newby, G. A. & Liu, D. R. Evaluation and minimization of Cas9-independent off-target DNA editing by cytosine base editors. Nat. Biotechnol. 38, 620-628 (2020).
82. Zhou, C. et al. Off-target RNA mutation induced by DNA base editing and its elimination by mutagenesis. Nature 571, 275-278 (2019).
83. Wang, L. et al. Eliminating base-editor-induced genome-wide and transcriptome-wide off-target mutations. Nat. Cell Biol. 23, 552-563 (2021).
84. Rees, H. A., Wilson, C., Doman, J. L. & Liu, D. R. Analysis and minimization of cellular RNA editing by DNA adenine base editors. Sci. Adv. 5, eaax5717 (2019).
85. Doman, J. L., Sousa, A. A., Randolph, P. B., Chen, P. J. & Liu, D. R. Designing and executing prime editing experiments in mammalian cells. Nat. Protoc. 17, 2431-2468 (2022).
86. Chen, P. J. et al. Enhanced prime editing systems by manipulating cellular determinants of editing outcomes. Cell 184, 5635-5652.e29 (2021).
87. Ferreira da Silva, J. et al. Prime editing efficiency and fidelity are enhanced in the absence of mismatch repair. Nat. Commun. 13, 760 (2022).
88. Doman, J. L. et al. Phage-assisted evolution and protein engineering yield compact, efficient prime editors. Cell 186, 3983-4002.e26 (2023).
89. Nelson, J. W. et al. Engineered pegRNAs improve prime editing efficiency. Nat. Biotechnol. 40, 402-410 (2022).
90. Kotulska, K., Fattal-Valevski, A. & Haberlova, J. Recombinant Adeno-Associated Virus Serotype 9 Gene Therapy in Spinal Muscular Atrophy. Front. Neurol. 12, 726468 (2021).
91. Davis, J. R. et al. Efficient prime editing in mouse brain, liver and heart with dual AAVs. Nat. Biotechnol. 42, 253-264 (2024).
92. Basila, M., Kelley, M. L. & Smith, A. van B. Minimal 2′-O-methyl phosphorothioate linkage modification pattern of synthetic guide RNAs for increased stability and efficient CRISPR-Cas9 gene editing avoiding cellular toxicity. PloS One 12, e0188593 (2017).
93. Hendel, A. et al. Chemically modified guide RNAs enhance CRISPR-Cas genome editing in human primary cells. Nat. Biotechnol. 33, 985-989 (2015).
94. Godbout, K. & Tremblay, J. P. Prime Editing for Human Gene Therapy: Where Are We Now? Cells 12, 536 (2023).
95. Philippidis, A. First Prime Editing Clinical Trial Expected in 2024. Hum. Gene Ther. 34, 1077-1080 (2023).
96. Alex Philippidis. J P M: Prime Medicine Eyes 2024 IND for First Prime Editing Therapy. GEN Edge (2024).
97. Schene, I. F. et al. Prime editing for functional repair in patient-derived disease models. Nat. Commun. 11, 5352 (2020).
98. Kim, D. Y., Moon, S. B., Ko, J.-H., Kim, Y.-S. & Kim, D. Unbiased investigation of specificities of prime editing systems in human cells. Nucleic Acids Res. 48, 10576-10589 (2020).
99. Liu, Y. et al. Efficient generation of mouse models with the prime editing system. Cell Discov. 6, 27 (2020).
100. Geurts, M. H. et al. Evaluating CRISPR-based prime editing for cancer modeling and CFTR repair in organoids. Life Sci. Alliance 4, e202000940 (2021).
101. Park, S.-J. et al. Targeted mutagenesis in mouse cells and embryos using an enhanced prime editor. Genome Biol. 22, 170 (2021).
102. Gao, P. et al. Prime editing in mice reveals the essentiality of a single base in driving tissue-specific gene expression. Genome Biol. 22, 83 (2021).
103. Lin, J. et al. Modeling a cataract disorder in mice with prime editing. Mol. Ther.-Nucleic Acids 25, 494-501 (2021).
104. Habib, O., Habib, G., Hwang, G.-H. & Bae, S. Comprehensive analysis of prime editing outcomes in human embryonic stem cells. Nucleic Acids Res. 50, 1187-1197 (2022).
105. Gao, R. et al. Genomic and Transcriptomic Analyses of Prime Editing Guide RNA-Independent Off-Target Effects by Prime Editors. CRISPR J. 5, 276-293 (2022).
106. Piel, F. B., Steinberg, M. H. & Rees, D. C. Sickle Cell Disease. N. Engl. J. Med. 376, 1561-1573 (2017).

107. Nickel, R. S. & Kamani, N. R. Ethical Challenges in Hematopoietic Cell Transplantation for Sickle Cell Disease. Biol. Blood Marrow Transplant. 24, 219-227 (2018).
108. Demirci, S., Leonard, A., Haro-Mora, J. J., Uchida, N. & Tisdale, J. F. CRISPR/Cas9 for Sickle Cell Disease: Applications, Future Possibilities, and Challenges. in Cell Biology and Translational Medicine, Volume 5 (ed. Turksen, K.) vol. 1144 37-52 (Springer International Publishing, Cham, 2019).
109. Kunz, J. B. & Kulozik, A. E. Gene Therapy of the Hemoglobinopathies. HemaSphere 4, (2020).
110. Davis, R., Gurumurthy, A., Hossain, M. A., Gunn, E. M. & Bungert, J. Engineering Globin Gene Expression. Mol. Ther.-Methods Clin. Dev. 12, 102-110 (2019).
111. Leonard, A., Tisdale, J. & Abraham, A. Curative options for sickle cell disease: haploidentical stem cell transplantation or gene therapy? Br. J. Haematol. 189, 408-423 (2020).
112. Magrin, E., Miccio, A. & Cavazzana, M. Lentiviral and genome-editing strategies for the treatment of β-hemoglobinopathies. Blood 134, 1203-1213 (2019).
113. Zeng, J. et al. Therapeutic base editing of human hematopoietic stem cells. Nat. Med. (2020) doi: 10.1038/s41591-020-0790-y.
114. Pattabhi, S. et al. In Vivo Outcome of Homology-Directed Repair at the HBB Gene in HSC Using Alternative Donor Template Delivery Methods. Mol. Ther.-Nucleic Acids 17, 277-288 (2019).
115. Weber, L. et al. Editing a y-globin repressor binding site restores fetal hemoglobin synthesis and corrects the sickle cell disease phenotype. Sci. Adv. 6, eaay9392 (2020).
116. Ribeil, J.-A. et al. Gene Therapy in a Patient with Sickle Cell Disease. N. Engl. J. Med. 376, 848-855 (2017).
117. Esrick, E. B. et al. Post-Transcriptional Genetic Silencing of BCLIIA to Treat Sickle Cell Disease. N. Engl. J. Med. 384, 205-215 (2021).
118. Frangoul, H. et al. CRISPR-Cas9 Gene Editing for Sickle Cell Disease and β-Thalassemia. N. Engl. J. Med. 384, 252-260 (2021).
119. Song, Y. et al. Large-Fragment Deletions Induced by Cas9 Cleavage while Not in the BEs System. Mol. Ther.-Nucleic Acids 21, 523-526 (2020).
120. Adikusuma, F. et al. Large deletions induced by Cas9 cleavage. Nature 560, E8-E9 (2018).
121. Webber, B. R. et al. Highly efficient multiplex human T cell engineering without double-strand breaks using Cas9 base editors. Nat. Commun. 10, 5222 (2019).
122. Dever, D. P. et al. CRISPR/Cas9 β-globin gene targeting in human haematopoietic stem cells. Nature 539, 384-389 (2016).
123. Sangkitporn, S., Rerkamnuaychoke, B., Sangkitporn, S., Mitrakul, C. & Sutivigit, Y. Hb G Makassar (beta 6: Glu-Ala) in a Thai family. J. Med. Assoc. Thail. Chotmaihet Thangphaet 85, 577-582 (2002).
124. Blackwell, R. Q., Oemijati, S., Pribadi, W., Weng, M. I. & Liu, C. S. Hemoglobin G Makassar: beta-6 Glu leads to Ala. Biochim. Biophys. Acta 214, 396-401 (1970).
125. Viprakasit, V., Wiriyasateinkul, A., Sattayasevana, B., Miles, K. L. & Laosombat, V. Hb G-MAKASSAR [β6 (A3)Glu→Ala; CODON 6 (GA G→G C G)]: MOLECULAR CHARACTERIZATION, CLINICAL, AND HEMATOLOGICAL EFFECTS. Hemoglobin 26, 245-253 (2002).
126. Chu, S. H. et al. Rationally Designed Base Editors for Precise Editing of the Sickle Cell Disease Mutation. CRISPR J. 4, 169-177 (2021).
127. Wu, L.-C. et al. Correction of sickle cell disease by homologous recombination in embryonic stem cells. Blood 108, 1183-1188 (2006).
128. Bae, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
129. Tsai, S. Q. et al. CIRCLE-seq: a highly sensitive in vitro screen for genome-wide CRISPR-Cas9 nuclease off-targets. Nat. Methods 14, 607-614 (2017).
130. McIntosh, B. E. et al. Nonirradiated NOD,B6.SCID I12rγ−/−KitW41/W41 (NBSGW) Mice Support Multilineage Engraftment of Human Hematopoietic Cells. Stem Cell Rep. 4, 171-180 (2015).
131. Kurup, S. P., Moioffer, S. J., Pewe, L. L. & Harty, J. T. p53 Hinders CRISPR/Cas9-Mediated Targeted Gene Disruption in Memory CD8 T Cells In Vivo. J. Immunol. 205, 2222-2230 (2020).
132. Luc, S. et al. Bcl11a Deficiency Leads to Hematopoietic Stem Cell Defects with an Aging-like Phenotype. Cell Rep. 16, 3181-3194 (2016).
133. Alanis-Lobato, G. et al. Frequent loss of heterozygosity in CRISPR-Cas9-edited early human embryos. Proc. Natl. Acad. Sci. 118, e2004832117 (2021).
134. Karimian, A., Ahmadi, Y. & Yousefi, B. Multiple functions of p21 in cell cycle, apoptosis and transcriptional regulation after DNA damage. DNA Repair 42, 63-71 (2016).
135. Demirci, S. et al. BT87Q-Globin Gene Therapy Reduces Sickle Hemoglobin Production, Allowing for Ex Vivo Anti-sickling Activity in Human Erythroid Cells. Mol. Ther.-Methods Clin. Dev. 17, 912-921 (2020).
136. Wu, Y. et al. Highly efficient therapeutic gene editing of human hematopoietic stem cells. Nat. Med. 25, 776-783 (2019).
137. Howe, S. J. et al. Insertional mutagenesis combined with acquired somatic mutations causes leukemogenesis following gene therapy of SCID-X1 patients. J. Clin. Invest. 118, 3143-3150 (2008).
138. Stein, S. et al. Genomic instability and myelodysplasia with monosomy 7 consequent to EVI1 activation after gene therapy for chronic granulomatous disease. Nat. Med. 16, 198-204 (2010).
139. Huang, T. P., Newby, G. A. & Liu, D. R. Precision genome editing using cytosine and adenine base editors in mammalian cells. Nat. Protoc. 16, 1089-1128 (2021).
140. Rees, H. A. et al. Improving the DNA specificity and applicability of base editing through protein engineering and protein delivery. Nat. Commun. 8, 15790 (2017).
141. Clement, K. et al. CRISPResso2 provides accurate and rapid genome editing sequence analysis. Nat. Biotechnol. 37, 224-226 (2019).
142. Vaidyanathan, S. et al. Uridine Depletion and Chemical Modification Increase Cas9 mRNA Activity and Reduce Immunogenicity without HPLC Purification. Mol. Ther.-Nucleic Acids 12, 530-542 (2018).
143. Zuris, J. A. et al. Cationic lipid-mediated delivery of proteins enables efficient protein-based genome editing in vitro and in vivo. Nat. Biotechnol. 33, 73-80 (2015). 144. Connelly, J. P. & Pruett-Miller, S. M. CRIS.py: A Versatile and High-throughput Analysis Program for CRISPR-based Genome Editing. Sci. Rep. 9, 4194 (2019).
145. Hu, J. et al. Isolation and functional characterization of human erythroblasts at distinct stages: implications for understanding of normal and disordered erythropoiesis in vivo. Blood 121, 3246-3253 (2013).
146. Lazzarotto, C. R. et al. Defining CRISPR-Cas9 genome-wide nuclease activities with CIRCLE-seq. Nat. Protoc. 13, 2615-2642 (2018).
147. Hwang, G.-H., Kim, J.-S. & Bae, S. Web-Based CRISPR Toolkits: Cas-OFFinder, Cas-Designer, and Cas-Analyzer. in CRISPR Guide RNA Design (eds. Fulga, T. A., Knapp, D. J. H. F. & Ferry, Q. R. V.) vol. 2162 23-33 (Springer US, New York, NY, 2021).
148. Heinz, S. et al. Simple Combinations of Lineage-Determining Transcription Factors Prime cis-Regulatory Elements Required for Macrophage and B Cell Identities. Mol. Cell 38, 576-589 (2010).
149. Kumar, S. & Geiger, H. HSC Niche Biology and HSC Expansion Ex Vivo. Trends Mol. Med. 23, 799-819 (2017).
150. Leonard, A. et al. Low-Dose Busulfan Reduces Human CD34+ Cell Doses Required for Engraftment in c-kit Mutant Immunodeficient Mice. Mol. Ther.-Methods Clin. Dev. 15, 430-437 (2019).
151. Khemani, K., Katoch, D. & Krishnamurti, L. Curative Therapies for Sickle Cell Disease. Ochsner J. 19, 131-137 (2019).
152. Kanter, J. et al. Biologic and Clinical Efficacy of LentiGlobin for Sickle Cell Disease. N. Engl. J. Med. 386, 617-628 (2022).
153. Lessard, S. et al. Zinc Finger Nuclease-Mediated Disruption of the BCL11A Erythroid Enhancer Results in Enriched Biallelic Editing, Increased Fetal Hemoglobin, and Reduced Sickling in Erythroid Cells Derived from Sickle Cell Disease Patients. Blood 134, 974-974 (2019).
154. Ferrari, S. et al. Choice of template delivery mitigates the genotoxic risk and adverse impact of editing in human hematopoietic stem cells. Cell Stem Cell 29, 1428-1444.e9 (2022).
155. Schiroli, G. et al. Precise Gene Editing Preserves Hematopoietic Stem Cell Function following Transient p53-Mediated DNA Damage Response. Cell Stem Cell 24, 551-565.e8 (2019).
156. Chen, P. J. & Liu, D. R. Prime editing for precise and highly versatile genome manipulation. Nat. Rev. Genet. (2022) doi: 10.1038/s41576-022-00541-1.
157. Jin, S. et al. Genome-wide specificity of prime editors in plants. Nat. Biotechnol. 39, 1292-1299 (2021).
158. Anzalone, A. V. et al. Programmable deletion, replacement, integration and inversion of large DNA sequences with twin prime editing. Nat. Biotechnol. 40, 731-740 (2022).
159. Choi, J. et al. Precise genomic deletions using paired prime editing. Nat. Biotechnol. 40, 218-226 (2022).
160. Lin, Q. et al. High-efficiency prime editing with optimized, paired pegRNAs in plants. Nat. Biotechnol. 39, 923-927 (2021).
161. De Dreuzy, E. et al. EDIT-301: An Experimental Autologous Cell Therapy Comprising Cas12a-RNP Modified mPB-CD34+Cells for the Potential Treatment of SCD. Blood 134, 4636-4636 (2019).
162. Li, C. et al. In vivo HSC prime editing rescues Sickle Cell Disease in a mouse model. Blood blood.2022018252 (2023) doi: 10.1182/blood.2022018252.
163. Peterson, C. W. et al. Intracellular RNase activity dampens zinc finger nuclease-mediated gene editing in hematopoietic stem and progenitor cells. Mol. Ther.-Methods Clin. Dev. 24, 30-39 (2022).
164. Fitzhugh, C. D. et al. At least 20% donor myeloid chimerism is necessary to reverse the sickle phenotype after allogeneic HSCT. Blood 130, 1946-1948 (2017).
165. Walters, M. C. et al. Stable mixed hematopoietic chimerism after bone marrow transplantation for sickle cell anemia. Biol. Blood Marrow Transplant. 7, 665-673 (2001).
166. Sankaran, V. G. & Orkin, S. H. The Switch from Fetal to Adult Hemoglobin. Cold Spring Harb. Perspect. Med. 3, a011643-a011643 (2013).
167. Ribeil, J.-A. et al. Ineffective Erythropoiesis in β-Thalassemia. Sci. World J. 2013, 1-11 (2013).
168. Wu, C. J. et al. Evidence for ineffective erythropoiesis in severe sickle cell disease. Blood 106, 3639-3645 (2005).
169. Jin, S. et al. Cytosine, but not adenine, base editors induce genome-wide off-target mutations in rice. Science 364, 292-295 (2019).
170. Zuo, E. et al. Cytosine base editor generates substantial off-target single-nucleotide variants in mouse embryos. Science 364, 289-292 (2019).
171. Kaczmarek, J. C., Kowalski, P. S. & Anderson, D. G. Advances in the delivery of RNA therapeutics: from concept to clinical reality. Genome Med. 9, 60 (2017).
172. Banskota, S. et al. Engineered virus-like particles for efficient in vivo delivery of therapeutic proteins. Cell 185, 250-265.e16 (2022).
173. Olson, H. E. et al. Cyclin-Dependent Kinase-Like 5 Deficiency Disorder: Clinical Review. Pediatr. Neurol. 97, 18-25 (2019).
174. Zhu, Y. & Xiong, Z. Molecular and Synaptic Bases of CDKL5 Disorder. Dev. Neurobiol. 79, 8-19 (2019).
175. Baltussen, L. L. et al. Chemical genetic identification of CDKL 5 substrates reveals its role in neuronal microtubule dynamics. EMBO J. 37, e99763 (2018).
176. Ricciardi, S. et al. CDKL5 ensures excitatory synapse stability by reinforcing NGL-1-PSD95 interaction in the postsynaptic compartment and is impaired in patient iPSC-derived neurons. Nat. Cell Biol. 14, 911-923 (2012).
177. Terzic, B. et al. Temporal manipulation of Cdk15 reveals essential post developmental functions and reversible CDKL5 deficiency disorder-related deficits. J. Clin. Invest. 131, e143655 (2021).
178. Scala, E. et al. CDKL5/STK9 is mutated in Rett syndrome variant with infantile spasms. J. Med. Genet. 42, 103-107 (2005).
179. Neul, J. L. et al. Rett syndrome: revised diagnostic criteria and nomenclature. Ann. Neurol. 68, 944-950 (2010).
180. Fehr, S. et al. The CDKL5 disorder is an independent clinical entity associated with early-onset encephalopathy. Eur. J. Hum. Genet. EJHG 21, 266-273 (2013).
181. Hector, R. D. et al. CDKL5 variants: Improving our understanding of a rare neurologic disorder. Neurol. Genet. 3, e200 (2017).
182. Weaving, L. S. Rett syndrome: clinical review and genetic update. J. Med. Genet. 42, 1-7 (2005).
183. Krishnaraj, R., Ho, G. & Christodoulou, J. RettBASE: Rett syndrome database update. Hum. Mutat. 38, 922-931 (2017).
184. Tao, J. et al. Mutations in the X-Linked Cyclin-Dependent Kinase-Like 5 (CDKL5/STK9) Gene Are Associated with Severe Neurodevelopmental Retardation. Am. J. Hum. Genet. 75, 1149-1154 (2004).
185. Kalscheuer, V. M. et al. Disruption of the Serine/Threonine Kinase 9 Gene Causes Severe X-Linked Infantile Spasms and Mental Retardation. Am. J. Hum. Genet. 72, 1401-1411 (2003).
186. Weaving, L. S. et al. Mutations of CDKL5 Cause a Severe Neurodevelopmental Disorder with Infantile Spasms and Mental Retardation. Am. J. Hum. Genet. 75, 1079-1093 (2004).
187. Bahi-Buisson, N. et al. Recurrent mutations in the CDKL5 gene: Genotype-phenotype relationships. Am. J. Med. Genet. A. 158A, 1612-1619 (2012). 188. Siri, B. et al. CDKL5 deficiency disorder in males: Five new variants and review of the literature. Eur. J. Paediatr. Neurol. 33, 9-20 (2021).
189. Kluckova, D. et al. Clinical manifestation of CDKL5 deficiency disorder and identified mutations in a cohort of Slovak patients. Epilepsy Res. 176, 106699 (2021).
190. Bahi-Buisson, N. et al. Key clinical features to identify girls with CDKL5 mutations. Brain 131, 2647-2661 (2008).
191. Christianto, A., Katayama, S., Kameshita, I. & Inazu, T. A novel CDKL5 mutation in a Japanese patient with atypical Rett syndrome. Clin. Chim. Acta 459, 132-136 (2016).
192. Fraser, H., Goldman, A., Wright, R., Deciphering Developmental Disorders Study & Banka, S. A maternally inherited frameshift CDKL5 variant in a male with global developmental delay and late-onset generalized epilepsy. Am. J. Med. Genet. A. 179, 507-511 (2019).
193. Muñoz, I. M. et al. Phosphoproteomic screening identifies physiological substrates of the CDKL 5 kinase. EMBO J. 37, e99559 (2018).
194. Van Bergen, N. J. et al. CDKL5 deficiency disorder: molecular insights and mechanisms of pathogenicity to fast-track therapeutic development. Biochem. Soc. Trans. 50, 1207-1224 (2022).
195. Frasca, A. et al. Not Just Loss-of-Function Variations: Identification of a Hypermorphic Variant in a Patient With a CDKL5 Missense Substitution. Neurol. Genet. 8, e666 (2022).
196. Rusconi, L. et al. CDKL5 Expression Is Modulated during Neuronal Development and Its Subcellular Distribution Is Tightly Regulated by the C-terminal Tail. J. Biol. Chem. 283, 30101-30111 (2008).
197. Bertani, I. et al. Functional Consequences of Mutations in CDKL5, an X-linked Gene Involved in Infantile Spasms and Mental Retardation. J. Biol. Chem. 281, 32048-32056 (2006).
198. Hong, W. et al. CDKL5 Deficiency Disorder-Related Epilepsy: A Review of Current and Emerging Treatment. CNS Drugs 36, 591-604 (2022).
199. Leonard, H. et al. CDKL5 deficiency disorder: clinical features, diagnosis, and management. Lancet Neurol. 21, 563-576 (2022).
200. Halmai, J. A. N. M. et al. Artificial escape from XCI by DNA methylation editing of the CDKL5 gene. Nucleic Acids Res. 48, 2372-2387 (2020).
201. Ultragenyx. UX055 AAV9 gene therapy for the potential treatment of CDKL5 Deficiency Disorder (CDD).
202. Cullot, G. et al. CRISPR-Cas9 genome editing induces megabase-scale chromosomal truncations. Nat. Commun. 10, 1136 (2019).
203. Chen, B. et al. Dynamic Imaging of Genomic Loci in Living Human Cells by an Optimized CRISPR/Cas System. Cell 155, 1479-1491 (2013).
204. Illumina. Measuring sequencing accuracy: Understand quality scores, or Q scores, and what they mean for your sequencing run.
205. Zhang, Y. et al. Comparison of non-canonical PAMs for CRISPR/Cas9-mediated DNA cleavage in human cells. Sci. Rep. 4, 5405 (2014).
206. Carvill, G. L., Matheny, T., Hesselberth, J. & Demarest, S. Haploinsufficiency, Dominant Negative, and Gain-of-Function Mechanisms in Epilepsy: Matching Therapeutic Approach to the Pathophysiology. Neurotherapeutics 18, 1500-1514 (2021).
207. Szafranski, P. et al. Neurodevelopmental and neurobehavioral characteristics in males and females with CDKL5 duplications. Eur. J. Hum. Genet. EJHG 23, 915-921 (2015). 208. Carpenter, J. C. & Lignani, G. Gene Editing and Modulation: the Holy Grail for the Genetic Epilepsies? Neurotherapeutics 18, 1515-1523 (2021).
209. Carrette, L. L. G., Blum, R., Ma, W., Kelleher, R. J. & Lee, J. T. Tsix-Mecp2 female mouse model for Rett syndrome reveals that low-level MECP2 expression extends life and improves neuromotor function. Proc. Natl. Acad. Sci. 115, 8185-8190 (2018).
210. Nathwani, A. C. et al. Long-Term Safety and Efficacy of Factor IX Gene Therapy in Hemophilia B. N. Engl. J. Med. 371, 1994-2004 (2014).
211. Kuo, C. Y. et al. Site-Specific Gene Editing of Human Hematopoietic Stem Cells for X-Linked Hyper-IgM Syndrome. Cell Rep. 23, 2606-2616 (2018).
212. Sleat, D. E. et al. Mutational Analysis of the Defective Protease in Classic Late-Infantile Neuronal Ceroid Lipofuscinosis, a Neurodegenerative Lysosomal Storage Disorder. Am. J. Hum. Genet. 64, 1511-1523 (1999).
213. Kohan, R. et al. Neuronal ceroid lipofuscinosis type CLN2: a new rationale for the construction of phenotypic subgroups based on a survey of 25 cases in South America. Gene 516, 114-121 (2013).
214. An, M. et al. Engineered virus-like particles for transient delivery of prime editor ribonucleoprotein complexes in vivo. Nat. Biotechnol. (2024) doi: 10.1038/s41587-023-02078-y.
215. Chen, P.-F. et al. Generation and characterization of human induced pluripotent stem cells (iPSCs) from three male and three female patients with CDKL5 Deficiency Disorder (CDD). Stem Cell Res. 53, 102276 (2021).

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method of correcting a 1412delA mutation in a CDKL5 gene by prime editing comprising contacting a target DNA sequence comprising the CDKL5 gene with a prime editor and a prime editing guide RNA (pegRNA), wherein the pegRNA comprises a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5) or TAGCTATGCCGACT (SEQ ID NO: 6).

2. The method of claim 1, wherein the pegRNA comprises the sequence of any of the pegRNAs provided in Tables 1, 2, and 4, or a sequence at least 80%, at least 85%, at least 90%, at least 95%, at least 96%, at least 97%, at least 98%, or at least 99% identical to any of the pegRNAs provided in Tables 1, 2, and 4.

3. The method of claim 1 or 2, wherein the pegRNA comprises a gRNA scaffold sequence of GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence at least 80%, at least 85%, at least 90%, at least 95%, at least 96%, at least 97%, at least 98%, or at least 99% identical to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence comprising one, two, three, four, or five mutations, insertions, or deletions relative to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577).

4. The method of any one of claims 1-3, wherein the pegRNA comprises a reverse transcription template (RTT) comprising the sequence of any one of SEQ ID NOs: 404-563, or a sequence at least 80%, at least 85%, at least 90%, at least 95%, at least 96%, at least 97%, at least 98%, or at least 99% identical to of any one of SEQ ID NOs: 404-563, or a sequence comprising one, two, three, four, or five mutations, insertions, or deletions relative to of any one of SEQ ID NOs: 404-563.

5. The method of any one of claims 1-4, wherein the pegRNA comprises a primer binding site comprising the sequence TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35, TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence at least 80%, at least 85%, at least 90%, at least 95%, at least 96%, at least 97%, at least 98%, or at least 99% identical to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35, TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence comprising one, two, three, four, or five mutations, insertions, or deletions relative to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35), TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6).

6. The method of any one of claims 1-5, wherein the pegRNA comprises a 3′ structured motif of the sequence CGCGGTTCTATCTAGTTACGCGTTAAACCAACTAGAA (SEQ ID NO: 14), or a sequence at least 80%, at least 85%, at least 90%, at least 95%, at least 96%, at least 97%, at least 98%, or at least 99% identical to CGCGGTTCTATCTAGTTACGCGTTAAACCAACTAGAA (SEQ ID NO: 14), or a sequence comprising one, two, three, four, or five mutations, insertions, or deletions relative to CGCGGTTCTATCTAGTTACGCGTTAAACCAACTAGAA (SEQ ID NO: 14).

7. The method of any one of claims 1-6, wherein the pegRNA comprises a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT.

8. The method of any one of claims 1-7, wherein the pegRNA comprises the structure 5′-[spacer]-[gRNA scaffold]-[RTT]-[PBS]-[3′ motif]-[terminator]-3′ or 5′-[spacer]-[gRNA scaffold]-[RTT]-[PBS]-[3′ motif]-3′.

9. The method of any one of claims 1-8, wherein the contacting further comprises using a nicking guide RNA (ngRNA).

10. The method of claim 9, wherein the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13).

11. The method of claim 9 or 10, wherein the ngRNA comprises the sequence of any one of the ngRNAs provided in Table 3, or a sequence at least 80%, at least 85%, at least 90%, at least 95%, at least 96%, at least 97%, at least 98%, or at least 99% identical to any one of the ngRNAs provided in Table 3.

12. The method of any one of claims 9-11, wherein the ngRNA comprises a gRNA scaffold sequence of GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence at least 80%, at least 85%, at least 90%, at least 95%, at least 96%, at least 97%, at least 98%, or at least 99% identical to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence comprising one, two, three, four, or five mutations, insertions, or deletions relative to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578).

13. The method of any one of claims 9-12, wherein the ngRNA comprises a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT.

14. The method of any one of claims 9-13, wherein the ngRNA comprises the structure 5′-[spacer]-[gRNA scaffold]-[terminator]-3′ or 5′-[spacer]-[gRNA scaffold]-3′.

15. The method of any one of claims 1-14, wherein the prime editor comprises (i) a napDNAbp domain, and (ii) a polymerase domain.

16. The method of claim 15, wherein the napDNAbp domain has a nickase activity.

17. The method of claim 15 or 16, wherein the napDNAbp domain is a Cas9 protein or a variant thereof.

18. The method of any one of claims 15-17, wherein the napDNAbp domain is a Cas9 nickase (nCas9).

19. The method of any one of claims 15-18, wherein the napDNAbp domain comprises a sequence of any one of the napDNAbps provided herein, or an amino acid sequence at least 80%, at least 85%, at least 90%, at least 95%, at least 96%, at least 97%, at least 98%, or at least 99% identical to any one of the napDNAbps provided herein.

20. The method of any one of claims 15-19, wherein the polymerase domain is a reverse transcriptase domain.

21. The method of any one of claims 15-20, wherein the polymerase domain comprises a sequence of any one of the polymerase domains provided herein, or an amino acid sequence at least 80%, at least 85%, at least 90%, at least 95%, at least 96%, at least 97%, at least 98%, or at least 99% identical to any one of the polymerase domains provided herein.

22. The method of any one of claims 1-21, wherein the prime editor is PE2, PE3, PE4, PE5, or a variant thereof.

23. The method of any one of claims 1-22, wherein the prime editor is PE4max-NRCH, or a variant thereof.

24. The method of any one of claims 1-21, wherein the prime editor is a PE6 prime editor.

25. The method of any one of claim 1-21 or 24, wherein the prime editor is PE6b-NRCH, PE6c-NRCH, or PE6d-NRCH.

26. The method of any one of claims 1-25, wherein the method is performed in vitro.

27. The method of any one of claims 1-25, wherein the method is performed in vivo.

28. The method of any one of claim 1-25 or 27, wherein the method is performed in a subject.

29. The method of claim 28, wherein the subject is a human.

30. The method of claim 28 or 29, wherein the method is a method of treating CDKL5 deficiency disorder in the subject.

31. The method of any one of claims 1-30, wherein the method achieves greater than 20%, greater than 25%, greater than 30%, greater than 35%, or greater than 40% correction of the 1412delA mutation.

32. The method of any one of claims 1-31, wherein the method results in introduction of less than 2%, less than 3%, less than 4%, or less than 5% indel byproducts in the target DNA sequence.

33. A prime editing guide RNA (pegRNA) comprising a spacer sequence of GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence at least 80%, at least 85%, at least 90%, at least 95%, at least 96%, at least 97%, at least 98%, or at least 99% identical to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), or TAGCTATGCCGACT (SEQ ID NO: 6), or a spacer sequence comprising one, two, three, four, or five mutations, insertions, or deletions relative to GCCGGGAGGACTGGGGAATGG (SEQ ID NO: 1), GCCAGATCTCAGCAGAACAGG (SEQ ID NO: 2), GCATTCATGGAAAGCTCCCAG (SEQ ID NO: 3), GCCAGAGCAAAGCTGGGACCC (SEQ ID NO: 4), GCAGAGTCGGCATAGCTATAT (SEQ ID NO: 5), TAGCTATGCCGACT (SEQ ID NO: 6).

34. The pegRNA of claim 33, wherein the pegRNA comprises the sequence of any of the pegRNAs provided in Tables 1, 2, and 4 herein, or a sequence at least 80%, at least 85%, at least 90%, at least 95%, at least 96%, at least 97%, at least 98%, or at least 99% identical to any of the pegRNAs provided in Tables 1, 2, and 4 herein.

35. The pegRNA of claim 33 or 34, wherein the pegRNA comprises a gRNA scaffold sequence of GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence at least 80%, at least 85%, at least 90%, at least 95%, at least 96%, at least 97%, at least 98%, or at least 99% identical to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577), or a sequence comprising one, two, three, four, or five mutations, insertions, or deletions relative to GTTTTAGAGCTAGAAATAGCAAGTTAAAATAAGGCTAGTCCGTTATCAACTTGAA AAAGTGGCACCGAGTCGGTGC (SEQ ID NO: 576) or GTTTCAGAGCTATGCTGGAAACAGCATAGCAAGTTGAAATAAGGCTAGTCCGTT ATCAACTTGAAAAAGTGGCACCGAGTCGGTGC (SEQ ID NO: 577).

36. The pegRNA of any one of claims 33-35, wherein the pegRNA comprises a reverse transcription template (RTT) comprising the sequence of any one of SEQ ID NOs: 404-563, or a sequence at least 80%, at least 85%, at least 90%, at least 95%, at least 96%, at least 97%, at least 98%, or at least 99% identical to of any one of SEQ ID NOs: 404-563, or a sequence comprising one, two, three, four, or five mutations, insertions, or deletions relative to of any one of SEQ ID NOs: 404-563.

37. The pegRNA of any one of claims 33-36, wherein the pegRNA comprises a primer binding site comprising the sequence TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35, TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence at least 80%, at least 85%, at least 90%, at least 95%, at least 96%, at least 97%, at least 98%, or at least 99% identical to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35, TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6), or a sequence comprising one, two, three, four, or five mutations, insertions, or deletions relative to TTCCCCAGT, TTCCCCAGTC (SEQ ID NO: 29), TTCCCCAGTCC (SEQ ID NO: 30), TTCCCCAGTCCT (SEQ ID NO: 31), TTCCCCAGTCCTC (SEQ ID NO: 32), TTCCCCAGTCCTCC (SEQ ID NO: 33), TTCCCCAGTCCTCCC (SEQ ID NO: 34), TAGCTATGC, TAGCTATGCC (SEQ ID NO: 35), TAGCTATGCCG (SEQ ID NO: 36), TAGCTATGCCGA (SEQ ID NO: 37), TAGCTATGCCGAC (SEQ ID NO: 38), or TAGCTATGCCGACT (SEQ ID NO: 6).

38. The pegRNA of any one of claims 33-37, wherein the pegRNA comprises a 3′ structured motif of the sequence CGCGGTTCTATCTAGTTACGCGTTAAACCAACTAGAA (SEQ ID NO: 14), or a sequence at least 80%, at least 85%, at least 90%, at least 95%, at least 96%, at least 97%, at least 98%, or at least 99% identical to CGCGGTTCTATCTAGTTACGCGTTAAACCAACTAGAA (SEQ ID NO: 14), or a sequence comprising one, two, three, four, or five mutations, insertions, or deletions relative to CGCGGTTCTATCTAGTTACGCGTTAAACCAACTAGAA (SEQ ID NO: 14).

39. The pegRNA of any one of claims 33-38, wherein the pegRNA comprises a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT.

40. The pegRNA of any one of claims 33-39, wherein the pegRNA comprises comprise the structure 5′-[spacer]-[gRNA scaffold]-[RTT]-[PBS]-[3′ motif]-[terminator]-3′ or 5′-[spacer]-[gRNA scaffold]-[RTT]-[PBS]-[3′ motif]-3′.

41. A composition comprising the pegRNA of any one of claims 33-40.

42. The composition of claim 41 further comprising a nicking guide RNA (ngRNA).

43. The composition of claim 42, wherein the ngRNA comprises a spacer sequence of GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence at least 80%, at least 85%, at least 90%, at least 95%, at least 96%, at least 97%, at least 98%, or at least 99% identical to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13), or a spacer sequence comprising one, two, three, four, or five mutations, insertions, or deletions relative to GCTGGGGAATTGTGTCAAGAT (SEQ ID NO: 10), GGCCTTGGTCCTGTAGGAGG (SEQ ID NO: 11), GTTGCTCACAGACTTGGAGT (SEQ ID NO: 12), or GCTGGGGAATTGTGTGAGGAT (SEQ ID NO: 13).

44. The composition of claim 42 or 43, wherein the ngRNA comprises the sequence of any one of the ngRNAs provided in Table 3, or a sequence at least 80%, at least 85%, at least 90%, at least 95%, at least 96%, at least 97%, at least 98%, or at least 99% identical to any one of the ngRNAs provided in Table 3.

45. The composition of any one of claims 42-44, wherein the ngRNA comprises a gRNA scaffold sequence of GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence at least 80%, at least 85%, at least 90%, at least 95%, at least 96%, at least 97%, at least 98%, or at least 99% identical to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578), or a sequence comprising one, two, three, four, or five mutations, insertions, or deletions relative to GTTTAAGAGCTAAGCTGGAAACAGCATAGCAAGTTTAAATAAGGCTAGTCCGTT ATCAACTTGAAAAAGTGGCACCGAGTCGGTGC (SEQ ID NO: 578).

46. The composition of any one of claims 42-45, wherein the ngRNA comprises a terminator sequence of TTTTTT, or a sequence at least 80%, at least 85%, at least 90%, at least 95%, at least 96%, at least 97%, at least 98%, or at least 99% identical to TTTTTT, or a sequence comprising one, two, three, four, or five mutations, insertions, or deletions relative to TTTTTT.

47. The composition of any one of claims 42-46, wherein the ngRNA comprises the structure 5′-[spacer]-[gRNA scaffold]-[terminator]-3′.

48. The composition of any one of claims 41-47 further comprising a prime editor.

49. The composition of claim 48, wherein the prime editor comprises (i) a napDNAbp domain, and (ii) a polymerase domain.

50. The composition of claim 49, wherein the napDNAbp domain has a nickase activity.

51. The composition of claim 49 or 50, wherein the napDNAbp domain is a Cas9 protein or a variant thereof.

52. The composition of any one of claims 49-51, wherein the napDNAbp domain is a Cas9 nickase (nCas9).

53. The composition of any one of claims 49-52, wherein the napDNAbp domain comprises a sequence of any one of the napDNAbps provided herein, or an amino acid sequence at least 80%, at least 85%, at least 90%, at least 95%, at least 96%, at least 97%, at least 98%, or at least 99% identical to any one of the napDNAbps provided herein.

54. The composition of any one of claims 49-53, wherein the polymerase domain is a reverse transcriptase domain.

55. The composition of any one of claims 49-54, wherein the polymerase domain comprises a sequence of any one of the polymerases provided herein, or an amino acid sequence at least 80%, at least 85%, at least 90%, at least 95%, at least 96%, at least 97%, at least 98%, or at least 99% identical to any one of the polymerases provided herein.

56. The composition of any one of claims 48-55, wherein the prime editor is PE2, PE3, PE4, PE5, or a variant thereof.

57. The composition of any one of claims 48-56, wherein the prime editor is PE4max-NRCH, or a variant thereof.

58. The composition of any one of claims 48-55, wherein the prime editor is a PE6 prime editor, or a variant thereof.

59. The composition of any one of claim 48-55 or 58, wherein the prime editor is PE6b-NRCH, PE6c-NRCH, PE6d-NRCH, or a variant thereof.

60. One or more polynucleotides encoding the pegRNA of any one of claims 33-40 and/or the ngRNA of the composition of any one of claims 41-59.

61. The one or more polynucleotides of claim 60, wherein the one or more polynucleotides encode the prime editor of the composition of any one of claims 48-59.

62. One or more vectors comprising the one or more polynucleotides of claim 60 or 61.

63. The one or more vectors of claim 62, wherein the vectors are AAV vectors.

64. One or more AAV particles comprising the pegRNA of any one of claims 33-40, the composition of any one of claims 41-59, the one or more polynucleotides of claim 60 or 61, or the one or more vectors of claim 62 or 63.

65. A virus-like particle (VLP) comprising the pegRNA of any one of claims 33-40, the composition of any one of claims 41-59, the one or more polynucleotides of claim 60 or 61, or the one or more vectors of claim 62 or 63.

66. A pharmaceutical composition comprising the pegRNA of any one of claims 33-40, the composition of any one of claims 41-59, the one or more polynucleotides of claim 60 or 61, the one or more vectors of claim 62 or 63, the one or more AAV particles of claim 64, or the VLP of claim 65, and a pharmaceutically acceptable excipient.

67. A cell comprising the pegRNA of any one of claims 33-40, the composition of any one of claims 41-59, the one or more polynucleotides of claim 60 or 61, the one or more vectors of claim 62 or 63, the one or more AAV particles of claim 64, or the VLP of claim 65.

68. A kit comprising the pegRNA of any one of claims 33-40, the composition of any one of claims 41-59, the one or more polynucleotides of claim 60 or 61, the one or more vectors of claim 62 or 63, the one or more AAV particles of claim 64, or the VLP of claim 65.

69. Use of the pegRNA of any one of claims 33-40, the composition of any one of claims 41-59, the one or more polynucleotides of claim 60 or 61, the one or more vectors of claim 62 or 63, the one or more AAV particles of claim 64, the VLP of claim 65, or the pharmaceutical composition of claim 66 in the treatment of CDKL5 deficiency disorder.

70. Use of the pegRNA of any one of claims 33-40, the composition of any one of claims 41-59, the one or more polynucleotides of claim 60 or 61, the one or more vectors of claim 62 or 63, the one or more AAV particles of claim 64, the VLP of claim 65, or the pharmaceutical composition of claim 66 in the manufacture of a medicament for the treatment of CDKL5 deficiency disorder.

71. A system comprising a pegRNA of Table 1 or Table 2, an ngRNA of Table 3, and a prime editor.

72. The system of claim 71, wherein the prime editor is PE6b-NRCH, PE6c-NRCH, or PE6d-NRCH.

73. A method of correcting a 1412delA mutation in a CDKL5 gene by prime editing comprising contacting a target DNA sequence comprising the CDKL5 gene with a prime editor and a prime editing guide RNA (pegRNA), wherein the pegRNA comprises a reverse transcription template (RTT) of the sequence of any one of SEQ ID NOs: 404-563, or a RTT at least 80%, at least 85%, at least 90%, at least 95%, at least 96%, at least 97%, at least 98%, or at least 99% identical to the sequence of any one of SEQ ID NOs: 404-563, or a RTT comprising one, two, three, four, or five mutations, insertions, or deletions relative to the sequence of any one of SEQ ID NOs: 404-563.

74. A prime editing guide RNA (pegRNA) comprising a reverse transcription template (RTT) of the sequence of any one of SEQ ID NOs: 404-563, or a RTT at least 80%, at least 85%, at least 90%, at least 95%, at least 96%, at least 97%, at least 98%, or at least 99% identical to the sequence of any one of SEQ ID NOs: 404-563, or a RTT comprising one, two, three, four, or five mutations, insertions, or deletions relative to the sequence of any one of SEQ ID NOs: 404-563.

75. A method of correcting a c.826-1 G-to-A point mutation in a CDKL5 gene by prime editing comprising contacting a target DNA sequence comprising the CDKL5 gene with a prime editor and a prime editing guide RNA (pegRNA), wherein the pegRNA comprises a spacer sequence of GCTGTCAAGTATCTGTCAGCT (SEQ ID NO: 7), or a spacer sequence at least 80%, at least 85%, at least 90%, at least 95%, at least 96%, at least 97%, at least 98%, or at least 99% identical to GCTGTCAAGTATCTGTCAGCT (SEQ ID NO: 7), or a spacer sequence comprising one, two, three, four, or five mutations, insertions, or deletions relative to GCTGTCAAGTATCTGTCAGCT (SEQ ID NO: 7).

76. The method of claim 75, wherein the pegRNA comprises the sequence GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence at least 80%, at least 85%, at least 90%, at least 95%, at least 96%, at least 97%, at least 98%, or at least 99% identical to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence comprising one, two, three, four, or five mutations, insertions, or deletions relative to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39).

77. The method of claim 75 or 76 further comprising contacting the target DNA sequence with a nicking guide RNA.

78. The method of claim 77, wherein the nicking guide RNA comprises the sequence GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence at least 80%, at least 85%, at least 90%, at least 95%, at least 96%, at least 97%, at least 98%, or at least 99% identical to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence comprising one, two, three, four, or five mutations, insertions, or deletions relative to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17).

79. A pegRNA comprising a spacer sequence of GCTGTCAAGTATCTGTCAGCT (SEQ ID NO: 7), or a spacer sequence at least 80%, at least 85%, at least 90%, at least 95%, at least 96%, at least 97%, at least 98%, or at least 99% identical to GCTGTCAAGTATCTGTCAGCT (SEQ ID NO: 7), or a spacer sequence comprising one, two, three, four, or five mutations, insertions, or deletions relative to GCTGTCAAGTATCTGTCAGCT (SEQ ID NO: 7).

80. The pegRNA of claim 79, wherein the pegRNA comprises the sequence GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence at least 80%, at least 85%, at least 90%, at least 95%, at least 96%, at least 97%, at least 98%, or at least 99% identical to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39), or a sequence comprising one, two, three, four, or five mutations, insertions, or deletions relative to GCTGTCAAGTATCTGTCAGCTGTTTTAGAGCTAGAAATAGCAAGTTAAAATAAGG CTAGTCCGTTATCAACTTGAAAAAGTGGCACCGAGTCGGTGCATTTCCTAAGAAC CTGTTGAAGTTGGACCCAGCTGACAGATACTCGCGGTTCTATCTAGTTACGCGTT AAACCAACTAGAATTTTTTT (SEQ ID NO: 39).

81. A nicking guide RNA comprising the sequence GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence at least 80%, at least 85%, at least 90%, at least 95%, at least 96%, at least 97%, at least 98%, or at least 99% identical to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17), or a sequence comprising one, two, three, four, or five mutations, insertions, or deletions relative to GATTTCAAACCCAGAGACTTC (SEQ ID NO: 15), GTTCTGGATCGTTCTCCTTCA (SEQ ID NO: 16), or GTACTGAAGTTGGACCCAGCT (SEQ ID NO: 17).

82. A method of correcting a cluster of mutations in exon 8 of CDKL5 by prime editing comprising contacting a target DNA sequence comprising the CDKL5 gene with a prime editor and a prime editing guide RNA (pegRNA), wherein the pegRNA comprises a spacer sequence of CAGAGTACGTTGCCACCAGA, or a spacer sequence at least 80%, at least 85%, at least 90%, at least 95%, at least 96%, at least 97%, at least 98%, or at least 99% identical to CAGAGTACGTTGCCACCAGA, or a spacer sequence comprising one, two, three, four, or five mutations, insertions, or deletions relative to CAGAGTACGTTGCCACCAGA.

83. The method of claim 82, wherein the pegRNA comprises the sequence CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence at least 80%, at least 85%, at least 90%, at least 95%, at least 96%, at least 97%, at least 98%, or at least 99% identical to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence comprising one, two, three, four, or five mutations, insertions, or deletions relative to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41).

84. The method of claim 82 or 83 further comprising contacting the target DNA sequence with a nicking guide RNA.

85. The method of claim 84, wherein the nicking guide RNA comprises the sequence GTGGAAATGTCATTCTATTTT (SEQ ID NO: 18), or a sequence at least 80%, at least 85%, at least 90%, at least 95%, at least 96%, at least 97%, at least 98%, or at least 99% identical to GTGGAAATGTCATTCTATTTT (SEQ ID NO: 18), or a sequence comprising one, two, three, four, or five mutations, insertions, or deletions relative to GTGGAAATGTCATTCTATTTT (SEQ ID NO: 18).

86. A pegRNA comprising a spacer sequence of CAGAGTACGTTGCCACCAGA (SEQ ID NO: 8), or a spacer sequence at least 80%, at least 85%, at least 90%, at least 95%, at least 96%, at least 97%, at least 98%, or at least 99% identical to CAGAGTACGTTGCCACCAGA (SEQ ID NO: 8), or a spacer sequence comprising one, two, three, four, or five mutations, insertions, or deletions relative to CAGAGTACGTTGCCACCAGA (SEQ ID NO: 8).

87. The pegRNA of claim 86, wherein the pegRNA comprises the sequence CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence at least 80%, at least 85%, at least 90%, at least 95%, at least 96%, at least 97%, at least 98%, or at least 99% identical to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41), or a sequence comprising one, two, three, four, or five mutations, insertions, or deletions relative to CAGAGTACGTTGCCACCAGAGTTTTAGAGCTAGAAATAGCAAGTTAAAATAAGG CTAGTCCGTTATCAACTTGAAAAAGTGGCACCGAGTCGGTGCAAGTAAGGGCTCT AGGGAGCGACACCGGCTGGTGGCAACCGCGGTTCTATCTAGTTACGCGTTAAAC CAACTAGAATTTTTTT (SEQ ID NO: 40) or CAGAGTACGTTGCCACCAGAGTTTTAGAGCTAGAAATAGCAAGTTAAAATAAGG CTAGTCCGTTATCAACTTGAAAAAGTGGCACCGAGTCGGTGCACCCAAGTAAGG GCTCTAGGGAGCGACACCGGCTGGTGGCAACCGCGGTTCTATCTAGTTACGCGTT AAACCAACTAGAATTTTTTT (SEQ ID NO: 41).

88. A nicking guide RNA comprising the sequence GTGGAAATGTCATTCTATTTT (SEQ ID NO: 18), or a sequence at least 80%, at least 85%, at least 90%, at least 95%, at least 96%, at least 97%, at least 98%, or at least 99% identical to GTGGAAATGTCATTCTATTTT (SEQ ID NO: 18), or a sequence comprising one, two, three, four, or five mutations, insertions, or deletions relative to GTGGAAATGTCATTCTATTTT (SEQ ID NO: 18).

89. A method for treating CDKL5 deficiency disorder (CDD) by prime editing comprising contacting a target DNA sequence comprising a CDKL5 gene comprising a pathogenic mutation with a prime editor, or a nucleotide sequence encoding a prime editor, and a pegRNA, or a nucleotide sequence encoding a prime editor, wherein the prime editor comprises an amino acid sequence of any one of SEQ ID NOs: 355-357, or an amino acid sequence at least 80%, at least 85%, at least 90%, at least 95%, at least 96%, at least 97%, at least 98%, or at least 99% identical to an amino acid sequence of any one of SEQ ID NOs: 355-357.

90. The method of claim 89, wherein the pegRNA is capable of correcting a c.1412delA mutation in the CDKL5 gene.

91. The method of claim 89 or 90, wherein the pegRNA is a pegRNA of any one of claims 33-40.

92. A complex comprising a pegRNA of any one of claims 1-40 and a prime editor.


SEQUENCE LISTING (604 sequences)

SEQ ID NO: 1 - DNA, 21 bp, synthetic construct
  source 1..21: mol_type = other DNA
gccgggaggactggggaatgg

SEQ ID NO: 2 - DNA, 21 bp, synthetic construct
  source 1..21: mol_type = other DNA
gccagatctcagcagaacagg

SEQ ID NO: 3 - DNA, 21 bp, synthetic construct
  source 1..21: mol_type = other DNA
gcattcatggaaagctcccag

SEQ ID NO: 4 - DNA, 21 bp, synthetic construct
  source 1..21: mol_type = other DNA
gccagagcaaagctgggaccc

SEQ ID NO: 5 - DNA, 21 bp, synthetic construct
  source 1..21: mol_type = other DNA
gcagagtcggcatagctatat

SEQ ID NO: 6 - DNA, 14 bp, synthetic construct
  source 1..14: mol_type = other DNA
tagctatgccgact

SEQ ID NO: 7 - DNA, 21 bp, synthetic construct
  source 1..21: mol_type = other DNA
gctgtcaagtatctgtcagct

SEQ ID NO: 8 - DNA, 20 bp, synthetic construct
  source 1..20: mol_type = other DNA
cagagtacgttgccaccaga

SEQ ID NO: 9 - DNA, 20 bp, synthetic construct
  source 1..20: mol_type = other DNA
gagagtcggcatagctatat

SEQ ID NO: 10 - DNA, 21 bp, synthetic construct
  source 1..21: mol_type = other DNA
gctggggaattgtgtcaagat

SEQ ID NO: 11 - DNA, 20 bp, synthetic construct
  source 1..20: mol_type = other DNA
ggccttggtcctgtaggagg

SEQ ID NO: 12 - DNA, 20 bp, synthetic construct
  source 1..20: mol_type = other DNA
gttgctcacagacttggagt

SEQ ID NO: 13 - DNA, 21 bp, synthetic construct
  source 1..21: mol_type = other DNA
gctggggaattgtgtgaggat

SEQ ID NO: 14 - DNA, 37 bp, synthetic construct
  source 1..37: mol_type = other DNA
cgcggttctatctagttacgcgttaaaccaactagaa

SEQ ID NO: 15 - DNA, 21 bp, synthetic construct
  source 1..21: mol_type = other DNA
gatttcaaacccagagacttc

SEQ ID NO: 16 - DNA, 21 bp, synthetic construct
  source 1..21: mol_type = other DNA
gttctggatcgttctccttca

SEQ ID NO: 17 - DNA, 21 bp, synthetic construct
  source 1..21: mol_type = other DNA
gtactgaagttggacccagct

SEQ ID NO: 18 - DNA, 21 bp, synthetic construct
  source 1..21: mol_type = other DNA
gtggaaatgtcattctatttt

SEQ ID NO: 19 - DNA, 21 bp, synthetic construct
  source 1..21: mol_type = other DNA
gattcttaggaaataaaattt

SEQ ID NO: 20 - DNA, 20 bp, synthetic construct
  source 1..20: mol_type = other DNA
gcagaaccgccactcattca

SEQ ID NO: 21 - protein, 7 aa, synthetic construct
PKKKRKV

SEQ ID NO: 22 - protein, 30 aa, synthetic construct
MDSLLMNRRKFLYQFKNVRWAKGRRETYLC

SEQ ID NO: 23 - protein, 18 aa, synthetic construct
KRTADGSEFESPKKKRKV

SEQ ID NO: 24 - protein, 17 aa, synthetic construct
KRTADGSEFEPKKKRKV

SEQ ID NO: 25 - protein, 19 aa, synthetic construct
NLSKRPAAIKKAGQAKKKK

SEQ ID NO: 26 - protein, 9 aa, synthetic construct
PAAKRVKLD

SEQ ID NO: 27 - protein, 11 aa, synthetic construct
RQRRNELKRSF

SEQ ID NO: 28 - protein, 38 aa, synthetic construct
NQSSNFGPMKGGNFGGRSSGPYGGGGQYFAKPRNQGGY

SEQ ID NO: 29 - DNA, 10 bp, synthetic construct
  source 1..10: mol_type = other DNA
ttccccagtc

SEQ ID NO: 30 - DNA, 11 bp, synthetic construct
  source 1..11: mol_type = other DNA
ttccccagtcc

SEQ ID NO: 31 - DNA, 12 bp, synthetic construct
  source 1..12: mol_type = other DNA
ttccccagtcct

SEQ ID NO: 32 - DNA, 13 bp, synthetic construct
  source 1..13: mol_type = other DNA
ttccccagtcctc

SEQ ID NO: 33 - DNA, 14 bp, synthetic construct
  source 1..14: mol_type = other DNA
ttccccagtcctcc

SEQ ID NO: 34 - DNA, 15 bp, synthetic construct
  source 1..15: mol_type = other DNA
ttccccagtcctccc

SEQ ID NO: 35 - DNA, 10 bp, synthetic construct
  source 1..10: mol_type = other DNA
tagctatgcc

SEQ ID NO: 36 - DNA, 11 bp, synthetic construct
  source 1..11: mol_type = other DNA
tagctatgccg

SEQ ID NO: 37 - DNA, 12 bp, synthetic construct
  source 1..12: mol_type = other DNA
tagctatgccga

SEQ ID NO: 38 - DNA, 13 bp, synthetic construct
  source 1..13: mol_type = other DNA
tagctatgccgac

SEQ ID NO: 39 - DNA, 185 bp, synthetic construct
  source 1..185: mol_type = other DNA
gctgtcaagtatctgtcagctgttttagagctagaaatagcaagttaaaataaggctagtccgttatcaacttgaaaaagtggcaccgagtcggtgcatttcctaagaacctgttgaagttggacccagctgacagatactcgcggttctatctagttacgcgttaaaccaactagaattttttt

SEQ ID NO: 40 - DNA, 179 bp, synthetic construct
  source 1..179: mol_type = other DNA
cagagtacgttgccaccagagttttagagctagaaatagcaagttaaaataaggctagtccgttatcaacttgaaaaagtggcaccgagtcggtgcaagtaagggctctagggagcgacaccggctggtggcaaccgcggttctatctagttacgcgttaaaccaactagaattttttt

SEQ ID NO: 41 - DNA, 183 bp, synthetic construct
  source 1..183: mol_type = other DNA
cagagtacgttgccaccagagttttagagctagaaatagcaagttaaaataaggctagtccgttatcaacttgaaaaagtggcaccgagtcggtgcacccaagtaagggctctagggagcgacaccggctggtggcaaccgcggttctatctagttacgcgttaaaccaactagaattttttt

SEQ ID NO: 42 - DNA, 157 bp, synthetic construct
  source 1..157: mol_type = other DNA
gccgggaggactggggaatgggttttagagctagaaatagcaagttaaaataaggctagtccgttatcaacttgaaaaagtggcaccgagtcggtgcctatattgacacaattccccagtcgcggttctatctagttacgcgttaaaccaactagaa

SEQ ID NO: 43 - DNA, 158 bp, synthetic construct
  source 1..158: mol_type = other DNA
gccgggaggactggggaatgggttttagagctagaaatagcaagttaaaataaggctagtccgttatcaacttgaaaaagtggcaccgagtcggtgcgctatattgacacaattccccagtcgcggttctatctagttacgcgttaaaccaactagaa

SEQ ID NO: 44 - DNA, 159 bp, synthetic construct
  source 1..159: mol_type = other DNA
gccgggaggactggggaatgggttttagagctagaaatagcaagttaaaataaggctagtccgttatcaacttgaaaaagtggcaccgagtcggtgcagctatattgacacaattccccagtcgcggttctatctagttacgcgttaaaccaactagaa

SEQ ID NO: 45 - DNA, 160 bp, synthetic construct
  source 1..160: mol_type = other DNA
gccgggaggactggggaatgggttttagagctagaaatagcaagttaaaataaggctagtccgttatcaacttgaaaaagtggcaccgagtcggtgccagctatattgacacaattccccagtcgcggttctatctagttacgcgttaaaccaactagaa

SEQ ID NO: 46 - DNA, 161 bp, synthetic construct
  source 1..161: mol_type = other DNA
gccgggaggactggggaatgggttttagagctagaaatagcaagttaaaataaggctagtccgttatcaacttgaaaaagtggcaccgagtcggtgcacagctatattgacacaattccccagtcgcggttctatctagttacgcgttaaaccaactagaa

SEQ ID NO: 47 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgccacagctatattgacacaattccccagtcgcggttctatctagttacgcgttaaaccaactagaa

SEQ ID NO: 48 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcgcggttctatctagttacgcgttaaaccaactagaa

SEQ ID NO: 49 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcgcggttctatctagttacgcgttaaaccaactagaa

SEQ ID NO: 50 - DNA, 158 bp, synthetic construct
  source 1..158: mol_type = other DNA
gccgggaggactggggaatgggttttagagctagaaatagcaagttaaaataaggctagtccgttatcaacttgaaaaagtggcaccgagtcggtgcctatattgacacaattccccagtccgcggttctatctagttacgcgttaaaccaactagaa

SEQ ID NO: 51 - DNA, 159 bp, synthetic construct
  source 1..159: mol_type = other DNA
gccgggaggactggggaatgggttttagagctagaaatagcaagttaaaataaggctagtccgttatcaacttgaaaaagtggcaccgagtcggtgcgctatattgacacaattccccagtccgcggttctatctagttacgcgttaaaccaactagaa

SEQ ID NO: 52 - DNA, 160 bp, synthetic construct
  source 1..160: mol_type = other DNA
gccgggaggactggggaatgggttttagagctagaaatagcaagttaaaataaggctagtccgttatcaacttgaaaaagtggcaccgagtcggtgcagctatattgacacaattccccagtccgcggttctatctagttacgcgttaaaccaactagaa

SEQ ID NO: 53 - DNA, 161 bp, synthetic construct
  source 1..161: mol_type = other DNA
gccgggaggactggggaatgggttttagagctagaaatagcaagttaaaataaggctagtccgttatcaacttgaaaaagtggcaccgagtcggtgccagctatattgacacaattccccagtccgcggttctatctagttacgcgttaaaccaactagaa

SEQ ID NO: 54 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgcacagctatattgacacaattccccagtccgcggttctatctagttacgcgttaaaccaactagaa

SEQ ID NO: 55 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgccacagctatattgacacaattccccagtccgcggttctatctagttacgcgttaaaccaactagaa

SEQ ID NO: 56 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtccgcggttctatctagttacgcgttaaaccaactagaa

SEQ ID NO: 57 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtccgcggttctatctagttacgcgttaaaccaactagaa

SEQ ID NO: 58 - DNA, 159 bp, synthetic construct
  source 1..159: mol_type = other DNA
gccgggaggactggggaatgggttttagagctagaaatagcaagttaaaataaggctagtccgttatcaacttgaaaaagtggcaccgagtcggtgcctatattgacacaattccccagtcccgcggttctatctagttacgcgttaaaccaactagaa

SEQ ID NO: 59 - DNA, 160 bp, synthetic construct
  source 1..160: mol_type = other DNA
gccgggaggactggggaatgggttttagagctagaaatagcaagttaaaataaggctagtccgttatcaacttgaaaaagtggcaccgagtcggtgcgctatattgacacaattccccagtcccgcggttctatctagttacgcgttaaaccaactagaa

SEQ ID NO: 60 - DNA, 161 bp, synthetic construct
  source 1..161: mol_type = other DNA
gccgggaggactggggaatgggttttagagctagaaatagcaagttaaaataaggctagtccgttatcaacttgaaaaagtggcaccgagtcggtgcagctatattgacacaattccccagtcccgcggttctatctagttacgcgttaaaccaactagaa

SEQ ID NO: 61 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgccagctatattgacacaattccccagtcccgcggttctatctagttacgcgttaaaccaactagaa

SEQ ID NO: 62 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgcacagctatattgacacaattccccagtcccgcggttctatctagttacgcgttaaaccaactagaa

SEQ ID NO: 63 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgccacagctatattgacacaattccccagtcccgcggttctatctagttacgcgttaaaccaactagaa

SEQ ID NO: 64 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcccgcggttctatctagttacgcgttaaaccaactagaa

SEQ ID NO: 65 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcccgcggttctatctagttacgcgttaaaccaactagaa

SEQ ID NO: 66 - DNA, 160 bp, synthetic construct
  source 1..160: mol_type = other DNA
gccgggaggactggggaatgggttttagagctagaaatagcaagttaaaataaggctagtccgttatcaacttgaaaaagtggcaccgagtcggtgcctatattgacacaattccccagtcctcgcggttctatctagttacgcgttaaaccaactagaa

SEQ ID NO: 67 - DNA, 161 bp, synthetic construct
  source 1..161: mol_type = other DNA
gccgggaggactggggaatgggttttagagctagaaatagcaagttaaaataaggctagtccgttatcaacttgaaaaagtggcaccgagtcggtgcgctatattgacacaattccccagtcctcgcggttctatctagttacgcgttaaaccaactagaa

SEQ ID NO: 68 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgcagctatattgacacaattccccagtcctcgcggttctatctagttacgcgttaaaccaactagaa

SEQ ID NO: 69 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgccagctatattgacacaattccccagtcctcgcggttctatctagttacgcgttaaaccaactagaa

SEQ ID NO: 70 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgcacagctatattgacacaattccccagtcctcgcggttctatctagttacgcgttaaaccaactagaa

SEQ ID NO: 71 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgccacagctatattgacacaattccccagtcctcgcggttctatctagttacgcgttaaaccaactagaa

SEQ ID NO: 72 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcctcgcggttctatctagttacgcgttaaaccaactagaa

SEQ ID NO: 73 - DNA, 167 bp, synthetic construct
  source 1..167: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcctcgcggttctatctagttacgcgttaaaccaactagaa

SEQ ID NO: 74 - DNA, 161 bp, synthetic construct
  source 1..161: mol_type = other DNA
gccgggaggactggggaatgggttttagagctagaaatagcaagttaaaataaggctagtccgttatcaacttgaaaaagtggcaccgagtcggtgcctatattgacacaattccccagtcctccgcggttctatctagttacgcgttaaaccaactagaa

SEQ ID NO: 75 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgcgctatattgacacaattccccagtcctccgcggttctatctagttacgcgttaaaccaactagaa

SEQ ID NO: 76 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgcagctatattgacacaattccccagtcctccgcggttctatctagttacgcgttaaaccaactagaa

SEQ ID NO: 77 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgccagctatattgacacaattccccagtcctccgcggttctatctagttacgcgttaaaccaactagaa

SEQ ID NO: 78 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgcacagctatattgacacaattccccagtcctccgcggttctatctagttacgcgttaaaccaactagaa

SEQ ID NO: 79 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgccacagctatattgacacaattccccagtcctccgcggttctatctagttacgcgttaaaccaactagaa

SEQ ID NO: 80 - DNA, 167 bp, synthetic construct
  source 1..167: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcctccgcggttctatctagttacgcgttaaaccaactagaa

SEQ ID NO: 81 - DNA, 168 bp, synthetic construct
  source 1..168: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcctccgcggttctatctagttacgcgttaaaccaactagaa

SEQ ID NO: 82 - DNA, 162 bp, synthetic construct
  source 1..162: mol_type = other DNA
gccgggaggactggggaatgggttttagagctagaaatagcaagttaaaataaggctagtccgttatcaacttgaaaaagtggcaccgagtcggtgcctatattgacacaattccccagtcctcccgcggttctatctagttacgcgttaaaccaactagaa

SEQ ID NO: 83 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgcgctatattgacacaattccccagtcctcccgcggttctatctagttacgcgttaaaccaactagaa

SEQ ID NO: 84 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgcagctatattgacacaattccccagtcctcccgcggttctatctagttacgcgttaaaccaactagaa

SEQ ID NO: 85 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgccagctatattgacacaattccccagtcctcccgcggttctatctagttacgcgttaaaccaactagaa

SEQ ID NO: 86 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgcacagctatattgacacaattccccagtcctcccgcggttctatctagttacgcgttaaaccaactagaa

SEQ ID NO: 87 - DNA, 167 bp, synthetic construct
  source 1..167: mol_type = other DNA
gccgggaggactggggaatgggttttagagctagaaatagcaagttaaaataaggctagtccgttatcaacttgaaaaagtggcaccgagtcggtgccacagctatattgacacaattccccagtcctcccgcggttctatctagttacgcgttaaaccaactagaa

SEQ ID NO: 88 - DNA, 168 bp, synthetic construct
  source 1..168: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcctcccgcggttctatctagttacgcgttaaaccaactagaa

SEQ ID NO: 89 - DNA, 169 bp, synthetic construct
  source 1..169: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcctcccgcggttctatctagttacgcgttaaaccaactagaa

SEQ ID NO: 90 - DNA, 163 bp, synthetic construct
  source 1..163: mol_type = other DNA
gccgggaggactggggaatgggttttagagctagaaatagcaagttaaaataaggctagtccgttatcaacttgaaaaagtggcaccgagtcggtgcctatattgacacaattccccagtcctccccgcggttctatctagttacgcgttaaaccaactagaa

SEQ ID NO: 91 - DNA, 164 bp, synthetic construct
  source 1..164: mol_type = other DNA
gccgggaggactggggaatgggttttagagctagaaatagcaagttaaaataaggctagtccgttatcaacttgaaaaagtggcaccgagtcggtgcgctatattgacacaattccccagtcctccccgcggttctatctagttacgcgttaaaccaactagaa

SEQ ID NO: 92 - DNA, 165 bp, synthetic construct
  source 1..165: mol_type = other DNA
gccgggaggactggggaatgggttttagagctagaaatagcaagttaaaataaggctagtccgttatcaacttgaaaaagtggcaccgagtcggtgcagctatattgacacaattccccagtcctccccgcggttctatctagttacgcgttaaaccaactagaa

SEQ ID NO: 93 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgccagctatattgacacaattccccagtcctccccgcggttctatctagttacgcgttaaaccaactagaa

SEQ ID NO: 94 - DNA, 167 bp, synthetic construct
  source 1..167: mol_type = other DNA
gccgggaggactggggaatgggttttagagctagaaatagcaagttaaaataaggctagtccgttatcaacttgaaaaagtggcaccgagtcggtgcacagctatattgacacaattccccagtcctccccgcggttctatctagttacgcgttaaaccaactagaa

SEQ ID NO: 95 - DNA, 168 bp, synthetic construct
  source 1..168: mol_type = other DNA
gccgggaggactggggaatgggttttagagctagaaatagcaagttaaaataaggctagtccgttatcaacttgaaaaagtggcaccgagtcggtgccacagctatattgacacaattccccagtcctccccgcggttctatctagttacgcgttaaaccaactagaa

SEQ ID NO: 96 - DNA, 169 bp, synthetic construct
  source 1..169: mol_type = other DNA
gccgggaggactggggaatgggttttagagctagaaatagcaagttaaaataaggctagtccgttatcaacttgaaaaagtggcaccgagtcggtgcgcacagctatattgacacaattccccagtcctccccgcggttctatctagttacgcgttaaaccaactagaa

SEQ ID NO: 97 - DNA, 170 bp, synthetic construct
  source 1..170: mol_type = other DNA
gccgggaggactggggaatgggttttagagctagaaatagcaagttaaaataaggctagtccgttatcaacttgaaaaagtggcaccgagtcggtgcggcacagctatattgacacaattccccagtcctccccgcggttctatctagttacgcgttaaaccaactagaa

SEQ ID NO: 98 - DNA, 170 bp, synthetic construct
  source 1..170: mol_type = other DNA
gccgggaggactggggaatgggtttaagagctaagctggaaacagcatagcaagtttaaataaggctagtccgttatcaacttgaaaaagtggcaccgagtcggtgcgctatattgacacaattccccagtcccgcggttctatctagttacgcgttaaaccaactagaa

SEQ ID NO: 99 - DNA, 168 bp, synthetic construct
  source 1..168: mol_type = other DNA
gccgggaggactggggaatgggtttaagagctaagctggaaacagcatagcaagtttaaataaggctagtccgttatcaacttgaaaaagtggcaccgagtcggtgcgctatattgacacaattccccagtcgcggttctatctagttacgcgttaaaccaactagaa

SEQ ID NO: 100 - DNA, 169 bp, synthetic construct
  source 1..169: mol_type = other DNA
gccgggaggactggggaatgggtttaagagctaagctggaaacagcatagcaagtttaaataaggctagtccgttatcaacttgaaaaagtggcaccgagtcggtgcgctatattgacacaattccccagtccgcggttctatctagttacgcgttaaaccaactagaa

SEQ ID NO: 101 - DNA, 97 bp, synthetic construct
  source 1..97: mol_type = other DNA
gccagatctcagcagaacagggttttagagctagaaatagcaagttaaaataaggctagtccgttatcaacttgaaaaagtggcaccgagtcggtgc

SEQ ID NO: 102 - DNA, 97 bp, synthetic construct
  source 1..97: mol_type = other DNA
gcattcatggaaagctcccaggttttagagctagaaatagcaagttaaaataaggctagtccgttatcaacttgaaaaagtggcaccgagtcggtgc

SEQ ID NO: 103 - DNA, 97 bp, synthetic construct
  source 1..97: mol_type = other DNA
gccagagcaaagctgggacccgttttagagctagaaatagcaagttaaaataaggctagtccgttatcaacttgaaaaagtggcaccgagtcggtgc

SEQ ID NO: 104 - DNA, 157 bp, synthetic construct
  source 1..157: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgccgcggttctatctagttacgcgttaaaccaactagaa

SEQ ID NO: 105 - DNA, 158 bp, synthetic construct
  source 1..158: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgccgcggttctatctagttacgcgttaaaccaactagaa

SEQ ID NO: 106 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgccgcggttctatctagttacgcgttaaaccaactagaa

SEQ ID NO: 107 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgccgcggttctatctagttacgcgttaaaccaactagaa

SEQ ID NO: 108 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgccgcggttctatctagttacgcgttaaaccaactagaa

SEQ ID NO: 109 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgccgcggttctatctagttacgcgttaaaccaactagaa

SEQ ID NO: 110 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgccgcggttctatctagttacgcgttaaaccaactagaa

SEQ ID NO: 111 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgccgcggttctatctagttacgcgttaaaccaactagaa

SEQ ID NO: 112 - DNA, 158 bp, synthetic construct
  source 1..158: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgcccgcggttctatctagttacgcgttaaaccaactagaa

SEQ ID NO: 113 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgcccgcggttctatctagttacgcgttaaaccaactagaa

SEQ ID NO: 114 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgcccgcggttctatctagttacgcgttaaaccaactagaa

SEQ ID NO: 115 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgcccgcggttctatctagttacgcgttaaaccaactagaa

SEQ ID NO: 116 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgcccgcggttctatctagttacgcgttaaaccaactagaa

SEQ ID NO: 117 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgcccgcggttctatctagttacgcgttaaaccaactagaa

SEQ ID NO: 118 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgcccgcggttctatctagttacgcgttaaaccaactagaa

SEQ ID NO: 119 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgcccgcggttctatctagttacgcgttaaaccaactagaa

SEQ ID NO: 120 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgccgcgcggttctatctagttacgcgttaaaccaactagaa

SEQ ID NO: 121 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgccgcgcggttctatctagttacgcgttaaaccaactagaa

SEQ ID NO: 122 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgccgcgcggttctatctagttacgcgttaaaccaactagaa

SEQ ID NO: 123 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgccgcgcggttctatctagttacgcgttaaaccaactagaa

SEQ ID NO: 124 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgccgcgcggttctatctagttacgcgttaaaccaactagaa

SEQ ID NO: 125 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgccgcgcggttctatctagttacgcgttaaaccaactagaa

SEQ ID NO: 126 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgccgcgcggttctatctagttacgcgttaaaccaactagaa

SEQ ID NO: 127 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgccgcgcggttctatctagttacgcgttaaaccaactagaa

SEQ ID NO: 128 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgccgacgcggttctatctagttacgcgttaaaccaactagaa

SEQ ID NO: 129 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgccgacgcggttctatctagttacgcgttaaaccaactagaa

SEQ ID NO: 130 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgccgacgcggttctatctagttacgcgttaaaccaactagaa

SEQ ID NO: 131 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgccgacgcggttctatctagttacgcgttaaaccaactagaa

SEQ ID NO: 132 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgccgacgcggttctatctagttacgcgttaaaccaactagaa

SEQ ID NO: 133 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgccgacgcggttctatctagttacgcgttaaaccaactagaa

SEQ ID NO: 134 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgccgacgcggttctatctagttacgcgttaaaccaactagaa

SEQ ID NO: 135 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgccgacgcggttctatctagttacgcgttaaaccaactagaa

SEQ ID NO: 136 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgccgaccgcggttctatctagttacgcgttaaaccaactagaa

SEQ ID NO: 137 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgccgaccgcggttctatctagttacgcgttaaaccaactagaa

SEQ ID NO: 138 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgccgaccgcggttctatctagttacgcgttaaaccaactagaa

SEQ ID NO: 139 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgccgaccgcggttctatctagttacgcgttaaaccaactagaa

SEQ ID NO: 140 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgccgaccgcggttctatctagttacgcgttaaaccaactagaa

SEQ ID NO: 141 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgccgaccgcggttctatctagttacgcgttaaaccaactagaa

SEQ ID NO: 142 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgccgaccgcggttctatctagttacgcgttaaaccaactagaa

SEQ ID NO: 143 - DNA, 168 bp, synthetic construct
  source 1..168: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgccgaccgcggttctatctagttacgcgttaaaccaactagaa

SEQ ID NO: 144 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcgaattgtgtcaagatagctatgccgactcgcggttctatctagttacgcgttaaaccaactagaa

SEQ ID NO: 145 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcggaattgtgtcaagatagctatgccgactcgcggttctatctagttacgcgttaaaccaactagaa

SEQ ID NO: 146 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcgggaattgtgtcaagatagctatgccgactcgcggttctatctagttacgcgttaaaccaactagaa

SEQ ID NO: 147 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcggggaattgtgtcaagatagctatgccgactcgcggttctatctagttacgcgttaaaccaactagaa

SEQ ID NO: 148 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgctggggaattgtgtcaagatagctatgccgactcgcggttctatctagttacgcgttaaaccaactagaa

SEQ ID NO: 149 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcctggggaattgtgtcaagatagctatgccgactcgcggttctatctagttacgcgttaaaccaactagaa

SEQ ID NO: 150 - DNA, 168 bp, synthetic construct
  source 1..168: mol_type = other DNA
gcagagtcggcatagctatatgttttagagctagaaatagcaagttaaaataaggctagtccgttatcaacttgaaaaagtggcaccgagtcggtgcactggggaattgtgtcaagatagctatgccgactcgcggttctatctagttacgcgttaaaccaactagaa

SEQ ID NO: 151 - DNA, 169 bp, synthetic construct
  source 1..169: mol_type = other DNA
gcagagtcggcatagctatatgttttagagctagaaatagcaagttaaaataaggctagtccgttatcaacttgaaaaagtggcaccgagtcggtgcgactggggaattgtgtcaagatagctatgccgactcgcggttctatctagttacgcgttaaaccaactagaa

SEQ ID NO: 152 - DNA, 157 bp, synthetic construct
  source 1..157: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgccgcggttctatctagttacgcgttaaaccaactagaa

SEQ ID NO: 153 - DNA, 158 bp, synthetic construct
  source 1..158: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgccgcggttctatctagttacgcgttaaaccaactagaa

SEQ ID NO: 154 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgccgcggttctatctagttacgcgttaaaccaactagaa

SEQ ID NO: 155 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgccgcggttctatctagttacgcgttaaaccaactagaa

SEQ ID NO: 156 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgccgcggttctatctagttacgcgttaaaccaactagaa

SEQ ID NO: 157 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgccgcggttctatctagttacgcgttaaaccaactagaa

SEQ ID NO: 158 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgccgcggttctatctagttacgcgttaaaccaactagaa

SEQ ID NO: 159 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgccgcggttctatctagttacgcgttaaaccaactagaa

SEQ ID NO: 160 - DNA, 158 bp, synthetic construct
  source 1..158: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgcccgcggttctatctagttacgcgttaaaccaactagaa

SEQ ID NO: 161 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgcccgcggttctatctagttacgcgttaaaccaactagaa

SEQ ID NO: 162 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgcccgcggttctatctagttacgcgttaaaccaactagaa

SEQ ID NO: 163 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgcccgcggttctatctagttacgcgttaaaccaactagaa

SEQ ID NO: 164 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgcccgcggttctatctagttacgcgttaaaccaactagaa

SEQ ID NO: 165 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgcccgcggttctatctagttacgcgttaaaccaactagaa

SEQ ID NO: 166 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgcccgcggttctatctagttacgcgttaaaccaactagaa

SEQ ID NO: 167 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgcccgcggttctatctagttacgcgttaaaccaactagaa

SEQ ID NO: 168 - DNA, 159 bp, synthetic construct
  source 1..159: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgccgcgcggttctatctagttacgcgttaaaccaactagaa

SEQ ID NO: 169 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgccgcgcggttctatctagttacgcgttaaaccaactagaa

SEQ ID NO: 170 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgccgcgcggttctatctagttacgcgttaaaccaactagaa

SEQ ID NO: 171 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgccgcgcggttctatctagttacgcgttaaaccaactagaa

SEQ ID NO: 172 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgccgcgcggttctatctagttacgcgttaaaccaactagaa

SEQ ID NO: 173 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgccgcgcggttctatctagttacgcgttaaaccaactagaa

SEQ ID NO: 174 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgccgcgcggttctatctagttacgcgttaaaccaactagaa

SEQ ID NO: 175 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgccgcgcggttctatctagttacgcgttaaaccaactagaa

SEQ ID NO: 176 - DNA, 160 bp, synthetic construct
  source 1..160: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgccgacgcggttctatctagttacgcgttaaaccaactagaa

SEQ ID NO: 177 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgccgacgcggttctatctagttacgcgttaaaccaactagaa

SEQ ID NO: 178 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgccgacgcggttctatctagttacgcgttaaaccaactagaa

SEQ ID NO: 179 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgccgacgcggttctatctagttacgcgttaaaccaactagaa

SEQ ID NO: 180 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgccgacgcggttctatctagttacgcgttaaaccaactagaa

SEQ ID NO: 181 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgccgacgcggttctatctagttacgcgttaaaccaactagaa

SEQ ID NO: 182 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgccgacgcggttctatctagttacgcgttaaaccaactagaa

SEQ ID NO: 183 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgccgacgcggttctatctagttacgcgttaaaccaactagaa

SEQ ID NO: 184 - DNA, 161 bp, synthetic construct
  source 1..161: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgccgaccgcggttctatctagttacgcgttaaaccaactagaa

SEQ ID NO: 185 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgccgaccgcggttctatctagttacgcgttaaaccaactagaa

SEQ ID NO: 186 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgccgaccgcggttctatctagttacgcgttaaaccaactagaa

SEQ ID NO: 187 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgccgaccgcggttctatctagttacgcgttaaaccaactagaa

SEQ ID NO: 188 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgccgaccgcggttctatctagttacgcgttaaaccaactagaa

SEQ ID NO: 189 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgccgaccgcggttctatctagttacgcgttaaaccaactagaa

SEQ ID NO: 190 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgccgaccgcggttctatctagttacgcgttaaaccaactagaa

SEQ ID NO: 191 - DNA, 168 bp, synthetic construct
  source 1..168: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgccgaccgcggttctatctagttacgcgttaaaccaactagaa

SEQ ID NO: 192 - DNA, 162 bp, synthetic construct
  source 1..162: mol_type = other DNA
gcagagtcggcatagctatatgttttagagctagaaatagcaagttaaaataaggctagtccgttatcaacttgaaaaagtggcaccgagtcggtgcgaattgtgtgaggatagctatgccgactcgcggttctatctagttacgcgttaaaccaactagaa

SEQ ID NO: 193 - DNA, 163 bp, synthetic construct
  source 1..163: mol_type = other DNA
gcagagtcggcatagctatatgttttagagctagaaatagcaagttaaaataaggctagtccgttatcaacttgaaaaagtggcaccgagtcggtgcggaattgtgtgaggatagctatgccgactcgcggttctatctagttacgcgttaaaccaactagaa

SEQ ID NO: 194 - DNA, 164 bp, synthetic construct
  source 1..164: mol_type = other DNA
gcagagtcggcatagctatatgttttagagctagaaatagcaagttaaaataaggctagtccgttatcaacttgaaaaagtggcaccgagtcggtgcgggaattgtgtgaggatagctatgccgactcgcggttctatctagttacgcgttaaaccaactagaa

SEQ ID NO: 195 - DNA, 165 bp, synthetic construct
  source 1..165: mol_type = other DNA
gcagagtcggcatagctatatgttttagagctagaaatagcaagttaaaataaggctagtccgttatcaacttgaaaaagtggcaccgagtcggtgcggggaattgtgtgaggatagctatgccgactcgcggttctatctagttacgcgttaaaccaactagaa

SEQ ID NO: 196 - DNA, 166 bp, synthetic construct
  source 1..166: mol_type = other DNA
gcagagtcggcatagctatatgttttagagctagaaatagcaagttaaaataaggctagtccgttatcaacttgaaaaagtggcaccgagtcggtgctggggaattgtgtgaggatagctatgccgactcgcggttctatctagttacgcgttaaaccaactagaa

SEQ ID NO: 197 - DNA, 167 bp, synthetic construct
  source 1..167: mol_type = other DNA
gcagagtcggcatagctatatgttttagagctagaaatagcaagttaaaataaggctagtccgttatcaacttgaaaaagtggcaccgagtcggtgcctggggaattgtgtgaggatagctatgccgactcgcggttctatctagttacgcgttaaaccaactagaa

SEQ ID NO: 198 - DNA, 168 bp, synthetic construct
  source 1..168: mol_type = other DNA
gcagagtcggcatagctatatgttttagagctagaaatagcaagttaaaataaggctagtccgttatcaacttgaaaaagtggcaccgagtcggtgcactggggaattgtgtgaggatagctatgccgactcgcggttctatctagttacgcgttaaaccaactagaa

SEQ ID NO: 199 - DNA, 169 bp, synthetic construct
  source 1..169: mol_type = other DNA
gcagagtcggcatagctatatgttttagagctagaaatagcaagttaaaataaggctagtccgttatcaacttgaaaaagtggcaccgagtcggtgcgactggggaattgtgtgaggatagctatgccgactcgcggttctatctagttacgcgttaaaccaactagaa

SEQ ID NO: 200 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtcgatgtagctatgccgact

SEQ ID NO: 201 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtcaatgtagctatgccgact

SEQ ID NO: 202 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtcgatatagctatgccgact

SEQ ID NO: 203 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtctatgtagctatgccgact

SEQ ID NO: 204 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtcaatatagctatgccgact

SEQ ID NO: 205 - DNA, 141 bp, synthetic construct
  source 1..141: mol_type = other DNA
gagagtcggcatagctatatgtttcagagctatgctggaaacagcatagcaagttgaaataaggctagtccgttatcaacttgaaaaagtggcaccgagtcggtgcgactggggaattgtgtctatatagctatgccgact

SEQ ID NO: 206 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatcgatgtagctatgccgact

SEQ ID NO: 207 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatcaatgtagctatgccgact

SEQ ID NO: 208 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatctatgtagctatgccgact

SEQ ID NO: 209 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtcaatatagctatgccgact

SEQ ID NO: 210 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatcgatatagctatgccgact

SEQ ID NO: 211 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtcgatatagctatgccgact

SEQ ID NO: 212 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatctatatagctatgccgact

SEQ ID NO: 213 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatcgtgtcaatatagctatgccgact

SEQ ID NO: 214 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggaatcgtatcaatatagctatgccgact

SEQ ID NO: 215 - DNA, 142 bp, synthetic construct
  source 1..142: mol_type = other DNA
gagagtcggcatagctatatgtttcagagctatgctggaaacagcatagcaagttgaaataaggctagtccgttatcaacttgaaaaagtggcaccgagtcggtgcggactggggaattgtatcaatatagctatgccgact

SEQ ID NO: 216 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtcaatatagctatgccgact

SEQ ID NO: 217 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtcgatgtagctatgccgact

SEQ ID NO: 218 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtcaatgtagctatgccgact

SEQ ID NO: 219 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtctatatagctatgccgact

SEQ ID NO: 220 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatcaatatagctatgccgact

SEQ ID NO: 221 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtcaatatagctatgccgact

SEQ ID NO: 222 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatcaatatagctatgccgact

SEQ ID NO: 223 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatcaatatagctatgccgact

SEQ ID NO: 224 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatcaatgtagctatgccgact

SEQ ID NO: 225 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtcaatgtagctatgccgact

SEQ ID NO: 226 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatcgatatagctatgccgact

SEQ ID NO: 227 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggaatcgtatcgatatagctatgccgact

SEQ ID NO: 228 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatggtgtcaatgtagctatgccgact

SEQ ID NO: 229 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggaatcgtatcaatgtagctatgccgact

SEQ ID NO: 230 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatcaatatagctatgccgact

SEQ ID NO: 231 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtcgatatagctatgccgact

SEQ ID NO: 232 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtcgatgtagctatgccgact

SEQ ID NO: 233 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtcaatgtagctatgccgact

SEQ ID NO: 234 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatcgatatagctatgccgact

SEQ ID NO: 235 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtcaatgtagctatgccgact

SEQ ID NO: 236 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatcaatgtagctatgccgact

SEQ ID NO: 237 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggatcgtgtcgatatagctatgccgact

SEQ ID NO: 238 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtcgatgtagctatgccgact

SEQ ID NO: 239 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtcaatatagctatgccgact

SEQ ID NO: 240 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatcgatgtagctatgccgact

SEQ ID NO: 241 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtcgatatagctatgccgact

SEQ ID NO: 242 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatcaatgtagctatgccgact

SEQ ID NO: 243 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatcgatatagctatgccgact

SEQ ID NO: 244 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggatcgtgtcgatgtagctatgccgact

SEQ ID NO: 245 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatggtgtcaatatagctatgccgact

SEQ ID NO: 246 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatcaatatagctatgccgact

SEQ ID NO: 247 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatcaatgtagctatgccgact

SEQ ID NO: 248 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggaatcgtatcgatgtagctatgccgact

SEQ ID NO: 249 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatctatatagctatgccgact

SEQ ID NO: 250 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatcgtgtcaatgtagctatgccgact

SEQ ID NO: 251 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtgtctatgtagctatgccgact

SEQ ID NO: 252 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatcgatgtagctatgccgact

SEQ ID NO: 253 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtcaatatagctatgccgact

SEQ ID NO: 254 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtctatatagctatgccgact

SEQ ID NO: 255 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatctatatagctatgccgact

SEQ ID NO: 256 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtctatgtagctatgccgact

SEQ ID NO: 257 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatcgtatcaatatagctatgccgact

SEQ ID NO: 258 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtgtctatatagctatgccgact

SEQ ID NO: 259 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactgggggatggtgtcgatatagctatgccgact

SEQ ID NO: 260 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtatctatatagctatgccgact

SEQ ID NO: 261 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatcgatgtagctatgccgact

SEQ ID NO: 262 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactgggggatggtgtcgatgtagctatgccgact

SEQ ID NO: 263 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatagtatctatgtagctatgccgact

SEQ ID NO: 264 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtgtctatgtagctatgccgact

SEQ ID NO: 265 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatcaatatagctatgccgact

SEQ ID NO: 266 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtcgatgtagctatgccgact

SEQ ID NO: 267 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtatcgatatagctatgccgact

SEQ ID NO: 268 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatcgatatagctatgccgact

SEQ ID NO: 269 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatcaatgtagctatgccgact

SEQ ID NO: 270 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatcaatgtagctatgccgact

SEQ ID NO: 271 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatcgatatagctatgccgact

SEQ ID NO: 272 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtcgatatagctatgccgact

SEQ ID NO: 273 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatcgatgtagctatgccgact

SEQ ID NO: 274 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatcgatgtagctatgccgact

SEQ ID NO: 275 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatggtatctatgtagctatgccgact

SEQ ID NO: 276 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtcgatatagctatgccgact

SEQ ID NO: 277 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatcgatatagctatgccgact

SEQ ID NO: 278 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatctatatagctatgccgact

SEQ ID NO: 279 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatcgatgtagctatgccgact

SEQ ID NO: 280 - DNA, 146 bp, synthetic construct
  source 1..146: mol_type = other DNA
gagagtcggcatagctatatgtttcagagctatgctggaaacagcatagcaagttgaaataaggctagtccgttatcaacttgaaaaagtggcaccgagtcggtgctagaggactggggaatcgtatctatgtagctatgccgact

SEQ ID NO: 281 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtcaatgtagctatgccgact

SEQ ID NO: 282 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatggtatcgatatagctatgccgact

SEQ ID NO: 283 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtgtcaatatagctatgccgact

SEQ ID NO: 284 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatctatatagctatgccgact

SEQ ID NO: 285 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtcgatgtagctatgccgact

SEQ ID NO: 286 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatggtatcaatatagctatgccgact

SEQ ID NO: 287 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatcgtgtctatatagctatgccgact

SEQ ID NO: 288 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtcaatatagctatgccgact

SEQ ID NO: 289 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtcgatgtagctatgccgact

SEQ ID NO: 290 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatcgtgtctatgtagctatgccgact

SEQ ID NO: 291 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatggtatctatgtagctatgccgact

SEQ ID NO: 292 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatctatatagctatgccgact

SEQ ID NO: 293 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtgtcaatgtagctatgccgact

SEQ ID NO: 294 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtcaatatagctatgccgact

SEQ ID NO: 295 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtcgatatagctatgccgact

SEQ ID NO: 296 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatagtatcaatatagctatgccgact

SEQ ID NO: 297 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtatcgatgtagctatgccgact

SEQ ID NO: 298 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatggtatcgatgtagctatgccgact

SEQ ID NO: 299 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatggtgtctatgtagctatgccgact

SEQ ID NO: 300 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatcgtatctatgtagctatgccgact

SEQ ID NO: 301 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatggtgtctatatagctatgccgact

SEQ ID NO: 302 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggatagtatctatgtagctatgccgact

SEQ ID NO: 303 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtcaatgtagctatgccgact

SEQ ID NO: 304 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtctatgtagctatgccgact

SEQ ID NO: 305 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatagtatcaatgtagctatgccgact

SEQ ID NO: 306 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtcaatgtagctatgccgact

SEQ ID NO: 307 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactgggggatagtgtctatatagctatgccgact

SEQ ID NO: 308 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatcgtatcaatgtagctatgccgact

SEQ ID NO: 309 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatggtatcaatgtagctatgccgact

SEQ ID NO: 310 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatctatatagctatgccgact

SEQ ID NO: 311 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatagtatcgatatagctatgccgact

SEQ ID NO: 312 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtattgtgtcgatgtagctatgccgact

SEQ ID NO: 313 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatcaatatagctatgccgact

SEQ ID NO: 314 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtattgtgtcgatatagctatgccgact

SEQ ID NO: 315 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactgggggattgtatctatgtagctatgccgact

SEQ ID NO: 316 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatggtgtcaatatagctatgccgact

SEQ ID NO: 317 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtcgatatagctatgccgact

SEQ ID NO: 318 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtctatgtagctatgccgact

SEQ ID NO: 319 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatagtatcgatgtagctatgccgact

SEQ ID NO: 320 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatcgatatagctatgccgact

SEQ ID NO: 321 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactgggggattgtgtctatatagctatgccgact

SEQ ID NO: 322 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatcaatgtagctatgccgact

SEQ ID NO: 323 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtcgatgtagctatgccgact

SEQ ID NO: 324 - DNA, 152 bp, synthetic construct
  source 1..152: mol_type = other DNA
gagagtcggcatagctatatgtttcagagctatgctggaaacagcatagcaagttgaaataaggctagtccgttatcaacttgaaaaagtggcaccgagtcggtgcgactcctagaggactggggtatggtgtcaatgtagctatgccgact

SEQ ID NO: 325 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatggtgtcgatatagctatgccgact

SEQ ID NO: 326 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatcgatgtagctatgccgact

SEQ ID NO: 327 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtctatatagctatgccgact

SEQ ID NO: 328 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtatctatatagctatgccgact

SEQ ID NO: 329 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatggtgtcgatgtagctatgccgact

SEQ ID NO: 330 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtctatatagctatgccgact

SEQ ID NO: 331 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatggtatctatatagctatgccgact

SEQ ID NO: 332 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatagtgtctatgtagctatgccgact

SEQ ID NO: 333 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtgtctatgtagctatgccgact

SEQ ID NO: 334 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtgtctatgtagctatgccgact

SEQ ID NO: 335 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatctatatagctatgccgact

SEQ ID NO: 336 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtgtctatatagctatgccgact

SEQ ID NO: 337 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatagtatctatatagctatgccgact

SEQ ID NO: 338 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatggtgtctatatagctatgccgact

SEQ ID NO: 339 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatggtatctatgtagctatgccgact

SEQ ID NO: 340 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtattgtatctatgtagctatgccgact

SEQ ID NO: 341 - DNA, 148 bp, synthetic construct
  source 1..148: mol_type = other DNA
gagagtcggcatagctatatgtttcagagctatgctggaaacagcatagcaagttgaaataaggctagtccgttatcaacttgaaaaagtggcaccgagtcggtgccctagaggactggggtatggtgtctatgtagctatgccgact

SEQ ID NO: 342 - DNA, 150 bp, synthetic construct
  source 1..150: mol_type = other DNA
gagagtcggcatagctatatgtttcagagctatgctggaaacagcatagcaagttgaaataaggctagtccgttatcaacttgaaaaagtggcaccgagtcggtgcctcctagaggactggggtatagtatctatgtagctatgccgact

SEQ ID NO: 343 - DNA, 149 bp, synthetic construct
  source 1..149: mol_type = other DNA
gagagtcggcatagctatatgtttcagagctatgctggaaacagcatagcaagttgaaataaggctagtccgttatcaacttgaaaaagtggcaccgagtcggtgctcctagaggactggggtatcgtatctatgtagctatgccgact

SEQ ID NO: 344 - DNA, 166 bp, synthetic construct
  source 1..166: mol_type = other DNA
gccgggaggactggggaatgggttttagagctagaaatagcaagttaaaataaggctagtccgttatcaacttgaaaaagtggcaccgagtcggtgcgctatattgacacaattccccagtccgcggttctatctagttacgcgttaaaccaactagaattttttt

SEQ ID NO: 345 - DNA, 173 bp, synthetic construct
  source 1..173: mol_type = other DNA
gagggactcctagaggactggttttagagctagaaatagcaagttaaaataaggctagtccgttatcaacttgaaaaagtggcaccgagtcggtgcatattgacacaattccccaatcctctaggagtccgcggttctatctagttacgcgttaaaccaactagaattttttt

SEQ ID NO: 346 - DNA, 132 bp, synthetic construct
  source 1..132: mol_type = other DNA
gagggactcctagaggactggttttagagctagaaatagcaagttaaaataaggctagtccgttatcaacttgaaaaagtggcaccgagtcggtgcatattgacacaattccccaatcctctaggagtcttt

SEQ ID NO: 347 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 348 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 349 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 350 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 351 - protein, 753 aa, synthetic construct
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 352 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQRNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 353 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRRRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIARQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 354 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFRRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEKTITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMVEERLKTYAHLFDNKVMKQLKRCRYTGWGRLSRKLINGIRDKQSGKTILDFLKSDGFANRNFMQLIHDDSLTFKEDIQKAQVSGQGDSLYEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 355 - protein, 1952 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 356 - protein, 1952 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIKRTADGSEFESPKKKRKVPAAKRVKLD

SEQ ID NO: 357 - protein, 1948 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFCEALHRDLADFRIQHPDLILLQYYDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDSGGSKRTADGSEFESPKKKRKVPAAKRVKLD

SEQ ID NO: 358 - protein, 2128 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPFVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYMDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVPAAKRVKLD

SEQ ID NO: 359 - protein, 2124 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPKRTADGSEFESPKKKRKVPAAKRVKLD

SEQ ID NO: 360 - protein, 1944 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLKREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDKRTADGSEFESPKKKRKVPAAKRVKLD

SEQ ID NO: 361 - DNA, 57 bp, synthetic construct
  source 1..57: mol_type = other DNA
acactctttccctacacgacgctcttccgatctnnnnggctggtgggagagttcaag

SEQ ID NO: 362 - DNA, 50 bp, synthetic construct
  source 1..50: mol_type = other DNA
tggagttcagacgtgtgctcttccgatctggtccaggcacaaagtacctc

SEQ ID NO: 363 - DNA, 60 bp, synthetic construct
  source 1..60: mol_type = other DNA
acactctttccctacacgacgctcttccgatctnnnncttcagaaggcccagggacaaag

SEQ ID NO: 364 - DNA, 51 bp, synthetic construct
  source 1..51: mol_type = other DNA
tggagttcagacgtgtgctcttccgatctctggtactgggctccgcaattt

SEQ ID NO: 365 - DNA, 65 bp, synthetic construct
  source 1..65: mol_type = other DNA
acactctttccctacacgacgctcttccgatctnnnngtgtttacttgatattctgcaatgactg

SEQ ID NO: 366 - DNA, 58 bp, synthetic construct
  source 1..58: mol_type = other DNA
tggagttcagacgtgtgctcttccgatctccaacactgcatctgtatttccttaaaag

SEQ ID NO: 367 - DNA, 57 bp, synthetic construct
  source 1..57: mol_type = other DNA
acactctttccctacacgacgctcttccgatctnnnnagcccatgcgagaacagtca

SEQ ID NO: 368 - DNA, 54 bp, synthetic construct
  source 1..54: mol_type = other DNA
tggagttcagacgtgtgctcttccgatcttgacaatagaatcagcagatgtgga

SEQ ID NO: 369 - DNA, 56 bp, synthetic construct
  source 1..56: mol_type = other DNA
acactctttccctacacgacgctcttccgatctnnnngtttgaatggagccgcctg

SEQ ID NO: 370 - DNA, 55 bp, synthetic construct
  source 1..55: mol_type = other DNA
acactctttccctacacgacgctcttccgatctnnnnccctggtgggtgcatacc

SEQ ID NO: 371 - DNA, 55 bp, synthetic construct
  source 1..55: mol_type = other DNA
acactctttccctacacgacgctcttccgatctnnnnctccacccagttccggag

SEQ ID NO: 372 - DNA, 61 bp, synthetic construct
  source 1..61: mol_type = other DNA
acactctttccctacacgacgctcttccgatctnnnnctgaaatgggatggacttgtgttg

SEQ ID NO: 373 - DNA, 56 bp, synthetic construct
  source 1..56: mol_type = other DNA
acactctttccctacacgacgctcttccgatctnnnngctgtgtggatgcctgtgc

SEQ ID NO: 374 - DNA, 57 bp, synthetic construct
  source 1..57: mol_type = other DNA
acactctttccctacacgacgctcttccgatctnnnngcctttagtctggctctcag

SEQ ID NO: 375 - DNA, 57 bp, synthetic construct
  source 1..57: mol_type = other DNA
acactctttccctacacgacgctcttccgatctnnnngtgtctctgctaggccaatc

SEQ ID NO: 376 - DNA, 56 bp, synthetic construct
  source 1..56: mol_type = other DNA
acactctttccctacacgacgctcttccgatctnnnncacaggcccagagtgaatc

SEQ ID NO: 377 - DNA, 56 bp, synthetic construct
  source 1..56: mol_type = other DNA
acactctttccctacacgacgctcttccgatctnnnncagccaggactggaatgcg

SEQ ID NO: 378 - DNA, 57 bp, synthetic construct
  source 1..57: mol_type = other DNA
acactctttccctacacgacgctcttccgatctnnnngcacttgccagacctatgac

SEQ ID NO: 379 - DNA, 55 bp, synthetic construct
  source 1..55: mol_type = other DNA
acactctttccctacacgacgctcttccgatctnnnngatgagtggcggagggac

SEQ ID NO: 380 - DNA, 56 bp, synthetic construct
  source 1..56: mol_type = other DNA
acactctttccctacacgacgctcttccgatctnnnncacagttcctacctgaccc

SEQ ID NO: 381 - DNA, 59 bp, synthetic construct
  source 1..59: mol_type = other DNA
acactctttccctacacgacgctcttccgatctnnnncaatactccagagcctacttcc

SEQ ID NO: 382 - DNA, 56 bp, synthetic construct
  source 1..56: mol_type = other DNA
acactctttccctacacgacgctcttccgatctnnnnccagggattggccatccag

SEQ ID NO: 383 - DNA, 57 bp, synthetic construct
  source 1..57: mol_type = other DNA
acactctttccctacacgacgctcttccgatctnnnngcatgctcttgacacatgcc

SEQ ID NO: 384 - DNA, 61 bp, synthetic construct
  source 1..61: mol_type = other DNA
acactctttccctacacgacgctcttccgatctnnnnctcctcaaagaccactgaatttgc

SEQ ID NO: 385 - DNA, 59 bp, synthetic construct
  source 1..59: mol_type = other DNA
acactctttccctacacgacgctcttccgatctnnnnttacacagcagctagaagagtg

SEQ ID NO: 386 - DNA, 50 bp, synthetic construct
  source 1..50: mol_type = other DNA
tggagttcagacgtgtgctcttccgatctcactgatacagtgtccaggac

SEQ ID NO: 387 - DNA, 45 bp, synthetic construct
  source 1..45: mol_type = other DNA
tggagttcagacgtgtgctcttccgatctggccagcctcacaccc

SEQ ID NO: 388 - DNA, 47 bp, synthetic construct
  source 1..47: mol_type = other DNA
tggagttcagacgtgtgctcttccgatctgtgggaggcagaacaggc

SEQ ID NO: 389 - DNA, 49 bp, synthetic construct
  source 1..49: mol_type = other DNA
tggagttcagacgtgtgctcttccgatctgttctctccttgctggtcac

SEQ ID NO: 390 - DNA, 50 bp, synthetic construct
  source 1..50: mol_type = other DNA
tggagttcagacgtgtgctcttccgatctcatctcaagcaggtgtgatcc

SEQ ID NO: 391 - DNA, 50 bp, synthetic construct
  source 1..50: mol_type = other DNA
tggagttcagacgtgtgctcttccgatctctccatatctcaggctgaagg

SEQ ID NO: 392 - DNA, 48 bp, synthetic construct
  source 1..48: mol_type = other DNA
tggagttcagacgtgtgctcttccgatctctctgccctatcccctctg

SEQ ID NO: 393 - DNA, 49 bp, synthetic construct
  source 1..49: mol_type = other DNA
tggagttcagacgtgtgctcttccgatctctgtgtccattctgtccctg

SEQ ID NO: 394 - DNA, 49 bp, synthetic construct
  source 1..49: mol_type = other DNA
tggagttcagacgtgtgctcttccgatctgaagtcctgcatcactgacc

SEQ ID NO: 395 - DNA, 47 bp, synthetic construct
  source 1..47: mol_type = other DNA
tggagttcagacgtgtgctcttccgatctcccttccctctcactccg

SEQ ID NO: 396 - DNA, 46 bp, synthetic construct
  source 1..46: mol_type = other DNA
tggagttcagacgtgtgctcttccgatctgggaccgaccacgactc

SEQ ID NO: 397 - DNA, 50 bp, synthetic construct
  source 1..50: mol_type = other DNA
tggagttcagacgtgtgctcttccgatctgttatcatggccctgagcaac

SEQ ID NO: 398 - DNA, 52 bp, synthetic construct
  source 1..52: mol_type = other DNA
tggagttcagacgtgtgctcttccgatctcttctaagaagcgagctgcatag

SEQ ID NO: 399 - DNA, 50 bp, synthetic construct
  source 1..50: mol_type = other DNA
tggagttcagacgtgtgctcttccgatctcttcactttggtgaggcagtc

SEQ ID NO: 400 - DNA, 53 bp, synthetic construct
  source 1..53: mol_type = other DNA
tggagttcagacgtgtgctcttccgatctgctacaccacagcatgtttgtttc

SEQ ID NO: 401 - DNA, 53 bp, synthetic construct
  source 1..53: mol_type = other DNA
tggagttcagacgtgtgctcttccgatctcagggaaagaagaaagggacaaag

SEQ ID NO: 402 - DNA, 49 bp, synthetic construct
  source 1..49: mol_type = other DNA
tggagttcagacgtgtgctcttccgatctcactgtccagactgtatgcc

SEQ ID NO: 403 - DNA, 142 bp, synthetic construct
  source 1..142: mol_type = other DNA
tacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 404 - DNA, 14 bp, synthetic construct
  source 1..14: mol_type = other DNA
ctatattgacacaa

SEQ ID NO: 405 - DNA, 15 bp, synthetic construct
  source 1..15: mol_type = other DNA
gctatattgacacaa

SEQ ID NO: 406 - DNA, 16 bp, synthetic construct
  source 1..16: mol_type = other DNA
agctatattgacacaa

SEQ ID NO: 407 - DNA, 17 bp, synthetic construct
  source 1..17: mol_type = other DNA
cagctatattgacacaa

SEQ ID NO: 408 - DNA, 18 bp, synthetic construct
  source 1..18: mol_type = other DNA
acagctatattgacacaa

SEQ ID NO: 409 - DNA, 19 bp, synthetic construct
  source 1..19: mol_type = other DNA
cacagctatattgacacaa

SEQ ID NO: 410 - DNA, 20 bp, synthetic construct
  source 1..20: mol_type = other DNA
gcacagctatattgacacaa

SEQ ID NO: 411 - DNA, 21 bp, synthetic construct
  source 1..21: mol_type = other DNA
ggcacagctatattgacacaa

SEQ ID NO: 412 - DNA, 14 bp, synthetic construct
  source 1..14: mol_type = other DNA
gaattgtgtcaaga

SEQ ID NO: 413 - DNA, 15 bp, synthetic construct
  source 1..15: mol_type = other DNA
ggaattgtgtcaaga

SEQ ID NO: 414 - DNA, 16 bp, synthetic construct
  source 1..16: mol_type = other DNA
gggaattgtgtcaaga

SEQ ID NO: 415 - DNA, 17 bp, synthetic construct
  source 1..17: mol_type = other DNA
ggggaattgtgtcaaga

SEQ ID NO: 416 - DNA, 18 bp, synthetic construct
  source 1..18: mol_type = other DNA
tggggaattgtgtcaaga

SEQ ID NO: 417 - DNA, 19 bp, synthetic construct
  source 1..19: mol_type = other DNA
ctggggaattgtgtcaaga

SEQ ID NO: 418 - DNA, 20 bp, synthetic construct
  source 1..20: mol_type = other DNA
actggggaattgtgtcaaga

SEQ ID NO: 419 - DNA, 21 bp, synthetic construct
  source 1..21: mol_type = other DNA
gactggggaattgtgtcaaga

SEQ ID NO: 420 - DNA, 21 bp, synthetic construct
  source 1..21: mol_type = other DNA
gactggggaattgtgtcgatg

SEQ ID NO: 421 - DNA, 21 bp, synthetic construct
  source 1..21: mol_type = other DNA
gactggggaattgtgtcaatg

SEQ ID NO: 422 - DNA, 21 bp, synthetic construct
  source 1..21: mol_type = other DNA
gactggggaattgtgtcgata

SEQ ID NO: 423 - DNA, 21 bp, synthetic construct
  source 1..21: mol_type = other DNA
gactggggaattgtgtctatg

SEQ ID NO: 424 - DNA, 21 bp, synthetic construct
  source 1..21: mol_type = other DNA
gactggggaattgtgtcaata

SEQ ID NO: 425 - DNA, 21 bp, synthetic construct
  source 1..21: mol_type = other DNA
gactggggaattgtgtctata

SEQ ID NO: 426 - DNA, 22 bp, synthetic construct
  source 1..22: mol_type = other DNA
ggactggggaattgtatcgatg

SEQ ID NO: 427 - DNA, 22 bp, synthetic construct
  source 1..22: mol_type = other DNA
ggactggggaattgtatcaatg

SEQ ID NO: 428 - DNA, 22 bp, synthetic construct
  source 1..22: mol_type = other DNA
ggactggggaattgtatctatg

SEQ ID NO: 429 - DNA, 26 bp, synthetic construct
  source 1..26: mol_type = other DNA
tagaggactggggaatcgtgtcaata

SEQ ID NO: 430 - DNA, 22 bp, synthetic construct
  source 1..22: mol_type = other DNA
ggactggggaattgtatcgata

SEQ ID NO: 431 - DNA, 26 bp, synthetic construct
  source 1..26: mol_type = other DNA
tagaggactggggaatcgtgtcgata

SEQ ID NO: 432 - DNA, 22 bp, synthetic construct
  source 1..22: mol_type = other DNA
ggactggggaattgtatctata

SEQ ID NO: 433 - DNA, 32 bp, synthetic construct
  source 1..32: mol_type = other DNA
gactcctagaggactgggggatcgtgtcaata

SEQ ID NO: 434 - DNA, 29 bp, synthetic construct
  source 1..29: mol_type = other DNA
tcctagaggactggggaatcgtatcaata

SEQ ID NO: 435 - DNA, 22 bp, synthetic construct
  source 1..22: mol_type = other DNA
ggactggggaattgtatcaata

SEQ ID NO: 436 - DNA, 26 bp, synthetic construct
  source 1..26: mol_type = other DNA
tagaggactggggaatagtgtcaata

SEQ ID NO: 437 - DNA, 26 bp, synthetic construct
  source 1..26: mol_type = other DNA
tagaggactggggaatcgtgtcgatg

SEQ ID NO: 438 - DNA, 26 bp, synthetic construct
  source 1..26: mol_type = other DNA
tagaggactggggaatcgtgtcaatg

SEQ ID NO: 439 - DNA, 26 bp, synthetic construct
  source 1..26: mol_type = other DNA
tagaggactggggaatcgtgtctata

SEQ ID NO: 440 - DNA, 26 bp, synthetic construct
  source 1..26: mol_type = other DNA
tagaggactggggaatagtatcaata

SEQ ID NO: 441 - DNA, 26 bp, synthetic construct
  source 1..26: mol_type = other DNA
tagaggactggggaatggtgtcaata

SEQ ID NO: 442 - DNA, 30 bp, synthetic construct
  source 1..30: mol_type = other DNA
ctcctagaggactgggggatcgtatcaata

SEQ ID NO: 443 - DNA, 30 bp, synthetic construct
  source 1..30: mol_type = other DNA
ctcctagaggactgggggatggtatcaata

SEQ ID NO: 444 - DNA, 30 bp, synthetic construct
  source 1..30: mol_type = other DNA
ctcctagaggactgggggatggtatcaatg

SEQ ID NO: 445 - DNA, 32 bp, synthetic construct
  source 1..32: mol_type = other DNA
gactcctagaggactgggggatagtgtcaatg

SEQ ID NO: 446 - DNA, 30 bp, synthetic construct
  source 1..30: mol_type = other DNA
ctcctagaggactgggggatcgtatcgata

SEQ ID NO: 447 - DNA, 29 bp, synthetic construct
  source 1..29: mol_type = other DNA
tcctagaggactggggaatcgtatcgata

SEQ ID NO: 448 - DNA, 32 bp, synthetic construct
  source 1..32: mol_type = other DNA
gactcctagaggactgggggatggtgtcaatg

SEQ ID NO: 449 - DNA, 29 bp, synthetic construct
  source 1..29: mol_type = other DNA
tcctagaggactggggaatcgtatcaatg

SEQ ID NO: 450 - DNA, 26 bp, synthetic construct
  source 1..26: mol_type = other DNA
tagaggactggggaatggtatcaata

SEQ ID NO: 451 - DNA, 26 bp, synthetic construct
  source 1..26: mol_type = other DNA
tagaggactggggaatggtgtcgata

SEQ ID NO: 452 - DNA, 26 bp, synthetic construct
  source 1..26: mol_type = other DNA
tagaggactggggaatagtgtcgatg

SEQ ID NO: 453 - DNA, 26 bp, synthetic construct
  source 1..26: mol_type = other DNA
tagaggactggggaatggtgtcaatg

SEQ ID NO: 454 - DNA, 26 bp, synthetic construct
  source 1..26: mol_type = other DNA
tagaggactggggaatagtatcgata

SEQ ID NO: 455 - DNA, 26 bp, synthetic construct
  source 1..26: mol_type = other DNA
tagaggactggggaatagtgtcaatg

SEQ ID NO: 456 - DNA, 26 bp, synthetic construct
  source 1..26: mol_type = other DNA
tagaggactggggaatagtatcaatg

SEQ ID NO: 457 - DNA, 28 bp, synthetic construct
  source 1..28: mol_type = other DNA
cctagaggactgggggatcgtgtcgata

SEQ ID NO: 458 - DNA, 26 bp, synthetic construct
  source 1..26: mol_type = other DNA
tagaggactggggaatggtgtcgatg

SEQ ID NO: 459 - DNA, 29 bp, synthetic construct
  source 1..29: mol_type = other DNA
tcctagaggactggggtatcgtgtcaata

SEQ ID NO: 460 - DNA, 26 bp, synthetic construct
  source 1..26: mol_type = other DNA
tagaggactggggaatagtatcgatg

SEQ ID NO: 461 - DNA, 26 bp, synthetic construct
  source 1..26: mol_type = other DNA
tagaggactggggaatagtgtcgata

SEQ ID NO: 462 - DNA, 30 bp, synthetic construct
  source 1..30: mol_type = other DNA
ctcctagaggactgggggatagtatcaatg

SEQ ID NO: 463 - DNA, 26 bp, synthetic construct
  source 1..26: mol_type = other DNA
tagaggactggggaatggtatcgata

SEQ ID NO: 464 - DNA, 28 bp, synthetic construct
  source 1..28: mol_type = other DNA
cctagaggactgggggatcgtgtcgatg

SEQ ID NO: 465 - DNA, 32 bp, synthetic construct
  source 1..32: mol_type = other DNA
gactcctagaggactgggggatggtgtcaata

SEQ ID NO: 466 - DNA, 30 bp, synthetic construct
  source 1..30: mol_type = other DNA
ctcctagaggactgggggatagtatcaata

SEQ ID NO: 467 - DNA, 26 bp, synthetic construct
  source 1..26: mol_type = other DNA
tagaggactggggaatggtatcaatg

SEQ ID NO: 468 - DNA, 29 bp, synthetic construct
  source 1..29: mol_type = other DNA
tcctagaggactggggaatcgtatcgatg

SEQ ID NO: 469 - DNA, 26 bp, synthetic construct
  source 1..26: mol_type = other DNA
tagaggactggggaatagtatctata

SEQ ID NO: 470 - DNA, 32 bp, synthetic construct
  source 1..32: mol_type = other DNA
gactcctagaggactgggggatcgtgtcaatg

SEQ ID NO: 471 - DNA, 26 bp, synthetic construct
  source 1..26: mol_type = other DNA
tagaggactggggaatcgtgtctatg

SEQ ID NO: 472 - DNA, 26 bp, synthetic construct
  source 1..26: mol_type = other DNA
tagaggactggggaatggtatcgatg

SEQ ID NO: 473 - DNA, 32 bp, synthetic construct
  source 1..32: mol_type = other DNA
gactcctagaggactgggggatagtgtcaata

SEQ ID NO: 474 - DNA, 26 bp, synthetic construct
  source 1..26: mol_type = other DNA
tagaggactggggaatggtgtctata

SEQ ID NO: 475 - DNA, 26 bp, synthetic construct
  source 1..26: mol_type = other DNA
tagaggactggggaatggtatctata

SEQ ID NO: 476 - DNA, 26 bp, synthetic construct
  source 1..26: mol_type = other DNA
tagaggactggggaatagtgtctatg

SEQ ID NO: 477 - DNA, 28 bp, synthetic construct
  source 1..28: mol_type = other DNA
cctagaggactggggtatcgtatcaata

SEQ ID NO: 478 - DNA, 26 bp, synthetic construct
  source 1..26: mol_type = other DNA
tagaggactggggaatagtgtctata

SEQ ID NO: 479 - DNA, 29 bp, synthetic construct
  source 1..29: mol_type = other DNA
tcctagaggactgggggatggtgtcgata

SEQ ID NO: 480 - DNA, 26 bp, synthetic construct
  source 1..26: mol_type = other DNA
tagaggactggggaatcgtatctata

SEQ ID NO: 481 - DNA, 30 bp, synthetic construct
  source 1..30: mol_type = other DNA
ctcctagaggactgggggatcgtatcgatg

SEQ ID NO: 482 - DNA, 29 bp, synthetic construct
  source 1..29: mol_type = other DNA
tcctagaggactgggggatggtgtcgatg

SEQ ID NO: 483 - DNA, 26 bp, synthetic construct
  source 1..26: mol_type = other DNA
tagaggactggggaatagtatctatg

SEQ ID NO: 484 - DNA, 26 bp, synthetic construct
  source 1..26: mol_type = other DNA
tagaggactggggaatggtgtctatg

SEQ ID NO: 485 - DNA, 30 bp, synthetic construct
  source 1..30: mol_type = other DNA
ctcctagaggactgggggattgtatcaata

SEQ ID NO: 486 - DNA, 32 bp, synthetic construct
  source 1..32: mol_type = other DNA
gactcctagaggactgggggatagtgtcgatg

SEQ ID NO: 487 - DNA, 29 bp, synthetic construct
  source 1..29: mol_type = other DNA
tcctagaggactggggtatcgtatcgata

SEQ ID NO: 488 - DNA, 30 bp, synthetic construct
  source 1..30: mol_type = other DNA
ctcctagaggactgggggatagtatcgata

SEQ ID NO: 489 - DNA, 30 bp, synthetic construct
  source 1..30: mol_type = other DNA
ctcctagaggactgggggatcgtatcaatg

SEQ ID NO: 490 - DNA, 30 bp, synthetic construct
  source 1..30: mol_type = other DNA
ctcctagaggactgggggattgtatcaatg

SEQ ID NO: 491 - DNA, 30 bp, synthetic construct
  source 1..30: mol_type = other DNA
ctcctagaggactgggggatggtatcgata

SEQ ID NO: 492 - DNA, 32 bp, synthetic construct
  source 1..32: mol_type = other DNA
gactcctagaggactgggggatagtgtcgata

SEQ ID NO: 493 - DNA, 30 bp, synthetic construct
  source 1..30: mol_type = other DNA
ctcctagaggactgggggatggtatcgatg

SEQ ID NO: 494 - DNA, 30 bp, synthetic construct
  source 1..30: mol_type = other DNA
ctcctagaggactgggggatagtatcgatg

SEQ ID NO: 495 - DNA, 26 bp, synthetic construct
  source 1..26: mol_type = other DNA
tagaggactggggaatggtatctatg

SEQ ID NO: 496 - DNA, 29 bp, synthetic construct
  source 1..29: mol_type = other DNA
tcctagaggactggggtatcgtgtcgata

SEQ ID NO: 497 - DNA, 30 bp, synthetic construct
  source 1..30: mol_type = other DNA
ctcctagaggactgggggattgtatcgata

SEQ ID NO: 498 - DNA, 30 bp, synthetic construct
  source 1..30: mol_type = other DNA
ctcctagaggactgggggatcgtatctata

SEQ ID NO: 499 - DNA, 30 bp, synthetic construct
  source 1..30: mol_type = other DNA
ctcctagaggactgggggattgtatcgatg

SEQ ID NO: 500 - DNA, 26 bp, synthetic construct
  source 1..26: mol_type = other DNA
tagaggactggggaatcgtatctatg

SEQ ID NO: 501 - DNA, 28 bp, synthetic construct
  source 1..28: mol_type = other DNA
cctagaggactgggggattgtgtcaatg

SEQ ID NO: 502 - DNA, 28 bp, synthetic construct
  source 1..28: mol_type = other DNA
cctagaggactggggtatggtatcgata

SEQ ID NO: 503 - DNA, 28 bp, synthetic construct
  source 1..28: mol_type = other DNA
cctagaggactggggtattgtgtcaata

SEQ ID NO: 504 - DNA, 30 bp, synthetic construct
  source 1..30: mol_type = other DNA
ctcctagaggactgggggatggtatctata

SEQ ID NO: 505 - DNA, 29 bp, synthetic construct
  source 1..29: mol_type = other DNA
tcctagaggactggggtatcgtgtcgatg

SEQ ID NO: 506 - DNA, 30 bp, synthetic construct
  source 1..30: mol_type = other DNA
ctcctagaggactggggtatggtatcaata

SEQ ID NO: 507 - DNA, 32 bp, synthetic construct
  source 1..32: mol_type = other DNA
gactcctagaggactgggggatcgtgtctata

SEQ ID NO: 508 - DNA, 28 bp, synthetic construct
  source 1..28: mol_type = other DNA
cctagaggactgggggattgtgtcaata

SEQ ID NO: 509 - DNA, 28 bp, synthetic construct
  source 1..28: mol_type = other DNA
cctagaggactgggggattgtgtcgatg

SEQ ID NO: 510 - DNA, 32 bp, synthetic construct
  source 1..32: mol_type = other DNA
gactcctagaggactgggggatcgtgtctatg

SEQ ID NO: 511 - DNA, 30 bp, synthetic construct
  source 1..30: mol_type = other DNA
ctcctagaggactgggggatggtatctatg

SEQ ID NO: 512 - DNA, 30 bp, synthetic construct
  source 1..30: mol_type = other DNA
ctcctagaggactgggggatagtatctata

SEQ ID NO: 513 - DNA, 28 bp, synthetic construct
  source 1..28: mol_type = other DNA
cctagaggactggggtattgtgtcaatg

SEQ ID NO: 514 - DNA, 29 bp, synthetic construct
  source 1..29: mol_type = other DNA
tcctagaggactggggtatagtgtcaata

SEQ ID NO: 515 - DNA, 28 bp, synthetic construct
  source 1..28: mol_type = other DNA
cctagaggactgggggattgtgtcgata

SEQ ID NO: 516 - DNA, 32 bp, synthetic construct
  source 1..32: mol_type = other DNA
gactcctagaggactggggtatagtatcaata

SEQ ID NO: 517 - DNA, 29 bp, synthetic construct
  source 1..29: mol_type = other DNA
tcctagaggactggggtatcgtatcgatg

SEQ ID NO: 518 - DNA, 28 bp, synthetic construct
  source 1..28: mol_type = other DNA
cctagaggactggggtatggtatcgatg

SEQ ID NO: 519 - DNA, 32 bp, synthetic construct
  source 1..32: mol_type = other DNA
gactcctagaggactgggggatggtgtctatg

SEQ ID NO: 520 - DNA, 30 bp, synthetic construct
  source 1..30: mol_type = other DNA
ctcctagaggactgggggatcgtatctatg

SEQ ID NO: 521 - DNA, 32 bp, synthetic construct
  source 1..32: mol_type = other DNA
gactcctagaggactgggggatggtgtctata

SEQ ID NO: 522 - DNA, 30 bp, synthetic construct
  source 1..30: mol_type = other DNA
ctcctagaggactgggggatagtatctatg

SEQ ID NO: 523 - DNA, 29 bp, synthetic construct
  source 1..29: mol_type = other DNA
tcctagaggactggggtatagtgtcaatg

SEQ ID NO: 524 - DNA, 32 bp, synthetic construct
  source 1..32: mol_type = other DNA
gactcctagaggactgggggatagtgtctatg

SEQ ID NO: 525 - DNA, 32 bp, synthetic construct
  source 1..32: mol_type = other DNA
gactcctagaggactggggtatagtatcaatg

SEQ ID NO: 526 - DNA, 29 bp, synthetic construct
  source 1..29: mol_type = other DNA
tcctagaggactggggtatcgtgtcaatg

SEQ ID NO: 527 - DNA, 32 bp, synthetic construct
  source 1..32: mol_type = other DNA
gactcctagaggactgggggatagtgtctata

SEQ ID NO: 528 - DNA, 28 bp, synthetic construct
  source 1..28: mol_type = other DNA
cctagaggactggggtatcgtatcaatg

SEQ ID NO: 529 - DNA, 30 bp, synthetic construct
  source 1..30: mol_type = other DNA
ctcctagaggactggggtatggtatcaatg

SEQ ID NO: 530 - DNA, 30 bp, synthetic construct
  source 1..30: mol_type = other DNA
ctcctagaggactgggggattgtatctata

SEQ ID NO: 531 - DNA, 32 bp, synthetic construct
  source 1..32: mol_type = other DNA
gactcctagaggactggggtatagtatcgata

SEQ ID NO: 532 - DNA, 29 bp, synthetic construct
  source 1..29: mol_type = other DNA
tcctagaggactggggtattgtgtcgatg

SEQ ID NO: 533 - DNA, 28 bp, synthetic construct
  source 1..28: mol_type = other DNA
cctagaggactggggtattgtatcaata

SEQ ID NO: 534 - DNA, 29 bp, synthetic construct
  source 1..29: mol_type = other DNA
tcctagaggactggggtattgtgtcgata

SEQ ID NO: 535 - DNA, 30 bp, synthetic construct
  source 1..30: mol_type = other DNA
ctcctagaggactgggggattgtatctatg

SEQ ID NO: 536 - DNA, 32 bp, synthetic construct
  source 1..32: mol_type = other DNA
gactcctagaggactggggtatggtgtcaata

SEQ ID NO: 537 - DNA, 29 bp, synthetic construct
  source 1..29: mol_type = other DNA
tcctagaggactggggtatagtgtcgata

SEQ ID NO: 538 - DNA, 28 bp, synthetic construct
  source 1..28: mol_type = other DNA
cctagaggactgggggattgtgtctatg

SEQ ID NO: 539 - DNA, 32 bp, synthetic construct
  source 1..32: mol_type = other DNA
gactcctagaggactggggtatagtatcgatg

SEQ ID NO: 540 - DNA, 28 bp, synthetic construct
  source 1..28: mol_type = other DNA
cctagaggactggggtattgtatcgata

SEQ ID NO: 541 - DNA, 28 bp, synthetic construct
  source 1..28: mol_type = other DNA
cctagaggactgggggattgtgtctata

SEQ ID NO: 542 - DNA, 28 bp, synthetic construct
  source 1..28: mol_type = other DNA
cctagaggactggggtattgtatcaatg

SEQ ID NO: 543 - DNA, 29 bp, synthetic construct
  source 1..29: mol_type = other DNA
tcctagaggactggggtatagtgtcgatg

SEQ ID NO: 544 - DNA, 32 bp, synthetic construct
  source 1..32: mol_type = other DNA
gactcctagaggactggggtatggtgtcaatg

SEQ ID NO: 545 - DNA, 30 bp, synthetic construct
  source 1..30: mol_type = other DNA
ctcctagaggactggggtatggtgtcgata

SEQ ID NO: 546 - DNA, 28 bp, synthetic construct
  source 1..28: mol_type = other DNA
cctagaggactggggtattgtatcgatg

SEQ ID NO: 547 - DNA, 29 bp, synthetic construct
  source 1..29: mol_type = other DNA
tcctagaggactggggtatcgtgtctata

SEQ ID NO: 548 - DNA, 29 bp, synthetic construct
  source 1..29: mol_type = other DNA
tcctagaggactggggtatcgtatctata

SEQ ID NO: 549 - DNA, 30 bp, synthetic construct
  source 1..30: mol_type = other DNA
ctcctagaggactggggtatggtgtcgatg

SEQ ID NO: 550 - DNA, 29 bp, synthetic construct
  source 1..29: mol_type = other DNA
tcctagaggactggggtatagtgtctata

SEQ ID NO: 551 - DNA, 28 bp, synthetic construct
  source 1..28: mol_type = other DNA
cctagaggactggggtatggtatctata

SEQ ID NO: 552 - DNA, 29 bp, synthetic construct
  source 1..29: mol_type = other DNA
tcctagaggactggggtatagtgtctatg

SEQ ID NO: 553 - DNA, 29 bp, synthetic construct
  source 1..29: mol_type = other DNA
tcctagaggactggggtatcgtgtctatg

SEQ ID NO: 554 - DNA, 28 bp, synthetic construct
  source 1..28: mol_type = other DNA
cctagaggactggggtattgtgtctatg

SEQ ID NO: 555 - DNA, 28 bp, synthetic construct
  source 1..28: mol_type = other DNA
cctagaggactggggtattgtatctata

SEQ ID NO: 556 - DNA, 28 bp, synthetic construct
  source 1..28: mol_type = other DNA
cctagaggactggggtattgtgtctata

SEQ ID NO: 557 - DNA, 30 bp, synthetic construct
  source 1..30: mol_type = other DNA
ctcctagaggactggggtatagtatctata

SEQ ID NO: 558 - DNA, 28 bp, synthetic construct
  source 1..28: mol_type = other DNA
cctagaggactggggtatggtgtctata

SEQ ID NO: 559 - DNA, 29 bp, synthetic construct
  source 1..29: mol_type = other DNA
tcctagaggactggggtatggtatctatg

SEQ ID NO: 560 - DNA, 28 bp, synthetic construct
  source 1..28: mol_type = other DNA
cctagaggactggggtattgtatctatg

SEQ ID NO: 561 - DNA, 28 bp, synthetic construct
  source 1..28: mol_type = other DNA
cctagaggactggggtatggtgtctatg

SEQ ID NO: 562 - DNA, 30 bp, synthetic construct
  source 1..30: mol_type = other DNA
ctcctagaggactggggtatagtatctatg

SEQ ID NO: 563 - DNA, 29 bp, synthetic construct
  source 1..29: mol_type = other DNA
tcctagaggactggggtatcgtatctatg

SEQ ID NO: 564 - DNA, 14 bp, synthetic construct
  source 1..14: mol_type = other DNA
gaattgtgtgagga

SEQ ID NO: 565 - DNA, 15 bp, synthetic construct
  source 1..15: mol_type = other DNA
ggaattgtgtgagga

SEQ ID NO: 566 - DNA, 16 bp, synthetic construct
  source 1..16: mol_type = other DNA
gggaattgtgtgagga

SEQ ID NO: 567 - DNA, 17 bp, synthetic construct
  source 1..17: mol_type = other DNA
ggggaattgtgtgagga

SEQ ID NO: 568 - DNA, 18 bp, synthetic construct
  source 1..18: mol_type = other DNA
tggggaattgtgtgagga

SEQ ID NO: 569 - DNA, 19 bp, synthetic construct
  source 1..19: mol_type = other DNA
ctggggaattgtgtgagga

SEQ ID NO: 570 - DNA, 20 bp, synthetic construct
  source 1..20: mol_type = other DNA
actggggaattgtgtgagga

SEQ ID NO: 571 - DNA, 21 bp, synthetic construct
  source 1..21: mol_type = other DNA
gactggggaattgtgtgagga

SEQ ID NO: 572 - protein, 407 aa, synthetic construct
GRPYVTLNLNGMFMDKFKPYSKSNAPITTLEKLSKALSISVEELKAIAELSLDEKYTLKKIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALDYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSNRDVAVIDAAIKSLELSYSKGNQNKHWYKRKYDLTRYKMIILTRSESFKEKLECFKSRLASLKPL

SEQ ID NO: 573 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 574 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 575 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFCEALHRDLADFRIQHPDLILLQYYDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 576 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 577 - DNA, 86 bp, synthetic construct
  source 1..86: mol_type = other DNA
gtttcagagctatgctggaaacagcatagcaagttgaaataaggctagtccgttatcaacttgaaaaagtggcaccgagtcggtgc

SEQ ID NO: 578 - DNA, 86 bp, synthetic construct
  source 1..86: mol_type = other DNA
gtttaagagctaagctggaaacagcatagcaagtttaaataaggctagtccgttatcaacttgaaaaagtggcaccgagtcggtgc

SEQ ID NO: 579 - DNA, 33 bp, synthetic construct
  source 1..33: mol_type = other DNA
tccgggaggactggggaatggtgcaatatagct

SEQ ID NO: 580 - protein, 11 aa, synthetic construct
GPPSPFPAIYS

SEQ ID NO: 581 - DNA, 33 bp, synthetic construct
  source 1..33: mol_type = other DNA
ccgggaggactggggaattgtgtcaatatagct

SEQ ID NO: 582 - protein, 11 aa, synthetic construct
RSSQPITDIYS

SEQ ID NO: 583 - protein, 18 aa, synthetic construct
QSAHSYIAQFPSPKGVPP

SEQ ID NO: 584 - DNA, 54 bp, synthetic construct
  source 1..54: mol_type = other DNA
aggagggactcctagaggactggggaattgtgcaatatagctatgccgactctg

SEQ ID NO: 585 - DNA, 54 bp, synthetic construct
  source 1..54: mol_type = other DNA
cagagtcggcatagctatattgcacaattccccagtcctctaggagtccctgga

SEQ ID NO: 586 - DNA, 54 bp, synthetic construct
  source 1..54: mol_type = other DNA
cagagtcggcatagctatattgacacaattccccaatcctctaggagtccctcc

SEQ ID NO: 587 - protein, 18 aa, synthetic construct
QSRHSYXDTIPQSSRSPS

SEQ ID NO: 588 - protein, 14 aa, synthetic construct
NLLKLDPADRYLTE

SEQ ID NO: 589 - DNA, 49 bp, synthetic construct
  source 1..49: mol_type = other DNA
tcctaaaaatttactgaagttggacccagctgacagatacttgacagaa

SEQ ID NO: 590 - DNA, 49 bp, synthetic construct
  source 1..49: mol_type = other DNA
ttctgtcaagtatctgtcagcttggtccaacttcagtaaatttttagga

SEQ ID NO: 591 - DNA, 49 bp, synthetic construct
  source 1..49: mol_type = other DNA
tcctaagaacctgttgaagttggacccagctgacagatacttgacagaa

SEQ ID NO: 592 - protein, 14 aa, synthetic construct
NLLKLDPADRTLTE

SEQ ID NO: 593 - DNA, 40 bp, synthetic construct
  source 1..40: mol_type = other DNA
cagagtacgttgccaccagatggtatcggtccccagaact

SEQ ID NO: 594 - DNA, 40 bp, synthetic construct
  source 1..40: mol_type = other DNA
cagagtacgttgccaccagccggtgtcgctccctagagcc

SEQ ID NO: 595 - DNA, 33 bp, synthetic construct
  source 1..33: mol_type = other DNA
tcgatataacgtggtaaggggtcaggagggcct

SEQ ID NO: 596 - DNA, 33 bp, synthetic construct
  source 1..33: mol_type = other DNA
tcgatataactgtgttaaggggtcaggaggccg

SEQ ID NO: 597 - DNA, 33 bp, synthetic construct
  source 1..33: mol_type = other DNA
tcgatataactgtgttaaggggtcaggagggcc

SEQ ID NO: 598 - DNA, 19 bp, synthetic construct
  source 1..19: mol_type = other DNA
ttgcacaattccccagtcc

SEQ ID NO: 599 - DNA, 16 bp, synthetic construct
  source 1..16: mol_type = other DNA
ggactggggaattgtg

SEQ ID NO: 600 - DNA, 20 bp, synthetic construct
  source 1..20: mol_type = other DNA
ttgacacaattccccaatcc

SEQ ID NO: 601 - DNA, 20 bp, synthetic construct
  source 1..20: mol_type = other DNA
ggattggggaattgtgtcaa

SEQ ID NO: 602 - protein, 15 aa, synthetic construct
NLLKLDPADRTYLTE

SEQ ID NO: 603 - DNA, 49 bp, synthetic construct
  source 1..49: mol_type = other DNA
ttctgtcaagtatctgtcagctgggtccaacttcagtaaatttttagga

SEQ ID NO: 604 - protein, 13 aa, synthetic construct
NLLKDPADRYLTE